(19) United States
(12) Patent Application Publication
(10) Pub. No.: US 2025/0333698 A1
(43) Pub. Date: Oct. 30, 2025

(54) SIRPa DEFICIENT MACROPHAGES FOR TREATING CANCER

(71) Applicant: GEORGIA STATE UNIVERSITY RESEARCH FOUNDATION, INC., Atlanta, GA (US)

(72) Inventors: Yuan LIU, Decatur, GA (US); Zhen BIAN, Atlanta, GA (US); Lei SHI, Decatur, GA (US); Ke ZEN, Decatur, GA (US); Koby KIDDER, Mohnton, PA (US)

(21) Appl. No.: 18/686,625

(22) PCT Filed: Oct. 18, 2022
(86) PCT No.: PCT/US2022/078248

§ 371 Date: Feb. 26, 2024

Related U.S. Application Data
(60) Provisional application No. 63/271,930, filed on Oct. 26, 2021.

(51) Int. Cl. C12N 5/0786; A61K 35/15; A61P 35/00; C12N 5/0783

(52) U.S. Cl. CPC C12N 5/0645; A61K 35/15; A61P 35/00; C12N 5/0636; C12N 2500/40; C12N 2501/2317; C12N 2501/24; C12N 2501/25; C12N 2501/999; C12N 2506/115

(57) ABSTRACT

As disclosed herein, SIRPα is integral to immuno-evasion by many different cancer types as well as cancer resistance to therapies, and reducing SIRPα levels on can bolster antigen acquisition, processing, and presentation, decrease TME immunosuppression and thereby promote tumor-specific T cell activation to eliminate tumors and generate an adaptive immune response consisting of memory T cells, circulating antibodies, and plasma cells, all of which may be specific for neo-antigens in the original cancer. Therefore, disclosed are activated SIRPα^{low} macrophages that are useful for treating cancers.

### CROSS-REFERENCE TO RELATED APPLICATIONS

This application claims benefit of U.S. Provisional Application No. 63/271,930, filed Oct. 26, 2021, which is hereby incorporated herein by reference in its entirety.

### STATEMENT REGARDING FEDERALLY SPONSORED RESEARCH OR DEVELOPMENT

This invention was made with Government Support under Grant Nos. A1106839 and CA241271 awarded by the National Institutes of Health. The Government has certain rights in the invention.

### BACKGROUND

Cancer remains a major threat to human health worldwide even with various therapeutic efforts. Given that immune evasion is a hallmark of cancer, new immunotherapies, such as immune checkpoint blockade (ICB), chimeric antigen receptor (CAR)-T, cancer vaccination and immune-regulatory radiation therapy (RT) have been developed to combat cancer; however, these endeavors have yet to fully meet the clinical need because of low-response rates and limited cancer types toward which these treatments are effective. Thus, there is an urgent need for additional approaches and therapeutic innovation to improve treatments for cancers that evade immune elimination and are resistant to current therapies.

### SUMMARY

As disclosed herein, SIRPα is integral to immuno-evasion by many different cancer types as well as cancer resistance to RT, ICB and other immune-regulatory therapies. Reducing SIRPα expression or diminishing SIRPα-mediated regulation can bolster antigen acquisition, processing, and presentation, decrease the tumor microenvironment (TME) immunosuppression, and thereby promote tumor-specific, T cell activation to eliminate tumors and generate an adaptive immune response consisting of T cells, circulating antibodies, and plasma cells, all of which may be specific for neo-antigens in the original cancer.

Therefore, disclosed herein are activated SIRPα^{low} macrophages for use in treating cancer. In some embodiments, these activated SIRPα^{low} macrophages are prepared by a method that involves obtaining a biological sample comprising peripheral blood mononuclear cells (PBMC) from the subject; isolating monocytes from the PBMC; differentiating the monocytes in vitro to produce macrophages; contacting the macrophages with SIRPα inhibitor; and contacting the macrophages with a macrophage activating agent, thereby generating a population of macrophages with marked reduction of SIRPα cell-surface expression (SIRPα^{low}), relative to untreated macrophages, and increased capacities of phagocytosis towards cancer cells, proinflammatory response and immunogenic antigen presentation that activate tumor-specific T cells, thereby producing a medicament for treating cancer comprising activated SIRPα^{low} macrophages.

In some embodiments, the SIRPα inhibitor and macrophage activating agent are administered sequentially. This can be in either order and can be minutes, hours, or days apart, such as 1, 2, 3, 4, 5, 6, 7, 8, 9, 10, 11, 12, 13, 14, 16, 16, 17, 18, 19, 20, 21, 22, 23, or 24 hours apart. In other embodiments, the SIRPα inhibitor and macrophage activating agent are administered simultaneously or concurrently.

In some embodiments, the SIRPα inhibitor and macrophage activating agent are present in the same composition. Therefore, in some embodiments, the method involves isolating monocytes from peripheral blood mononuclear cells (PBMC) in a biological sample; differentiating the monocytes in vitro to produce macrophages; and contacting the macrophages with an SIRPα expression inhibitor and a macrophage activating agent to generate a population of activated macrophages with reduced SIRPα cell-surface expression and increased activities of phagocytosis, proinflammatory activity and antigen presentation (activated SIRPα^{low} macrophages) relative to untreated macrophages.

In some embodiments, the disclosed compositions and methods are used with any professional antigen presenting cell. Professional antigen presenting cells (APCs) are immune cells that specialize in presenting an antigen to a T-cell. The main types of professional APCs are dendritic cells (DC), macrophages, and B cells, but can also include endothelial cells, and in some embodiments granulocytes.

Therefore, also disclosed is a method for treating cancer in a subject that involves administering to the subject a therapeutically effective amount of the activated SIRPα^{low} macrophages. In some embodiments, the therapeutically effective amount of the activated SIRPα^{low} macrophages is administered directly into the tumor (intratumoral administration) followed by tumor-directed in situ radiation therapy (FIG. 13A). In some embodiments, the therapeutically effective amount of the activated SIRPα^{low} macrophages is administered directly into the tumor preceded by tumor-directed in situ radiation therapy (FIG. 13B). In some embodiments, the therapeutically effective amount of the activated SIRPα^{low} macrophages is administered directly into the tumor without any tumor-directed in situ radiation therapy (FIG. 13C).

In some embodiments, the therapeutically effective amount of the activated SIRPα^{low} macrophages is administered directly into the tumor followed by tumor-directed in situ radiation therapy and by intravenous (IV) administration of ICB therapy (FIG. 13D). In some embodiments, the therapeutically effective amount of the activated SIRPα^{low} macrophages is administered directly into the tumor preceded by tumor-directed in situ radiation therapy and followed by IV administration of ICB (FIG. 13E). In some embodiments, the therapeutically effective amount of the activated SIRPα^{low} macrophages is administered directly into the tumor followed by IV administration of ICB without any tumor-directed in situ radiation therapy (FIG. 13F).

In some embodiments, the therapeutically effective amount of the activated SIRPα^{low} macrophages is administered IV followed by tumor-directed in situ radiation therapy (FIG. 13G). In some embodiments, the therapeutically effective amount of the activated SIRPα^{low} macrophages is administered IV followed by tumor-directed in situ radiation therapy and by IV administration of ICB (FIG. 13H).

In some embodiments, a therapeutically effective amount of the SIRPα^{low} macrophages which have not been activated in in vitro culture are administered IV followed by tumor-directed in situ radiation therapy (FIG. 13I). In some embodiments, a therapeutically effective amount of the SIRPα^{low} macrophages which have not been activated in in vitro culture is administered IV followed by tumor-directed in situ radiation therapy and by IV administration of ICB (FIG. 13J).

Also disclosed herein are in vitro expanded tumor-specific peripheral blood T (PBT) cells for use in treating cancer that are produced by a method that involves obtaining a biological sample comprising peripheral blood mononuclear cells (PBMC) from the subject; isolating monocytes from the PBMC; isolating peripheral blood T cells from the blood or PBMCs; differentiating the monocytes in vitro to produce macrophages; contacting the macrophages with SIRPα expression inhibitor; contacting macrophages with activating agent, thereby generating a population of macrophages with marked reduction of SIRPα cell-surface expression (SIRPα^{low}), relative to untreated macrophages, and increased capacities of phagocytosis towards cancer cells, proinflammatory response and immunogenic antigen presentation; obtaining a biological sample comprising a tumor biopsy or a surgery tumor resection from the subject; in vitro co-culturing the activated SIRPα^{low} macrophages with cells from the tumor to allow phagocytosis of tumor antigens (tumor-fed SIRPα^{low} macrophages); in vitro co-culturing the tumor-fed SIRPα^{low} macrophages with the isolated PBT cells to expand the number of tumor-specific T cells; thereby producing a medicament for treating cancer comprising in vitro expanded tumor-specific PBT cells.

Therefore, also disclosed is a method for treating cancer in a subject that involves administering to the subject a therapeutically effective amount of the in vitro expanded tumor-specific PBT cells. In some embodiments, the in vitro expanded PBT cells are administered to the subject by IV administration (FIG. 13K). In some embodiments, the in vitro expanded PBT cells are administered to the subject by IV administration followed by tumor-directed in situ radiation therapy (FIG. 13L). In some embodiments, the in vitro expanded PBT cells are administered to the subject by IV administration followed by IV administration of ICB (FIG. 13N). In some embodiments, the in vitro expanded PBT cells are administered to the subject by IV administration followed by tumor-directed in situ radiation therapy and by IV administration of ICB (FIG. 13M). In some embodiments, the in vitro expanded PBT cells are administered to the subject by IV administration preceded by tumor-directed in situ radiation therapy. In some embodiments, the in vitro expanded PBT cells are administered to the subject by IV administration preceded by tumor-directed in situ radiation therapy and followed by IV administration of ICB.

Also disclosed herein are in vitro tumor-specific T cells from TIL cells that are produced by a method that involves obtaining a biological sample comprising peripheral blood mononuclear cells (PBMC) from the subject; isolating monocytes from the PBMC; differentiating the monocytes in vitro to produce macrophages; contacting the macrophages with SIRPα expression inhibitor; contacting macrophages with activating agent, thereby generating a population of macrophages with marked reduction of SIRPα cell-surface expression (SIRPα^{low}), relative to untreated macrophages, and increased capacities of phagocytosis towards cancer cells, proinflammatory response and immunogenic antigen presentation; collecting from the subject a biological sample comprising a tumor biopsy or a surgery tumor resection; isolating tumor infiltrating T lymphocyte (TIL) cells from the tumor biopsy; in vitro co-culturing the activated SIRPα^{low} macrophages with tumor cells from the tumor sample to allow phagocytosis and obtain tumor antigens (tumor-fed SIRPα^{low} macrophages); in vitro co-culturing the tumor-fed SIRPα^{low} macrophages with the isolated TIL cells to expand the number of tumor-specific T cells; thereby producing a medicament for treating cancer comprising in vitro expanded tumor-specific T cells from TIL.

Also disclosed herein is a method for treating cancer in a subject that involves administering to the subject to a therapeutically effective amount of the in vitro expanded tumor-specific T cells from TIL. In some embodiments, the in vitro expanded tumor-specific T cells from TIL are administered to the subject by IV administration (FIG. 13O). In some embodiments, the in vitro expanded tumor-specific T cells from TIL are administered to the subject by IV administration followed by tumor-directed in situ radiation therapy (FIG. 13P). In some embodiments, the in vitro expanded tumor-specific T cells from TIL are administered to the subject by IV administration followed by IV administration of ICB (FIG. 13R). In some embodiments, the in vitro expanded tumor-specific T cells from TIL are administered to the subject by IV administration followed by tumor-directed in situ radiation therapy and by IV administration of ICB (FIG. 13Q). In some embodiments, the in vitro expanded tumor-specific T cells from TIL are administered to the subject by IV administration preceded by tumor-directed in situ radiation therapy. In some embodiments, the in vitro expanded tumor-specific T cells from TIL are administered to the subject by IV administration preceded by tumor-directed in situ radiation therapy and followed by IV administration of ICB.

In some embodiments, the “SIRPα inhibitor” suppresses the expression of SIRPα, inhibits the activity of SIRPα, diminishes the abundance of SIRPα on the surface of a cell, disrupts the interaction between SIRPα and CD47, activates phagocytosis, promotes antigen processing and presentation to T cells, promotes activation of T cells, or a combination thereof.

In some embodiments, the macrophage activating agent increases phagocytosis by macrophages, increases the antigen processing and presentation activities and functions of macrophages, increases the immunostimulatory capacity of macrophages, improves the T cell stimulation function of macrophages, promotes a pro-inflammatory (so-called M1) phenotype of macrophages, or enables macrophages to change the TME to promote immune responses against cancer cells.

Also disclosed herein is a method for treating cancer in a subject that involves administering to the subject to a therapeutically effective amount of a SHP-1 inhibitor in combination with RT, ICB, an oncolytic virus, or any combination thereof.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a schematic illustrating activation and inhibition mechanisms controlling phagocytosis toward self/tumor cells, with special reference to the role played by the SIRPα-CD47 signaling axis.

FIGS. 2A and 2B show effects of intratumoral anti-PD-L1 on s.c. MC38 tumors in WT and SIRPα^{-/-} mice. Two doses of anti-PD-L1 Ab (50 μg, BioXcell, clone10F.9G2) were given when tumors were just formed (≤50 mm³, d8/d11) (FIG. 2A), or grew larger (>200 mm³, d12/d15), the latter being given ±IFNγ and CpG (100 ng and 20 μg, respectively) (FIG. 2B).

FIG. 3 shows effects of intratumoral anti-PD-1/L1 on subcutaneous PDA tumors Panc02 and KPC. Two doses of anti-PD-L1 Ab (50 μg each) were given via i.t. to Panc02 and KPC tumors of approximately 100 mm³.

FIG. 4 shows effects of αPD-L1 combined with tumor radiation. s.c. MC38, Pan02 and KPC tumors of 150-400 mm3 were given 8Gy X-ray radiation followed by αPD-L1 Ab (50 μg, i.t.) once to SIRPα^{-/-} mice, or 2× to WT mice (3 d apart).

FIGS. 5A to 5C show Sirpα^{-/-} mice after MC38 tumor eradication by treatment with αPD-L1+IFNγ/CpG (2×), or αPD-L1+8Gy radiation, developed long-lasting immunity that prevented tumor re-engraftments even with increased MC38 cells (FIG. 5A). Transfer of serum (FIG. 5B) or spleen T cells (FIG. 5C) from tumor-eradicated Sirpα^{-/-} mice to WT recipients conferred MC38 tumor resistance. The serum samples positively stained MC38 cell surface.

FIGS. 6A to 6D show Sirpα^{-/-} mice demonstrate enhanced anti-tumor CD8 Tc in TME by treatment with αPD-L1±IFNγ/CpG or 8Gy radiation (all data were 5 d post-treatment). FIG. 6B shows p15E specificity and GranzB expression, and detection of Tem. FIG. 6C shows ex vivo cytotoxicity assay by co-incubating Tc isolated from tumor with MC38 o/n. FIG. 6D shows statistics of total Tc, GranzB+, and P15E+subpopulations.

FIGS. 7A to 7D show SIRPα^{-/-} mice upon treatment by αPD-L1+IFNγ/CpG or 8Gy RT/IR (radiation treatment/irradiation) displayed diminishment of CD4+Foxp3+Tregs in TME. FIG. 7C shows significant Ly6C+ monocytes/MDSC infiltration in tumors after αPD-L1+8Gy RT in WT mice but absent in SIRPα^{-/-} mice. FIG. 7D shows tumor-associated leukocytes before and after αPD-L1+8Gy RT treatment. Data collected 3 d post-treatment.

FIGS. 8A to 8C show Sirpα^{-/-} MØ (BMDM, 0.5×10⁶) activated with IFNγ/CpG ex vivo were i.t. injected into MC38 tumors along with αPD-L1 Ab (2×) successfully induced tumor elimination. FIG. 8B shows increased tumor-specific Tc in TME after i.t. αPD-L1+Sirpα^{-/-} MØ, or various amounts of Sirpα^{-/-} MØ. FIG. 8C shows i.t. injection of Sirpα^{-/-} MØ (2×) to MC38 tumors in WT mice.

FIGS. 9A and 9B show CD47-triggered SIRPα signaling inhibits MØ antigen presentation machinery and proinflammatory cytokine production. WT and Sirpα^{-/-} BMDM were stimulated with IFNγ/CpG in the presence or absence of CD47 (mCD47.ex) for 12 h followed by FACS and ELISA detections for cell surface protein expression and cytokines secreted into medium.

FIG. 10 is a schematic demonstrating two-step inhibition by SIRPα^{-/-}: 1) tumor CD47-phagocyte SIRPα via SHP-1 suppresses antigen presentation machinery; 2) APC SIRPα→CD47 on T inhibits T cell activation.

FIG. 11 shows the disclosed macrophage therapy treatment drastically reduces SIRPα in human PBMC-derived MØ (FIG. 11A); phagocytosis-activation of the treated SIRPα^{low} MØ induces uptake of self-RBC (FIG. 11B) and human intestinal cancer cells HT29, T84, and Caco2, and THP1 leukemia cells (FIG. 11C).

FIG. 12 is a schematic showing an embodiment of the disclosed macrophage therapy treatment.

FIGS. 13A to 13R are schematics depicting the steps of various embodiments of the disclosed methods. As used in FIGS. 13A to 13R, the term “reagent A” means SIRPα inhibitor and the term “reagent B” means macrophage activator.

FIGS. 14A to 14F show local RT eliminates MC38 and PDA tumors Sirpα^{-/-} mice but not WT mice. FIG. 14A shows RT scheme. MC38, Pan02 or KPC cells were engrafted (5×10⁵, s.c.) into the right frank of WT or Sirpα^{-/-} mice and X-ray irradiation (IR) of various doses was given when tumors reached >150 mm³. FIGS. 14B to 14D show change in tumor volume and survival. Either a single fraction (FIGS. 14B and 14C) or three fractions (FIG. 14D) of IR were given when tumors were 150-400 mm³>500 mm³, respectively, except some Sirpα^{-/-} mice (purple lines, FIG. 14D) treated with two fractions. Blue lines indicate WT mice treated with 2×8Gy (FIG. 4C) or 8Gy-4Gy-4Gy (FIG. 14D) and with anti-PD-L1 Ab (50 μg, intratumoral) given after each fraction. Data are representative of at least three independent experiments of different tumors (n=3-12/group). The survival data were the record up to 1.5-year post IR. FIG. 14E contains representative images of MC38 and luciferase-expressing KPC tumors in WT and Sirpα^{-/-} mice before and after a single 8 Gy IR. FIG. 14D shows serum cytokines assessed at indicated times after IR (n=5/group).

FIGS. 15A to 15G show Sirpα^{-/-} mice exhibit RT-induced abscopal effects and long-lasting anti-tumor immunity. FIGS. 15A and 15B show abscopal effect in mice with MC38 (FIG. 15A) or KPC (FIG. 15B) tumors. Primary tumors (>150 mm³) were irradiated (8Gy), and 8-10 days later, a subset of mice whose abscopal tumor lingered were given anti-PD-L1 Ab (αPD-L1; 100 μg, i.p. 2×, 3 d apart). Tumor volume and survival were recorded. Representative images (FIG. 15B) show WT and Sirpα^{-/-} mice with luciferase-expressing KPC tumors engrafted in both flanks, dorsal area or peritoneal cavities before and after RT±αPD-L1. Data are representative of five independent experiments (n=3-8/group). FIGS. 15C to 15D show long-lasting anti-tumor immunity. After eradicating MC38 or PDA tumors (3 w post-RT), Sirpα^{-/-} mice were challenged with three rounds of dose-escalating inoculums of the same tumor (FIG. 15C). Tumor volume and survival were recorded (FIG. 15D). Data represent four independent experiments (n=3-8/group). Ten days after the last inoculum, their serum was examined for anti-tumor IgG by cell surface immunostaining of respective of tumor cells (FIG. 15E) and assessed for complement-dependent cytotoxicity (CDC) and macrophage phagocytosis (FIG. 15F); sera from MC38-resistant (containing anti-MC38 IgG) or tumor-naïve Sirpα^{-/-} mice are shown. Splenic T cells from the same MC38-resistant or tumor-naïve Sirpα^{-/-} mice were transferred to WT recipient prior to MC38 engraftment; tumor growth was recorded. FIG. 15G shows data represent three independent experiments (n=3/group) and are presented as mean±SD of triplicated tests.

FIGS. 16A to 16I show Sirpα^{-/-} macrophages but not CD47-blockade confer complete response after IR. FIGS. 16A and 16B show depletion of intratumoral macrophages diminished RT efficacy in Sirpα^{-/-} mice. MC38 or PDA tumors (>200 mm³) in Sirpα^{-/-} mice were administrated with CI2MDA-liposomes or an anti-CSF receptor antibody (αCSF1R) to deplete macrophages 2 days before and immediately after tumor 8Gy IR. Data are representative of two independent experiments (n=3-4/group). FIGS. 16C to 16F show combining RT with adoptive Sirpα^{-/-} BMDM infusion conferred tumor elimination in WT mice. MC38 tumors in WT mice were treated once (1×) or twice (2×, 3 d interval) with intratumoral (i. t.) injection of Sirpα^{-/-} BMDM (1×10⁴ per mm³ tumor mass), anti-murine CD47 blocking antibody (αCD47, miap301, 100 μg), soluble murine SIRPα extracellular domain (mSIRPα, ex, 100 μg), anti-MC38 serum (100 μl, undiluted), or αCD47 plus anti-MC38 serum with out (FIG. 16H) or with a further 8Gy IR given 3 h later. The same treatment was repeated 3 d later. Tumor volume and animal survival were recorded. Data are representative of two independent experiment (n=3-5/group).

FIGS. 17A to 17I show irradiation-activated Sirpα^{-/-} macrophages drive a proinflammatory TME. FIGS. 17A to 17D show MC38 tumors in WT and Sirpα^{-/-} mice prior to and after a single 8Gy IR were analyzed for CD45+ tumor-infiltrated leukocyte populations and CD45− non-leukocytes by flow cytometry. Frequency of intratumoral F4/80^{high} macrophages (MØ) before and after IR were visualized by t-SNE (FIG. 17B) and calculated per mg of tumor mass (FIG. 17D). Data are representative of at least six independent experiments (FIGS. 17A-17B) or pooled from three experiments (FIGS. 17C-17D, n-12-16/group). FIG. 17E shows GFP-positive Sirpα^{-/-} BMDM (GFP-Sirpα^{-/-} M) intratumorally infused (i.t., 1×10⁴/mm³) into WT recipients were analyzed prior to IR and at different time points post-IR. Data are pooled from two independent experiments and are presented as mean±S.D. (n=3/group). FIGS. 17F-17G show MC38-intratumoral F4/80′9 h macrophages in WT and Sirpα^{-/-} mice (FIG. 17F) or in GFP-Sirpα^{-/-} BMDM-infused WT recipients (FIG. 17G) were analyzed for antigen presentation and inflammatory phenotypes prior to (-IR) and 12 h post IR (8Gy). Cell-surface staining: MHC I/II, CD80/86 and PX40L; intracellular staining: IL-12, IFNα and IL-10. Data are representative of three independent experiments (n=3-4/group).

FIGS. 17A and 17I show mRNA profiling of bulk tumors before and 12 h after IR by Nanostring (nCounter Mouse Immunology Panel). Heatmap (FIG. 17H) and scatterplot (FIG. 17I) depicting differential expression of antigen presentation, pro-inflammatory and anti-inflammatory-associated genes (n=3 mice/group).

FIGS. 18A to 18H show Sirpα^{-/-} macrophages drive robust tumor-specific Tc expansion following RT. FIG. 18A shows TME analyses of CD8+ Tc and CD4+ Th among CD45+ tumor-infiltrated leukocytes in MC38, PanO2 or KPC tumors before and after a single fraction 8Gy IR. FIG. 18B shows IHC and IF staining of CD8+ Tc in MC38 tumors 3 d after IR. FIG. 18C shows frequency of granzyme B^{high} (GranzB) and p15E+ Tc in MC38 TME. Frequency of CD44+CD62L− effector memory T cells (T_{EM}) in p15E+ Tc were also determined. FIG. 18D is a summary of intratumoral GranzB^{high} and p15E+ Tc before and after IR. Data in FIGS. 18A to 18D are representative of at least six independent experiments (n=4-6/group). FIG. 18E shows frequency of P15E+ Tc and p15E+CD44+CD62L− TEM in peripheral blood and spleen of MC38-eradicated Sirpα^{-/-} mice. Data are representative of three independent experiments (n=5/group). FIG. 18F show WT mice with MC38 tumors were intratumorally infused with Sirpα^{-/-} BMDM via i.t. (total 2×10⁶, tumor size˜200 mm₃) and i.v. route (1×10⁷ per mouse) followed by IR (8Gy) for two rounds (3 d interval). 3 d after the second round, the frequency of p15+ and GranzB+Tc was determined. Data are representative of three independent experiments (n=2-4/group). FIG. 18G shows Tc from irradiated tumors were isolated (3 d post-IR) and co-cultured with MC38 cells at various effector:target ratios as indicated for 6 h or 24 h. Death of MC38 cells was determined by propidium iodide (PI) staining. Data are represented as mean±SD and represent three independent experiments (n=3/group). FIG. 18H shows depletion of CD8 Tc (αCD8) or CD4 Th (αCD4) in MC38 tumors of Sirpα^{-/-} mice prior to IR. Data are representative of two independent experiments (n=4/group).

FIGS. 19A to 19L show Sirpα^{-/-} macrophages reduce tumor immunosuppression after RT. MC38 tumors before and 3 d after IR were resected and analyzed for intratumoral immune populations for their cell numbers (FIG. 19A) and percentages (FIG. 19B). Note: data of WT mice were without Sirpα^{-/-} macrophage infusion. Data are pooled from four independent experiments and are presented as mean±S.D. (n=12/group). FIGS. 19C to 19D show Foxp3+ Treg and IFNγ-preducing Th1 among intratumoral CD4 T cells. Data are representative of three independent experiments (n=10-12/group). FIG. 19E shows GranzB expression in NK cells with mean fluorescent intensity (MFI) presented as mean±S.D. Data are representative of two independent experiments (n=5/group). FIGS. 19F to 19J shows differential intratumoral infiltration of monocytes and PMN in WT and Sirpα^{-/-} mice after IR. Gating strategies (FIG. 19F, FIG. 19I) determine monocytes (Ly6C+) and PMN (Ly6G+) and their numbers (FIG. 19G) among CD11b+ myeloid cells. Inhibition of T cell proliferation (FIG. 19H) was assayed in the presence of intratumoral myeloid cells. ROS production (FIG. 19J) by PMN was assayed in the presence of DCFDA and 1 μM PMA. Data are representative of three independent experiments and presented as mean±S.D. (n=3-5/group). FIGS. 19K to 19L show PMN infiltration promotes tumor regression. Intratumoral PMN and other leukocytes in 15 MC38 tumors of Sirpα^{-/-} mice 3 d post IR (K) and regression analysis of intratumoral PMN and the percentage of tumor regression (L). Data are representative of three independent experiments and are presented as mean±S.D. of triplicate assays (n=15/group).

FIGS. 20A to 20J show phagocytic Sirpα^{-/-} macrophages act as APC and activate tumor-specific Tc. FIG. 20A shows MC38 tumors (˜300 mm³) were excised immediately after IR (8Gy), minced and then cultured ex vivo; some WT MC38 tumors were i.t. injected with Sirpα^{-/-} BMDM (1×10⁶/mm³) immediately after excision prior to culture. After 4 d, single-cell suspensions were analyzed for Tc and Th in the CD45+ population. Data represent two independent experiments (n=3-5/group). FIGS. 20B-20G show in vitro expansion of tumor-specific Tc from TIL by tumor-phagocytosed Sirpα^{-/-} BMDM. FIG. 20B shows experimental scheme. FIG. 20C shows images of Tc (red, CD8 staining) forming conjugates with tumor antigen-loaded Sirpα^{-/-} BMDM (grey). Activation of Tc after 2 d of TIL-Sirpα^{-/-} BMDM co-culture was evident by cell size enlargement) increases in SSC and FSC) and GranzB expression (FIG. 20D), and robust Tc (but not Th) proliferation indicated by CSFE dilution (FIG. 20E) and summarized as frequency (FIG. 20F) and number (FIG. 20G) increases. Data are representative of at least five independent experiments, each with TIL pooled form three tumors and triplicate co-cultures. FIG. 20H shows cytotoxicity of Tc expanded by M38- or KPC-loaded Sirpα^{-/-} BMDM assessed by co-culture with MC38 or KPC cells, respectively, at indicated effector:target ratios for 24 h. TIL in which ˜70% being Tc expanded by αCD3/CD28 were used as comparison. Data represent three independent experiments and are presented as mean±S.D. (n=3/group). FIGS. 20I and 20J show effectiveness of Tc-MC38 and Tc-KPC in vivo. WT mice bearing MC38 (FIG. 20I) or KPC (FIG. 20J) tumors treated with Tc-MC38 or Tc-KPC (i.v. 5×10⁶), ±whole body radiation (WBI; 5Gy) and recombinant human IL-2 (i.p. 25,000 IU, 2× daily for 5 d), or the same number of TIL activated by αCD3/CD28. MC38-Tc exhibited an activated/migratory morphology compared to αCD3/CD28-TIL. Data represent five independent experiments (n=3-4 mice/group).

FIGS. 21A to 21C are schemes for controlling macrophage phagocytosis of cancer cells. FIGS. 21A and 21 B show tumor-associated macrophages are dominantly inhibited by immunosuppressive cytokines/factors in TEMs where the CD47-SIRPα axis is dispensable; thereby CD47-blockade alone (FIG. 21B) does not induce phagocytosis. FIG. 21C shows SIRPANT's proprietary reagent Phago-Act™ simultaneously downregulates SIRPα expression and activates macrophage phagocytosis, producing SIRPANT-M with capability to potently phagocytose tumor cells, and conduct antigen presentation to activate tumor-specific T cell cytotoxicity and long-lasting adaptive immunity.

FIGS. 22A to 22D shows tumor upregulates SIRPα expression. FIGS. 22A-22B show tumor-associated macrophages (TAMs), tumor-infiltrating dendritic, cells (DCs) and myeloid-derived suppressor cells (MDSCs) display increased SIRPα expression when tumors grew larger, as detected by flowcytometry. MC38: murine colorectal carcinoma; KPC: murine pancreatic ductal adenocarcinoma; EL4: murine T cell lymphoma. FIG. 22C shows IF staining of MC38 tumor sections. Note: CD47 (also PD-L1, FIG. 22A) exhibits increases on, tumor cells along tumor growth, indicative of stronger CD47-SIRPa regulation and much enhanced immunosuppression in, large tumors. FIG. 22D shows treating human PBMC-derived macrophages (human M) with various cancer cells-conditioned medium, increased SIRPa expression. HT29, Caco2 and T84: human colorectal cancer cells; MDA231, MDA-435, BT549 and, T47D: human breast cancer cells, etc.

FIGS. 23A to 23D show high SIRPα expression (SIRPα^{high}) confers macrophages strong immunosuppressive phenotype and tumor resistance to therapy. FIG. 23A shows comparing tumor-conditioned SIRPα^{high}-M and SIRPα^{-/-}-M for producing pro- and anti-inflammatory cytokines induced by IFNγ/LPS±the presence of tumor medium (TME) and/or CD47 ligation (CD47.ex). FIG. 23B shows SIRPα^{high}-M increased arginase-1 expression induced by IL-4 and decreased iNOS by IFNγ/LPS, whereas SIRPα^{-/-}-M displayed opposite expression. FIG. 23C shows transcription analyses of SIRPα^{high} and SIRPα^{-/-} tumors for responses to radiotherapy (RT): SIRPα^{high} tumors had poorly induced antigen presentation or proinflammatory response, but had enhanced immunosuppression indicated by increased TGFB and chemokines that attract MDSC for wound-healing and T cell inhibition; SIRPα^{-/-} tumors exhibited opposite response with their immune landscape indicative of strong inflammatory response and immunogenic antigen presentation that activated T cell tumor-killing activities. MC38: colorectal carcinoma; KPC & PanO2: pancreatic ductal adenocarcinoma. FIG. 23 D shows comparison of tumor-conditioned SIRPα^{high}-M and Phago-Act™-produced SIRPα^{Low}/SIRPANT-M for expression of antigen presentation machinery on cell surface.

FIGS. 24A to 24D show SIRPα regulation mechanisms. FIG. 24A shows tumor immunosuppressive signals upregulate SIRPα, whose cytoplasmic ITIMs are phosphorylated by Btk, resulting in recruitment of SHP-2 and reinforcement of TME immunosuppression. FIG. 24B shows under therapies, SIRPα via SFK-mediated ITIMs phosphorylation recruits/activates SHP-1, which inhibits multi-pathway proinflammatory signals, conferring therapeutic resistance. FIG. 24C shows under pro- or anti-inflammatory stimulation, phosphorylated SIRPα ITIMs in macrophages mediate discretely binding to either SHP-1 or SHP-2, respectively. FIG. 24D shows SIRPα regulation is independent of, but enhanced by CD47 extracellular ligation.

FIGS. 25A and 25B show activation of Sirpα-deficient macrophages to phagocytose cancer cells. FIG. 25A shows IL-17, LPS and IL-6 (each 10 ng/ml) activate SIRPα^{-/-}-M to phagocytose B16 melanoma cells in co-culture. The figure also shows that SIRPα^{-/-}-M had no phagocytosis in the absence of activation and that WT-M did not phagocytose in the presence or absence of activation. B) SIRPα^{-/-}-M treated with a cocktail comprising IL-6 (10 ng/ml), CpG and PolyI:C (each 100 ng/ml) exhibit aggressive phagocytosis of LLC lung cancer cells, MC38 colorectal adenocarcinoma, EL4 lymphoma, and PanO2 pancreatic cancer cells.

FIG. 26A shows IL-17A-treated SIRPα^{-/-} mice eliminated B16 melanoma. FIG. 26B shows melanoma-eradicated SIRPα^{-/-} mice developed anti-cancer immunity with anti-B16 Ab and capability to resist re-engraftment. WB: detecting B16 membrane proteins with ctl serum or anti-B16 serum from melanoma-eradicated SIRPα^{-/-} mice. FIG. 26C shows WT mice receiving anti-B16 serum demonstrated resistance to melanoma engraftment.

FIGS. 27A and 27B show tumor elimination by RT in SIRPα^{-/-} mice. MC38, PanO2 or KPC were s.c. engrafted into WT or SIRPα^{-/-} mice. After tumor well-formed (≥200 mm³), a fraction of X-ray RT (4-15Gy) was given followed by recording tumor volume changes and animal survival. Gray lines: WT mice resisted 2× applications of 8Gy RT plus anti-PD-L1 (100 μg, i.p), 3 d apart. FIG. 27C shows intratumoral depletion of SIRPα^{-/-}-M abrogated RT efficacy in SIRPα^{-/-} mice. FIG. 27D shows adoptive transfer of bone marrow-derived SIRPα^{-/-}-M into tumors in WT mice conferred tumor regression by RT.

FIGS. 28A to 28D show tumor elimination in SIRPα^{-/-} mice by IR was associated with expansion of anti-tumor Tc (FIG. 28A) that expressed nigh GranzB and tumor antigen (p15E) specificity of which a fraction had differentiated to T_{EM} (CD44⁺CD62L⁻) (FIG. 28B). SIRPα^{-/-} tumors also diminished Foxp3 Tregs (FIG. 28C) and reduced Ly6C+ MDSC infiltration but increased NK after IR (FIG. 28D).

FIGS. 29A to 29C show up- and down-regulation of SIRPα expression in macrophages by cytokines, TLR agonists, steroids, and tumor-conditioned medium. FIGS. 29A and 29B show murine bone marrow-derived macrophages and FIG. 29C shows human PBMC-derived macrophages. FIG. 29D is a scheme of ex vivo producing SIRPα^{low} activated macrophages, SIRPANT-M, by Phago-Act™. FIG. 29E shows human SIRPANT-M resist phenotypic change (re-express SIRPa) in tumor conditions and maintain longevity. FIG. 29F shows human SIRPANT-M directly phagocytose human cancer cells.

FIGS. 30A to 30D show murine SIRPANT-M directly phagocytose syngeneic cancer cells. FIG. 30A shows an experimental scheme. FIG. 30B shows sample microscopy results of SIRPANT-M phagocytosing EL4 lymphoma and MC38 colorectal adenocarcinoma cells. FIG. 30C shows sample flow cytometry showing SIRPANT-M phagocytosis of MC38 cells. BMDM or SIRPANT-M were gated by CD11b+. FIG. 30D shows phagocytosis of syngeneic cancer cells in 4 h. ****p<0.0001.

FIG. 31A shows human PBMC-derived macrophages (SIRPa*-M) were treated by TNFα and IL-17, or INFγ, or Phag-Act (SIRPANT-M) for 2 d before testing for phagocytosis towards various human cancer cells. Only SIRPANT-M exhibited positive phagocytosis. FIG. 31B shows time-course SIRPANT-M phagocytosis. FIG. 31C shows SIRPANT-M phagocytosis of NCI-60 human cancer panel in 4 h. FIG. 31D shows microscopic images showing SIRPANT-M phagocytosis of HT29, T84, Caco2 and THP-1. FIG. 31E shows SIRPANT-M mediate phagocytosis irrelevant to CD47 expression on cancer cells.

FIGS. 32A and 32B show human SIRPANT-M display enhanced phagocytosis towards X-ray radiation-treated human cancer cells. Human PBMC-derived SIRPANT-M (FIG. 32A) or SIRPα⁺-M (FIG. 32B) were incubated with various non-irradiated (-IR) or irradiated (8Gy) human cancer cells for 4 h, followed by assessing phagocytosis. Sample fluorescence microscopy images showing SIRPANT-M but not SIRPα⁺-M (CD11b staining) aggressively phagocytosing irradiated OVCAR3 ovarian cancer cells and UACC-62 melanoma cells (CFSE).

FIGS. 33A to 33E show murine SIRPANT-M enhanced phagocytosis towards radiation-treated cancer cells. FIG. 33A is a comparison of BMDM (SIRPα⁺) and SIRPANT-M for phagocytosis of non-irradiated (-IR) and irradiated (8Gy) syngeneic tumor cells. B) Microscopy and flow cytometry showing SIRPANT-M but not BMDM aggressively phagocytosing irradiated MC-38 cells. FIG. 33C shows time-course assays showing SIRPANT-M were enhanced of phagocytosing EL4 irradiated at varied dosages. FIGS. 33D to 33E show non-ablative radiation did not induced apoptosis (PI/YO-PRO-1) or changes of cell surface CD47, but increased calreticulin (CRT).

FIGS. 34A to 34C show SIRPANT-M activation phenotype and antigen presentation capacity. Freshly derived murine BMDM (SIRPα⁺-M) were further treated with Phago-Act™ for 48 h to induce SIRPANT-M. FIG. 34A shows SIRPα expression on SIRPα⁺-M and SIRPANT-M before and after Phago-Act™ treatment. B) The capacity of SIRPANT-M versus SIRPα⁺-M as antigen presenting cells (APC) assessed by their expression of MHC-I, MHC-II and costimulatory molecules CD80 and CD86. FIG. 34C shows inflammatory features of SIRPANT-M versus SIRPα⁺-M assessed by their production of pro- and anti-inflammatory cytokines. FIG. 34D shows transcription analyses of genes involved in antigen presentation and proinflammatory response in SIRPANT-M compared to SIRPα⁺-M by Nanostring MRNA profiling.

FIGS. 35A to 35C show mapping mRNA transcription of seven human PBMC-derived SIRPANT-M compared to donor-matched SIRPα⁺-M. FIG. 35A is a heatmap transcription analyses of genes involving in antigen presentation and pro- and anti-inflammatory responses. FIG. 35B shows gene expression programs induced in SIRPANT-M by Phago-Act™. Display shows differentially regulated genes (2029 total, 1093 upregulated, 936 downregulated), categorized per known or predicted function(s), literature and sequence similarity. FIG. 35C is a scatterplot showing gene expression differences in SIRPANT-M compared to SIRPα⁺-M.

FIG. 36A to 36LK show in vitro SIRPANT-M activating MC38- and KPC-specific T cells from intratumoral TIL. FIG. 36A is an example scheme. FIGS. 36B-36D show SIRPANT-M but not SIRPα⁺-M (FIG. 36B) fed with tumor antigen (FIG. 36C) induced CD8+ T cell expansion from TIL. Minimal CD4+ T cell expansion was detected (FIG. 36D). FIGS. 36E to 36G show SIRPANT-M following phagocytosis of tumor antigens mediated engagement with CD8 T cells (CD8 staining) for antigen presentation (FIG. 36E), a process that induced CD8 T cell enlargement (increase SSC and FSC on day 2 (D2)) and proliferation (FIG. 36G). FIG. 36H to 36I show SIRPANT-M-activated CD8 T cells against MC38 displayed increased reactivities with MC38-specific p15E and ADPGK epitopes and highly expressed granzyme B. FIG. 36J shows in vitro SIRPANT-M-activated CD8 T cells cytotoxicity against cancer. CD8 T cells that were expanded from MC38 TIL and KPC TIL, termed T^{MC38} and T^{KPC} were co-incubated (12 h) with healthy cultured MC38 and KPC cells, respectively, at the T: cancer cell ratio of 1:1 or 1:3, followed by analyses of cancer cell death (J) compared to MC38 and KPC cells without T cell co-incubation (Ctl.). FIG. 36K shows real-time imaging snapshots of T^{MC38} (arrowhead) killing MC38 cells.

FIG. 37 shows SIRPANT-M induce B16-gp33 antigen specific CD8 T cell activation in vitro. Left: the experimental scheme. Right: Only B16gp33-fed SIRPANT-M robustly induced antigen (gp33)-specific T cell activation.

FIGS. 38A to 38F show SIRPANT-M intratumoral monotherapy treating early stage (small tumor) and late stage (large tumor) colorectal cancer MC38 and pancreatic ductal adenocarcinoma KPC (both s.c.). Dose-dependent studies. FIG. 38A shows intratumoral injection (i.t.) dosing strategy. FIG. 38B shows tracing SIRPANT-M in MC38 tumor after i.t. injection and the dynamics shows SIRPANT-M presence in the tumor for approximately 2 days. FIG. 38C shows treating MC38 of varied sizes (dash lines) with SIRPANT-M by i.t. Data show one of two-three cohorts of each size of MC38 tumors treated with D½ and D1 doses, 3×, every three day, starting on day 10, day 12, day 14 and day 16 post MC38 engraftment. FIG. 38D shows overall survival of MC38-engrafted mice treated with vehicle (PBS) control or 3× SIRPANT-M i.t. at D½ and D1 doses. Data summarize two-three cohorts of each treatment group, n=10-22. FIG. 38E shows treating KPC of varied sizes (dash lines) with SIRPANT-M by i.t. Data show one of two-three cohorts of each KPC tumor sizes treated with D1 dose for 3×, every three day, starting on day 14, day 16 and day 18 post-KPC engraftment. Note: MC38 tumors generally grow faster than KPC tumors by s.c.; treatment doses calculated according to tumor sizes. FIG. 38F shows overall survival of KPC-engrafted mice treated with vehicle (PBS) control or 3× SIRPANT-M i.t. at D1 dose. Data summarize two-three cohorts of total n=15-20 in each treatment group.

FIGS. 39A to 39C show SIRPANT-M therapy is tumor-agnostic. FIG. 39A shows colorectal (MC38), pancreatic (PanO2), lung (LLC) or lymphoma (EL4) tumors (sizes 150-400 mm³) were treated with SIRPANT-M at the D2 dose (i.t., 3×, every third day). One of two-three cohorts of each type of cancer is shown. FIG. 39B shows overall survival of tumor-engrafted mice treated with vehicle control (PBS) or D2 dose SIRPANT-M by i.t. Data summarize multiple cohorts of each type of cancer with treatment applied at different stages (tumor sizes). FIG. 39C shows SIRPANT-M treating spontaneous triple negative mammary gland cancer in MMTV-PyMT mice (n=20). SIRPANT-M at the D1 dose were intratumorally injected into the first arising tumor on day 62 and 66, and the largest later arising tumor on day 70, 74, 76 and 82 and 80. Only one tumor was treated at a time. Overall survival is shown the number of mice alive as fractions. Median overall survival and Kaplan Meier analysis are shown.

FIGS. 40A to 40F show SIRPANT-M i.t. and RT combination eliminates RT-refractory MC38 colorectal and KPC and PanO2 pancreatic cancers. FIG. 40A shows mice with MC38, KPC and PanO2 cancers of different sizes were treated with two rounds of RT or RT plus SIRPANT-M i.t. at D2 dose. The treatment schemes for relatively small tumors were either 4Gy and 4Gy (tumors≤200 mm³, 3 d apart), or 8Gy and 8Gy (tumors 200-400 mm³, 3 d apart), without or with immediate SIRPANT-M i.t. following each RT fraction. For large tumors, 15Gy was used for the first treatment and then 8Gy for the second treatment. A group of tumor-bearing mice was set as control without treatment. FIGS. 40B and 40C show MC38 colorectal cancer progression or regression (FIG. 40B) and the overall survival (FIG. 40C) of cancer-engrafted mice after receiving treatments to tumors of different sizes. FIGS. 40D and 40E show KPC pancreatic cancer progression or regression (FIG. 40D) and the overall survival of mice (FIG. 40E) after receiving treatments to their tumors of different sizes. FIGS. 40F and 40G show Pan02 pancreatic cancer progression or regression (FIG. 40F) and the overall survival of mice (FIG. 40G) after receiving treatments to their tumors of different sizes.

FIGS. 41A and 41B show dose-dependent SIRPANT-M efficacy in combination with RT treating MC38 colorectal and KPC and Pan02 pancreatic cancers. FIG. 41A shows well-established MC38, KPC and Pan02 tumors of sizes<250 mm³ (blue line) or larger (>300 mm³, red line) were treated with a fraction of 8Gy X-ray irradiation followed by immediate (<30 min) i.t. administration of SIRPANT-M at D½ (open circle) or D2 dose (closed square). The same treatment was repeated three days later (total 2×). Records of tumor volume changes. FIG. 41B shows survival records of mice without treatment, with only 8Gy RT, or 8Gy RT plus varied doses of SIRPANT-M i.t. The data include mice given SIRPANT-M i.t. at D½, D1 and D2 doses.

FIGS. 42A to 42C show SIRPANT-Mi.t and RT combination induces strong abscopal effects and systemically eliminates KPC cancer lesions. Mice were engrafted with KPC/Luc pancreatic adenocarcinoma at multiple locations (FIG. 40A). After tumor formation, one or two largest palpable tumors (red circle, all >200 mm³) were treated with 8Gy RT and SIRPANT-M i.t. at D1 dose for the first round, followed by two rounds of 4Gy RT and SIRPANT-M i.t. at D1 dose. (Each round given with three days in between). Control group (left) was given three rounds of 8Gy RT without SIRPANT-M. Whole body luminescence imaging was conducted prior to and after each treatment to record tumor growth or regression. Total tumor volumes (FIG. 42B) were calculated by the in vivo luminescence intensity of KPC/Luc cells, and animal survival (FIG. 42C) was recorded.

FIGS. 43A to 43E show SIRPANT-M plus RT induces strong abscopal effects that systemically clear MC38 colorectal cancer lesions. Mice were engrafted with MC38 tumors in both flanks with the right side to be the primary, where SIRPANT-M i.t. plus RT treatments were given. FIG. 43A shows an experimental scheme. FIGS. 43B and 43C show tumor volume changes on both flanks when the right side primary tumor received treatments. FIGS. 43D and 43E show survival records of mice with small and large primary and abscopal tumors correlated to FIGS. 43B and 43C, respectively. Note: A single dose (20 μg, i.p.) anti-PD-L1 was given to mice that initially harbored large abscopal tumor in FIG. 43C to facilitate abscopal clearance.

FIGS. 44A and 44B show efficacy of SIRPANT-M i.t. administration before or after RT. FIG. 44A shows MC38 colorectal cancer and EL4 lymphoma established in C57BL6 mice were treated with SIRPANT-M i.t. (D1 dose) either immediately (<3 h), or 24 h, or 48 h before a fraction of 8Gy RT, or the same time length after the RT. Tumor volume changes in response to different treatments were recorded and compared to no treatment controls and tumors treated by RT only. FIG. 44B shows survival records of mice treated with SIRPANT-M i.t. and RT of different orders.

FIGS. 45A to 45D show dose-dependent SIRPANT-M efficacies when combining with RT to treat lung cancer (LLC), lymphoma (EL4) and two forms of triple negative breast cancer (4T1 and PyMT). LLC lung cancer and EL4 lymphoma were s.c. engrafted into C57BL6 mice. 4T1 breast cancer was implanted orthotopically into Balb C mouse mammary gland. Female MMTV-PyMT mice spontaneously developed breast cancer at approximately 50 day of age. After palpable tumor formation, tumors were treated with their syngeneic SIRPANT-M at D½, D1 and D2 doses via i.t. immediately following a fraction of 8Gy RT. The treatment was repeated 3 d later (total 2×). For PyMT mice, the SIRPANT-M i.t. and 8Gy RT treatment was applied to the first palpable tumor, followed by additional treatments to other tumors appeared later, though only largest tumor was treated at each time. A total of 6× SIRPANT-M i.t. and RT combination treatments applied.

FIG. 46 shows timing and sequence of generating human SIRPα^{low} macrophages from PBMC.

FIGS. 47A and 47B show treatment of KPC (FIG. 47A) and of MC38 (FIG. 47B) cancers with TPI-1 or TPI-1+RT.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, medicine, and the like, which are within the skill of the art.

Descriptions of the methods of the invention may include routine steps, e.g., collecting or obtaining a biological sample from a subject or delivering or administering a composition to a subject that accompany the processing steps of the invention. In such cases, it is understood that the methods of the invention may exclude any or all steps of collecting or obtaining a biological sample or administering or delivering a composition to a subject.

The following examples are put forth so as to provide those of ordinary skill in the art with a complete disclosure and description of how to perform the methods and use the therapies disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise. The term “about”, when immediately preceding a number or numeral, means that the number or numeral ranges plus or minus 10%.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used that is of sufficient quantity to achieve an outcome, for example, a beneficial or desired result, such as, amelioration of one or more causes or symptoms of a disease or disorder. Such amelioration only requires a reduction or alteration, not necessarily elimination. The therapeutically effective amount may vary depending upon one or more of: the subject and disease condition being treated, the weight and age of the subject, the severity of the disease condition, the manner of administration and the like.

The term “pharmaceutically acceptable” refers to those compounds, materials, compositions, and/or dosage forms which are, within the scope of sound medical judgment, suitable for use in contact with the tissues of human beings and animals. In general, a pharmaceutically acceptable moiety has one or more benefits that outweigh any deleterious effect that the moiety may have. Deleterious effects may include, for example, toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “agent” or “compound” as used herein refers to one or more chemical entities or biological products (e.g. a protein, a peptide, a nucleic acid, a polynucleotide, a carbohydrate moiety), or combination of chemical entities and/or biological products. Depending on the identity of the “agent”, it may be contacted with cells in vitro, or administered to a subject (e.g., to treat or prevent or control a disease or condition). In some embodiments, the agent is a protein, such as, for example, a cytokine, or an antibody. In some embodiments, the agent is a carbohydrate moiety, such as, lipopolysaccharide (LPS). In some embodiments, the agent is a chemical entity, such as, polyinosinic:polycytidylic acid (poly I:C). In some embodiments, the agent is a nucleic acid, such as, CpG oligonucleotide (ODN). The chemical entity or biological product is preferably, but not necessarily a low molecular weight compound, but may also be a larger compound, or any organic or inorganic molecule, including modified and unmodified nucleic acids such as antisense nucleic acids, RNAi, such as siRNA or shRNA, peptides, peptidomimetics, receptors, ligands, and antibodies, aptamers, polypeptides, nucleic acid analogues or variants thereof. For example, an agent can be an oligomer of nucleic acids, amino acids, or carbohydrates including, but not limited to proteins, peptides, oligonucleotides, ribozymes, DNAzymes, glycoproteins, RNAi agents (e.g., siRNAs), lipoproteins, aptamers, and modifications and combinations thereof. The agent can also be a naturally occurring cell or a modified cell. In some embodiments, an active agent is a nucleic acid, e.g., miRNA or a derivative or variant thereof.

As used herein, a “SIRPα inhibitor” is an agent that is capable of promoting a reduction in the expression levels (e.g. protein, mRNA), reduction in function (e.g. signaling function) and/or reduction in interaction capability (e.g. interaction with CD47) of SIRPα. In some embodiments, the SIRPα inhibitor physically associates with SIRPα. In some embodiments, upon contact with a SIRPα-expressing cell, the SIRPα inhibitor is capable of reducing the expression of SIRPα (e.g. the cell-surface expression of SIRPα), inhibiting the activity of SIRPα, disrupting the interaction between SIRPα and CD47, or any combination thereof.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “radiation” refers to ionizing radiation consisting of energetic subatomic particles, ions, or atoms moving at high speeds or high-energy electromagnetic waves. Herein the term “radiation” is used in the medical context and is used synonymously with “ionizing radiation,” “irradiation,” “radiation therapy,” and “radiotherapy.” The term “tumor-directed radiation” refers to the medical use of a beam of radiation that is pointed directly at the tumor of a patient.

### Compositions and Methods

Disclosed herein are methods for treating cancer in a subject that comprise administering to the subject a therapeutically effective amount of activated SIRPα^{low} macrophages. These activated SIRPα^{low} macrophages can in some embodiments be produced by a method that comprises collecting a biological sample comprising peripheral blood mononuclear cells (PBMC) from the subject; isolating monocytes from the PBMC; culturing the monocytes in vitro to produce macrophages; contacting the macrophages with a SIRPα inhibitor to generate a population of macrophages with reduced SIRPα cell-surface expression or activity (SIRPα^{low} macrophages) relative to untreated macrophages; and contacting the SIRPα^{low} macrophages with an macrophage activating agent to activate the SIRPα^{low} macrophages, and thereby produce activated SIRPα^{low} macrophages.

In some embodiments, the SIRPα inhibitor and macrophage activating agent are contacted with the macrophages sequentially. This can be in either order and can be minutes, hours, or days apart, such as 1, 2, 3, 4, 5, 6, 7, 8, 9, 10, 11, 12, 13, 14, 16, 16, 17, 18, 19, 20, 21, 22, 23, or 24 hours apart. In other embodiments, the SIRPα inhibitor and macrophage activating agent are contacted with the macrophages simultaneously or concurrently.

In some embodiments, the SIRPα inhibitor and macrophage activating agent are present in the same composition. Therefore, in some embodiments, the methods comprise isolating monocytes from peripheral blood mononuclear cells (PBMC) in a biological sample; differentiating the monocytes in vitro to produce macrophages; and contacting the macrophages with a composition, comprising an SIRPα inhibitor and a macrophage activating agent, to generate a population of activated SIRPα^{low} macrophages. In some embodiments, the activated SIRPα^{low} macrophages exhibit reduced SIRPα cell-surface expression relative to control untreated macrophages. In some embodiments, the activated SIRPα^{low} macrophages exhibit increased activities of phagocytosis, proinflammatory activity, antigen presentation, or any combination thereof relative to untreated macrophages.

In some embodiments, SIRPα^{low} macrophages have reduced SIRPα cell-surface expression or activity that is reduced by about 90% compared to untreated macrophages, including reduced by about 80%, 81%, 82%, 83%, 84%, 85%, 86%, 87%, 88%, 89%, 90%, 91%, 92%, 93%, 94%, 95%, 96%, 97%, 98%, 99%, or 100% compared to untreated macrophages. In some embodiments, the expression of SIRPα in activated SIRPα^{low} macrophages is lower than the expression of SIRPα in control untreated macrophages. In some embodiments, the expression is cell-surface expression of SIRPα. In some embodiments, the expression of SIRPα in activated SIRPα^{low} macrophages is at least about 50% (for example, about 60%, about 70%, about 80%, about 90%, about 95%, about 98%, about 99%, or 100%, including all values and subranges that lie therebetween) lower than the expression of SIRPα in control untreated macrophages.

In some embodiments, the activity of SIRPα in activated SIRPα^{low} macrophages is lower than the activity of SIRPα in control untreated macrophages. In some embodiments, the activity of SIRPα in activated SIRPα^{low} macrophages is at least about 50% (for example, about 60%, about 70%, about 80%, about 90%, about 95%, about 98%, about 99%, or 100%, including all values and subranges that lie therebetween) lower than the activity of SIRPα in control untreated macrophages.

In some embodiments, the phagocytic activity, the proinflammatory activity, and/or the antigen presentation activity of activated SIRPα^{low} macrophages is higher than the phagocytic activity, the proinflammatory activity, and/or the antigen presentation activity, respectively, of control untreated macrophages. In some embodiments, the phagocytic activity, the proinflammatory activity, and/or the antigen presentation activity of activated SIRPα^{low} macrophages is at least about 2% (for example, about 3%, about 4%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 200%, about 300%, about 400%, about 500%, about 600%, about 700%, about 800%, about 900%, about 1000%, about 10,000%, about 100,000%, about 1,000,000%, or about 10,000,000%, including all values and subranges that lie therebetween) higher than the corresponding the phagocytic activity, the proinflammatory activity, and/or the antigen presentation activity, respectively, of control untreated macrophages.

In some embodiments, the phagocytic activity of the one or more activated SIRPα^{low} macrophages is higher than the phagocytic activity of control untreated macrophages. In some embodiments, the phagocytic activity of the one or more activated SIRPα^{low} macrophages is at least about 2% (for example, about 3%, about 4%, about 5%, about 10%, about 15%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, about 10,000%, about 100,000%, about 1,000,000%, or about 10,000,000%, including all values and subranges that lie therebetween) higher than the phagocytic activity of the control untreated macrophages.

In some embodiments, the proinflammatory activity of the one or more activated SIRPα^{low} macrophages is higher than the proinflammatory activity of control untreated macrophages. In some embodiments, the proinflammatory activity of the one or more activated SIRPα^{low} macrophages is at least about 2% (for example, about 3%, about 4%, about 5%, about 10%, about 15%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, about 10,000%, about 100,000%, about 1,000,000%, or about 10,000,000%, including all values and subranges that lie therebetween) higher than the proinflammatory activity of the control untreated macrophages.

In some embodiments, the antigen presentation activity of the one or more activated SIRPα^{low} macrophages is higher than the antigen presentation activity of control untreated macrophages. In some embodiments, the antigen presentation activity of the one or more activated SIRPα^{low} macrophages is at least about 2% (for example, about 3%, about 4%, about 5%, about 10%, about 15%, about 20%, about 30%, about 40%, about 50%, about 60%, about 70%, about 80%, about 90%, about 100%, about 200%, about 300%, about 400%, about 500%, about 600%, about 700%, about 800%, about 900% or about 1000%, about 10,000%, about 100,000%, about 1,000,000%, or about 10,000,000%, including all values and subranges that lie therebetween) higher than the antigen presentation activity of the untreated control macrophages.

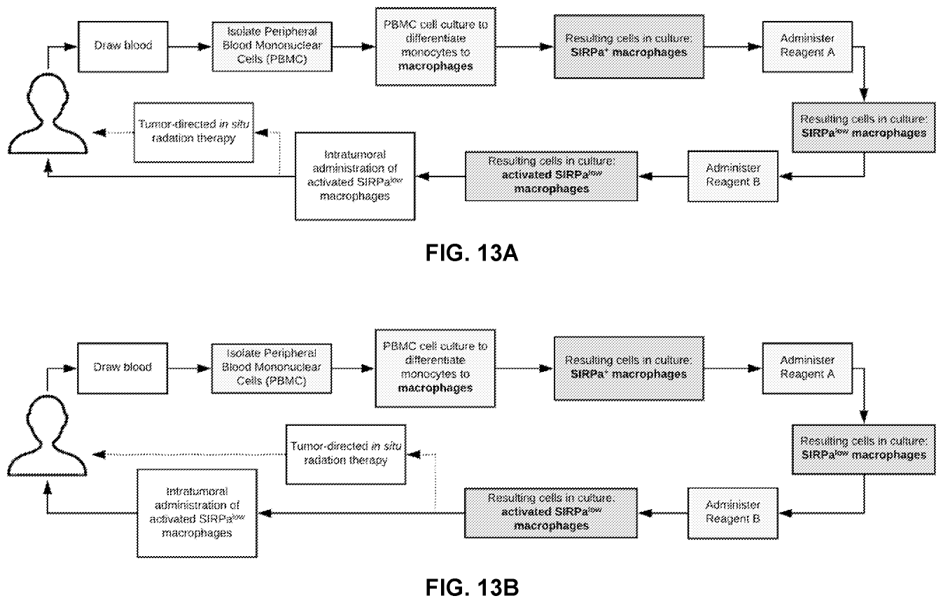

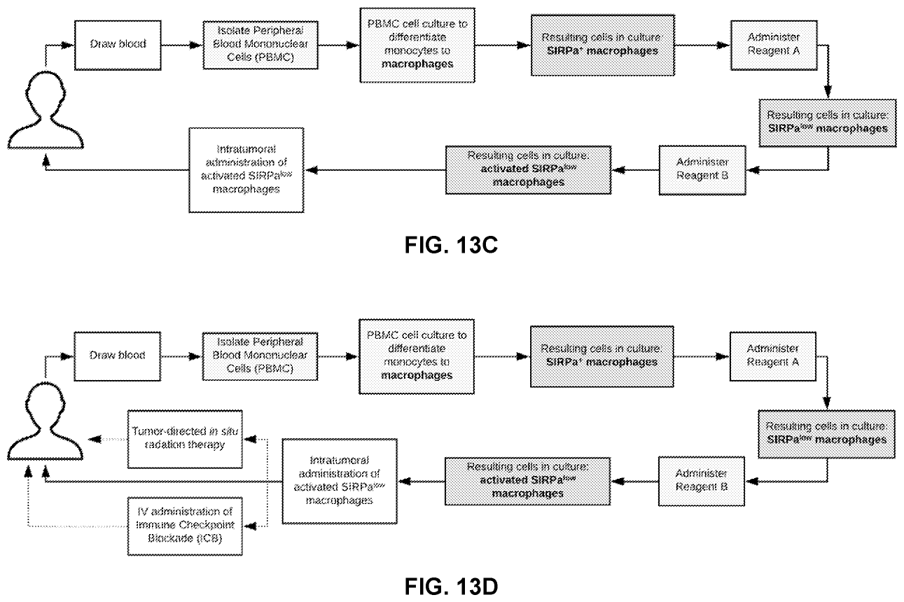

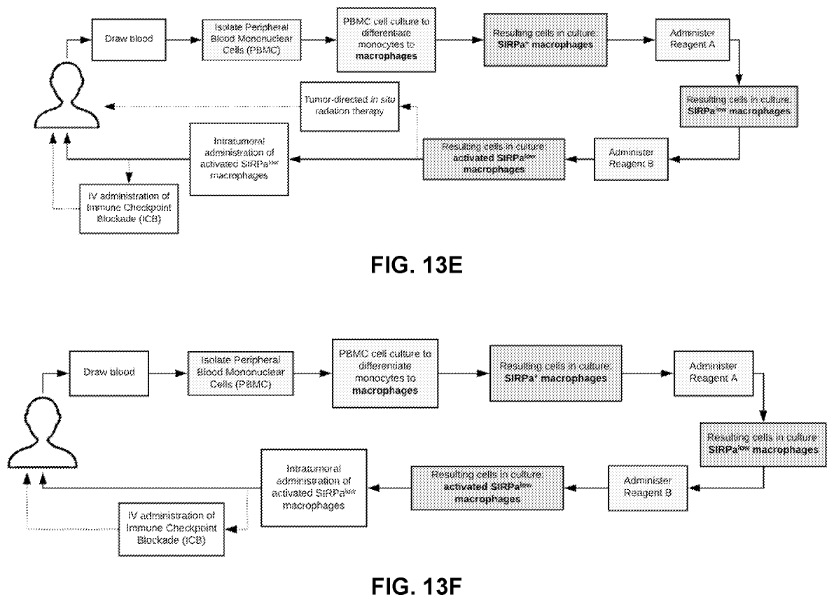

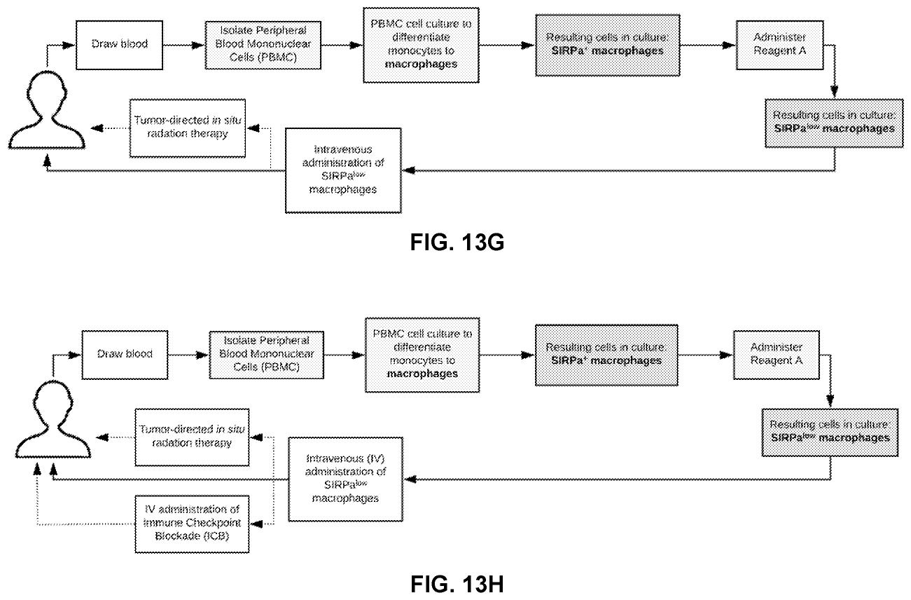

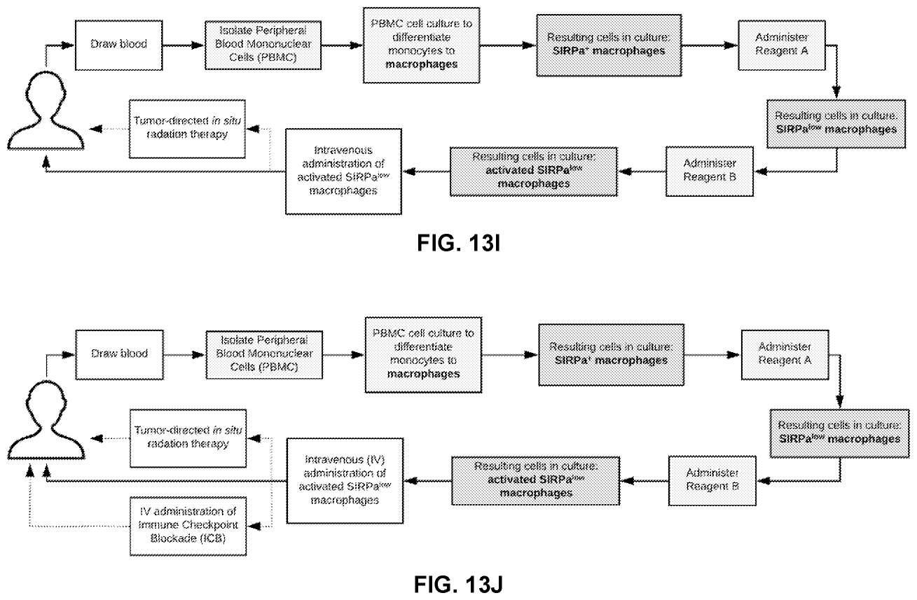

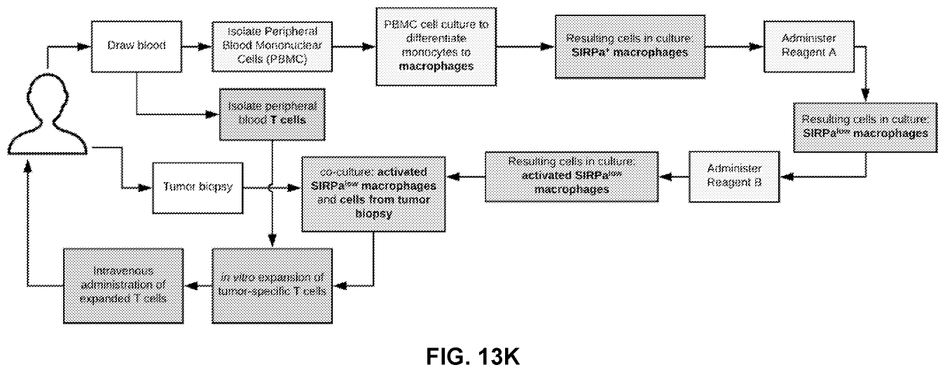

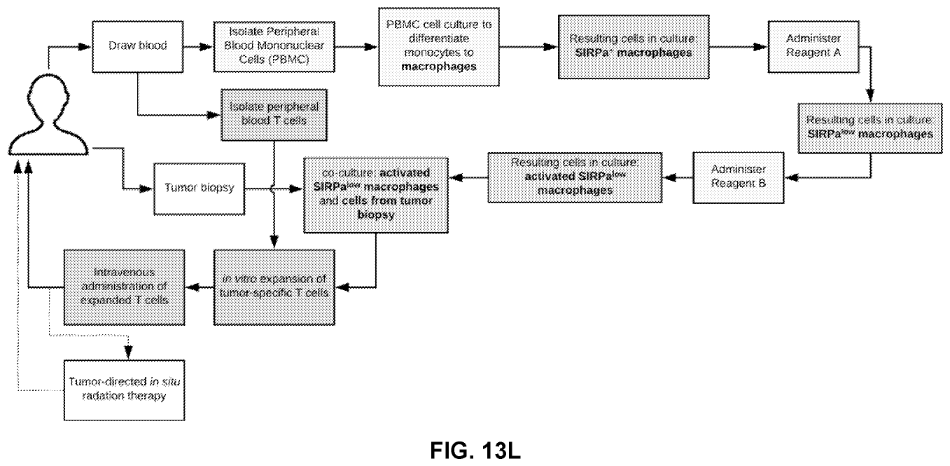

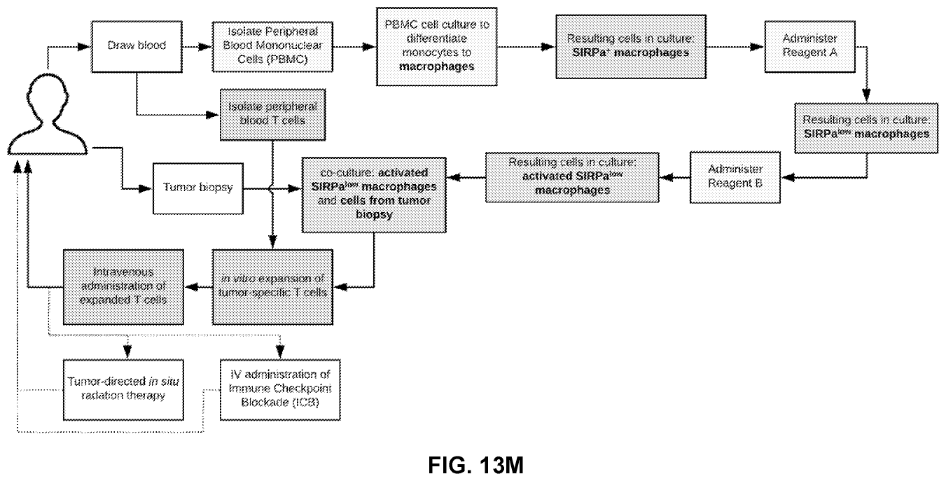

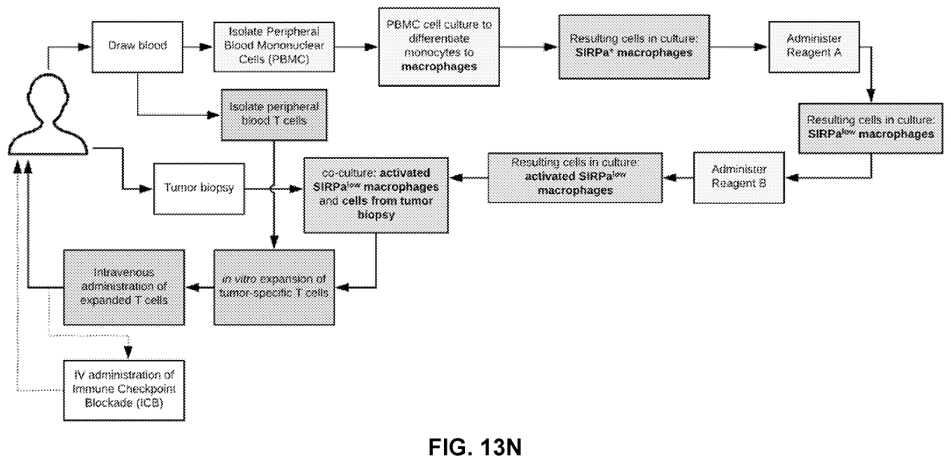

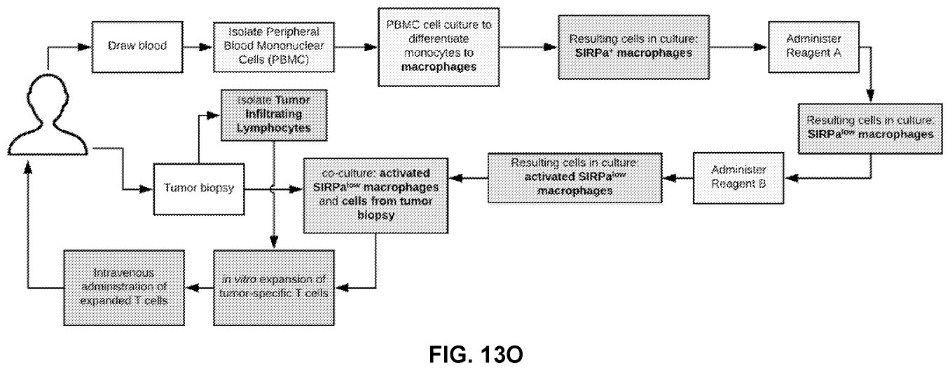

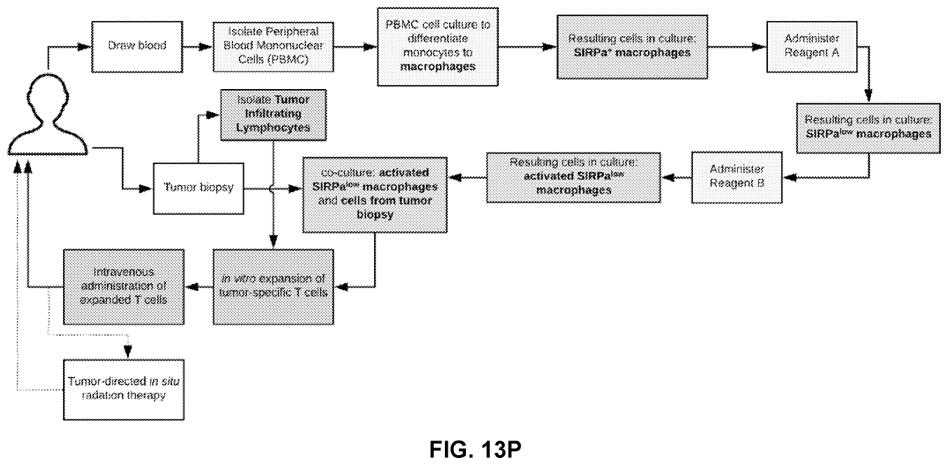

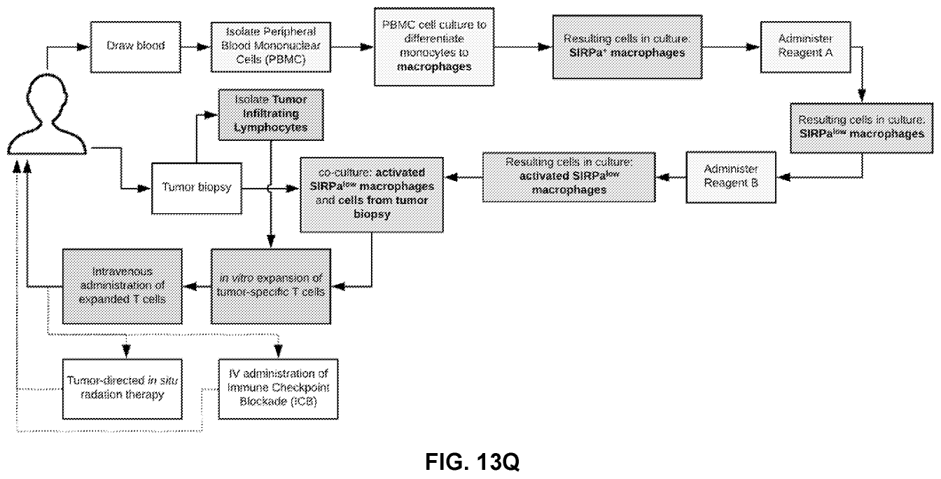

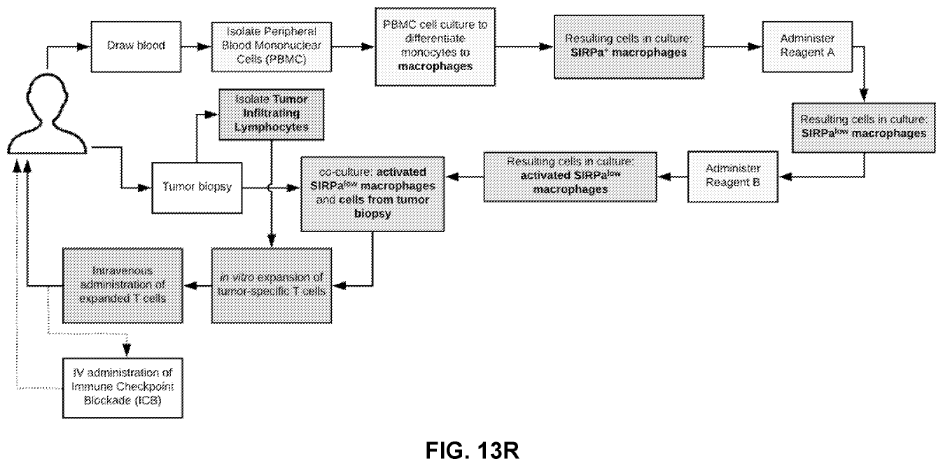

Various embodiments of the disclosed methods are illustrated in FIGS. 13A to 13R. For example, in some embodiments, the therapeutically effective amount of the activated SIRPα^{low} macrophages are administered directly into the tumor and this administration is followed by tumor-directed in situ radiation therapy (FIG. 13A). In some embodiments, the therapeutically effective amount of the activated SIRPα^{low} macrophages are administered directly into the tumor and this administration is preceded by tumor-directed in situ radiation therapy (FIG. 13B). In some embodiments, the therapeutically effective amount of the activated SIRPα^{low} macrophages are administered directly into the tumor without any tumor-directed in situ radiation therapy (FIG. 13C).

In some embodiments, the therapeutically effective amount of the activated SIRPα^{low} macrophages are administered directly into the tumor and this administration is followed by tumor-directed in situ radiation therapy and by intravenous (IV) administration of ICB (FIG. 13D). In some embodiments, the therapeutically effective amount of the activated SIRPα^{low} macrophages are administered directly into the tumor and this administration is preceded by tumor-directed in situ radiation therapy and followed by IV administration of ICB (FIG. 13E). In some embodiments, the therapeutically effective amount of the activated SIRPα^{low} macrophages are administered directly into the tumor and this administration is followed by IV administration of ICB without any tumor-directed in situ radiation therapy (FIG. 13F).

In some embodiments, a therapeutically effective amount of the SIRPα^{low} macrophages which have not been activated in in vitro culture are administered IV and this administration is followed by tumor-directed in situ radiation therapy (FIG. 13G). In some embodiments, a therapeutically effective amount of the SIRPα^{low} macrophages which have not been activated in in vitro culture are administered IV and this administration is followed by tumor-directed in situ radiation therapy and by IV administration of ICB (FIG. 13H).

In some embodiments, the therapeutically effective amount of the activated SIRPα^{low} macrophages are administered IV and this administration is followed by tumor-directed in situ radiation therapy (FIG. 13I). In some embodiments, the therapeutically effective amount of the activated SIRPα^{low} macrophages are administered IV and this administration is followed by tumor-directed in situ radiation therapy and by IV administration of ICB (FIG. 13J).

As shown in FIGS. 13K to 13R, activated SIRPα^{low} macrophages can also be co-cultured with cells from a tumor biopsy to produce tumor-specific peripheral blood T (PBT) cells (FIGS. 13K to 13N) or tumor infiltrating T lymphocyte (TIL) cells (FIGS. 13O to 13R).

In some embodiments, as alternatives to collecting a biological sample comprising PBMCs from the subject, the method will involve collecting a biological sample comprising blood from the subject, or collecting a biological sample comprising peripheral blood leukocytes from the subject, or collecting a biological sample comprising apheresis products from the subject, or collecting a biological sample comprising bone marrow from the subject, or collecting a biological sample comprising resected healthy tissue from the subject. Such biological samples may be used for isolating monocytes, for isolating macrophages, for isolating T cells, or for isolating other cells.

Methods for isolating monocytes from biological samples are well known in the art. Methods for isolating macrophages from biological samples are well known in the art. Methods for culturing monocytes in vitro to produce macrophages are well known in the art.

Disclosed herein are agents that inhibit the activity of SIRPα or disrupt its interaction with CD47. Without being bound by a theory, it is thought that inhibiting the activity or expression of SIRPα, or disrupting its interaction with CD47 enhances the phagocytic activity of a SIRPα-expressing cell and enhances the production of T cell-mediated adaptive immune responses.

The agent (SIRPα inhibitor) can be a chemical compound or an antibody (e.g., an anti-SIRPα monoclonal antibody) or other protein that suppresses the activity of SIRPα or disrupts its interaction with CD47. For example, the antibody or other protein can specifically bind a target such as SIRPα or a downstream component within a SIRPα-mediated pathway without activating the bound target. The agent can be, for example, a soluble CD47 extracellular domain or a fragment thereof that is engineered by molecular techniques to be the same as or different from a naturally occurring CD47 extracellular domain. Such agents can bind but not activate SIRPα, thereby disrupting SIRPα's interaction with CD47. The agent can be, for example, a soluble SIRPα extracellular domain or a fragment thereof that is engineered by molecular techniques to be the same as or different from a naturally occurring SIRPα extracellular domain. Such agents can bind but not activate CD47, thereby disrupting SIRPα's interaction with CD47. The agent can be a chemical compound or an antibody or other protein that causes a reduction in the amount of SIRPα that is present on the surface of a cell. The agent can be a chemical compound or an antibody or other protein that causes a reduction in the amount of SIRPα that is present on the surface of a cell by driving endocytosis of the surface-expressed SIRPα. The agent can be a chemical compound or an antibody or other protein that causes a reduction in the amount of SIRPα that is present on the surface of a cell by reducing the level of expression of the gene encoding SIRPα. The agent can be a cytokine, a growth factor, or a chemokine.

SIRPα can also be inhibited by inhibiting the SIRPα signaling pathway. Several tyrosine kinase inhibitors (e.g. those targeting a Src family tyrosine kinase and/or Btk) inhibit SIRPα cytoplasmic domain phosphorylation and recruitment of SHP-1/2. Accordingly, these agents are useful in the present methods. SIRPα can also be inhibited by inhibiting the SIRPα signaling pathway or elements thereof that lie further downstream than SHP-1/2.

Non-limiting examples of SHP-1 inhibitors that can be used in the disclosed methods includes: TPI-1 (0.1-5 mg/kg, 2-(2,5-Dichlorophenyl)-1,4-benzoquinone), TPI-1a1 (0.1-5 mg/kg, 2-(2,5-Dichlorophenyl)-2,4-benzoquinone), TPI-1a2 (0.1-5 mg/kg, 2-(3-chlorophenyl)-1,4-benzoquinone), TPI-1a3 (0.1-5 mg/kg, 2-phenylnaphthoquinone), TPI-1a4 (0.1-5 mg/kg, 2-(4-ethoxyphenyl)-1,4-benzoquinone), TPI-1a5 (0.1-5 mg/kg, 2-(4-methoxyphenyl)-1,4-benzoquinone), SSG (0.5-10 mg/kg, Sodium Stibogluconate), PTP Inhibitor I (0.5-10 mg/kg, 2-bromo-1-(4-hydroxyphenyl)-ethanone), PTP Inhibitor II (0.5-10 mg/kg, 2-bromo-1-(4-methoxyphenyl)-ethanone), PTP Inhibitor III (0.5-10 mg/kg, 2-[4-(2-bromoacetyl)phenoxy]-acetic acid), PTP Inhibitor IV (0.5-10 mg/kg, N,N′-[1,4-phenylenebis[(1-methylethylidene)-4,1-phenylene]]bis[1,1,1-trifluoro-methanesulfonamide), NSC 23922 (0.5-10 mg/kg, 3-Aminocholestane), and NSC 87877 (0.5-10 mg/kg, 8-hydroxy-7-[2-(6-sulfo-2-naphthalenyl)diazenyl]-5-quinolinesulfonic acid).

In some embodiments, the SIRPα inhibitor suppresses the expression of SIRPα, inhibits the activity of SIRPα, diminishes the abundance of SIRPα on the surface of a cell, disrupts the interaction between SIRPα and CD47, activates phagocytosis, or any combination thereof. Methods for knocking down expression of SIRPα in macrophages include in vitro treatment of macrophages with a cytokine or cocktail of cytokines, with a chemokine or cocktail of chemokines, with a growth factor or cocktail of growth factors, with a cocktail of cytokines, chemokines, and/or growth factors, with immune stimulatory molecules, with cell signaling proteins or other cell signaling molecules, or with combinations of any of the above. Knocking down expression of SIRPα in macrophages may also be done by stimulating cell surface receptors or other cell receptors. Such stimulation may be by cross-linking the receptors. Receptor crosslinking may be mediated by an antibody or cocktail of antibodies. Stimulation of cell receptors may also occur by treatment with a small molecule or drug.

Non-limiting examples of SIRPα inhibitors include: IFNγ, IL-6, IL-1 family cytokines (e.g. IL-1α, IL-1β, IL-18, IL-33, IL-36α, IL-36β, IL-36γ, IL-36Ra, IL-37, IL-38), IL-12, IFNα, IFNβ, tumor necrosis factor-alpha (TNFα), a Toll-like receptor (TLR) agonist or other molecules containing pathogen-associated molecular patterns (PAMPs) or damage-associated molecular patterns (DAMPs) (e.g. LPS, CpG, Poly I:C, LTA, PGN, flagellin, HMGB1, etc), Pam3CSK4, zymosan, a cytokine, a chemokine, a growth factor, and glucocorticoids such as methylprednisolone and dexamethasone. SIRPα inhibition may also be done by stimulating cell surface receptors or other cell receptors. Such stimulation may be by cross-linking the receptors. Receptor crosslinking may be mediated by an antibody or cocktail of antibodies. In some embodiments, the SIRPα inhibitor may be a combination of any of the SIRPα inhibitor agents listed.

In some embodiments, the SIRPα inhibitor is a mixture of 100 ng/mL IFNγ, 100 ng/mL IL-6, and 1 μg/mL CpG. In other embodiments, the SIRPα inhibitor is a mixture of IFNγ, IL-6, and CpG, wherein the concentration of IFNγ is 1, 5, 10, 20, 30, 40, 50, 60, 70, 80, 90, 110, 120, 130, 140, 150, 160, 170, 180, 190, 200, 300, 400, 500, or 1000 ng/mL, the concentration of IL-6 is 1, 5, 10, 20, 30, 40, 50, 60, 70, 80, 90, 110, 120, 130, 140, 150, 160, 170, 180, 190, 200, 300, 400, 500, or 1000 ng/mL, and the concentration of CpG is 1, 5, 10, 20, 30, 40, 50, 60, 70, 80, 90, 110, 120, 130, 140, 150, 160, 170, 180, 190, 200, 300, 400, or 500 nm/mL, or 1, 5, 10, 20, 30, 40, 50, 60, 70, 80, 90, or 100 μg/mL.

In some embodiments, the macrophage activating agent increases phagocytosis by macrophages, increases the antigen processing and presentation activities and functions of macrophages, increases the immunostimulatory capacity of macrophages, improves the T cell stimulation function of macrophages, promotes a pro-inflammatory (so-called M1) phenotype of macrophages, enables macrophages to change the TME to promote immune responses against cancer cells, or any combination thereof.

Non-limiting examples of macrophage activating agents include: IL-1 family cytokines (e.g. IL-1α, IL-1β, IL-18, IL-33, IL-36α, IL-36β, IL-36γ, IL-36Ra, IL-37, IL-38, or others that may be identified in the future), IL-12, IFNα, IFNβ, tumor necrosis factor-alpha (TNFα), a Toll-like receptor (TLR) agonist (e.g. LPS, CpG, Poly I:C, LTA, PGN, flagellin, Pam3CSK4, zymosan, HMGB1, etc) or other molecules containing pathogen-associated molecular patterns (PAMPs) or damage-associated molecular patterns (DAMPs), a cytokine, a chemokine, a growth factor, or glucocorticoids such as methylprednisolone and dexamethasone. Activating macrophages may also be done by stimulating cell surface receptors or other cell receptors. Such stimulation may be by cross-linking the receptors. Receptor crosslinking may be mediated by an antibody or cocktail of antibodies. Stimulation of cell receptors may also occur by treatment with a small molecule or drug (such as PKC activator phorbol 12-myristate 13-acetate (PMA), and protein tyrosine phosphatase inhibitors such as pervanadate). Macrophages may also be activated by PMA. As PMA is a PKC stimulator, it is an agent that activates macrophages by stimulating the PKC-Syk pathway. Biologically active variants of these activating agents can be used as well. The macrophage activating agent can also be a ligand for a TLR (e.g., lipopolysaccharide (LPS), polyinosinic:polycytidylic acid (poly I:C), lipoteichoic acid (LTA), flagellin, GARDIQUIMOD™ (an imidazoquinoline compound currently manufactured by InvivoGen; CAS number 1020412-43-4), IMIQUIMOD™ (1-isobutyl-1H-imidazo[4,5-c]quinoline-4-amine; CAS number 99011-02-6), peptidoglycan (PDG), or a CpG oligonucleotide). In some embodiments, the CpG oligonucleotide is a Class A oligonucleotide (ODN), a Class B ODN, a Class C ODN, or any combination thereof. In some embodiments, the CpG oligonucleotide is a Class B ODN. In some embodiments, the CpG oligonucleotide is ODN1826. In some embodiments, the CpG oligonucleotide is ODN BW006 (also known as ODN 684). Because both macrophages and some cancer cells (e.g., breast cancer cells) express TLRs, ligands for TLRs or agents that activate TLRs can be used as either a SIRPα inhibitor or macrophage activating agent in compositions and methods for activating macrophages and subsequently treating cancer. In some embodiments, the agent that activates macrophages, perhaps by disrupting the interaction between SIRPα and CD47 can be Surfactant Protein (e.g., Surfactant Protein A, B or D). Macrophages may also be activated by ionizing radiation.

In some embodiments, the macrophage activating agent is 20 nM phorbol 12-myristate 13-acetate (PMA). In other embodiments, the macrophage activating agent is PMA at a concentration of 0.5, 1, 2, 3, 4, 5, 6, 7, 8, 9, 10, 11, 12, 13, 14, 15, 16, 17, 18, 19, 25, 30, 40, 50, 60, 70, 80, 90, 110, 120, 130, 140, 150, 160, 170, 180, 190, 200, 300, 400, 500, or 1000 nM.

The agent that activates macrophage phagocytosis of cancer cells can be a small molecule, an amino acid, a peptide, a nucleic acid (e.g., RNAs or DNAs), a protein (e.g., an antibody) or a combination of one or more thereof. The agent can be naturally occurring, derived from a naturally existing agent, or synthesized. In some embodiments, the agent activates the PKC-Syk pathway in the subject. For example, the agent can be a cytokine (e.g., IL-17, IL-1β, IFNγ, IL-6, or a biologically active variant thereof). The agent can also be a lipopolysaccharide (LPS) or a biologically active variant thereof. In some embodiments, the agent can be IL-1, TNFα, PMA (phorbol 12-myristate 13-acetate), or a biologically active variant thereof. In certain embodiments, the disclosed method can include a step of identifying an agent that activates macrophage phagocytosis of cancer cells.

Where an agent is a nucleic acid, it can be a deoxyribonucleic acid (DNA), ribonucleic acid (RNA), or can be a DNA or RNA sequence that contains one or more and up to all artificial nucleic acid analogs. Agents comprising DNA sequences can include a plurality of nucleobases including cytosine, guanine, adenine, and thymine, as well as other natural or synthetic nucleobases, or combinations thereof. The nucleobases can also include derivatives of C, G, A, or T, or synthesized nucleobases. In certain embodiments, the DNA sequences can be in one or more conformations including A-DNA, B-DNA and Z-DNA. The DNA sequences can also be linear or branched. In certain embodiments, the DNA sequences can be single-stranded, double-stranded, or multiple-stranded.

In some embodiments, the RNA can be a messenger RNA (mRNA), transfer RNA (tRNA), ribosomal RNA (rRNA), transfer-messenger RNA (tmRNA), microRNA (miRNA), small interfering RNA (siRNA), CRISPR RNA, antisense RNA, pre-mRNA, or small nuclear RNAs (snRNA). The RNAs can also include a plurality of nucleobases including adenine, cytosine, guanine, or uracil, other natural nucleobases, or combinations thereof. In certain embodiments, the nucleobases can include derivatives of A, C, G, U, or synthesized nucleobases. The RNAs can also be in linear or branched. In certain embodiments, the RNAs can be single-stranded, double-stranded, or multi-stranded.

In some embodiments, the artificial nucleic acid analogs can include backbone analogues (e.g., hydrolysis resistant RNA-analogues, precursors to RNA world (e.g., TNA, GNA, PNA)) or base analogues (e.g., nucleobase structure analogues, fluorophores, fluorescent base analogues, natural non-canonical bases, base-pairs, metal-base pairs).

In some embodiments, the proteins can be antibodies including but not limited to antibodies of the IgG class, monoclonal antibodies, antibody fragments, single-chain antibodies or a single-chain variable fragment. The antibody can be naturally occurring or non-naturally occurring.

In some embodiments, CD47, SIRPα or the interaction therebetween can inhibit or deactivate one or more receptors. Thus, by inhibiting the expression or activity of SIRPα or suppressing the interaction between CD47 and SIRPα the agent can activate the one or more receptors. In certain embodiments, the one or more receptors can also be activated by the macrophage activating agent. Accordingly, by inhibiting the expression or activity of SIRPα or suppressing the interaction between CD47 and SIRPα the agent can enhance the activity of the one or more receptors.

The disclosure provides methods of producing activated SIRPα^{low} macrophages, comprising: (a) providing macrophages; and (b) bringing the macrophages in contact with a composition, wherein the composition comprises a SIRPα inhibitor, and an agent that enhances the phagocytic activity of the macrophages. In some embodiments, the SIRPα inhibitor is an agent that suppresses the expression of SIRPα. In some embodiments, step (a) comprises one or more of the following steps: (i) collecting a biological sample comprising peripheral blood mononuclear cells (PBMC) from the subject; (ii) isolating monocytes from the PBMC; and (iii) culturing the monocytes in vitro to produce macrophages.

In some embodiments, the methods comprise: (i) collecting a biological sample comprising peripheral blood mononuclear cells (PBMC) from the subject; (ii) isolating monocytes from the PBMC; (iii) culturing the monocytes in vitro to produce macrophages; and (iv) bringing the macrophages in contact with a composition, wherein the composition comprises an agent that suppresses the expression of SIRP-alpha, and an agent that enhances the phagocytic activity of the macrophages.

The disclosure also provides the activated SIRPα^{low} macrophages produced by the any one of the methods disclosed herein.

Further, the disclosure provides a composition comprising, an agent that suppresses the expression of SIRP-alpha, an agent that enhances the phagocytic activity of a macrophage, or a combination thereof. In some embodiments, the composition comprises an agent that suppresses the expression of SIRP-alpha and an agent that enhances the phagocytic activity of a macrophage.

In some embodiments, the agent that suppresses the expression of SIRP-alpha is a cytokine. In some embodiments, the cytokine is an inflammatory cytokine, such as, for example, an interferon. In some embodiments, the inflammatory cytokine is IFNγ, IFNα, IL-1, IL-6, or any combination thereof.

In some embodiments, the agent that enhances the phagocytic activity of a macrophage is a ligand for a Toll-like receptor, an interleukin, tumor necrosis factor-alpha (TNFα), or phorbol 12-myristate 13-acetate (PMA). In some embodiments, the ligand for a Toll-like receptor is a lipopolysaccharide (LPS), polyinosinic:polycytidylic acid (poly I:C), lipoteichoic acid (LTA), flagellin, imidazoquinoline, I-isobutyl-I H-imidazo[4,5-c]quinoline-4-amine, or a CpG oligonucleotide. In some embodiments, the ligand for a Toll-like receptor is polyinosinic:polycytidylic acid (poly I:C), a CpG oligonucleotide, or a combination thereof. In some embodiments, the interleukin is IL-1, IL-1α, IL-1β, IL-6, or IL-17.

In some embodiments, the composition comprises IFNγ, IFNα, CpG, poly I:C, or any combination thereof. In some embodiments, the composition comprises IFNγ, IFNα, a CpG oligonucleotide (e.g. ODN1826 and ODN BW006), and poly I:C. In some embodiments, the concentration of IFNγ in the composition is in the range of about 40 ng/ml to about 200 ng/ml, for example, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, or about 200 ng/ml, including all values and subranges that lie therebetween.

In some embodiments, the concentration of IFNα in the composition is in the range of about 40 ng/ml to about 200 ng/ml, for example, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, or about 200 ng/ml, including all values and subranges that lie therebetween.

In some embodiments, the concentration of the CpG oligodeoxynucleotide in the composition is in the range of about 1 μg/ml and about 10 μg/ml, for example, about 1.5 μg/ml, about 2 μg/ml, about 2.5 μg/ml, about 3 μg/ml, about 3.5 μg/ml, about 4 μg/ml, about 4.5 μg/ml, about 5 μg/ml, about 5.5 μg/ml, about 6 μg/ml, about 6.5 μg/ml, about 7 μg/ml, about 7.5 μg/ml, about 8 μg/ml, about 8.5 μg/ml, about 9 μg/ml, about 9.5 μg/ml, or about 10 μg/ml, including all values and subranges that lie therebetween.

In some embodiments, the concentration of Poly I:C in the composition is in the range of about 1 μg/ml and about 10 μg/ml, for example, about 1.5 μg/ml, about 2 μg/ml, about 2.5 μg/ml, about 3 μg/ml, about 3.5 μg/ml, about 4 μg/ml, about 4.5 μg/ml, about 5 μg/ml, about 5.5 μg/ml, about 6 μg/ml, about 6.5 μg/ml, about 7 μg/ml, about 7.5 μg/ml, about 8 μg/ml, about 8.5 μg/ml, about 9 μg/ml, about 9.5 μg/ml, or about 10 μg/ml, including all values and subranges that lie therebetween.

In some embodiments, the composition comprises about 40 ng/ml to about 200 ng/ml IFNγ, about 40 ng/ml to about 200 ng/ml IFNα, about 1 μg/ml and about 10 μg/ml CpG oligodeoxynucleotide and about 1 μg/ml and about 10 μg/ml Poly I:C. In some embodiments, the composition comprises about 100 ng/ml IFNγ, about 100 ng/ml IFNα, about 2 μg/ml CpG oligodeoxynucleotide, and about 2 μg/ml Poly I:C.

In some embodiments, the therapeutically effective amount of macrophages is 50 million macrophages, 150 million macrophages, or 450 million macrophages. In some embodiments, the therapeutically effective amount of macrophages is in the range of about 1 million to about 1000 million (for example, about 1 million, about 5 million, about 10 million, about 20 million, about 30 million, about 40 million, about 60 million, about 70 million, about 80 million, about 90 million, about 100 million, about 125 million, about 175 million, about 200 million, about 250 million, about 300 million, about 350 million, about 400 million, about 500 million, about 600 million, about 750 million, or about 1000 million, including all values and subranges that lie therebetween) macrophages. In some embodiments, the therapeutically effective amount of macrophages is a function of the size of the tumor mass. In some embodiments, the therapeutically effective amount of macrophages is a function of the weight of the patient. In some embodiments, the therapeutically effective amount of macrophages is a function of the age of the patient. In some embodiments, the therapeutically effective amount of macrophages is a function of a combination of the size of the tumor mass, the weight of the patient, and the age of the patient.

In some embodiments, the methods for treating cancer in a subject in need thereof, comprise administering to the subject a therapeutically effective amount of activated SIRPα^{low} macrophages, in combination with a secondary therapy (or secondary therapeutic) targeting cancer. In some embodiments, the secondary therapeutic targeting cancer promotes inflammation. The term administered “in combination,” as used herein, is understood to mean that two (or more) different treatments are delivered to the subject during the course of the subject's affliction with the disorder (such as, cancer), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” delivery.

In some embodiments, the treatment is more effective because of combined administration. For example, the second treatment is more effective; for e.g., an equivalent effect is seen with less of the second treatment, or the second treatment reduces symptoms to a greater extent, than would be seen if the second treatment were administered in the absence of the first treatment, or the analogous situation is seen with the first treatment. The effect of the two treatments can be partially additive, wholly additive, or greater than additive (synergistic).

In some embodiments, the secondary therapeutic that promotes inflammation is one or more damage-associated molecular patterns (DAMPs). Non-limiting examples of DAMPs include high-mobility group box 1 protein (HMGB1), heat shock protein (HSP), SNAP-associated protein (SNAPIN), versican, biglycan, decorin, eosinophil-derived neurotoxin, surfactant protein A/D, β-defensin 3, histone, serum amyloid A (SAA), β amyloid (Aβ), β2-glycoprotein I, mRNA, tenascin-C, S100 proteins, high-mobility group box 1 protein (HMGN1), biglycan, decorin, heparin sulfate, hyaluronic acid, fibrinogen, fibronectin, β-defensin 2, surfactant protein A/D, lactoferrin, neutrophil elastase, peroxiredoxin, histone, serum amyloid A (SAA), ox-LDL, IgG-ribonucleoprotein complex, microRNAs, mtDNA, F-actin, Sin3A-associated protein 130, β-glucosylceramide, N-glycans, monosodium urate (MSU), glucose, cholesterol crystals, ATP, oxidized 1-palmitoyl-2-arachidonylsn-glycero-3-phosphocholine (ox-PAPC), RNA transcribed from Alu elements (Alu-RNA), endogenous 5′ppp RNA, unedited long self-dsRNA, endogenous retroviral RNA, cytoplasmic DNA, damaged DNA in the nucleus, advanced glycation end products (AGEs), DNA, HSP70, peptidoglycan recognition protein 1 (PGLYRP1), actin, phosphatidic acid (PA), phosphatidylcholine (PC), phosphatidylethanolamine (PE), phosphatidylglycerol (PG), phosphatidylinositol (PI), phosphatidylserine (PS), cardiolipin, sulfatide, sphingomyelin, apolipoprotein A1 (APOA1), apolipoprotein A2 (APOA2), apolipoprotein B (APOB), apolipoprotein E (APOE), apolipoprotein J (APOJ), low-density lipoprotein (LDL), high-density lipoprotein (HDL), very-low-density lipoprotein (VLDL), Lp(a), HSP60, N-formylated peptides, cathepsin G, FAM19A4, annexin 1, Aβ42, serum amyloid A (SAA), low-density lipoprotein (LL-37) and other peptides, ATP, UTP, UDP, ADP, cyclic-GMP-AMP (cGAMP), Calcium ion, and ROS. In some embodiments, the DAMP comprises any DAMP described in Gong et al., Nature Reviews Immunology, Volume 20, February 2020, which is incorporated herein by reference in its entirety for all purposes.

In some embodiments, the secondary therapeutic that promotes inflammation is one or more ligands or other activators of a DAMP-sensing receptor. Non-limiting examples of DAMP-sensing receptors include toll-like receptor (TLR) (e.g., TLR2, TLR3, TLR4, TLR7, TLR9); C-type lectin receptor (CLR) (e.g., DNGR1, MINCLE, Dectin-1); NOD-like receptor (NLR) (e.g., NLR-family pyrin domain-containing 3); RIG-1 like receptor (RLR) (e.g., RIG-1, MDA5); cytosolic DNA sensors (CDS) (e.g., cyclic-GMP-AMP synthase (cGAS), AIM2); RAGE receptor; TREM (e.g. TREM1, TREM2); GPCR (e.g., FPR1, FPR2, P2Y2R, P2Y6R, P2Y12R, CaSR, GPRC6A); Stimulator of Interferon Genes (STING or transmembrane protein 173 (TMEM173)); and ion channels (e.g. TRPM2, other TRPs, P2X7R).

The disclosure further provides compositions comprising (a) the activated SIRPα^{low} macrophages disclosed herein, and (b) any one of the DAMPs disclosed herein, a ligand of any one of the DAMP-sensing receptors disclosed herein, or a combination thereof.

In some embodiments, the secondary therapeutic targets and/or inhibits the function of one or more T-cell inhibitory receptors (IRs). In some embodiments, the secondary therapeutic comprises one or more immunotherapeutics targeting one or more T-cell inhibitory receptors (IRs). Further details on T-cell inhibitory receptors (IRs) are found in Chauvin J-M, Zarour H M. TIGIT in cancer immunotherapy. Journal for ImmunoTherapy of Cancer 2020; 8:e000957, which is incorporated herein by reference in its entirety for all purposes. In some embodiments, the secondary therapeutic disrupts or inhibits the interaction between the T-cell inhibitory receptor (IR) and its ligand. In some embodiments, the secondary therapeutic is capable of binding to the T-cell inhibitory receptor (IR). In some embodiments, the secondary therapeutic is capable of binding to the ligand of the T-cell inhibitory receptor (IR). In some embodiments, the secondary therapeutic targeting one or more T-cell inhibitory receptors (IRs) is a small molecule.

In some embodiments, the secondary therapeutic is an antibody, or an antigen binding fragment thereof (e.g. a monoclonal antibody) that is capable of binding to the T-cell inhibitory receptor (IR). Non-limiting examples of T-cell inhibitory receptors (IRs) are programmed cell death receptor 1 (PD-1), programmed death-ligand 1 (PD-L1), anticytotoxic T lymphocyte-associated antigen 4 (CTLA-4), CD96/TACTILE, CD112R/PVRIG, DNAM-1/CD226, T cell immunoreceptor with immunoglobulin and ITIM domain (TIGIT), T cell immunoglobulin and mucin domain-containing molecule-3 (TIM-3) and lymphocyte activation gene 3 (LAG-3).

In some embodiments, the secondary therapeutic is an anti-PD-1 antibody, an anti-CTLA-4 antibody, an anti-TIGIT antibody, an anti-CD96 antibody, an anti-CD112R antibody, a anti-DNAM-1 antibody, an anti-TIM-3 antibody, an anti-PD-L1 antibody, an anti-LAG3 antibody, an anti-OX40 antibody, and anti-OX40L antibody, an antibody targeting a member of the Tumor Necrosis Factor (TNF) Receptor family, or any combination thereof. In some embodiments, the anti-TIGIT antibody is capable of binding to an immunoglobulin tail-tyrosine (ITT)-like motif of TIGIT. In some embodiments, the anti-TIGIT antibody is capable of binding to an immunoreceptor tyrosine-based inhibitory motif (ITIM) of TIGIT.

In some embodiments, the secondary therapeutic is an antibody, or an antigen binding fragment thereof (e.g. a monoclonal antibody) that is capable of binding to a ligand of the T-cell inhibitory receptor (IR). In some embodiments, the secondary therapeutic is an antibody, or an antigen binding fragment thereof (e.g. a monoclonal antibody) that is capable of binding to a ligand of TIGIT, such as, for example, CD155 (PVR/NECL-5), CD112 (PVRL2/nectin-2), or Fap2 protein from Fusobacterium nucleatum, an anaerobic Gram-commensal bacteria. Thus, in some embodiments, the secondary therapeutic is an anti-CD155 antibody, an anti-CD112 antibody, or an anti-Fap2 antibody.

In some embodiments, the secondary therapeutic comprises one or more immunotherapeutics targeting one or more different T-cell inhibitory receptors (IRs) and/or one or more ligands of the T-cell inhibitory receptors (IRs). Thus, in some embodiments, the secondary therapeutic comprises an anti-CD155 antibody, an anti-CD112 antibody, an anti-Fap2 antibody, an anti-PD-1 antibody, an anti-CTLA-4 antibody, an anti-TIGIT antibody, an anti-CD96 antibody, an anti-CD112R antibody, a anti-DNAM-1 antibody, an anti-TIM-3 antibody, an anti-PD-L1 antibody, an anti-LAG3 antibody, or any combination thereof.

In some embodiments, the anti-PD-1 antibody is pembrolizumab, nivolumab, or cemiplimab-rwlc. In some embodiments, the anti-PD-L1 antibody is atezolizumab, avelumab, or durvalumab. In some embodiments, the anti-CTLA-4 antibody ipilimumab. In some embodiments, the anti-TIGIT antibody is Tiragolumab, BMS-986207 (Bristol Myers Squibb), BGB-A1217 (BeiGene), OP-313M32 (Oncomed), AB154 (Arcus Biosciences), ASP8374 (Astella Pharma Global Development), MK-7684 (Merck Sharp & Dohme), or any combination thereof. In some embodiments, the anti-CD112R antibody is COM701 (Compugen). In some embodiments, the secondary therapeutic targeting DNAM-1 is LY3435151 (Eli Lilly and Company). In some embodiments the secondary therapeutic targeting OX40 is GSK998 (GSK).

In some embodiments, the secondary therapeutic is an antibody that can act as an agonist. In some embodiments, the secondary therapeutics is an antibody that can act as an antagonist.

The disclosure further provides compositions comprising (a) the activated SIRPα^{low} macrophages disclosed herein, and (b) any one or more of the immunotherapeutics targeting: one or more T-cell inhibitory receptors (IRs) and/or one or more ligands of the T-cell inhibitory receptors (IRs) disclosed herein. In some embodiments, the compositions comprise (a) the activated SIRPα^{low} macrophages disclosed herein, and (b) any one or more of: an anti-CD155 antibody, an anti-CD112 antibody, an anti-Fap2 antibody, an anti-PD-1 antibody, an anti-CTLA-4 antibody, an anti-TIGIT antibody, an anti-CD96 antibody, an anti-CD112R antibody, an anti-DNAM-1 antibody, an anti-TIM-3 antibody, an PD-L1 antibody, an anti-LAG3 antibody, an anti-OX40 antibody, an anti-OX40L antibody, or any combination thereof.

In some embodiments, the secondary therapy is radiation. In some embodiments, the method further involves treating the subject with an effective amount of tumor-directed in situ radiation therapy. For example, tumor-directed radiation may be administered in amounts of 0.1, 0.2, 0.3, 0.4, 0.5, 0.6, 0.7, 0.8, 0.9, 1, 1.5, 2, 3, 4, 5, 6, 7, 8, 9, 10, 12, 15, 20, or 25 Grays. Tumor-directed radiation may be administered in a single dose or may be administered in multiple doses. As disclosed herein, irradiation is done immediately before, immediately after, or concomitantly with the administration of macrophages. For example, irradiation can be administered 0, 0.5, 1, 2, 3, 4, 5, 6, 7, 8, 9, 10, 11, 12, 13, 14, 15, 16, 17, 18, 19, 20, 21, 22, 23, 24 hours before or after administration of macrophages. As other examples, irradiation can be administered 1.5, 2, 3, 4, 5, 6, 7, 8, 9, 10, 11, 12, 13, 14, 15, 16, 17, 18, 19, or 20 days before or after administration of macrophages.

In some embodiments, the radiation therapy is any form of energy or particle radiation commonly used in cancer treatment. In some embodiments, the radiation therapy is ionizing radiation. In some embodiments, the radiation is non-ionizing radiation. Non-ionizing radiation includes visible light, heat, radar, microwaves, and radio waves. Ionizing radiation includes x-rays, which is more energetic than non-ionizing radiation. Particle radiation includes alpha particles, beta particles, gamma rays, photons, carbon ions, heavy ions, muons, protons, electrons, and neutrons.

In some embodiments, the secondary therapy is an immune checkpoint inhibitor. In some embodiments, the method further involves treating the subject with an immune checkpoint inhibitor, also known as immune checkpoint blockade. Treating a subject with an immune checkpoint inhibitor is also known as “immune checkpoint inhibitor therapy” or “immune checkpoint blockade therapy.” In any of the present methods, the macrophages and the immune checkpoint inhibitor can be administered simultaneously by the same or different routes of administration or can be administered sequentially by the same or different routes of administration. For example, immune checkpoint inhibitor can be administered 0, 0.5, 1, 2, 3, 4, 5, 6, 7, 8, 9, 10, 11, 12, 13, 14, 15, 16, 17, 18, 19, 20, 21, 22, 23, 24 hours before or after administration of macrophages. As other examples, immune checkpoint inhibitor can be administered 1.5, 2, 3, 4, 5, 6, 7, 8, 9, 10, 11, 12, 13, 14, 15, 16, 17, 18, 19, or 20 days before or after administration of macrophages.

Where the agents are administered simultaneously by the same route of administration, the agents may be contained within a single formulation. Examples of immune checkpoint inhibitors include monoclonal antibodies targeted to PD-1 (e.g. KEYTRUDA® (pembrolizumab), OPDIVO® (nivolumab), or LIBTAYO® (cemiplimab-rwlc)), PD-L1 (e.g. TECENTRIQ® (atezolizumab), Bavencio® (avelumab), or IMFINZI® (durvalumab)), CTLA-4 (e.g. YERVOY® (ipilimumab)), or other immune checkpoint proteins that may be identified or approved for use in humans in the future.

In some embodiments, the secondary therapy is a chemotherapeutic agent. In some embodiments, the method further involves treating the subject with a chemotherapeutic agent. In some embodiments, the chemotherapeutic agent is one that increases tumor damaging signal. Non-limiting examples of known cancer drugs includes Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Breyanzi (Lisocabtagene Maraleucel), Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPOX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Danyelza (Naxitamab-gqgk), Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil—Topical), Fam-Trastuzumab Deruxtecan-nxki, Fareston (Toremifene), Farydak (Panobinostat Lactate), Faslodex (Fulvestrant), FEC, Fedratinib Hydrochloride, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, Fulphila (Pegfilgrastim), FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane 1131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, Ixempra (Ixabepilone), Jakafi (Ruxolitinib Phosphate), JEB, Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lisocabtagene Maraleucel, Lomustine, Lonsurf (Trifluridine and Tipiracil Hydrochloride), Lorbrena (Lorlatinib), Lorlatinib, Lumoxiti (Moxetumomab Pasudotox-tdfk), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Margenza (Margetuximab-cmkb), Margetuximab-cmkb, Marqibo (Vincristine Sulfate Liposome), Matulane (Procarbazine Hydrochloride), Mechlorethamine Hydrochloride, Megestrol Acetate, Mekinist (Trametinib Dimethyl Sulfoxide), Mektovi (Binimetinib), Melphalan, Melphalan Hydrochloride, Mercaptopurine, Mesnex (Mesna), Methotrexate Sodium, Methylnaltrexone Bromide, Midostaurin, Mitomycin, Mitoxantrone Hydrochloride, Mogamulizumab-kpkc, Monjuvi (Tafasitamab-cxix), Moxetumomab Pasudotox-tdfk, Mozobil (Plerixafor), MVAC, Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Naxitamab-gqgk, Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Nyvepria (Pegfilgrastim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Orgovyx (Relugolix), Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat Lactate, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perjeta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Pomalidomide, Pomalyst (Pomalidomide), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), Relugolix, R-EPOCH, Retacrit (Epoetin Alfa), Retevmo (Selpercatinib), Revlimid (Lenalidomide), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Siltuximab, Sipuleucel-T, Soltamox (Tamoxifen Citrate), Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosylate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Disodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomide, Temsirolimus, Tepadina (Thiotepa), Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib Dimethyl Sulfoxide, Trastuzumab, Trastuzumab and Hyaluronidase-oysk, Treanda (Bendamustine Hydrochloride), Trexall (Methotrexate Sodium), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ukoniq (Umbralisib Tosylate), Ultomiris (Ravulizumab-cwvz), Umbralisib Tosylate, Undencyca (Pegfilgrastim), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib, Zarxio (Filgrastim), Zejula (Niraparib Tosylate Monohydrate), Zelboraf (Vemurafenib), Zepzelca (Lurbinectedin), Zevalin (Ibritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), and Zytiga (Abiraterone Acetate).

In any of the present methods, the macrophages and the chemotherapeutic agent can be administered simultaneously by the same or different routes of administration or can be administered sequentially by the same or different routes of administration. For example, chemotherapeutic agent can be administered 0, 0.5, 1, 2, 3, 4, 5, 6, 7, 8, 9, 10, 11, 12, 13, 14, 15, 16, 17, 18, 19, 20, 21, 22, 23, 24 hours before or after administration of macrophages. As other examples, chemotherapeutic agent can be administered 1.5, 2, 3, 4, 5, 6, 7, 8, 9, 10, 11, 12, 13, 14, 15, 16, 17, 18, 19, or 20 days before or after administration of macrophages.

In some embodiments, the secondary therapy is an oncolytic virus therapy. In some embodiments, the method further involves treating the subject with an oncolytic virus therapy. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune system responses. Adenoviruses, herpes viruses, measles viruses, coxsackie viruses, polioviruses, reoviruses, poxviruses, filoviruses, coronaviruses, equine encephalitis viruses, flaviviruses, arenaviruses, influenza viruses, and Newcastle disease viruses, among others, are some of the oncolytic viruses under preclinical and clinical development for cancer therapy. In some embodiments, the oncoviruses is a Vaccinia virus (VACV) or Vesicular stomatitis virus (VSV).

In any of the present methods, the macrophages and the oncolytic virus therapy can be administered simultaneously by the same or different routes of administration or can be administered sequentially by the same or different routes of administration. For example, oncolytic virus therapy can be administered 0, 0.5, 1, 2, 3, 4, 5, 6, 7, 8, 9, 10, 11, 12, 13, 14, 15, 16, 17, 18, 19, 20, 21, 22, 23, 24 hours before or after administration of macrophages. As other examples, oncolytic virus therapy can be administered 1.5, 2, 3, 4, 5, 6, 7, 8, 9, 10, 11, 12, 13, 14, 15, 16, 17, 18, 19, or 20 days before or after administration of macrophages.

Also disclosed herein is a method for treating cancer in a subject that involves collecting a biological sample comprising peripheral blood mononuclear cells (PBMC) from the subject; isolating monocytes from the PBMC; isolating peripheral blood T (PBT) cells from the PBMC; culturing the monocytes in vitro to produce macrophages; contacting the macrophages with an SIRPα inhibitor to generate a population of macrophages with reduced SIRPα cell-surface expression or activity (SIRPα^{low} macrophages) relative to untreated macrophages; contacting the SIRPα^{low} macrophages with an macrophage activating agent to activate the SIRPα^{low} macrophages; collecting from the subject a biological sample comprising a tumor biopsy; in vitro co-culturing the activated SIRPα^{low} macrophages with cells from the tumor biopsy (tumor-fed SIRPα^{low} macrophages); in vitro co-culturing the tumor-fed SIRPα^{low} macrophages with the isolated PBT cells to expand the number of tumor-specific T cells; and administering to the subject to a therapeutically effective amount of the in vitro expanded PBT cells.

In some embodiments, the in vitro expanded PBT cells are administered to the subject by IV administration. In some embodiments, the in vitro expanded PBT cells are administered to the subject by IV administration followed by tumor-directed in situ radiation therapy. In some embodiments, the in vitro expanded PBT cells are administered to the subject by IV administration followed by IV administration of ICB. In some embodiments, the in vitro expanded PBT cells are administered to the subject by IV administration followed by tumor-directed in situ radiation therapy and by IV administration of ICB. In some embodiments, the in vitro expanded PBT cells are administered to the subject by IV administration preceded by tumor-directed in situ radiation therapy. In some embodiments, the in vitro expanded PBT cells are administered to the subject by IV administration preceded by tumor-directed in situ radiation therapy and followed by IV administration of ICB.

In some embodiments, the in vitro expanded PBT cells are administered to the subject by IV administration. In other embodiments, the in vitro expanded PBT cells are administered to the subject by intra-tumoral injection. In other embodiments, the in vitro expanded PBT cells are administered to the subject by injection in the tissue surrounding the tumor.

Also disclosed herein is a method for treating cancer in a subject that involves collecting a biological sample comprising peripheral blood mononuclear cells (PBMC) from the subject; isolating monocytes from the PBMC; culturing the monocytes in vitro to produce macrophages; contacting the macrophages with an SIRPα inhibitor to generate a population of macrophages with reduced SIRPα cell-surface expression or activity (SIRPα^{low} macrophages) relative to untreated macrophages; contacting the SIRPα^{low} macrophages with an macrophage activating agent to activate the SIRPα^{low} macrophages; collecting from the subject a biological sample comprising a tumor biopsy; isolating tumor infiltrating lymphocyte (TIL) cells from the tumor biopsy; in vitro co-culturing the activated SIRPα^{low} macrophages with tumor cells from the tumor biopsy (tumor-fed SIRPα^{low} macrophages); in vitro co-culturing the tumor-fed SIRPα^{low} macrophages with the isolated TIL cells to expand the number of tumor-specific T cells; and administering to the subject to a therapeutically effective amount of the in vitro tumor-specific T cells from TIL cells.

In some embodiments, the in vitro tumor-specific T cells from TIL cells are administered to the subject by IV administration. In some embodiments, the in vitro tumor-specific T cells from TIL cells are administered to the subject by IV administration followed by tumor-directed in situ radiation therapy. In some embodiments, the in vitro tumor-specific T cells from TIL cells are administered to the subject by IV administration followed by IV administration of ICB. In some embodiments, the in vitro tumor-specific T cells from TIL cells are administered to the subject by IV administration followed by tumor-directed in situ radiation therapy and by IV administration of ICB. In some embodiments, the in vitro tumor-specific T cells from TIL cells are administered to the subject by IV administration preceded by tumor-directed in situ radiation therapy. In some embodiments, the in vitro tumor-specific T cells from TIL cells are administered to the subject by IV administration preceded by tumor-directed in situ radiation therapy and followed by IV administration of ICB.

In some embodiments the TIL cells are tumor infiltrating T lymphocytes. In some embodiments, the in vitro tumor-specific T cells from TIL cells are administered to the subject by IV administration. In other embodiments, the in vitro tumor-specific T cells from TIL cells are administered to the subject by intra-tumoral injection. In other embodiments, the in vitro tumor-specific T cells from TIL cells are administered to the subject by injection in the tissue surrounding the tumor.

Various types of cancers and their metastases can be treated by the methods described herein. For example, the cancer can be adrenal cancer, anal cancer, bile duct cancer, bladder cancer, bone cancer, brain cancer, breast cancer, triple negative breast cancer, carcinoma, Castleman disease, cervical cancer, colon/rectum (colorectal) cancer, endometrial cancer, esophageal cancer, eye cancer, gallbladder cancer, gastrointestinal carcinoid tumors, gastrointestinal stromal tumor (gist), gestational trophoblastic disease, Hodgkin disease, Kaposi sarcoma, kidney cancer, laryngeal and hypopharyngeal cancer, leukemia, liver cancer, lung cancer, lymphoma, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma, skin cancer, small intestine cancer, stomach cancer, testicular cancer, thymus cancer, thyroid cancer, uterine sarcoma, vaginal cancer, vulvar cancer, Waldenstrom macroglobulinemia, Wilms tumor, melanoma, adenoma, carcinoma of solid tissue, carcinoma in situ, adenocarcinoma, hypoxic tumor, genitourinary cancer, head and neck cancer, nervous system cancer, benign lesion, or any combination thereof.

In some embodiments, the cancer is refractory to one or more of irradiation therapy, chemotherapy, or immunotherapy (e.g. checkpoint blockade). In some embodiments, the cancer is colorectal cancer, pancreatic cancer, ovarian, metastatic triple negative breast cancer, lung, or brain cancer.

The disclosed macrophages and/or immune checkpoint inhibitor (“agents”) can be administered orally or parenterally. Where the administration is parenteral, the agents can be administered intravenously, intratumorally, intramuscularly, subcutaneously, intraperitoneally, intrapleurally, intrabronchially, vaginally, topically, via the ear, eye, or nose, sublingually, intrathecally, rectally, intracranially, or into the cerebrospinal fluid.

In some embodiments of the treatment methods disclosed herein, the activated SIRPα^{low} macrophages, or compositions comprising the activated SIRPα^{low} macrophages disclosed herein are administered by injection, for example, by injection to the tumor site.

The mode of administration employed for the secondary therapeutic (e.g. any one of the DAMPs disclosed herein, any one of the ligands of the DAMP-sensing receptors disclosed herein, any one or more of the immunotherapeutics targeting: T-cell inhibitory receptors (IRs) and/or ligands of the T-cell inhibitory receptors (IRs) disclosed herein) may depend on the nature (e.g. site of cancer) and severity of the condition being treated, and be determined by the physician.

In some embodiments, the administration routes for any one of the secondary therapeutics, the activated SIRPα^{low} macrophages, or compositions comprising the activated SIRPα^{low} macrophages disclosed herein include oral, enteral, transmucosal, rectal, intranasal, buccal (e.g., sublingual), vaginal, intrathecal, intraocular, transdermal, in utero (or in ovo), parenteral (e.g., intravenous, subcutaneous, intradermal, intramuscular (including administration to skeletal, diaphragm and/or cardiac muscle), intradermal, intrapleural, intracerebral, intraarticular, intravascular or via infusion), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain).

In some embodiments, the administration of any one of the secondary therapeutics, the activated SIRPα^{low} macrophages, or compositions comprising activated SIRPα^{low} macrophages and one or more secondary therapeutics disclosed herein is via injection, for example, by injection to the tumor site.

In various embodiments, the compositions disclosed herein (e.g. compositions comprising the activated SIRPα^{low} macrophages) may be formulated in the form of a pill, a capsule, a granule, a tablet, a pallet, a suspension, an injection, an infusion, a suppository, a continuous delivery system, a syrup, a tincture, an ointment, a cream, eye drops, eardrops, a flush, a lavage, a slow absorbing depot, a dressing, a lozenge, or any pharmaceutically acceptable application or as a nutritional supplement.

The agents, as disclosed herein, can be formulated with conventional carriers and excipients, which can be selected in accord with ordinary practice. Tablets can typically contain excipients, glidants, fillers, binders and the like. Aqueous formulations can be prepared in sterile form, and when intended for delivery by other than oral administration generally can be isotonic. Formulations can contain excipients (e.g., excipients set forth in the Handbook of Pharmaceutical Excipients, 5th Ed.; Rowe, Sheskey, and Owen, Eds.; American Pharmacists Association; Pharmaceutical Press: Washington, D C, 2006). Excipients can include ascorbic acid or other antioxidants, chelating agents such as EDTA, carbohydrates such as dextrin, hydroxyalkylcellulose, hydroxyalkylmethylcellulose, stearic acid or the like.

When used for oral use, tablets, troches, lozenges, aqueous or oil suspensions, dispersible powders or granules, emulsions, hard or soft capsules, syrups or elixirs can be prepared. Compositions intended for oral use can be prepared according to any method known to the art for the manufacture of pharmaceutical compositions and such compositions may contain one or more agents including sweetening agents, flavoring agents, coloring agents and preserving agents, in order to provide a palatable preparation.

In some embodiments, the activated SIRPα^{low} macrophages, or the compositions comprising the activated SIRPα^{low} macrophages disclosed herein are in the form of a sterile injectable preparation (e.g., a sterile injectable aqueous or oleaginous suspension). The suspension can be formulated according to methods known in the art using suitable dispersing or wetting agents and suspending agents. The sterile injectable preparation can also be a sterile injectable solution or suspension in a non-toxic parenterally acceptable diluent or solvent (e.g., a solution in 1,3-butane-diol or prepared as a lyophilized powder). Among the acceptable vehicles and solvents that can be employed are water, Ringer's solution and isotonic sodium chloride solution. In some embodiments, the vehicle is a buffer, such as, for example, phosphate buffered saline (PBS). In some embodiments, the PBS comprises at least about 0.1% (for example, about 0.3%, about 0.5%, about 0.7%, about 0.9%, about 1.1% or about 1.3%, including all values and subranges that lie therebetween) sodium chloride. In some embodiments, the PBS has a pH in the range of about 7 to 8, for example, about 7.2, about 7.4, about 7.6, or about 7.8, including all values and subranges that lie therebetween. In some embodiments, the PBS comprises about 0.9% sodium chloride, and a pH of about 7.4. In addition, sterile fixed oils can be conventionally employed as a solvent or suspending medium. For this purpose, any bland fixed oil can be employed (e.g., synthetic mono- or diglycerides). Fatty acids (e.g., oleic acid) can also be used in the preparation of injectables. In some embodiments, any of the disclosed compositions may further comprise serum, such as, human serum. In some embodiments, the serum is GMP manufactured human AB serum.

The formulations can be presented in unit dose or multi-dose containers (e.g., sealed ampoules and vials) and can be stored in a freeze-dried (lyophilized) condition requiring the addition of the sterile liquid carrier (e.g., water) for injection, immediately prior to use. Extemporaneous injection solutions and suspensions can be prepared from sterile powders, granules and tablets of the kind previously described. Preferred unit dosage formulations can be those containing a daily dose or unit daily sub-dose, as herein above recited, or an appropriate fraction thereof, of the active ingredient.

The formulations can be presented in unit dose or multi-dose containers (e.g., sealed ampoules and vials) and can be stored in a frozen (wet) condition requiring the thawing of the formulation for injection, immediately prior to use. Extemporaneous injection solutions and suspensions can be prepared by thawing frozen formulations. The formulated suspension can be presented in unit dose or multi-dose containers (e.g., sealed ampoules and vials) and can be stored in a frozen (wet) condition requiring the thawing of the formulated suspension, followed by centrifugation of the suspension and resuspension of the centrifugated pellet in fresh sterile injectable vehicle, diluent, or solvent. Preferred unit dosage formulations can be those containing a daily dose or unit daily sub-dose, as herein above recited, or an appropriate fraction thereof, of the active ingredient.

If desired, the compounds of the presently disclosed subject matter can be applied in conjunction with one or more inert or inactive ingredients. The first agent and/or the second agent, as disclosed herein, can be administered by any route appropriate to the condition to be treated. Suitable routes can include oral, rectal, nasal, topical (including buccal and sublingual), vaginal and parenteral (including subcutaneous, intramuscular, intravenous, intradermal, intrathecal and epidural), and the like.

In some embodiment, the disclosed SIRPα inhibitors, macrophage activators, and radiation can also be used in combination with other active ingredients. The combinations can be selected based on the condition to be treated, cross-reactivities of ingredients and pharmaco-properties of the combination. The agents can also be combined with one or more other active ingredients in a unitary dosage form for simultaneous or sequential administration to a patient. The combination therapy can be administered as a simultaneous or sequential regimen. When administered sequentially, the combination can be administered in two or more administrations.

In general, during alternation therapy, an effective dosage of each active ingredient can be administered sequentially (i.e., serially), whereas in combination therapy, effective dosages of two or more active ingredients can be administered together. The combination therapy may provide “synergy” or a “synergistic effect” (i.e., the effect achieved when the active ingredients used together is greater than the sum of the effects that results from using the compounds separately). In certain embodiments, a synergistic effect can be attained when the active ingredients are: (1) co-formulated and administered or delivered simultaneously in a combined formulation; (2) delivered by alternation or in parallel as separate formulations; or (3) by some other regimen. In alternation therapy, the synergistic effect can also be attained when the compounds are administered or delivered sequentially (e.g., in separate tablets, pills, or capsules, or by different injections in separate syringes).

### Aspects of the Disclosure

Aspect 1. A method for producing activated SIRPα^{low} macrophages, comprising
(a) isolating monocytes from peripheral blood mononuclear cells (PBMC) in a biological sample;
(b) differentiate the monocytes in vitro to produce macrophages; and
(c) contacting the macrophages with an SIRPα inhibitor; and
(d) contacting the macrophages with macrophage activating agent,
thereby generating a population of macrophages with marked reduction of SIRPα cell-surface expression (SIRPα^{low}), relative to untreated macrophages,
wherein the SIRPα^{low} macrophages have activated phagocytosis towards cancer cells, increased proinflammatory response, and increased immunogenic antigen presentation.

Aspect 2. The method of aspect 1, wherein the SIRPα inhibitor suppresses the expression of SIRPα, diminishes the abundance of SIRPα on the surface of a cell, inhibits the activity of SIRPα, disrupts the interaction between SIRPα and CD47, or a combination thereof.

Aspect 3. The method of aspect 1 or 2, wherein the SIRPα inhibitor comprises a cytokine, a TLR ligand, a glucocorticoid, or a combination thereof.

Aspect 4. The method of aspect 3, wherein the SIRPα inhibitor is selected from the group consisting of IFNα, IFNβ, IFNγ, IL-1, IL-6, IL-12, IL-18, LPS, CpG, Poly I:C, LTA, PGN, flagellin, Pam3CSK4, zymosan, and HMGB1.

Aspect 5. The method of any one of aspects 1 to 4, wherein the macrophage activating agent comprises a cytokine, a phorbol ester, a TLR ligand, or a combination thereof.

Aspect 6. The method of aspect 5, wherein the cytokine is selected from the group consisting of IFNα, IFNβ, IL-6, IL-1, IL-17, IL-18, TNFα, and IL-12.

Aspect 7. The method of aspect 5 or 6, wherein the phorbol ester comprises phorbol 12-myristate 13-acetate (PMA).

Aspect 8. The method of any one of aspects 5 to 7, wherein the TLR ligand is selected from the group consisting of LPS, CpG, Poly I:C, LTA, PGN, flagellin, Pam3CSK4, zymosan, and HMGB1.

Aspect 9. The method of any one of aspects 8 to 11, wherein the glucocorticoid comprises methylprednisolone or dexamethasone.

Aspect 10. The method of any one of aspects 1 to 10, wherein the SIRPα inhibitor and macrophage activating agent are contacted with the macrophages sequentially.

Aspect 11. The method of any one of aspects 1 to 10, wherein the SIRPα inhibitor and macrophage activating agent are contacted with the macrophages simultaneously or concurrently.

Aspect 12. The method of any one of aspects 1 to 10, wherein the SIRPα inhibitor and macrophage activating agent are present in the same composition.

Aspect 13. The method of aspect 12, wherein the composition comprises recombinant human interferon-gamma (IFNγ), recombinant human interferon-alpha A2 (IFNα), CpG oligodeoxynucleotide, and polyinosinic:polycytidylic acid (Poly I:C).

Aspect 14. The method of any one of aspects 1 to 13, wherein the SIRPα inhibitor comprises a SHP-1 inhibitor.

Aspect 15. The method of aspect 14, wherein the SHP-1 inhibitor is selected from the group consisting of TPI-1 (2-(2,5-Dichlorophenyl)-1,4-benzoquinone), TPI-1a1 (2-(2,5-Dichlorophenyl)-2,4-benzoquinone), TPI-1a2 (2-(3-chlorophenyl)-1,4-benzoquinone), TPI-1a3 (2-phenylnaphthoquinone), TPI-1a4 (2-(4-ethoxyphenyl)-1,4-benzoquinone), TPI-1a5 (2-(4-methoxyphenyl)-1,4-benzoquinone), SSG (Sodium Stibogluconate), PTP Inhibitor I (2-bromo-1-(4-hydroxyphenyl)-ethanone), PTP Inhibitor II (2-bromo-1-(4-methoxyphenyl)-ethanone), PTP Inhibitor III (2-[4-(2-bromoacetyl)phenoxy]-acetic acid), PTP Inhibitor IV (N,N′-[1,4-phenylenebis[(1-methylethylidene)-4,1-phenylene]]bis[1,1,1-trifluoro-methanesulfonamide), NSC 23922 (3-Aminocholestane), and NSC 87877 (8-hydroxy-7-[2-(6-sulfo-2-naphthalenyl)diazenyl]-5-quinolinesulfonic acid).

Aspect 16. The method of any one of aspects 1 to 13, further comprising contacting the macrophages with a SHP-1 inhibitor.

Aspect 17. The method of aspect 16, wherein the SHP-1 inhibitor is an irreversible SHP-1 inhibitor.

Aspect 18. A composition comprising activated SIRPα^{low} macrophages produced by the method of any one of aspects 1 to 12.

Aspect 19. A method for producing in vitro expanded tumor-specific peripheral blood T (PBT) cells, comprising:
(a) isolating peripheral blood T (PBT) cells from a biological sample;
(b) in vitro co-culturing activated SIRPα^{low} macrophages produced by the method of claim 1 with cells from the tumor biopsy to produce tumor-fed SIRPα^{low} macrophages;
(c) in vitro co-culturing the tumor-fed SIRPα^{low} macrophages with isolated PBT cells to expand the number of tumor-specific T cells, thereby producing in vitro expanded tumor-specific PBT cells.

Aspect 20. A composition comprising in vitro expanded tumor-specific PBT cells produced by the method of aspect 19.

Aspect 21. A method for producing in vitro expanded tumor infiltrating T lymphocyte (TIL) cells, comprising:
(a) isolating tumor infiltrating T lymphocyte (TIL) cells from a tumor biopsy;
(b) in vitro co-culturing activated SIRPα^{low} macrophages produced by the method of claim 1 with tumor cells from the tumor biopsy to produce tumor-fed SIRPα^{low} macrophages;
(c) in vitro co-culturing the tumor-fed SIRPα^{low} macrophages with isolated TIL cells to expand the number of tumor-specific T cells, thereby producing in vitro expanded tumor-specific T cells from TIL.

Aspect 22. A composition comprising in vitro tumor-specific T cells from TIL cells produced by the method of aspect 21.

Aspect 23. A method for treating a tumor in a subject, comprising administering to the subject to a therapeutically effective amount of the activated macrophages aspect claim 18, the in vitro expanded tumor-specific PBT cells of aspect 20, the in vitro tumor-specific T cells from TIL cells of aspect 22, or any combination thereof.

Aspect 24. The method of 23, further comprising treating the subject with tumor-directed irradiation.

Aspect 25. The method of aspect 23 or 24, further comprising administering to the subject to a therapeutically effective amount of an immune checkpoint inhibitor.

Aspect 26. The method of aspect 25, wherein the immune checkpoint inhibitor comprises anti-PD1, anti-PD-L1, anti-CTLA4 antibodies, or a combination thereof.

Aspect 27. The method of any one of aspects 23 to 26, wherein the subject is refractory to PD-1 blockade.

Aspect 28. The method of any one of aspects 23 to 27, further comprising treating the subject with an oncolytic virus.

Aspect 29. The method of aspect 23, wherein the oncolytic virus is a vesicular stomatitis virus.

Aspect 30. A composition comprising recombinant human interferon-gamma (IFNγ), recombinant human interferon-alpha A2 (IFNα), a CpG oligodeoxynucleotide, and polyinosinic:polycytidylic acid (Poly I:C).

Aspect 31. The composition of aspect 30, wherein the IFNγ is present at a concentration of 40-200 ng/ml.

Aspect 32. The composition of aspect 30 or 31, wherein the IFNα is present at a concentration of 40-200 ng/ml.

Aspect 33. The composition of any one of aspect 25 to 27, wherein the CpG oligodeoxynucleotide is present at a concentration of 1-5 μg/ml.

Aspect 34. The composition of any one of aspect 30 to 33, wherein the Poly I:C is present at a concentration of 1-5 μg/ml.

Aspect 35. A composition comprising activated SIRPα^{low} macrophages produced by a method comprising contacting macrophages from a subject with an effective amount of the composition of any one of aspect 30 to 34.

Aspect 36. The method of aspect 35, wherein the macrophages are bone marrow-derived macrophages or monocyte-derived macrophages.

Aspect 37. A method for treating a tumor in a subject, comprising administering to the subject to a therapeutically effective amount of a SH-domain containing tyrosine phosphatase-1 (SHP-1) inhibitor and a therapeutically effective amount of radiation therapy, an immune checkpoint inhibitor, an oncolytic virus, or a combination thereof.

Aspect 38. The method of aspect 37, wherein the immune checkpoint inhibitor comprises anti-PD1, anti-PD-L1, anti-CTLA4 antibodies, or a combination thereof.

Aspect 39. The method of aspect 37, wherein the SHP-1 inhibitor is selected from the group consisting of TPI-1 (2-(2,5-Dichlorophenyl)-1,4-benzoquinone), TPI-1a1 (2-(2,5-Dichlorophenyl)-2,4-benzoquinone), TPI-1a2 (2-(3-chlorophenyl)-1,4-benzoquinone), TPI-1a3 (2-phenylnaphthoquinone), TPI-1a4 (2-(4-ethoxyphenyl)-1,4-benzoquinone), TPI-1a5 (2-(4-methoxyphenyl)-1,4-benzoquinone), SSG (Sodium Stibogluconate), PTP Inhibitor I (2-bromo-1-(4-hydroxyphenyl)-ethanone), PTP Inhibitor II (2-bromo-1-(4-methoxyphenyl)-ethanone), PTP Inhibitor III (2-[4-(2-bromoacetyl)phenoxy]-acetic acid), PTP Inhibitor IV (N,N′-[1,4-phenylenebis[(1-methylethylidene)-4,1-phenylene]]bis[1,1,1-trifluoro-methanesulfonamide), NSC 23922 (3-Aminocholestane), and NSC 87877 (8-hydroxy-7-[2-(6-sulfo-2-naphthalenyl)diazenyl]-5-quinolinesulfonic acid).

Aspect 40. The method of any one of aspects 23-29, further comprising administering to the subject one or more damage-associated molecular patterns (DAMPs).

Aspect 41. The method of aspect 40, wherein the one or more DAMPs comprises HMGB1, heat shock protein (HSP), SNAP-associated protein (SNAPIN), versican, biglycan, decorin, eosinophil-derived neurotoxin, surfactant protein A/D, β-defensin 3, histone, serum amyloid A (SAA), β amyloid (Aβ), β2-glycoprotein I, mRNA, tenascin-C, S100 proteins, high-mobility group box 1 protein (HMGN1), biglycan, decorin, heparin sulfate, hyaluronic acid, fibrinogen, fibronectin, β-defensin 2, surfactant protein A/D, lactoferrin, neutrophil elastase, peroxiredoxin, histone, serum amyloid A (SAA), ox-LDL, IgG-ribonucleoprotein complex, microRNAs, mtDNA, F-actin, Sin3A-associated protein 130, β-glucosylceramide, N-glycans, monosodium urate (MSU), glucose, cholesterol crystals, ATP, oxidized 1-palmitoyl-2-arachidonylsn-glycero-3-phosphocholine (ox-PAPC), RNA transcribed from Alu elements (Alu-RNA), endogenous 5′ppp RNA, unedited long self-dsRNA, endogenous retroviral RNA, cytoplasmic DNA, damaged DNA in the nucleus, advanced glycation end products (AGEs), DNA, HSP70, peptidoglycan recognition protein 1 (PGLYRP1), actin, phosphatidic acid (PA), phosphatidylcholine (PC), phosphatidylethanolamine (PE), phosphatidylglycerol (PG), phosphatidylinositol (PI), phosphatidylserine (PS), cardiolipin, sulfatide, sphingomyelin, apolipoprotein A1 (APOA1), apolipoprotein A2 (APOA2), apolipoprotein B (APOB), apolipoprotein E (APOE), apolipoprotein J (APOJ), low-density lipoprotein (LDL), high-density lipoprotein (HDL), very-low-density lipoprotein (VLDL), Lp(a), HSP60, N-formylated peptides, cathepsin G, FAM19A4, annexin 1, Aβ42, serum amyloid A (SAA), low-density lipoprotein (LL-37) and other peptides, ATP, UTP, UDP, ADP, cyclic-GMP-AMP (cGAMP), Calcium ion, ROS, or any combination thereof.

42. The method of any one of aspects 23-29, 40 and 41, further comprising administering to the subject, an anti-CD155 antibody, an anti-CD112 antibody, an anti-Fap2 antibody, an anti-TIGIT antibody, an anti-CD96 antibody, an anti-CD112R antibody, a anti-DNAM-1 antibody, an anti-TIM-3 antibody, an anti-LAG3 antibody, or any combination thereof.

43. The method of aspect 42, wherein the anti-TIGIT antibody is tiragolumab, BMS-986207, BGB-A1217, OP-313M32, AB154, ASP8374, MK-7684, or any combination thereof.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

Immune checkpoint blockade (ICB) is lauded for its exceptional efficacy in several types of cancers (Wei, S. C., et al. Cancer Discov., 2018. 8(9):1069-1086). Unfortunately, many cancer patients fail to respond or become refractory to ICB, which has been attributed to tumors and the tumor microenvironment (TME) co-opting mechanisms to subvert T cell immunity (Jenkins, R. W., et al. British Journal Of Cancer, 2018. 118:9). Particularly, colorectal cancer (CRC) and pancreatic cancer, especially pancreatic ductal adenocarcinoma (PDA), are well-known for exhibiting limited, poor responses to ICB (<11% for CRC, <4% for PDA) (Brahmer, J. R., et al. N Engl J Med., 2012. 366(26):2455-2465). Although CRC and PDA are associated with a high mutational burden and therefore should be immunogenic, both CRC and PDA exhibit a paucity of cytotoxic CD8 T cells (Tc) and strong immunosuppressive TMEs highly populated by T_{REGS} and myeloid-derived suppressor cells (MDSC), thereby undermining the efficacy of ICB (Kabacaoglu, D., et al. Frontiers in Immunology, 2018. 9 (1878); Emambux, S., et al. Expert Opin Biol Ther, 2018. 18(5): 561-573). Thus, there is an urgent need for therapeutic innovation to improve ICB efficacy in ICB-resistant cancers such as CRC and PDA.

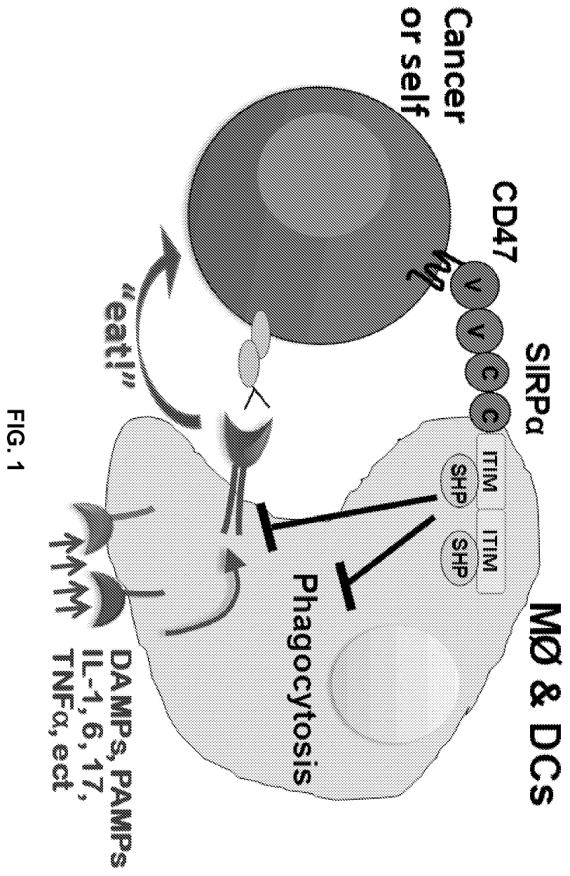

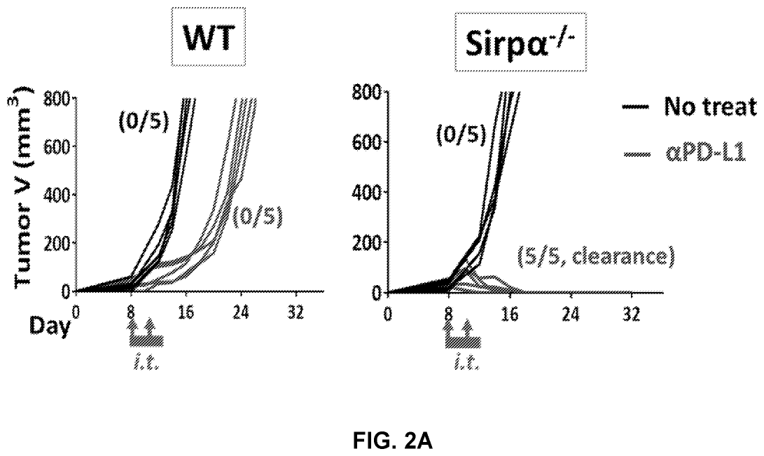

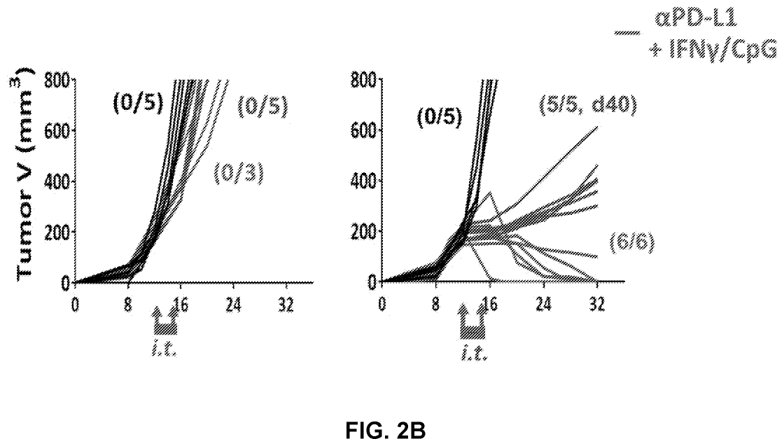

Anti-PD-L1 antibody (αPD-L1 Ab) administration in SIRPα-deficient mice (Sirpα^{-/-}) led to profound anti-tumor immunity, achieving complete elimination of CRC and PDA in situ, with robustness that was not observed in WT mice and rarely reported elsewhere. SIRPα is an immunoreceptor tyrosine-based inhibitory motif (ITIMs)-containing signaling receptor whose canonical function, via interacting with the self-marker CD47, is to inhibit professional phagocytes (e.g. macrophages (MØs) dendritic cells (DCs)) from phagocytosing self/tumor-cells (FIG. 1) (Veillette, A., et al. Trends Immunol, 2018. 39(3):173-184). Despite the fact that many cancers exploit this mechanism by increasing CD47 expression (Willingham, S. B., et al. Proc Natl Acad Sci USA, 2012. 109(17):6662-7), mere depletion of the CD47-SIRPα axis or SIRPα signaling does not lead to phagocytosis—an activity that can not occur unless phagocytes are simultaneously stimulated by activation mechanisms such as those mediated by TLR agonists or proinflammatory cytokines (depicted in FIG. 1) (Bian, Z., et al. Proc Natl Acad Sci USA, 2016. 113(37):E5434-43). Indeed, Sirpα^{-/-} mice showed minimal immune control in the absence of ICB against syngeneic, non-immunogenic MC38 (CRC) and Panc02 and KPC (PDA). All these tumors were tolerated and grew to form palpable primary tumors after subcutaneous (s.c.) engraftment similar to that in WT mice (FIGS. 2 & 3). However, tumor-engrafted Sirpα^{-/-} mice exhibited a higher basal number of tumor-infiltrated T cells than WT mice. Given that phagocytes, especially MØs, are found to be abundant in these tumors (Cassetta, L., et al. Nature Reviews Drug Discovery, 2018. 17:887), there was a question whether ICB—which would further activate Tc to induce cytotoxic cell injury, DAMP release, and locally increase proinflammatory cytokines (e.g. IFN-γ, TNFα and IL-17)—would activate tumor-associated phagocytes in Sirpα^{-/-} mice to eliminate their tumors given their absence of SIRPα.

Two doses of αPD-L1 (50 μg each, BioXcell clone 10F.9G2) given to MC38 tumors (s.c.) induced robust anti-tumor immune responses in Sirpα^{-/-} mice (FIG. 2), resulting in direct elimination of tumors with sizes≤50 mm³ or strong suppression of growth when tumors were relatively larger (>200 mm³). Although increasing the number of doses of αPD-L1 to eliminate larger tumors in Sirpα^{-/-} mice would likely confer a similar result, IFN-γ plus CpG was used along with αPD-L1 for two reasons: 1) it was believed that addition of pro-inflammatory cytokines/TLR agonists would ensure robust activation of tumor-associated Sirpα^{-/-} phagocytes and 2) focusing on activation of intratumoral phagocytes and their activity would demonstrate the magnitude of impact Sirpα^{-/-} phagocytes contribute to tumor elimination rather than further reinvigoration of exhausted T cells. Indeed, combining IFN-γ and CpG with αPD-L1 enhanced immunogenicity and led to complete elimination of the larger MC38 tumors in Sirpα^{-/-} mice (FIG. 2B). The same αPD-L1 treatment(s) in WT mice produced only partial effects when tumors were small, or otherwise had nearly no beneficial effects when tumors grew large, even with CpG combination. (Note: these preliminary studies were done giving αPD-L1 Ab via intratumoral (i.t.) injection, instead of intraperitoneal (i.p.), for the sake of saving reagents).

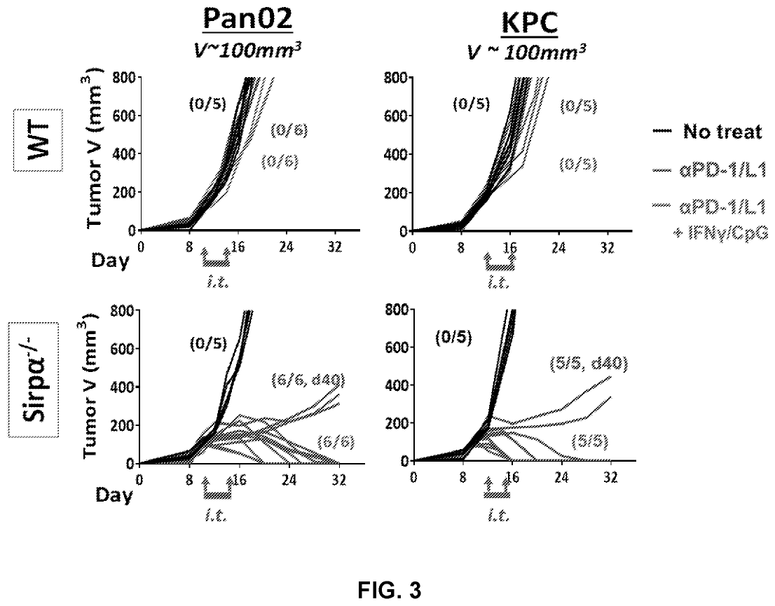

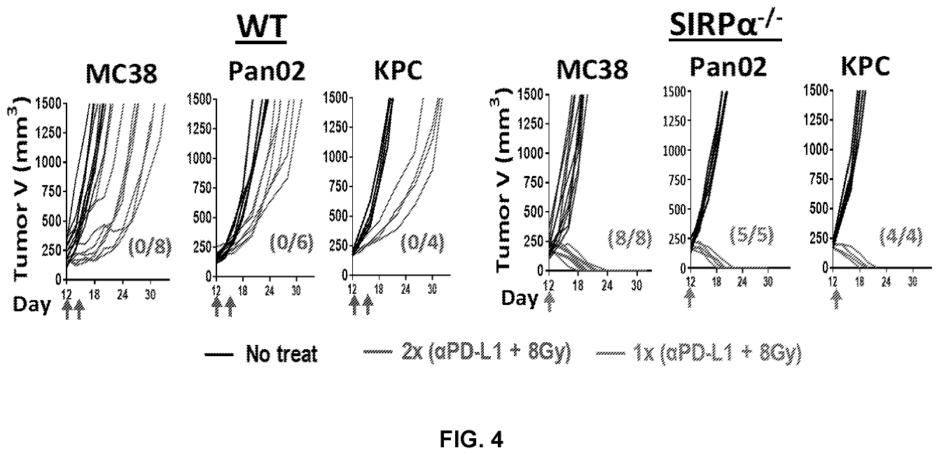

αPD-L1 treatment was also tested against PDA tumors Panc02 and KPC engrafted (s.c.) in Sirpα^{-/-} mice and, again, complete responses were observed (FIG. 3). In these experiments, tumors were ˜100 mm³ when the first of two doses of αPD-L1±IFNγ/CpG were given, while the second dose was given 3 d later. In Sirpα^{-/-} mice, αPD-L1 alone strongly suppressed Panc02 and KPC tumor growth, and in some cases was sufficient for complete remission, whereas combining IFNγ/CpG consistently eliminated these tumors completely. The same treatments, however, showed trivial effects in WT mice, in which tumors continued growing and soon reached the humane endpoint. Since clinical trials have been assessing the combination of tumor radiation with checkpoint blockade (Gong, J., et al. J Immunother Cancer, 2018 6(1):46), this method for enhancing αPD-L1 efficacy was tested. Remarkably, treating Sirpα^{-/-} mice with a single X-ray fraction of 8Gy (RT) followed by αPD-L1 administration immediately after led to complete elimination of MC38, Panc02 and KPC tumors of sizes even >250 mm³ (FIG. 4). In stark contrast, WT mice treated with two rounds of radiation plus αPD-L1 only achieved minor control of tumor progression. These tumor models, especially the MC38 tumor, are extensively studied in preclinical immunotherapy and radiation therapy (Deng, L., et al. J Clin Invest, 2014. 124(2):687-695; Vatner, R. E., et al. Semin Radiat Oncol., 2015. 25(1):18-27; Ahn, G.-O., et al. Proc Natl Acad Sci USA., 2010. 107(18):8363-8368), partially due to their notoriously low immunogenicity, thereby facilitating the identification of interventions that might be useful for those hard-to-treat cancers in the clinical setting. Except for efficacies shown only against relatively small sized tumors (Goding, S. R., et al. J Immunol., 2018. 200(9):3304-3311; Smilowitz, H. M., et al. Cancer Immunol Immunother., 2016. 65(2):127-139; Filatenkov, A., et al. Clin Cancer Res., 2015. 21(16):3727-3739; Twyman-Saint Victor, C., et al. Nature, 2015. 520(7547):373-7; Azad, A., et al. EMBO Mol Med., 2017. 9(2): 167-180), this magnitude of drastic tumor regression and remission of even large MC38 and PDA tumors in Sirpα^{-/-} mice is rarely seen or found in the literature.

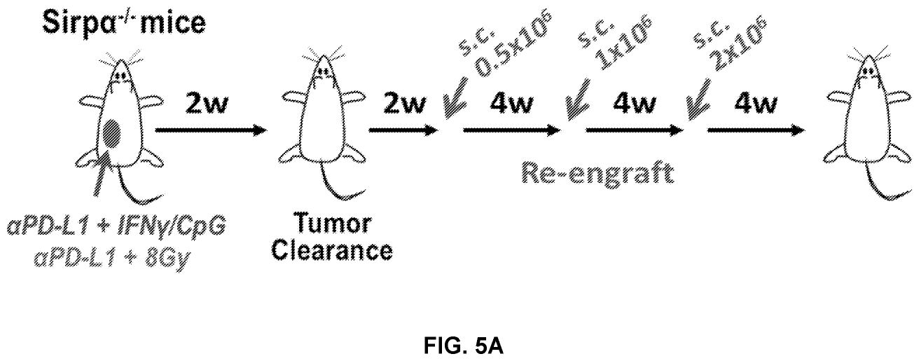

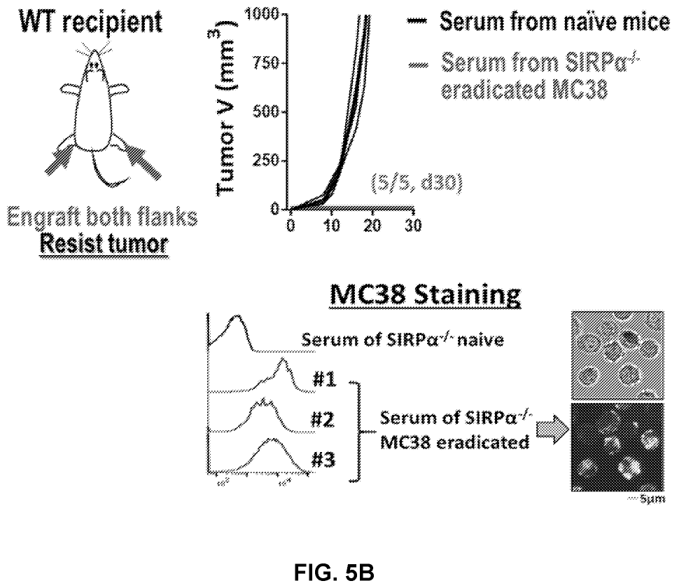

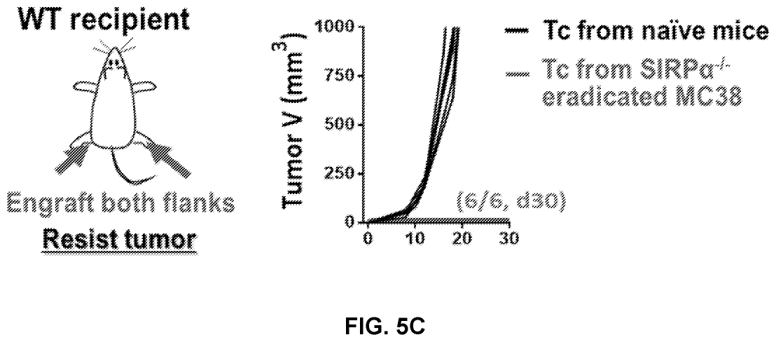

One study (Deng, L., et al. J Clin Invest, 2014. 124(2):687-695) treating small MC38 tumors of ˜50 mm³ with 20Gy radiation and four doses of αPD-L1 combination induced durable tumor regression; however, relapse occurred 10 days post-cessation of treatment. While another study treating Panc02 and KPC tumors of ˜100 mm³ with 12Gy radiation and four doses of αPD-L1 only achieved tumor growth delay (Azad, A., et al. EMBO Mol Med., 2017. 9(2): 167-180). Notably, all Sirpα^{-/-} mice treated with either αPD-L1 alone (small tumors, 50 mm³), or αPD-L1 plus IFNγ/CpG or 8Gy radiation (larger tumors, 100-250 mm³), had survived (100%) and remained tumor-free, and were confirmed to have attained strong, long-lasting adaptive immunity against their cancer. Two direct effects were observed: first, these mice were resistant to multiple attempts at tumor re-engraftment (FIG. 5A); second, adoptive transfer of serum or splenic T cells from tumor-resistant Sirpα^{-/-} mice to WT mice conferred tumor resistance in WT recipients (FIG. 5B, 5C). Evaluation of serum samples from the donor mice revealed the presence of IgG that directly labeled MC38 cells (FIG. 5B).

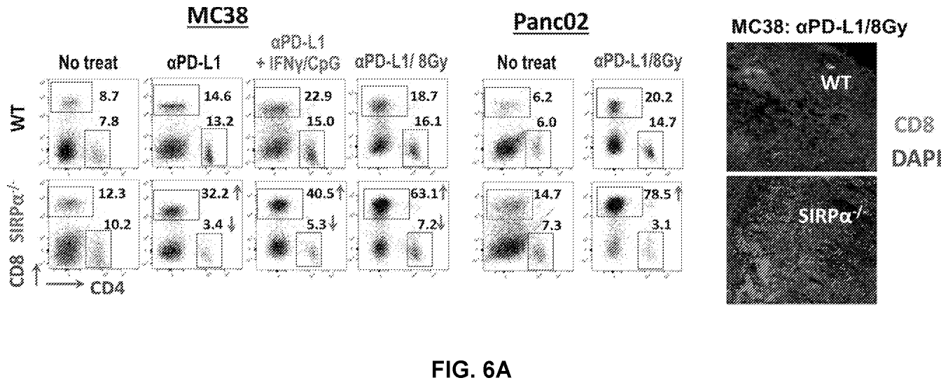

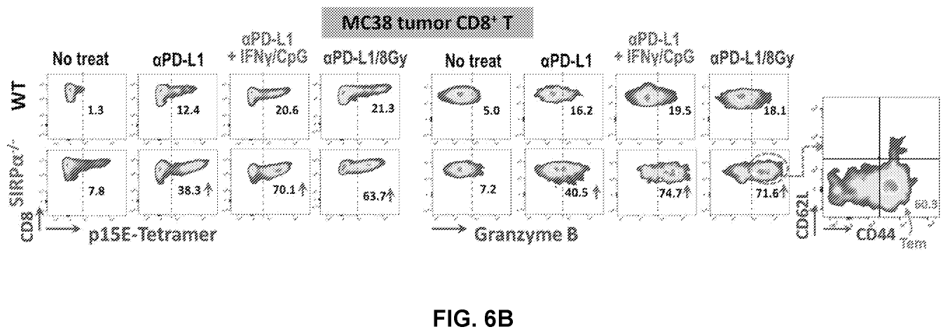

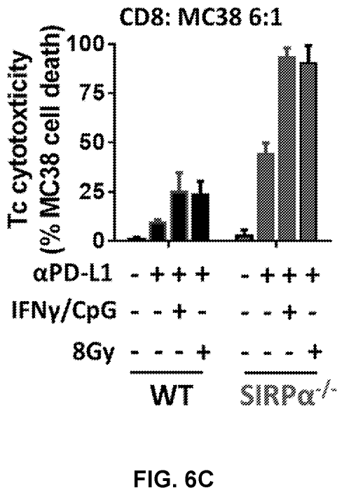

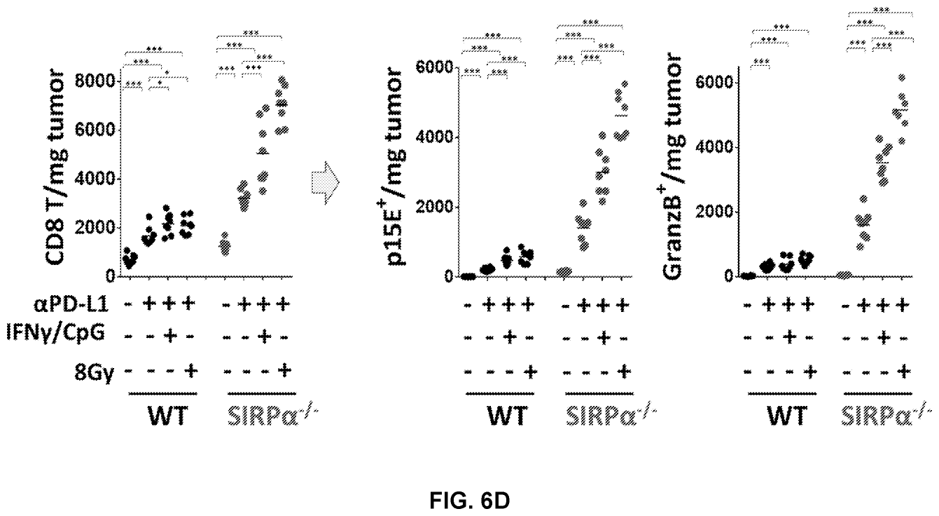

Given that Tc are crucial for PD-L1 blockade to mediate anti-cancer effects (Wei, S. C., et al. Cancer Discov., 2018. 8(9):1069-1086), Tc infiltration was analyzed in MC38 tumors prior to and after αPD-L1 administration to WT and Sirpα^{-/-} mice. As shown in FIG. 6A, Sirpα^{-/-} mice were capable of expanding much greater numbers of Tc in the tumor after αPD-L1 administration than WT mice; even before treatment, Sirpα^{-/-} tumors displayed a higher basal level of Tc (FIG. 6A). These facts thus explained why αPD-L1 alone exerted better efficacy in Sirpα^{-/-} mice than it had in WT mice. Treating Sirpα^{-/-} mice with αPD-L1 together with IFNγ/CpG, or 8Gy RT, further increased tumor infiltrating Tc to a much greater number, even reaching >50% of the total infiltrated leukocytes (CD45⁺). In contrast, Tc in WT tumors were only moderately increased after two doses of αPD-L1±IFNγ/CpG or 8Gy RT.

Not only were Tc rapidly proliferating and infiltrating in large numbers in Sirpα^{-/-} tumors, but they also exhibited a high level of granzyme B (GranzB) expression, suggestive of their robust activation and potent cytotoxicity, and a striking specificity toward tumor cells (FIG. 6B), the latter assessed by reactivity to the p15E-MHCI tetramer. MuLV p15E is an epitope specifically expressed in MC38 tumor cells while absent in host animals (Kershaw, M. H., et al. Cancer Research, 2001. 61(21):7920-7924; Bronte, V., et al. J Immunol., 2003. 171(12):6396-6405; Kim, E.-K., et al. Cancer Research, 2014. 74(22):6705-6716); hence it represents a tumor-specific antigen useful to assess Tc tumor-specificity. 30-40% Tc in αPD-L1-treated Sirpα^{-/-} tumors were p15E-reactive, and this number was further increased to a stunning >70% once IFNγ/CpG or RT was combined. Among these p15E-reactive Tc, a significant fraction was found to be CD44⁺CD62L⁻, indicating differentiation into effector memory cells (T_{EM}). The p15E-reactive Tc and T_{EM} were also found in the peripheral blood of αPD-L1-treated Sirpα^{-/-} mice. Conversely, αPD-L1-treated WT mice generated notably less numbers of p15E-reactive Tc/T_{EM} out of the already smaller total Tc population, which were also mostly GranzB^{low}, representing poor Tc activation and cytotoxicity. Ex vivo cytotoxicity assays (FIG. 6C) confirmed this fact, showing that Tc isolated from treated Sirpα^{-/-} tumors had a strong killing capacity towards MC38, whereas those from WT tumors exhibited weak activity. Collectively, these findings implicate that SIRPα expression negatively affects both the quantity and the quality of anti-tumor Tc, especially in response to αPD-L1.

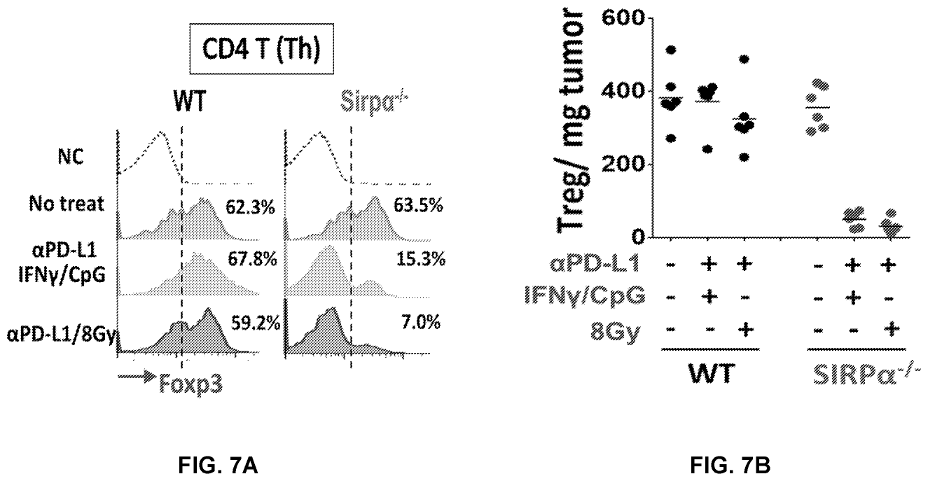

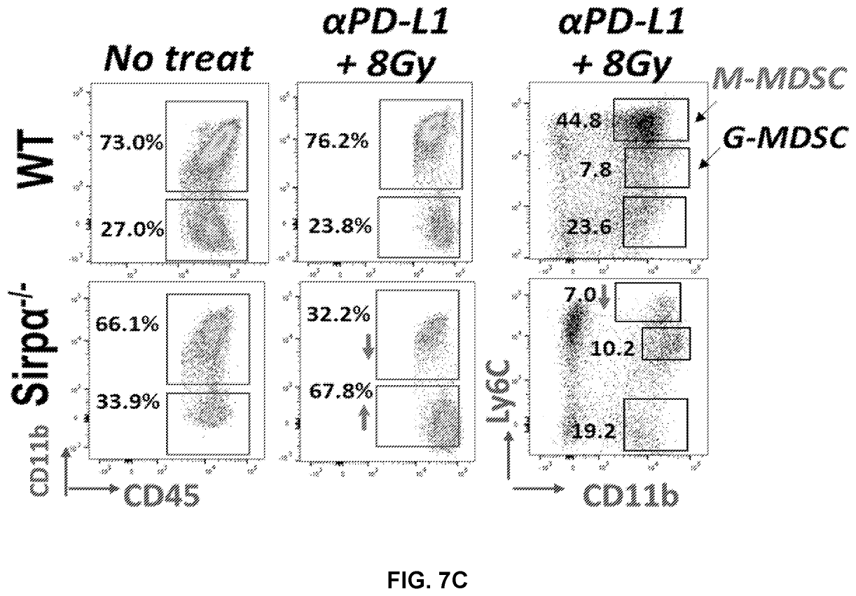

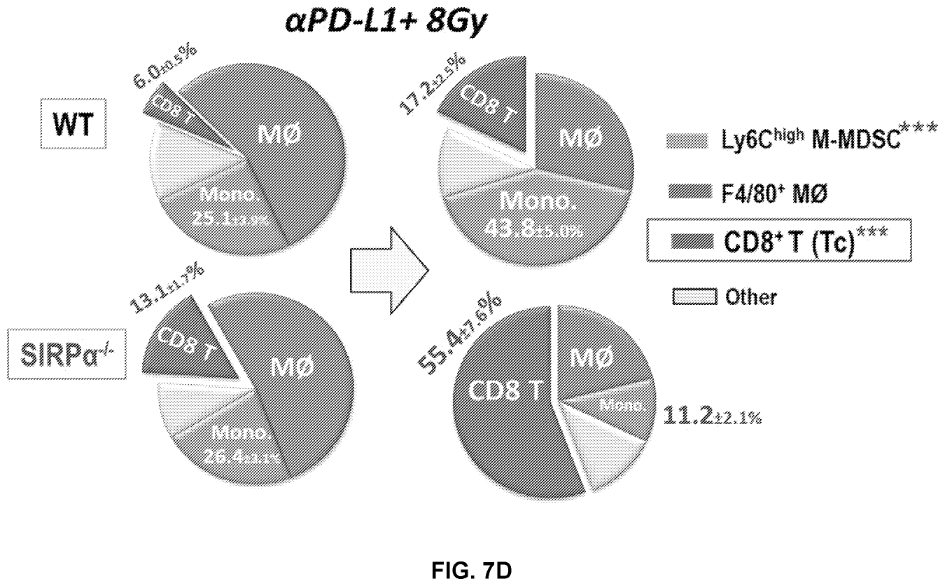

Additional characterization of the MC38 TME revealed other differences between Sirpα^{-/-} and WT mice following αPD-L1 treatment, especially in combination with IFNγ/CpG or RT. These included: 1) CD4 FoxP3⁺ T_{REGS} were reduced at a much greater scale in the Sirpα^{-/-} TME than that in WT mice; 2) there were many Ly6C^{high} monocytes/MDSC infiltrating tumors in WT mice after αPD-L1+RT treatment, but this did not occur in Sirpα^{-/-} mice. As shown in FIG. 7A, Sirpα^{-/-} tumors treated with αPD-L1+IFNγ/CpG or 8Gy RT displayed stunning diminishment of FoxP3⁺ T_{REGS}, from comprising >50% of the total CD4 T helper cell (Th) population to representing a minute fraction (<10%). This drastic T_{REG} reduction suggests that irradiated Sirpα^{-/-} tumors had altered their immunogenicity and possibly removed many immunosuppressive barriers. WT mice, however, only moderately reduce T_{REGS}.

A significant number of Ly6C⁺ monocytes were found infiltrating tumors in WT mice after tumors were treated with αPD-L1 plus RT (FIG. 7C), suggesting that WT tumors following radiation/Tc-mediated damage had produced strong wound-healing signaling that recruited monocytes, which function as MDSC (Bian, Z., et al. Eur J Immunol., 2018. 48(3):532-542), to suppress Tc cytotoxicity while promoting tumor growth (Gabrilovich, D. I., et al. Cancer Immunol Res., 2017. 5(1):3-8). Interestingly, this important tumor-supporting mechanism was explicitly absent in Sirpα^{-/-} mice after the same treatment. Again, these differences between WT versus Sirpα^{-/-} mice were stunning and will be further investigated.

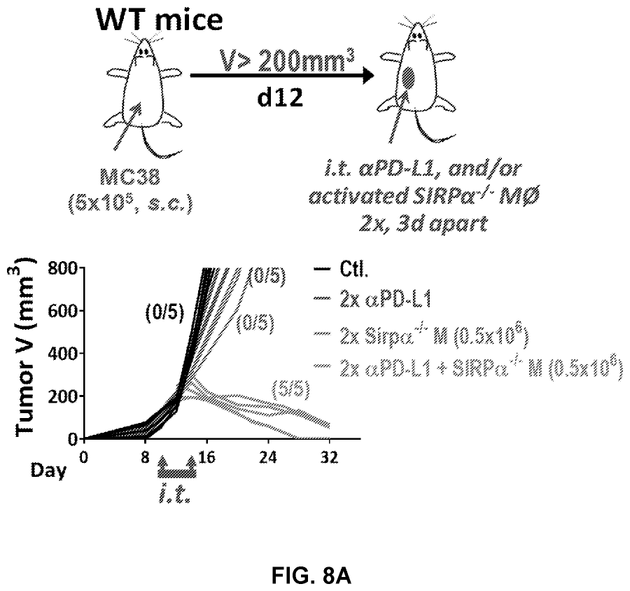

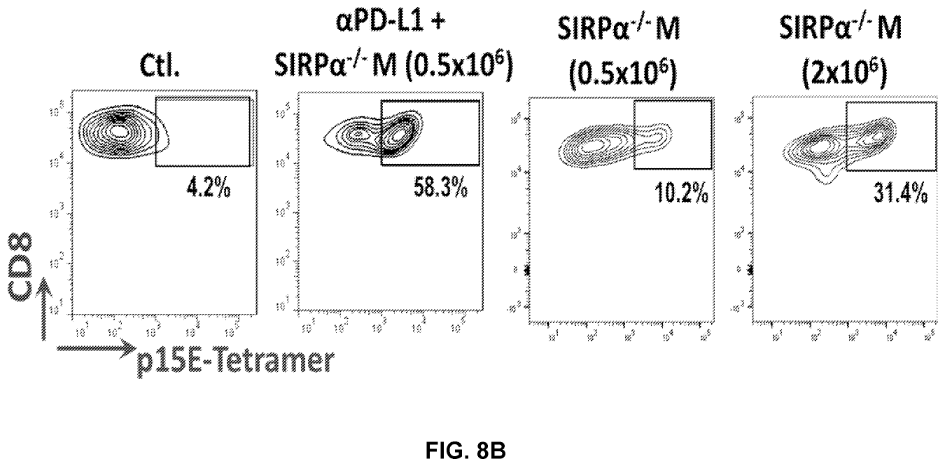

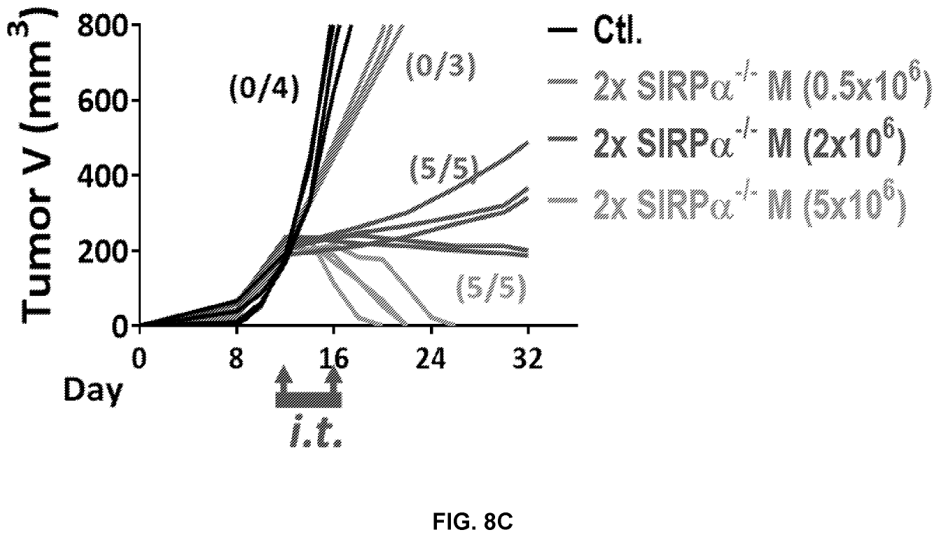

Given that Sirpa is expressed in myeloid phagocytes, these data thus suggest that intratumoral Sirpα^{-/-} phagocytes played a central role in conferring αPD-L1 sensitivity and reprogramming the tumor immune landscape. The deficiency of SIRPα depletes an ITIMs-SHP1/2 mediated inhibitory pathway (Weiskopf, K., Eur J Cancer, 2017. 76:100-109), a manipulation that likely promotes phagocytes, as well as the entire TME, towards pro-inflammation and antigen presentation. In contrast, SIRPα signaling, triggered by increased CD47 on surrounding tumor cells, strongly suppresses this activation. In order to explore if Sirpα^{-/-} phagocytes would bring about similar immunogenicity changes and enhance αPD-L1 efficacy, if transferred into WT tumors, Sirpα^{-/-} MØs (derived from bone marrow, BMDM, 5×10⁵ or 2×10⁶) were ex vivo treated with IFNγ/CpG (6-12 h) to activate their phagocytic capacity and enhance antigen presentation (Kranzer, K., et al. Immunology, 2000. 99(2):170-8), and then were intratumorally injected (infusion) into large, αPD-L1-refractory MC38 tumors (≥200 mm³) in WT mice. After 2 h, one dose of αPD-L1 was given. As shown in FIG. 8A, Sirpα^{-/-} MØ infusion dramatically reversed WT mice resistance to αPD-L1 and, with two doses of this combination, completely eliminated MC38 tumors. Again, this potent anti-cancer effect was associated with tumor-specific Tc expansion (FIG. 8B). These results support the postulation that Sirpα^{-/-} phagocytes are integral for sensitization to ICB, and also reveal promise in using Sirpα^{-/-} phagocytes as a therapeutic modality to reverse ICB refractory conditions, especially those present in cancers with low immunogenicity which are currently incurable.

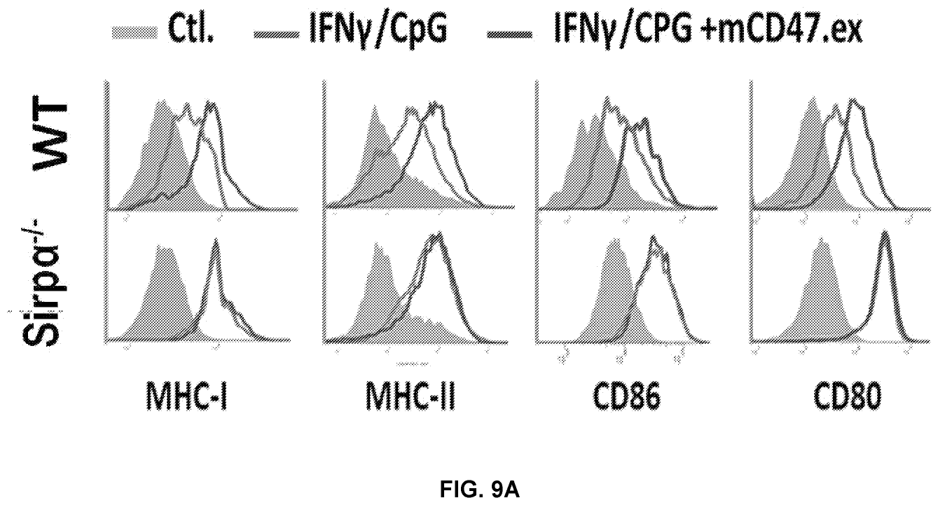

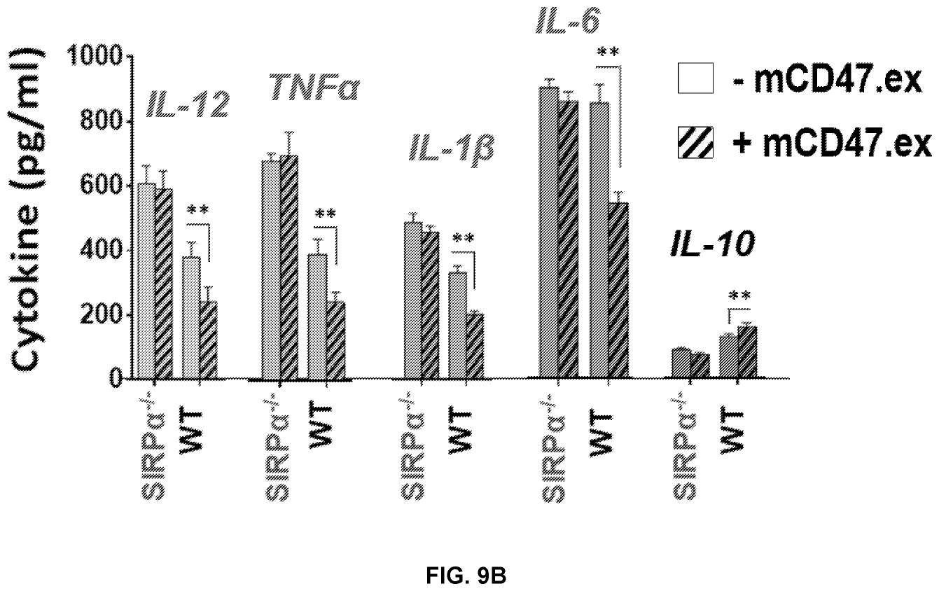

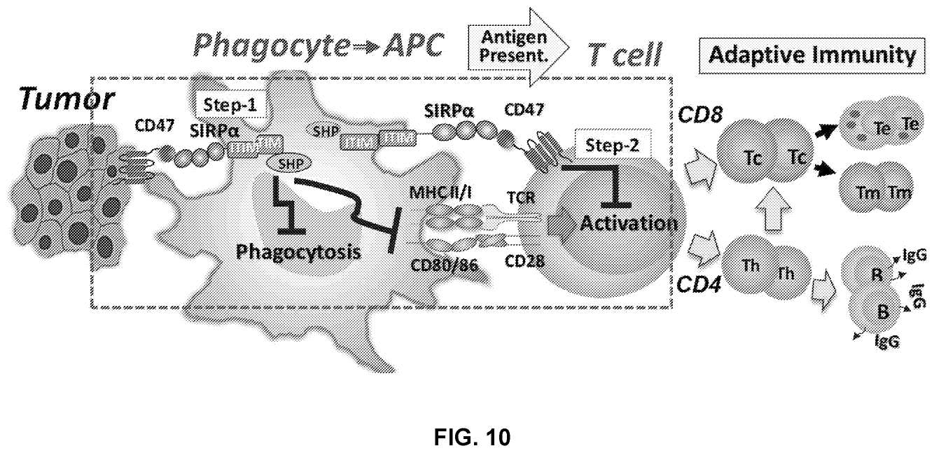

Interestingly, transfer of activated Sirpα^{-/-} MØs (2-5×10⁶) alone into WT tumors also induced significant Tc expansion and tumor regression (FIG. 8C), suggesting these MØs following phagocytosis of cancer cells/antigens (a predicted function) conducted immunogenic antigen presentation. To test if Sirpα^{-/-} MØs had an enhanced capacity for antigen presentation and whether SIRPα signaling negatively regulates it, MØ expression of MHC-I and MHC-II and co-stimulatory molecules CD80 and CD86 upon IFNγ/CpG stimulation were examined. As shown in FIG. 9, while IFNγ/CpG induced MHC-1/II and CD80/CD86 expression on both MØs, ligation of SIRPα by CD47 (mCD47.ex) on WT MØs strongly inhibited their expression. Furthermore, the CD47-SIRPα interaction also significantly inhibited WT MØs for production of inflammatory cytokines essential for immunogenic antigen presentation, such as IL-12, TNFα and IL-6, while depletion of SIRPα increased their release.

These studies produced compelling results that revealed new anti-cancer mechanisms mediated by Sirpα^{-/-} phagocytes. The discoveries point to a central role of phagocytes/APCs in the induction of anti-cancer immunity, against which SIRPα functions as a critical “brake”/barrier that dictates innate phagocytosis towards tumor cells, phagocytic APC antigen presentation, Tc activation, and TME immunosuppression. Remarkably, depleting SIRPα unleashes the full capacity of phagocytes/APCs to activate Tc, even reshaping the TME to favor immunogenicity, collectively empowering ICB to eliminate cancer. Indeed, the effectiveness of infused Sirpα^{-/-} MØs together with αPD-L1 in eliminating the poorly immunogenic MC38 tumor in WT mice is quite extraordinary.

### Example 2

### Results

### Focal RT Achieves Curative Responses in Sirpα^{-/-} Mice Against Poorly Immunogenic Tumors

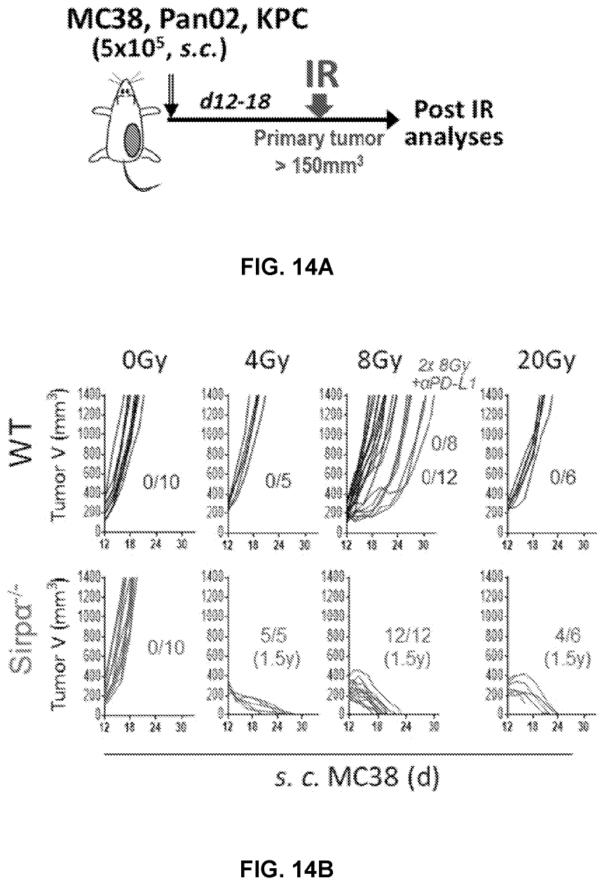

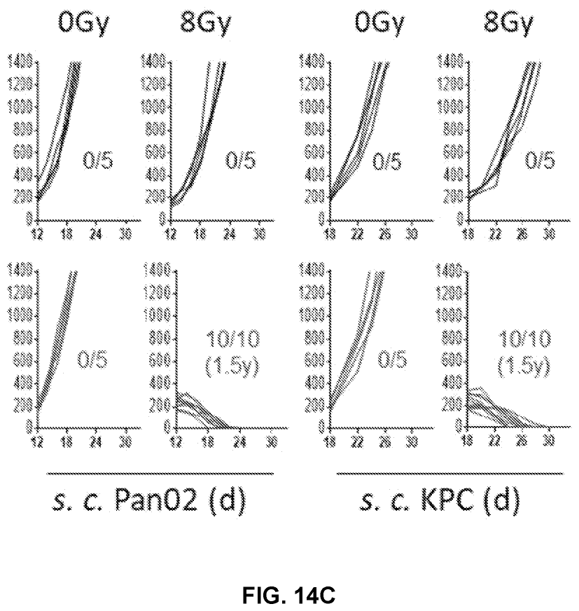

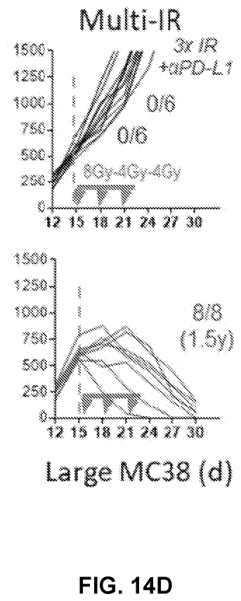

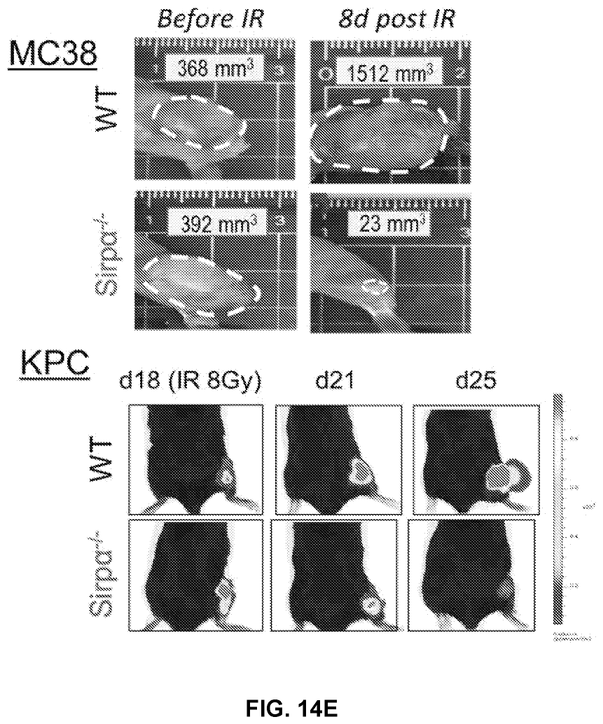

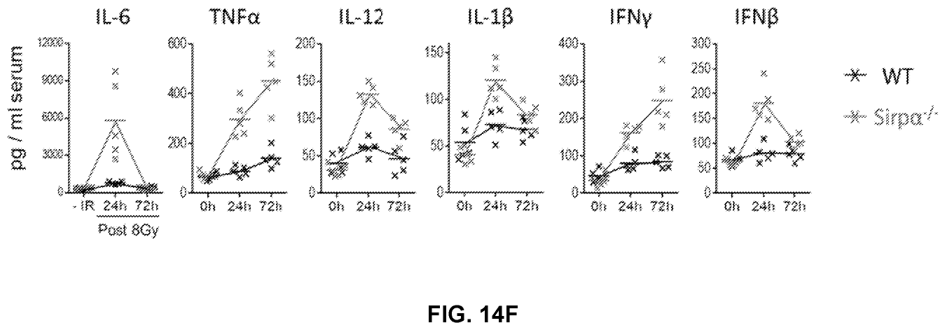

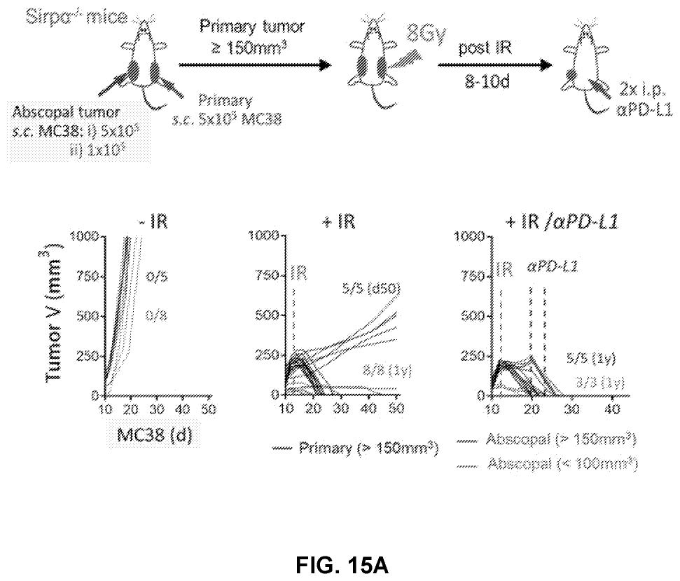

Subcutaneously engrafted MC38 or PDA (PanO2 or KPC) grew similarly in WT and Sirpα^{-/-} mice. Once tumors were well-established (>150 mm³), a single- or multi-fraction of X-ray radiation was given to treat the tumor. As shown (FIG. 14A-14D), these RT regimens, even high-dose hypofractionated RT with PD-1-blockade (2×[8Gy+αPD-L1]; FIG. 14C), failed to control the tumor burden in WT mice, in which tumors rapidly progressed beyond the humane endpoint. These results were consistent with studies by others, indicating that these low-immunogenic tumors highly resisted available therapies. However, a single fraction of X-ray radiation (1×4, 8 or 20Gy) conferred complete responses in Sirpα^{-/-} mice, inducing elimination of all traces of MC38, Pan02 or KPC tumors—not only small tumors (<200 mm³) but also rather large ones (>300 mm³). In most cases, cessation of tumor growth in Sirpα^{-/-} mice occurred immediately after irradiation (IR), followed by durable regression and complete clearance in a few days (5-12 d). Even for MC38 tumors>600 mm³, a multi-fraction regimen (8Gy-4Gy or 8Gy-4Gy-4Gy; 3 d interval) achieved complete elimination in Sirpα^{-/-} mice, whereas the same strategy with αPD-L1 only slightly benefited WT mice (FIG. 14E).

All of the tumor-engrafted Sirpα^{-/-} mice treated with 4Gy or 8Gy had survived (100%) without apparent adverse effects and remained tumor-free for the rest of the study (>1.5 y) (FIG. 14). Sirpα^{-/-} mice that received 20Gy, despite displaying rapid tumor regression, developed a severe adverse response resembling systemic inflammatory response syndrome (SIRS) with high release of proinflammatory cytokines and resulted in 33% mortality, albeit the mice that survived (67%) recovered and remained tumor-free (>1.5 y). Not only those treated with 20Gy, but all irradiated Sirpα^{-/-} mice exhibited elevated levels of TNFα, IL-6, IL-12 and IL-2 in their serum (FIG. 14F), whereas WT mice after RT did not show similar cytokine increases. These differences again indicated the exceptional responsiveness of Sirpα^{-/-} mice to RT, resulting in a markedly enhanced pro-inflammatory anti-tumor response.

### Abscopal Effects and Long-Lasting Immunity in Irradiated Sirpα^{-/-} Mice

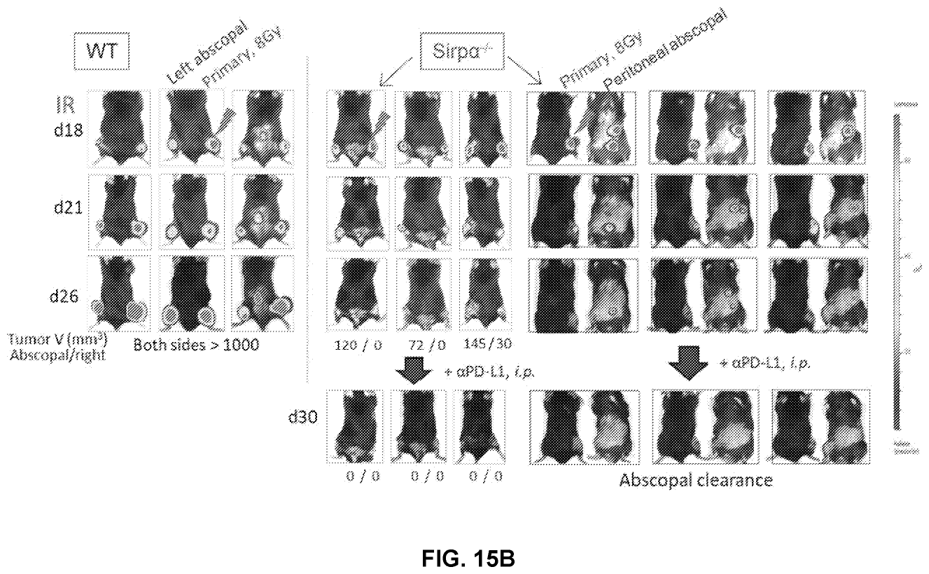

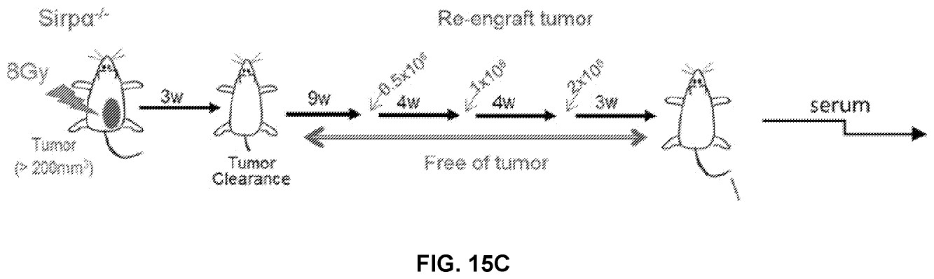

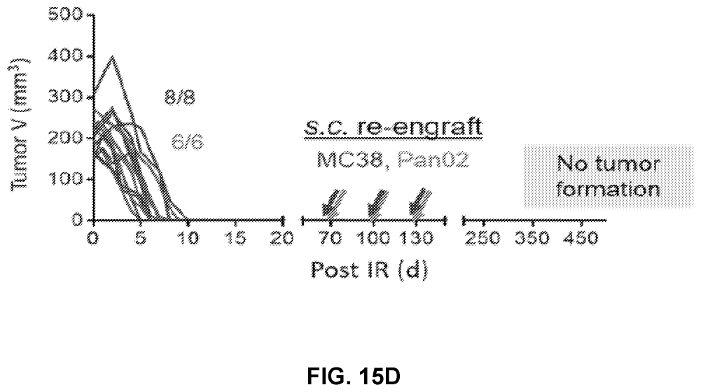

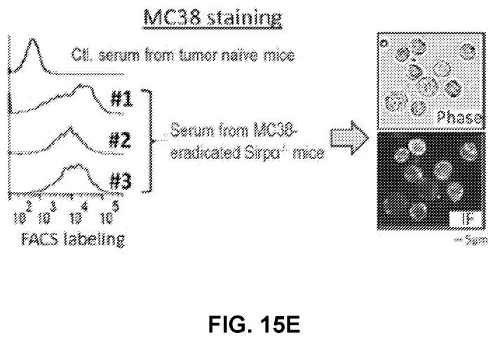

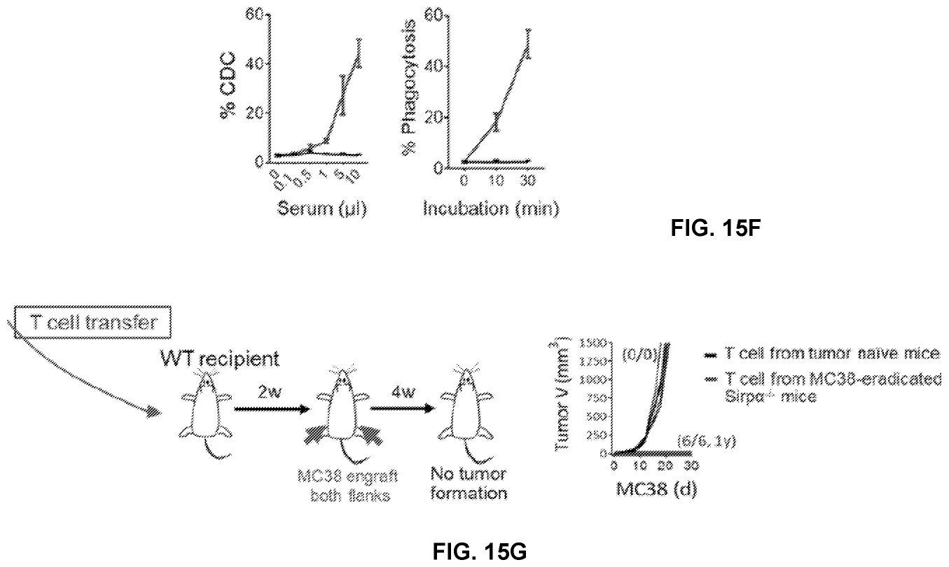

Further studies revealed that tumor-eradicated Sirpα^{-/-} mice acquired robust, long-lasting anti-tumor cellular and humoral immunity. Two direct effects were observed: first, the RT-treated Sirpα^{-/-} mice demonstrated effective abscopal tumor suppression; second, the tumor-eradicated Sirpα^{-/-} mice were resistant to recurrence (FIG. 15D-15F).

In rare instances, RT drives an endogenous immune response robust enough to control tumor burden outside the irradiated area, i.e., abscopal effect. To assess whether irradiation of primary lesions could induce control of unirradiated tumors, mice were engrafted with MC38 or PDA in both flanks (some also in dorsal areas), and when the primary tumor (right flank) reached >150 mm³, a fraction of 8Gy was given. As shown (FIG. 15A-15C), in Sirpα^{-/-} mice the RT treatment not only eliminated the primary tumor but also greatly hindered the growth of, or induced regression of, unirradiated tumors in other areas. In a subset experiment, the abscopal KPC tumors were engrafted orthotopically to the upper peritonea/liver areas, which as well displayed regression along with the primary tumor elimination by RT (FIG. 15D). Although the abscopal response was evident in Sirpα^{-/-} mice, the regression of abscopal tumors was usually slower or incomplete compared to that of irradiated tumors. Given that abscopal response is largely dependent on the effect of cytotoxic CD8 T cell (Tc)-mediated tumoricidality, anti-PD-L1 was administered to enhance the Tc function, and this regimen significantly accelerated abscopal tumor elimination, achieving complete clearance in a few days (FIG. 15B-15D). In contrast, WT mice did not exhibit abscopal response in all experiments (FIG. 5).

Following tumor elimination, potent anti-tumor immunologic memory was evident in Sirpα^{-/-} mice. Despite challenging with three rounds of inoculum-escalating MC38 or PanO2 re-engraftment, each attempt failed to establish tumors in Sirpα^{-/-} mice previously eliminated the same tumor (FIG. 15E-15F). Serum samples from these tumor-resistant mice revealed anti-tumor immunoglobulin (polyclonal IgG) that directly labeled the tumor cells (FIG. 15G) and mediated tumor cell killing through complement-dependent cytotoxicity (CDC) and Fc-mediated phagocytosis (FIG. 15H). Adoptive transfer of splenic T cells from the same tumor-resistant Sirpα^{-/-} mice to WT mice also conferred the latter immunologic protection, precluding tumor formation in recipients after attempted engraftments (FIG. 15I).

### Intratumoral Sirpα^{-/-} Macrophages Predicate Complete Responses to Local Irradiation

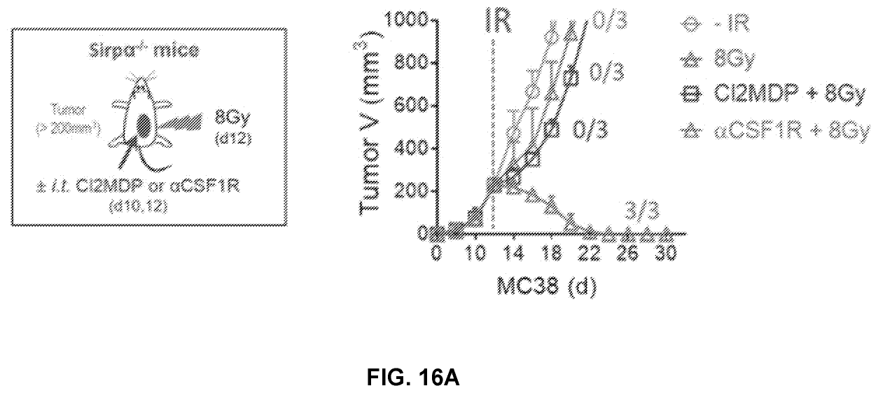

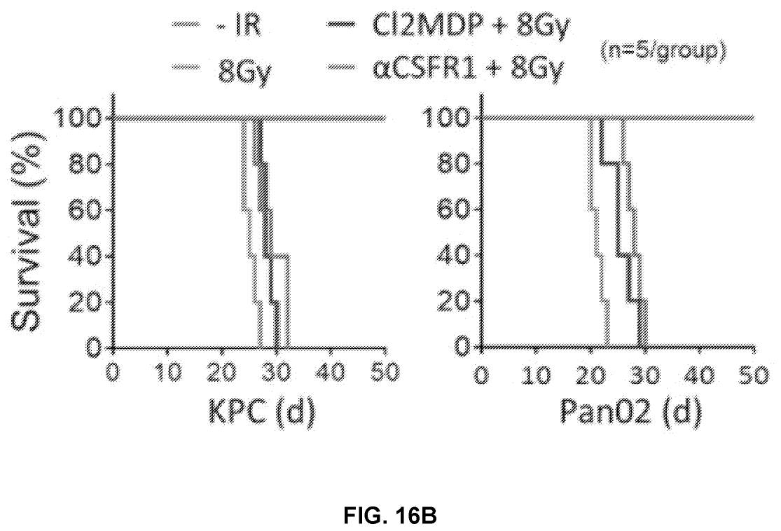

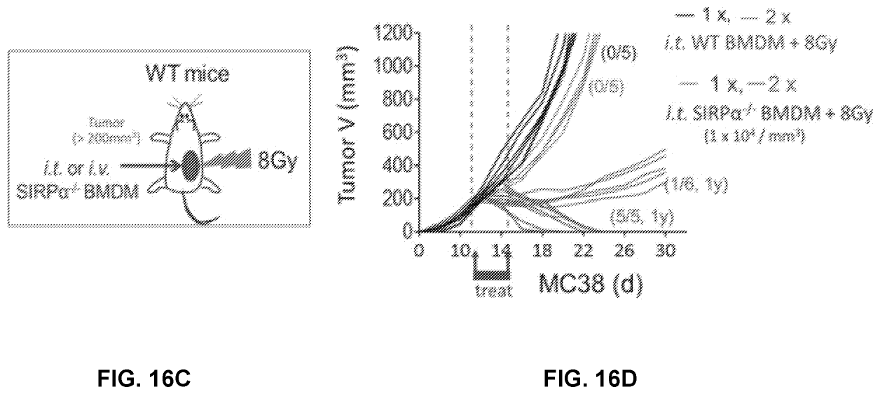

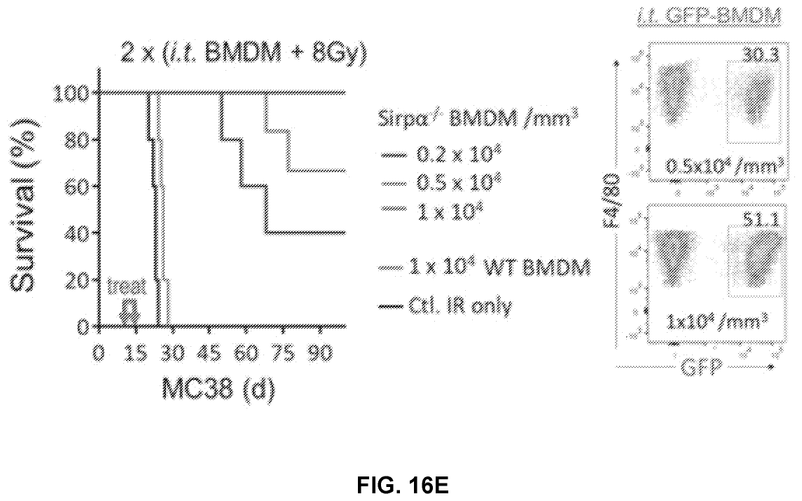

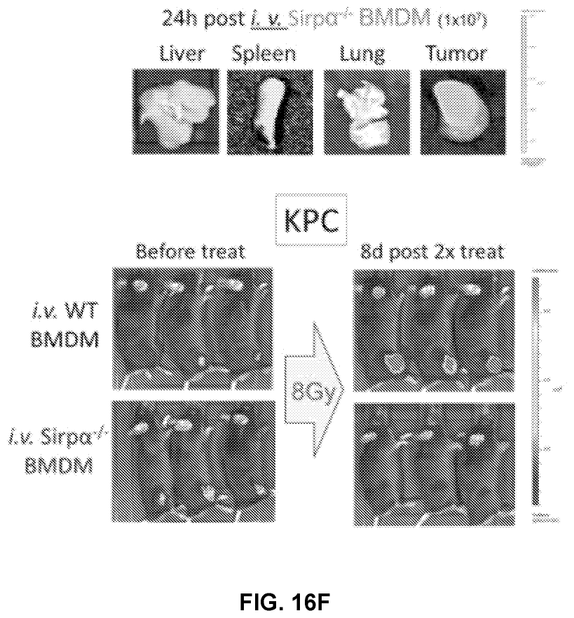

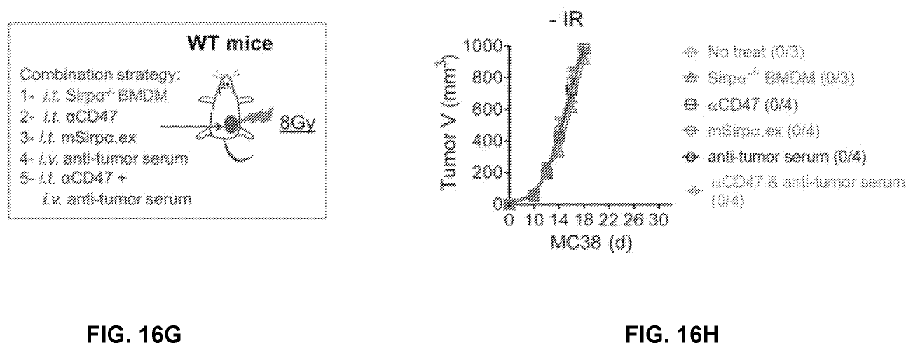

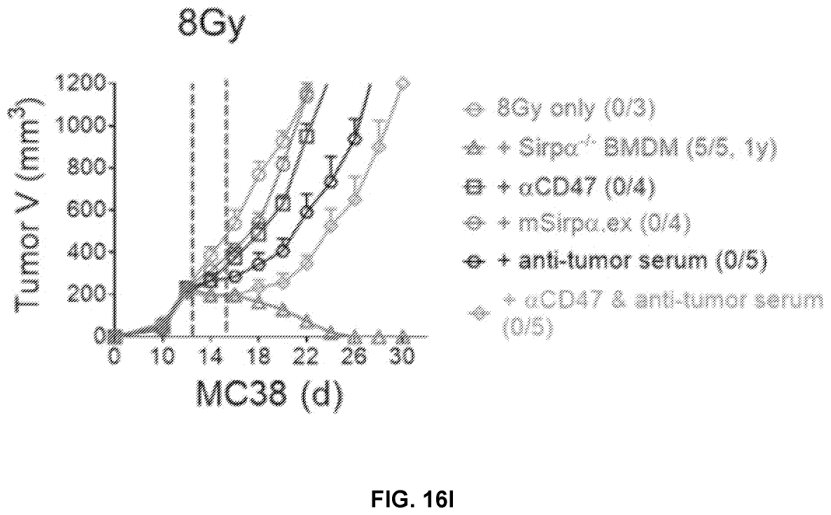

To determine whether intratumoral Sirpα^{-/-} macrophages underlay the efficacy of RT in Sirpα^{-/-} mice, intratumoral macrophages were depleted in these mice by CI2MDA-liposomes or antibodies against the CSF receptor (αCSF1R), and found that either strategy abrogated the efficacy of RT (FIGS. 16A-16B). Moreover, in WT mice bone marrow-derived Sirpα^{-/-} macrophages (Sirpα^{-/-} BMDM) were infused into MC38 or PDA tumors and tested RT responses (FIG. 16C). For these experiments, Sirpα^{-/-} BMDM were injected directly into tumors (i.t. multi-point fashion) or administrated intravenously (i.v.), the latter method was based on the fact that Sirpa deficiency alone does not drive macrophages to phagocytose self-cells. Both methods achieved intratumoral Sirpα^{-/-} macrophage infusion in WT mice, while neither route of administration caused adverse reactions such as anemia. Particularly, pharmacokinetic analyses following i.v. administration (1-2×10⁷ in 200 μl PBS) found that the majority of Sirpα^{-/-} BMDM extravasated within 12 h, with approximately 20-30% infiltrating tumor tissues, a phenomenon that has been shown to associate with the tumor-expressed monocyte/macrophage chemoattractant CCL2.

Intratumoral Sirpα^{-/-} macrophage infusion dose-dependently, radically enhanced the efficacy of RT in recipient WT mice (FIG. 16D-16F). Two rounds of 8Gy irradiation plus Sirpα^{-/-} BMDM administration, with the number of infused Sirpα^{-/-} BMDM commensurate to the endogenous intratumoral WT (Sirpα⁺) macrophages (˜1×10⁴ per mm³ tumor mass, FIG. 16E, inset), led to rapid regression and complete elimination of MC38, Pan02 and KPC tumors (all >200 mm³), resulting in 100% survival of the treated WT mice (FIG. 16D-16F). These experiments also showed that Sirpα^{-/-} BMDM administered 1-3 h before or immediately after IR had similar anti-tumor efficacy. Together, these results confirmed that Sirpα^{-/-} macrophages predicate tumor responses to RT. The fact that similar tumor-eliminating efficacies were achieved by Sirpα^{-/-} macrophage intratumoral infusion in otherwise RT-refractory WT mice lends credence to future RT therapeutic regimens combining engineered SIRPα-deficient macrophages to improve efficacy and abscopal effects.

CD47 Blockade does not Recapitulate Sirpa Deficiency in RT

The compelling anti-tumor efficacy following RT conferred by Sirpα-macrophages could not be recapitulated by CD47 blockade, despite that both modalities disrupt the CD47-SIRPα axis. Two CD47-blockade reagents, an antagonistic CD47 antibody (αCD47; miap301) and a soluble murine SIRPα extracellular domain (mSIRPα.ex) and rabbit Fc fusion protein, were combined with RT to treat MC38 and PDA tumors (all >200 mm³) in WT mice. To assess their impact, these reagents were administered prior to or immediately after IR, following the dosing schedules of Sirpα^{-/-} BMDM infusion. As shown (FIG. 16G-16I), neither αCD47 nor mSIRPα.ex, even at high doses, were as effective as Sirpα^{-/-} BMDM, as combining IR with these CD47-blockade agents only slightly delayed tumor progression. IR and αCD47 were also further combined with high-titer anti-sera against MC38 or PDA tumors obtained from tumor-eliminated Sirpα^{-/-} mice after three rounds of re-engraftment challenge (FIG. 2). Despite substantially delaying tumor progression, an effect largely attributed to anti-sera combined with RT (FIG. 16I), this triple-combination regimen was again incapable of inducing a complete response. It was also noted that neither the CD47-blockade reagents nor anti-tumor sera alone, nor their combination, inhibited tumor growth in the absence of IR. These results suggest that mere blockade of CD47 binding does not fully relinquish Sirpa signaling, which even with only partial activity appears to significantly impede the RT-induced anti-tumor response.

### Irradiation-Activated Sirpα^{-/-} Macrophages Reshape the Tumor Microenvironment

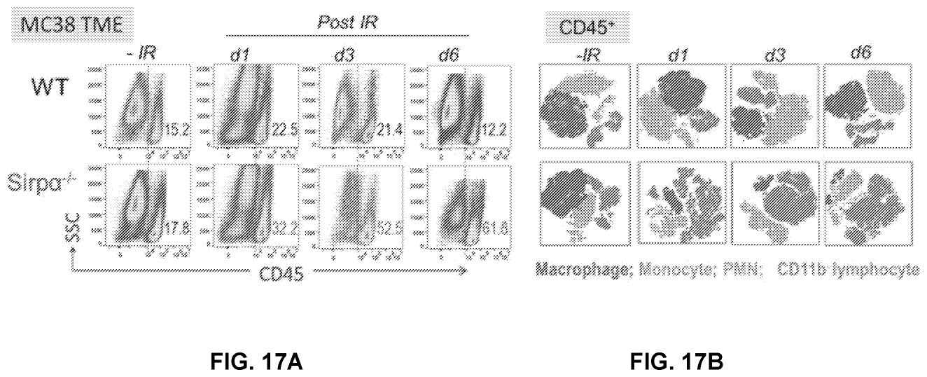

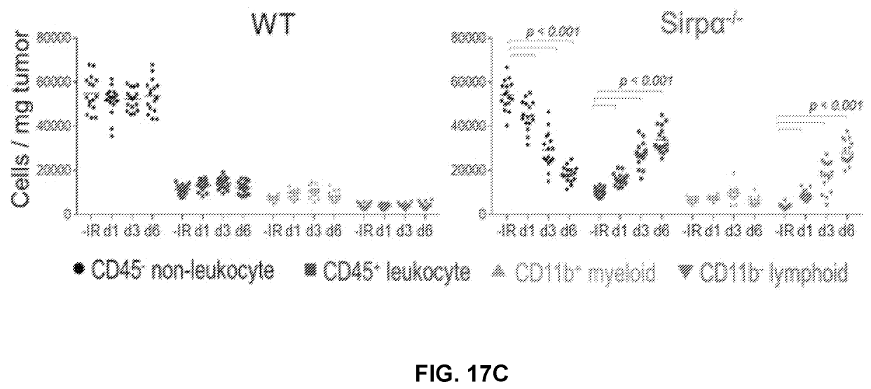

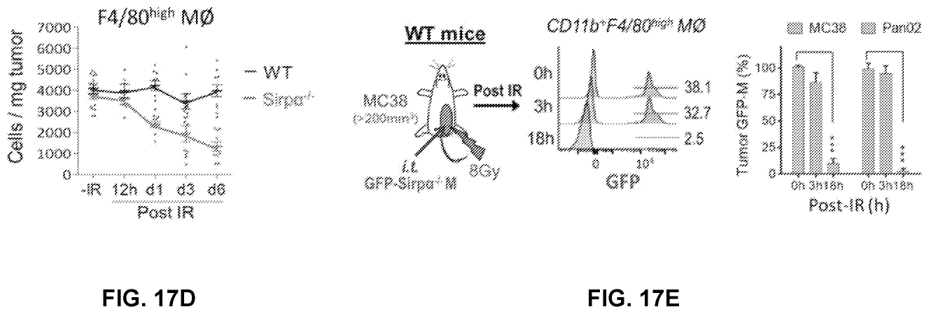

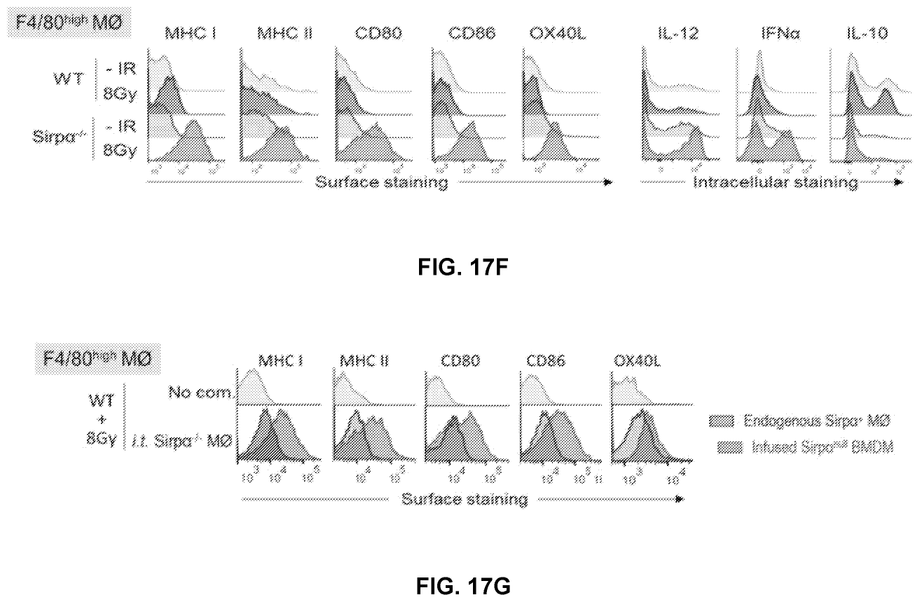

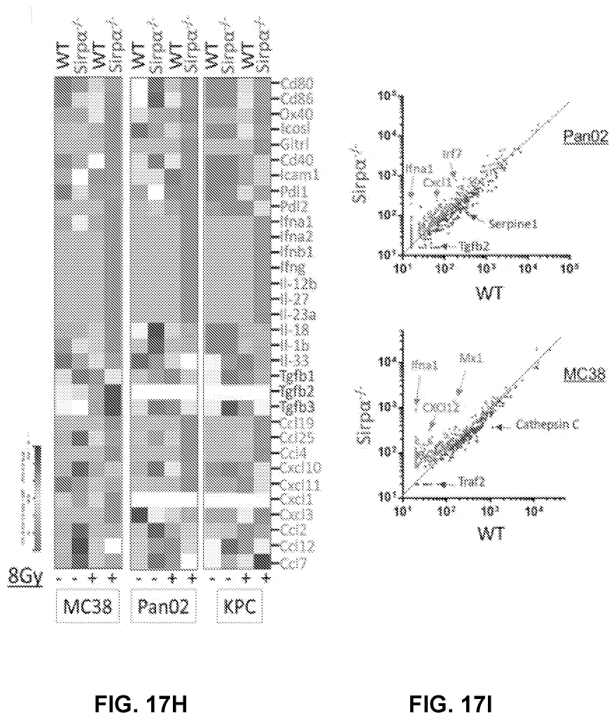

Analyses of the tumor microenvironment (TME) with and without Sirpα^{-/-} macrophages following an 8Gy treatment revealed that changes in the underlying immune landscape correlated with their differing responses to IR. As shown (FIG. 17A-17C), MC38 and PDA tumors comprising Sirpα^{-/-} macrophages, either in Sirpα^{-/-} mice or in Sirpα^{-/-} macrophages-infused, tumor-bearing WT recipients, were rapidly infiltrated by greater numbers of leukocytes (CD45⁺) after IR as compared to tumors that had no Sirpα^{-/-} macrophages (WT mice). By d6 post-IR, the infiltrated leukocytes in the Sirpα^{-/-} TME, of which the majority were lymphocytes, often outnumbered tumor cells, which had reduced as tumor volume rapidly shrank. Interestingly, although >35% of the total tumor leukocytes in Sirpα^{-/-} mice before IR were Sirpα^{-/-} macrophages, this population rapidly diminished after IR prior to detectable tumor regression (FIGS. 17B and 17D). Similarly, intratumorally infused Sirpα^{-/-} macrophages in WT recipients were undetectable in a day following IR (FIG. 17E), albeit the endogenous WT (Sirpα⁺) macrophages in the same tumor did not reduce in number. However, when Sirpα⁺ macrophages were infused instead, or if IR was withheld, the intratumoral macrophage population did not decrease. Further time-course analysis during the 24 h window following IR revealed that the intratumoral Sirpα^{-/-} macrophage population remained unchanged until 12 h post-IR but rapidly reduced thereafter (FIG. 17D). These findings were surprising given the indispensable role of Sirpα^{-/-} macrophages in RT-induced tumor elimination (FIG. 3), and also suggested that mechanisms other than activated Sirpα^{-/-} macrophage phagocytosis of tumor cells were underlying the durable tumor regression.

Despite the disappearance of Sirpα^{-/-} macrophages, their initial response to IR-induced tumor damage kindled a series of events that culminated in immunogenic repolarization of the TME and ultimately tumor elimination. As shown (FIG. 17F-17G), shortly after IR (<12 h), intratumoral Sirpα^{-/-} macrophages both in Sirpα^{-/-} mice and tumor-bearing WT recipients manifested robust proinflammatory signatures and immunogenic antigen presentation machinery with increased cell surface MHC-I, MHC-II, CD80, CD86 and OX40L and the expression of IL-12 and IFNα. Profiling the inflammatory signatures of bulk MC38, Pan02 and KPC tumors by Nanostring transcription analyses (FIG. 17H-17I) revealed similar strikingly altered TMEs after IR, with wide-ranging increases in the transcription of proinflammatory cytokines (IFNα/β/γ, IL-1α/β, IL-12, IL-18 and IL-33), immunogenic antigen presentation co-stimulatory molecules (CD80, CD86, OX40L, IcosL, GITRL and CD40), T cell and neutrophil chemokines (CXCL1/2, CXCL8, etc.), and other notable molecules (CX3CR1, CCR7, IRF3, IRF7, etc.) essential for tumor resistance. Meanwhile, the immunosuppressive cytokines such as TGFβ1/2/3 were substantially downregulated, signifying the irradiated Sirpα^{-/-} TME phenotypically shifting toward pro-inflammation and away from wound-healing. In contrast, irradiated tumors in WT mice without Sirpα^{-/-} macrophage infusion showed only weak proinflammatory transcription but prominent induction of TGFβs, and their associated Sirpα⁺ macrophages manifested a limited capacity for immunogenic antigen presentation but increased expression of IL-10, together suggestive of an increasingly immunosuppressive TME. These studies also revealed minor differences among the transcription profiles of non-irradiated MC38, Pan02 and KPC tumors in Sirpα^{-/-} or WT mice (FIG. 17H).

### SIRPα Deficiency Robustly Induces Tumor-Specific Cytotoxic CD8 T Cells

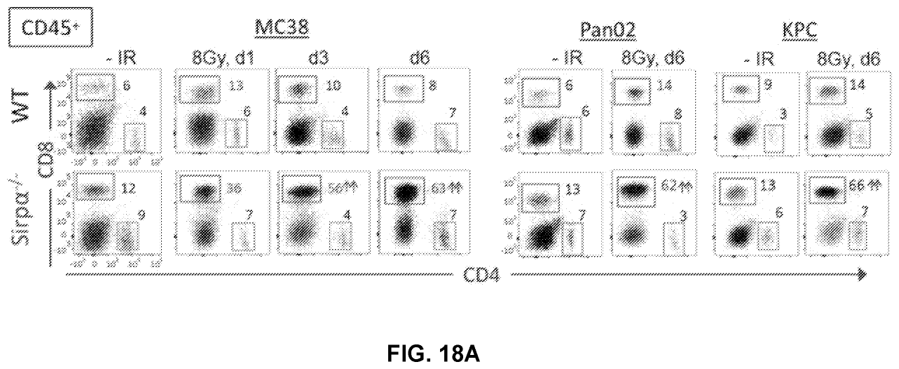

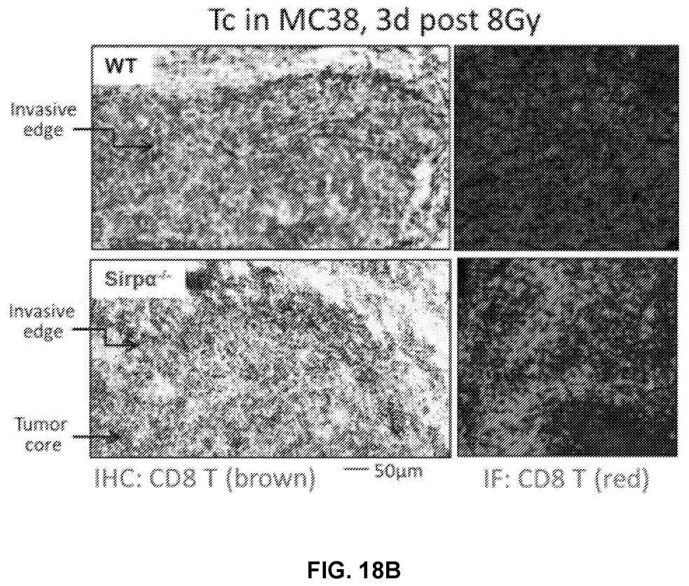

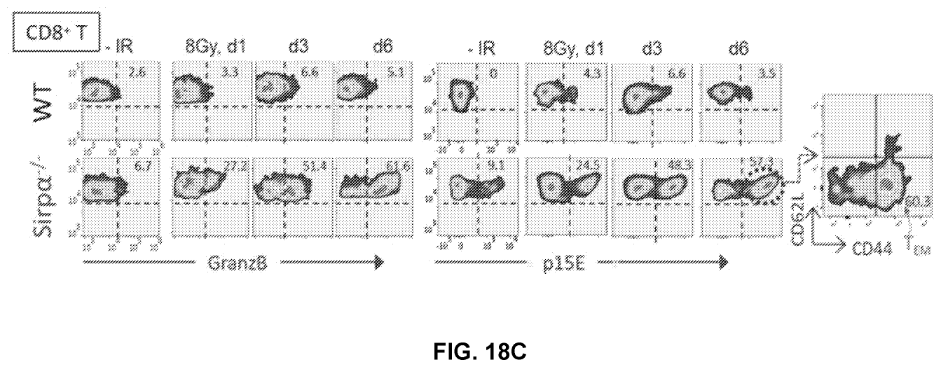

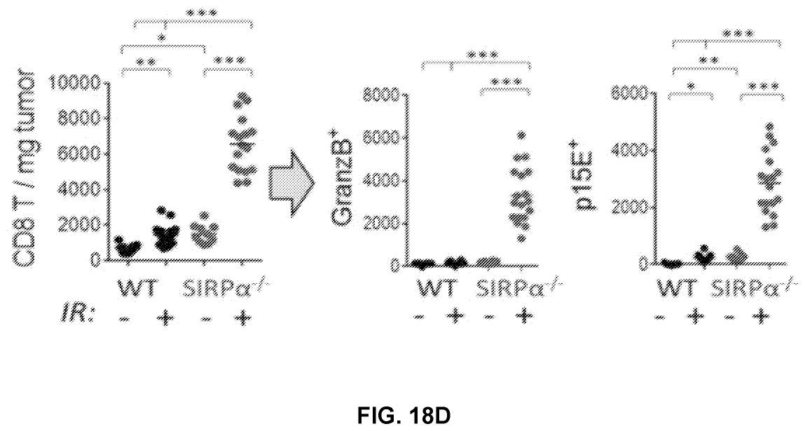

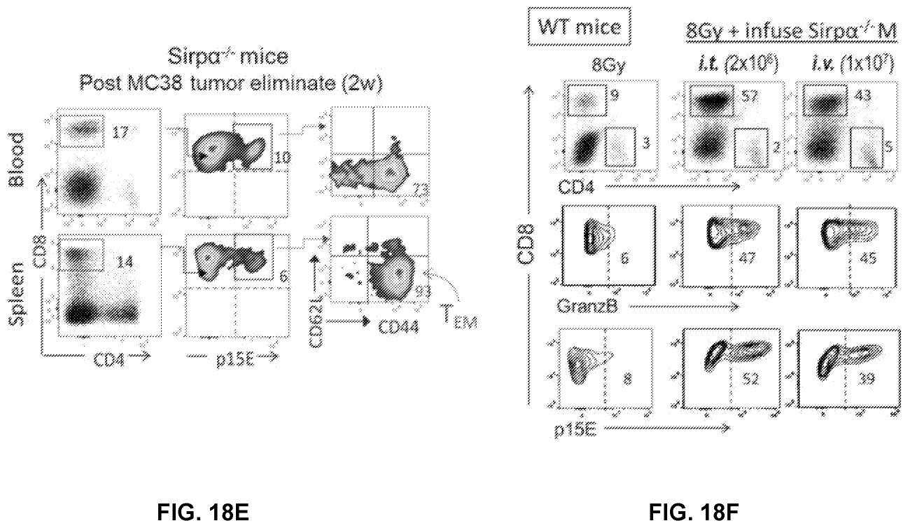

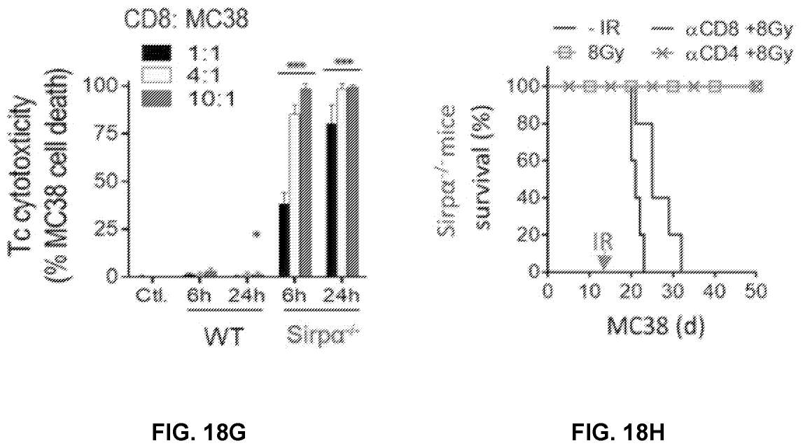

Among the many significant differences between tumor milieus comprising Sirpα^{-/-} macrophages versus those without, the population of tumor-infiltrated CD8 T cells (Tc) was strikingly larger in the former. As shown (FIG. 18A), irradiation of MC38 or PDA tumors in Sirpα^{-/-} mice led to the rapid expansion of intratumoral Tc, which represented nearly 40% of the total tumor-infiltrated leukocytes within 24 h post-IR, and were further increased to 50-70% by d3-6. These large number Tc in Sirpα^{-/-} TME distributed throughout the tumor core and along the invasive edge (FIG. 18B), and demonstrated high cytotoxicity and tumor-specificity, indicative by their high granzyme B (GranzB) expression and reactivity with the MuLV p15E-H2Kb tetramer, respectively (FIGS. 18C and 18D). MuLV p15E is an antigen expressed in MC38, Pan02 and KPC tumor cells, but is absent in host animals. Approximately 30-50% of expanded Tc in Sirpα^{-/-} TME were tumor-specific, and among them, a significant fraction was CD44⁺CD62L⁻, indicating differentiation into effector memory T cells (T_{EM}). The tumor-specific (p15E⁺) Tc and T_{EM} persisted in Sirpα˜˜mice and were readily detectable in the peripheral blood and spleen two weeks after tumor eradication (FIG. 18E). Increased Tc against another, MC38-specific tumor antigen, ADPGK, were also detected in MC38 tumor-irradiated Sirpα^{-/-} mice. Notably, irradiation of tumors infused with Sirpα^{-/-} macrophages in WT recipients similarly induced robust expansion of GranzB^{high}p15E⁺ Tc (FIG. 18E). In stark contrast, WT mice without Sirpα^{-/-} macrophages after IR only generated a small population of intratumoral Tc, which largely lacked tumor-specificity (p15E⁺) and were mostly non-cytotoxic (GranzB^{low}). Ex vivo cytotoxicity assays confirmed that Tc isolated from irradiated, Sirpα^{-/-} macrophages-comprising tumors were highly cytotoxic and capable of rapidly eliminating (<3 h) cancer cells at a low effector:target cell ratio (FIG. 18G), whereas Tc from non-IR, or non-Sirpα^{-/-} macrophage-infused tumors of WT mice were inert against tumor cells. Apparently, the large expansion of tumor-specific Tc was critical for IR-induced durable tumor regression; the depletion of intratumoral Tc (αCD8), but not Th cells (αCD4), diminished the efficacy of RT in Sirpα^{-/-} mice (FIG. 18H).

### Activated SIRPα^{-/-} Macrophages Preclude Compensatory Immunosuppression

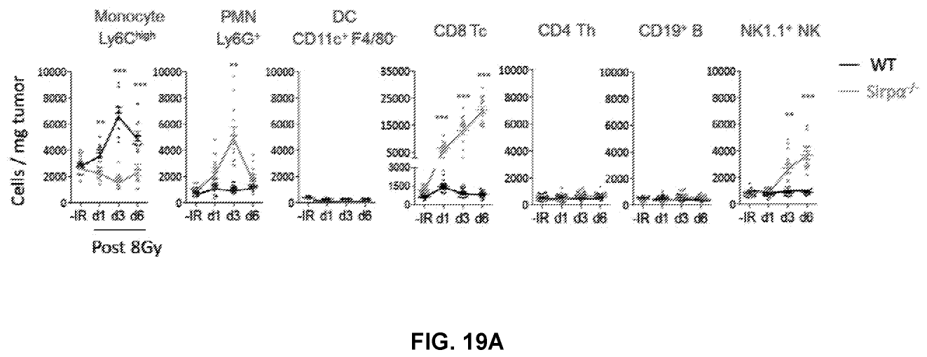

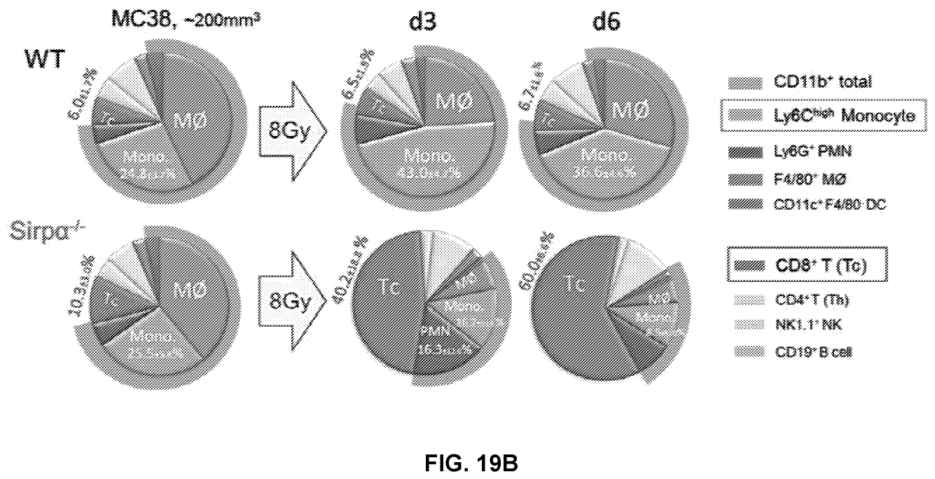

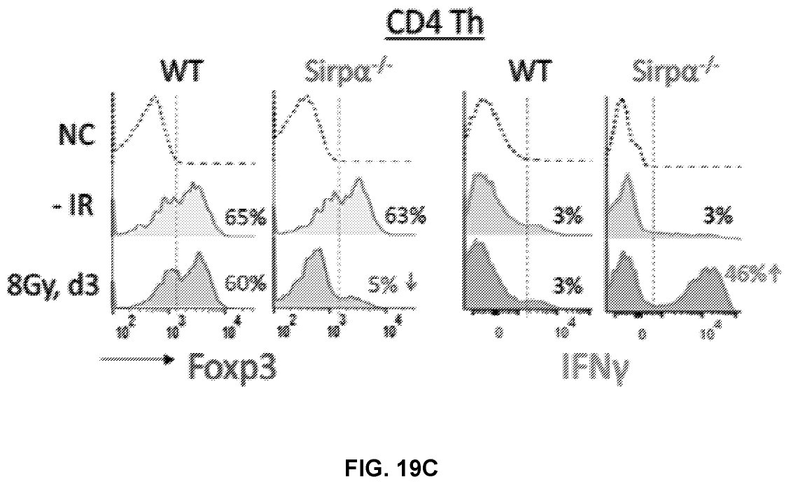

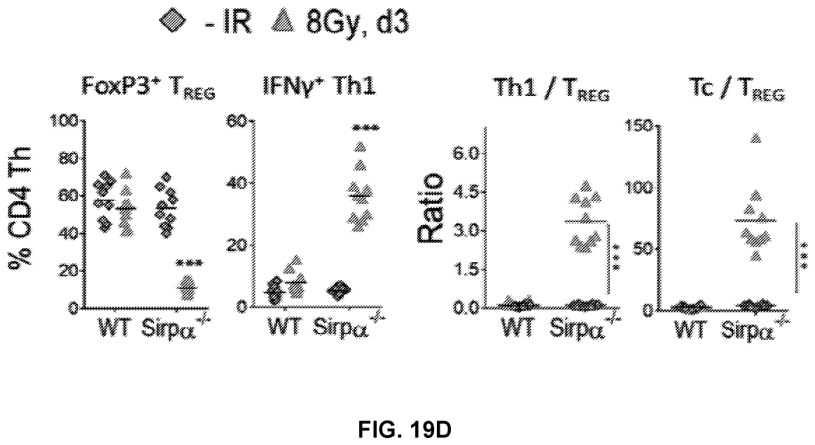

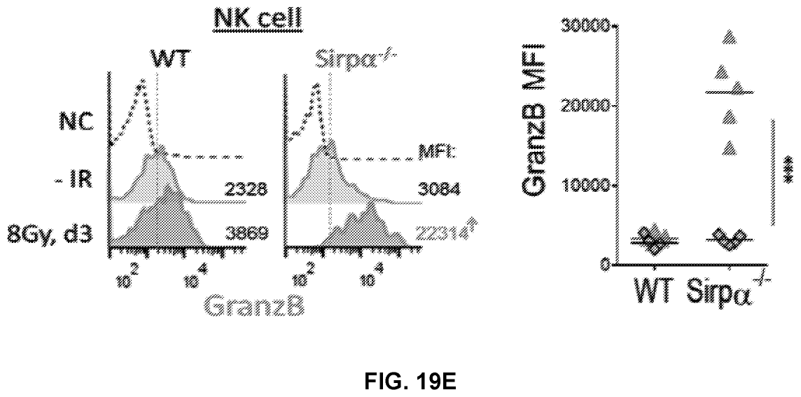

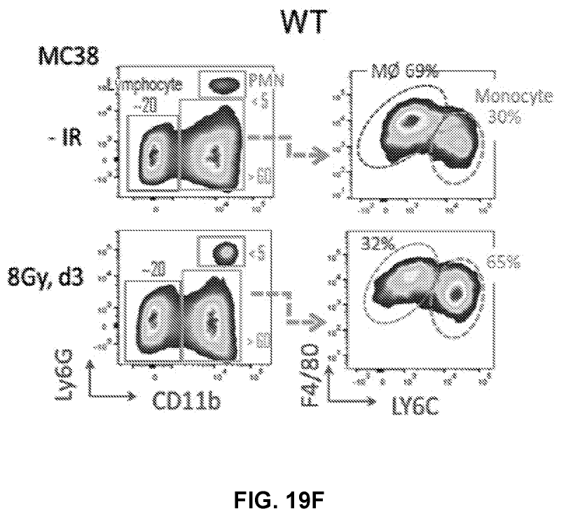

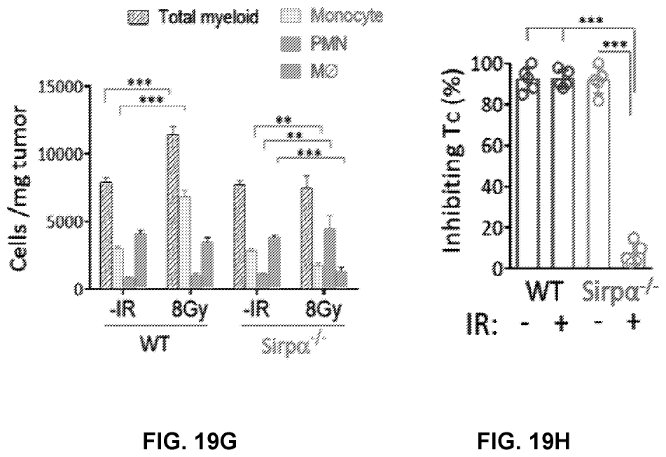

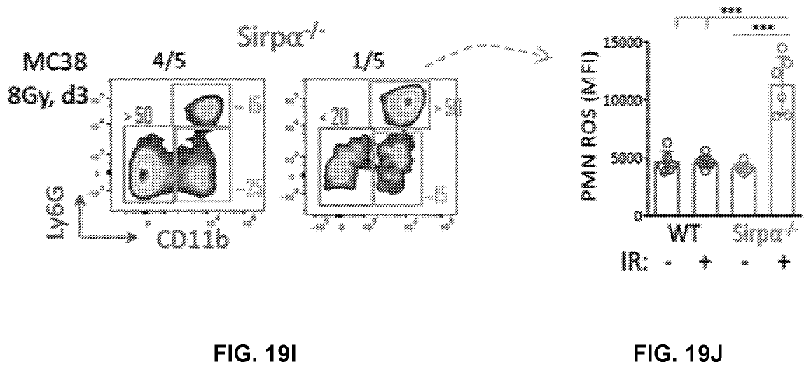

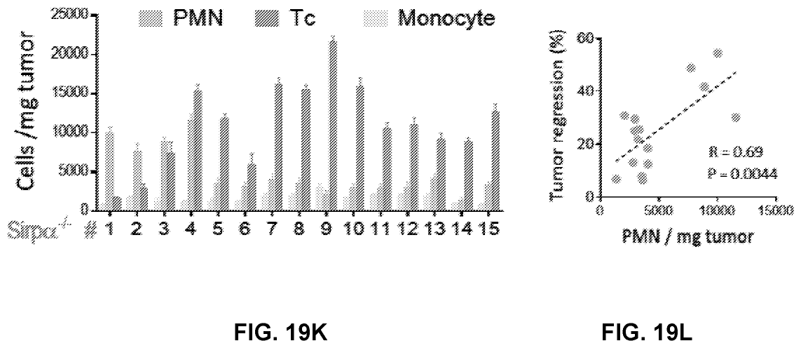

Further analyses revealed other prominent immune features synergistically augmenting tumoricidal activity in irradiated TMEs comprising Sirpα^{-/-} macrophages. These included: 1) diminishment of CD4 FoxP3⁺ T_{REGS} and an expansion of IFNγ⁺ Th1; 2) significant increases in NK cells; 3) marked infiltration of proinflammatory PMN (polymorphonuclear leukocytes, neutrophils) and a notable lack of Ly6C^{high} monocytes/MDSC. FIG. 19A-19B),

Despite maintaining similar total populations of intratumoral CD4 T cells (Th) (FIG. 19B), tumors in Sirpα^{-/-} mice exhibited marked reduction of FoxP3⁺ T_{REGS} after IR, which from comprising >50% of the total Th to a minute number (<10%); meanwhile, the IFNγ⁺ Th1 population expanded (FIG. 19C-19D). This Th phenotypic switch (T_{REG}→Th1), which failed to arise in WT mice, suggests an immunogenic shift within the TME that favors tumor elimination. Furthermore, these anti-tumor TMEs in Sirpα^{-/-} mice exhibited a 4-fold increase in NK cells, which also had high GranzB expression (FIG. 19E).

Consistent with reports showing that IR-incurred tumor damage drives a strong wound-healing response characterized by the recruitment of monocytes, which function as MDSC to suppress Tc immunity and promote tumor recovery and growth, all irradiated MC38 and PDA tumors in WT mice, not with Sirpα^{-/-} macrophage infusion, were highly infiltrated by Ly6C^{high} monocytes that strongly inhibited Tc proliferation (FIG. 19F-19H). Remarkably, this compensatory pro-tumor mechanism was explicitly absent in Sirpα^{-/-} mice, which instead reduced tumor-infiltrating monocytes/MDSC after IR. Rather, these Sirpα^{-/-} mice displayed a characteristic pro-inflammatory response accompanied by the release of pro-inflammatory cytokines (see Nanostring profiling and FIG. 14) and increased tumor infiltration of PMN (Ly6G^{high}), which produced high-level reactive oxygen species (ROS) but had no inhibition on Tc proliferation (FIG. 19H-19J). Approximately 20% of irradiated tumors in Sirpα^{-/-} mice were extensively infiltrated by PMN, and this phenomenon correlated with much faster tumor regression (FIG. 19K-19L). Chemokine profiling corroborated these results, showing that tumors of Sirpα^{-/-} mice secreted high levels of neutrophil-attracting CXCL8 after IR, whereas those from WT mice highly produced CCL2 to attract monocytes (see FIG. 17G). Ex vivo chemotaxis assays further confirmed differential neutrophil and monocyte chemotaxis towards Sirpα^{-/-} and WT tumors, respectively, after IR.

### Phagocytic SIRPα^{-/-} Macrophages Activate Tumor-Specific Tc In Situ

The high expression of immunogenic antigen presentation machinery, including MHC I/II and costimulatory molecules (FIG. 17), on intratumoral Sirpα^{-/-} macrophages after IR suggested that these macrophages, following phagocytosis of tumor cells, functioned as antigen presenting cells (APC), which through presenting tumor antigens activated tumor-specific Tc. Given the scale and kinetics of Tc expansion in irradiated tumors comprising Sirpα^{-/-} macrophages, it was postulated that this antigen presentation event occurred chiefly in situ and led to an anamnestic response of tissue-resident tumor-specific memory T cells (i.e., T_{EM} and T_{RM}).

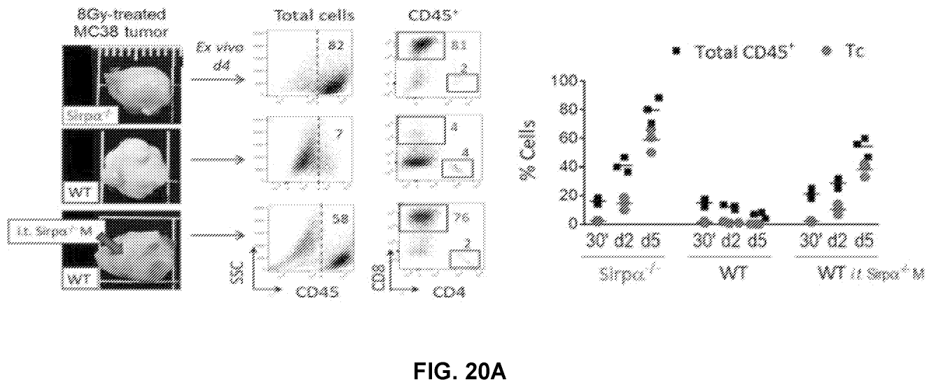

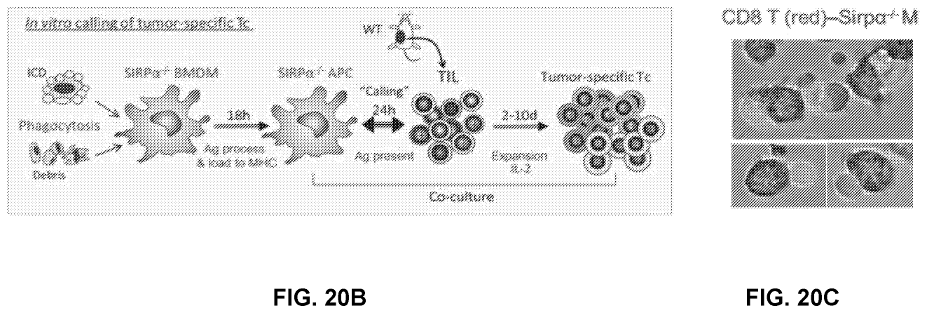

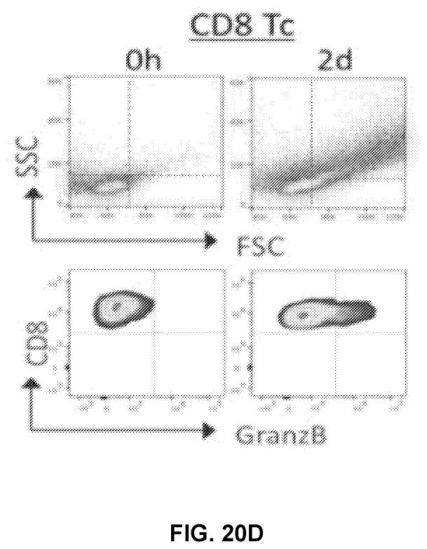

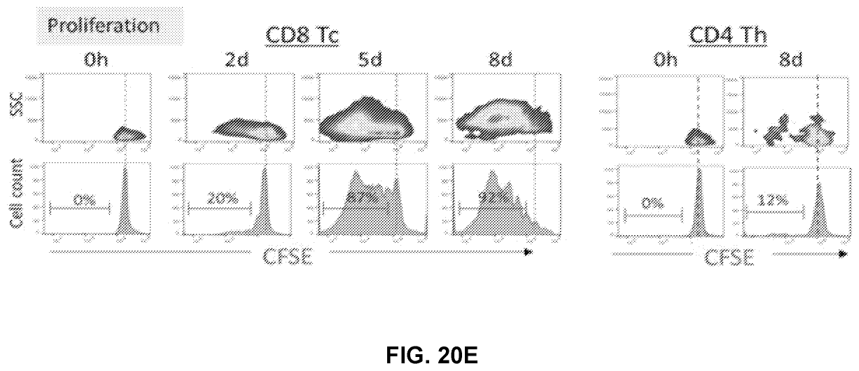

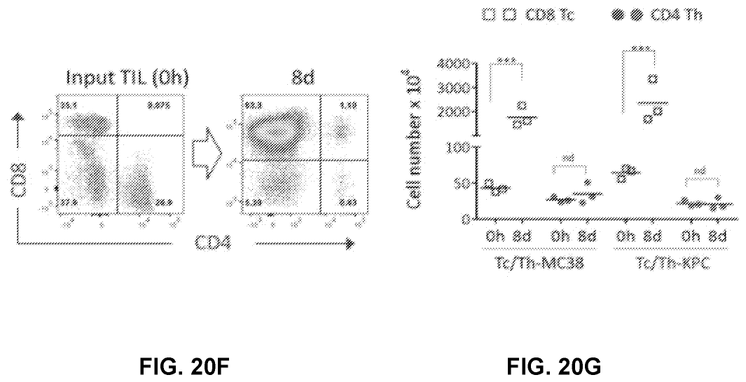

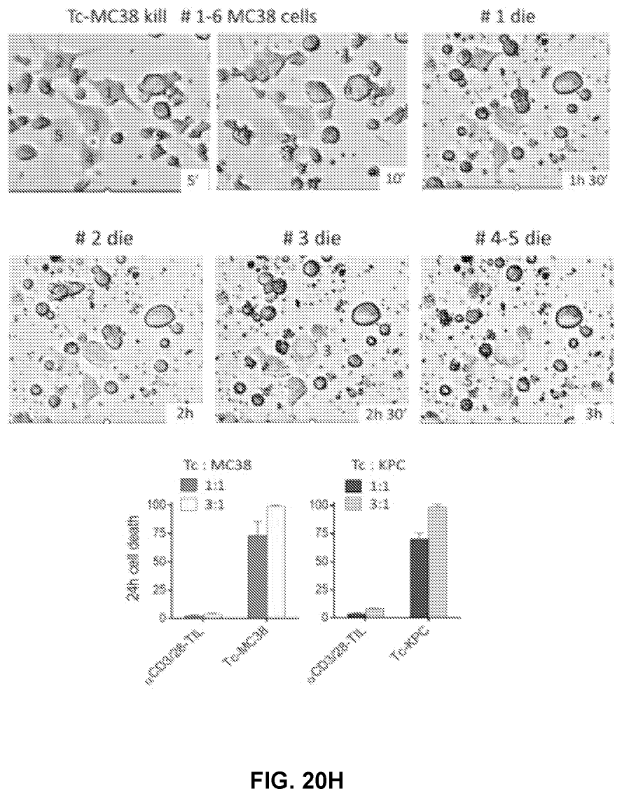

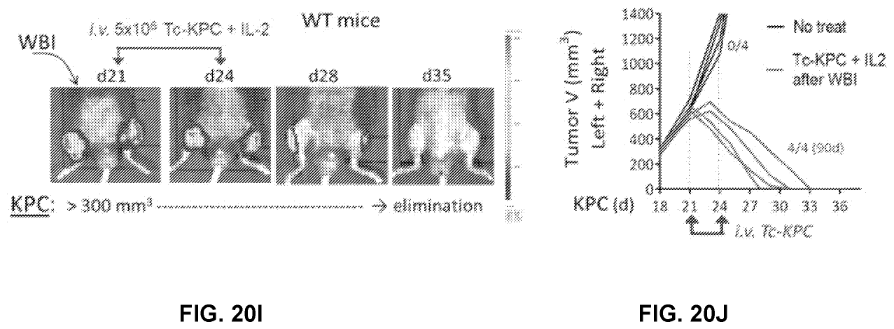

Two experiments were performed to test this hypothesis. First, tumor explants without tumor-draining lymph nodes (TDLN) from Sirpα^{-/-} mice immediately after IR (<30 min) were cultured ex vivo (FIG. 20A). Despite the absence of the TDLN, these cultured tumor explants exhibited expansion of Tc similar as those in vivo. Infusing Sirpα^{-/-} macrophages into tumor explants from WT mice also induced intratumoral Tc expansion. Second, an in vitro macrophage-TIL (tumor-infiltrated T cells) co-culture was established to ascertain the capacity of Sirpα^{-/-} macrophages for presenting tumor antigens and activating tumor-specific Tc. In these experiments (depicted in FIG. 20B), Sirpα^{-/-} BMDM were first incubated with irradiated MC38 or PDA tumor dissociates, comprising tumor cells and debris of ICD, for phagocytosis of tumor antigens. After overnight incubation (16-18 h) for antigen processing, by then Sirpα^{-/-} BMDM displaying proinflammatory characteristics and increased immunogenic antigen presentation machinery, the tumor antigen-loaded Sirpα^{-/-} BMDM then were co-cultured with TIL isolated from the same type, non-irradiated tumor. As shown (FIG. 20C), clear engagement between Sirpα^{-/-} BMDM and Tc was seen within 1 h of their co-culture, in a fashion reminiscent of APC antigen presentation. These engaged Tc represented less than 5% of total applied Tc, suggesting most other cells likely to be “by standers”, remaining distant or non-adherence. Following 1-3 days of co-culture, activation of Tc was apparent by cellular enlargement (i.e. blasting), robust Tc proliferation and expression of GranzB (FIG. 20D-20E). Interestingly, Sirpα^{-/-} APCs exclusively induced proliferation of Tc, but not Th, within the TIL population, a phenomenon which mirrored Tc expansion in irradiated TMEs with Sirpα^{-/-} macrophages. After 8-10 d culture in the presence of IL-2, a large number of Tc, which were produced after more than 10 cycles of proliferation, were harvested from the Sirpα^{-/-} BMDM/APC-TIL co-culture (FIG. 20F-20G).

Tumor cell-killing assays confirmed the tumor specificity and potent cytotoxicity of these in vitro-expanded Tc, which at low effector:target ratios (1-3:1) rapidly induced MC38 or PDA cell death (FIG. 20H). Interestingly, despite their exceptional cytotoxicity, only a fraction of these Tc (<5%) were p15E⁺, suggesting that the Tc population was polyclonal and the majority recognized tumor cells through other tumor-associated antigens. The ability of Tc to eliminate established tumors in vivo was further assessed. In these experiments, in vitro-expanded Tc against MC38 or KPC (termed Tc-MC38 and Tc-KPC, respectively) were i.v. administered (5×10⁶, 2×, 3 d interval) to WT mice bearing the same tumors. Prior to Tc infusion, a subset of mice were pre-conditioned with whole-body irradiation (WBI; 5Gy), then followed by i.v. injection of Tc along with IL-2 (i.p., 50,000 IU per day for consecutive 5 days). As shown (FIG. 20I-20J), two rounds of Tc infusion in WBI-conditioned mice plus IL-2 led to complete clearance of MC38 and KPC tumors larger than 400 mm³ and 100% survival. Similar Tc infusion without WBI and IL-2 achieved partial responses that significantly delayed tumor progression. For comparison, infusion of Tc/TIL expanded by antibody-ligation of CD3 and CD28, a non-tumor specific method, was largely inefficacious against established tumors or when cultured with tumor cells in vitro (FIG. 20H-20I).

### Discussion

Despite that WT TME by itself was incompetent of robustly activate Tc following IR, infusion of Sirpα^{-/-} macrophages brought about potent reaction to IR, leading to rapid expansion of GranzBhighp15E+ Tc in tumor-bearing WT recipients (FIG. 18F).

### Example 2

### Overview

SIRPANT technology comprises an innovative approach to engineer autologous SIRPα^{low} activated macrophages (SIRPANT-M) for driving powerful anticancer innate and adaptive immunity to eliminate cancer. Patient monocytes (peripheral blood mononuclear cells [PBMC]s) obtained from peripheral blood apheresis are manipulated ex vivo with SIRPANT's proprietary reagent, Phago-Act™, to produce macrophages with drastically reduced signal regulatory protein alpha (SIRPα) expression (ie, SIRPα^{low}) and inherent augmented capacity of phagocytosis, proinflammatory activity, and immunogenic antigen presentation. Upon administration into the tumorous mass, SIRPANT-M exerts potent anticancer activities including ingesting tumor cells, reprograming the tumor microenvironment (TME) towards proinflammatory thereby reducing immunosuppression, and presenting tumor-associated neoantigens to activate T cells in an immunogenic manner. Consequently, large numbers of tumor-specific polyclonal cytotoxic T cells are activated to eliminate tumor and distal metastases, a response that also leads to long-lasting cellular and humoral immunity that prevent cancer recurrence.

Since its development, SIRPANT-M as a cancer therapeutic approach has been thoroughly vetted in murine cancer models of lymphoma and various solid tumors including colorectal adenocarcinoma, pancreatic ductal adenocarcinoma, melanoma, lung cancer, and metastatic breast cancer. Among these tested cancers, some were late stage and had large tumors with multiple distal lesions (metastases) that resisted combinatorial therapies of immune checkpoint inhibitors (ICI), radiotherapy (RT), CD47 blockade, tumor vaccine and anti-tumor antibodies. However, treatment of these tumors with SIRPANT-M in all cases demonstrated high effectiveness, leading to systemic elimination of tumor lesions and survival rates up to 100%. Treated animals also exhibited the hallmarks of long-lasting immune memory that effectively prevented cancer recurrence.

In addition to in vivo proof of principle and efficacy studies completed in murine cancer models, ex vivo human studies using human SIRPANT-M have been conducted to assess their phagocytosis against the National Cancer Institute (NCI)-60 human tumor cell lines panel and activation of tumor-killing T cells from tumor infiltrating lymphocytes (TIL) obtained from patient specimens. The results confirmed human SIRPANT-M has the potential for rapid elimination of cancer cells both through phagocytosis and potent induction of tumor-specific cytotoxic T cells.

The goal of SIRPANT is to translate these research findings into clinical testing as an effective cellular immunotherapy for treating cancer. This cellular therapy approach was chosen based on extensive preclinical studies demonstrating that the effect of SIRPANT-M, especially for treating solid tumors, cannot be recapitulated or even approximated using ICI, RT, chemotherapy, CD47-blockade reagents, or other treatments.

### Summary of Studies

In vivo proof of principle and efficacy studies have been completed in murine cancer models of lymphoma and various solid tumors including syngeneic colorectal adenocarcinoma (MC38 cell line), pancreatic ductal adenocarcinoma (KPC and PanO2 cell lines), Lewis lung cancer (LLC), melanoma (B16 cell line), breast cancer 4T1 cell line (orthotopic engraftment), and metastatic breast cancer (mouse mammary tumor virus-polyoma middle tumor-antigen [MMTV-PyMT]). In all cases, SIRPANT-M treatment, especially when combined with local tumor radiation (TR), led to durable complete response with abscopal effects, eliminating late-stage primary tumors with distal lesions. All treated mice survived the treatment without apparent adverse effects and achieved long-term posttreatment survival rates comparable to healthy mice housed in the same facility (>90%, >1 yr). Data from these studies are summarized in Table 1.

Human studies have been conducted ex vivo to assess Phago-Act™-produced human SIRPANT-M for: a) phagocytosis against the entire NCI-60 panel of human tumor cell lines and other human cancer cells, b) the ability to produce inflammatory cytokines thereby driving proinflammatory response, and c) the expression of immunogenic antigen presentation machinery and the capacity of activation of tumor-killing T cells from tumor infiltrating lymphocytes (TIL) obtained from patient specimens. The results show that SIRPANT-M aggressively phagocytose both healthy and irradiated cancer cells, towards which regulate PBMC-derived macrophages failed to phagocytose. These studies also confirmed that human SIRPANT-M has the ability to drive strong proinflammatory response and immunogenic antigen presentation that activates tumor-killing cytotoxic T cells.

Further transcription profiling of SIRPANT-M prepared from 6 healthy volunteers of different sex and race/ethnicity demonstrated biased proinflammatory expression and augmented immunogenic antigen presentation machinery.

In summary, the preclinical in vitro and in vivo studies together point to the potentially high efficacy of SIRPANT-M as a cancer-agnostic immunotherapy that empowers both innate and adaptive immunity to eliminate cancer.

### Background and Mechanisms of Action

Macrophages are the most abundant leukocytes in the tumor microenvironment (TME) and play a pivotal role in the ability of the immune system to either eliminate or tolerate cancer cells. One critical mechanism regulating macrophage activity is governed by SIRPα-mediated signaling, which in one aspect executes via activation of SHP-1 to inhibit: i) phagocytosis of cancer cells; ii) proinflammatory activation by toll-like receptor (TLR) agonists, interferons (IFNs), and other proinflammatory cytokines and cancer therapy-induced factors; and iii) expression of immunogenic machinery for antigen presentation to induce anticancer adaptive immunity. Conversely, SIRPα via sequestrating the cytokine receptor inhibitory SHP-2 promotes signal transduction induced by immunosuppressive IL-4/13, IL-10 and TGFβ, thereby strengthening immunosuppression within the TME and tolerance for cancer. Details of these mechanisms are described in the following sections.

### Regulation of Macrophage Phagocytosis Toward Cancer Cells—

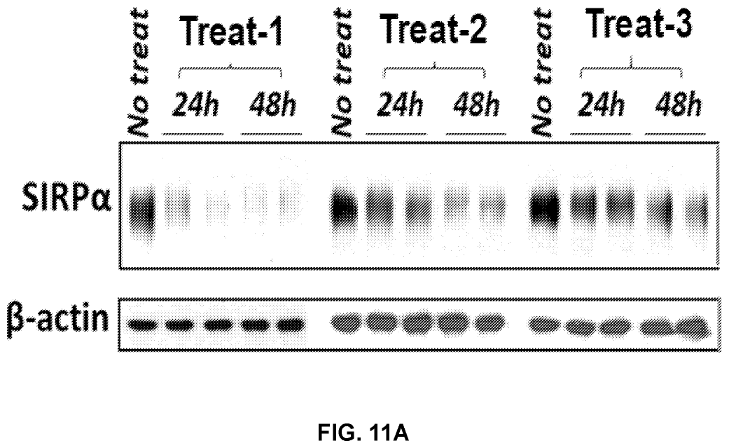

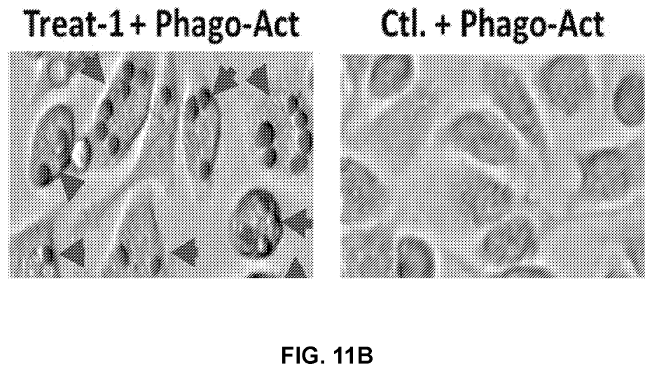

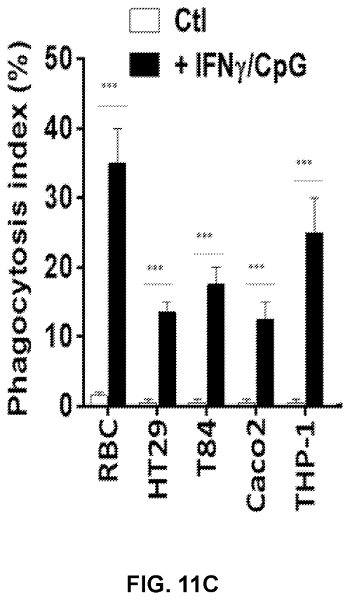

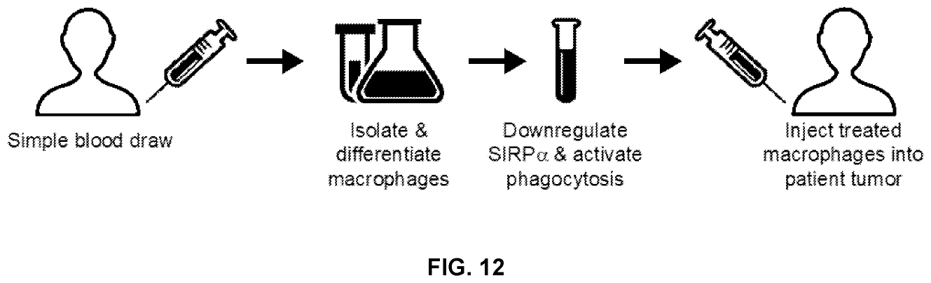

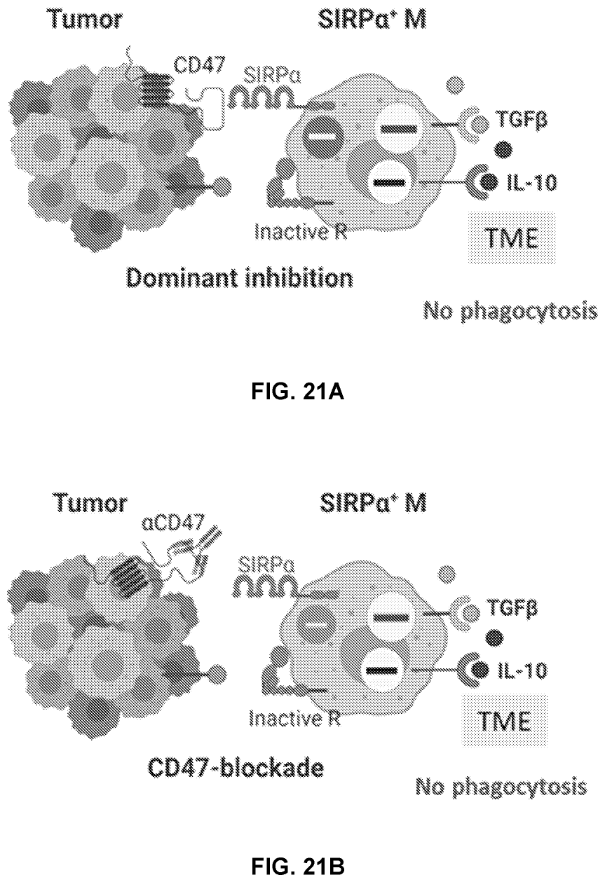

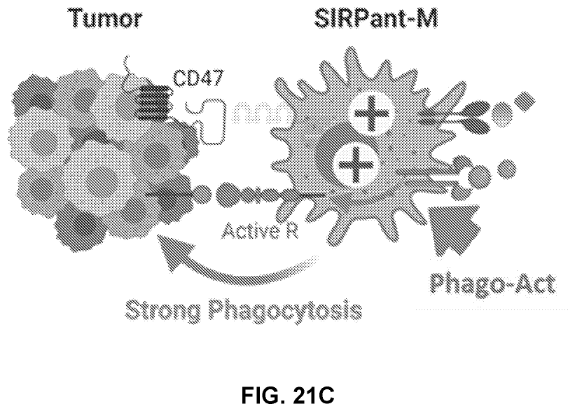

CD47 is a ubiquitous marker of self-cells and the cellular ligand for SIRPα. Cancer cells escape phagocytic elimination by triggering strong SIRPα-mediated inhibition when their CD47 extracellularly ligates SIRPα on macrophages. However, despite that some cancers exhibit high CD47 expression, more cases (>50%), which broadly represent different cancer types, poorly or do not express CD47 (The Human Pathology Atlas: CD47); yet these cancers avoid immune elimination in vivo even though their TMEs comprise an abundance of macrophages. Indeed, mere depletion of CD47 or cognate SIRPα signaling does not lead to phagocytosis; instead, additional phagocytosis activation mechanism(s) posed on macrophages is required to elicit their phagocytic activity. These studies were initially conducted in mice genetically lacking CD47 (Cd47^{-/-}) or SIRPα (Sirpα^{-/-}), both of which are generally healthy but manifest aggressive hemophagocytosis-induced anemia when exposed to virus infections or under inflammatory conditions. Similarly, ex vivo studies using Sirpα^{-/-} macrophages found that these macrophages, despite lacking SIRPα-mediated inhibition, are quiescent unless treated by certain proinflammatory cytokines or TLR agonists, which then renders them phagocytic toward self- and cancer cells. Along this line, it was found that inflammatory cytokines including the IL-1 family (e.g., IL-1β and IL-18), IL-6, IL-17, TNFα and type I IFNs (IFNα and IFNβ), but not IFNγ, and all TLR agonists (LPS, CpG, LTA, Poly I:C, flagellin, etc.) activate macrophage phagocytosis, whereas immunosuppressive cytokines IL-10 and TGFβ and steroid glucocorticoids counteract these proinflammatory factors by inhibiting macrophage phagocytic activation. Though detailed underlying mechanisms remain undefined, this process likely involves a specific phagocytic receptor that requires proinflammatory cytokine/TLR-induced signaling for inside-out activation, after which it mediates “universal” macrophage phagocytosis towards self-/cancer cells in the absence of CD47-SIRPα inhibition (FIG. 21C). These studies collectively suggest that macrophage phagocytosis is controlled by multiple layers of activation and inhibition mechanisms—a forefront control that determines macrophages either staying quiescence or being activated for phagocytosis, and the subsequent control via the CD47-SIRPα axis that determines the phagocytic target. These knowledges explain in part why tumor-associated macrophages (TAMs) are generally non-phagocytic even around CD47-low/negative cancer cells. In parallel, this also provides understanding why blockade of CD47 (e.g., anti-CD47 antibodies) alone is insufficient to treat cancer (FIG. 11B). Indeed, this treatment strategy requires combination with a modality that activates phagocytosis, such as cancer-specific antibodies (e.g., Rituximab for B cell lymphoma), which activate phagocytosis via Fc receptors, or chemotherapy reagents (e.g., azacytidine for myelodysplastic syndrome [MDS] or acute myeloid leukemia [AML]), which increase cellular expression of calreticulin that in turn ligates macrophage-expressed LRP1 to trigger phagocytosis.

### Example 3: SIRPα is Instrumental in TME Immunosuppression

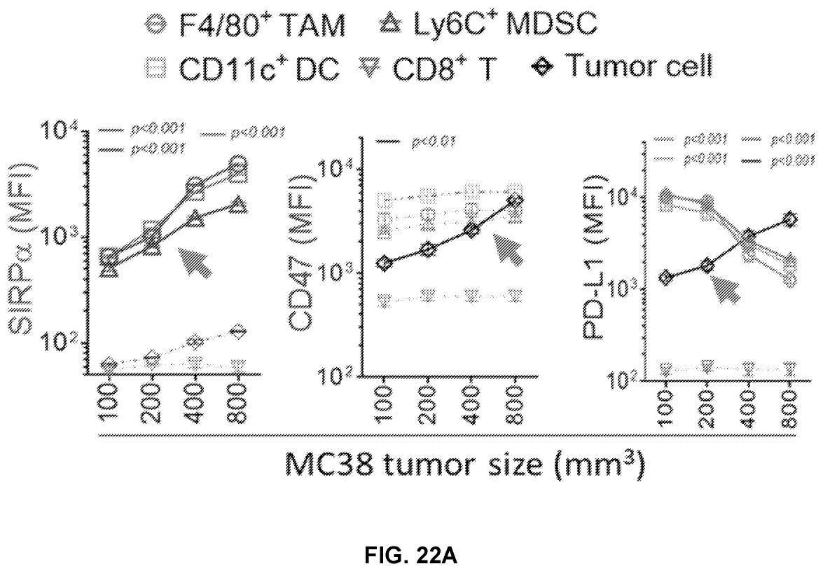

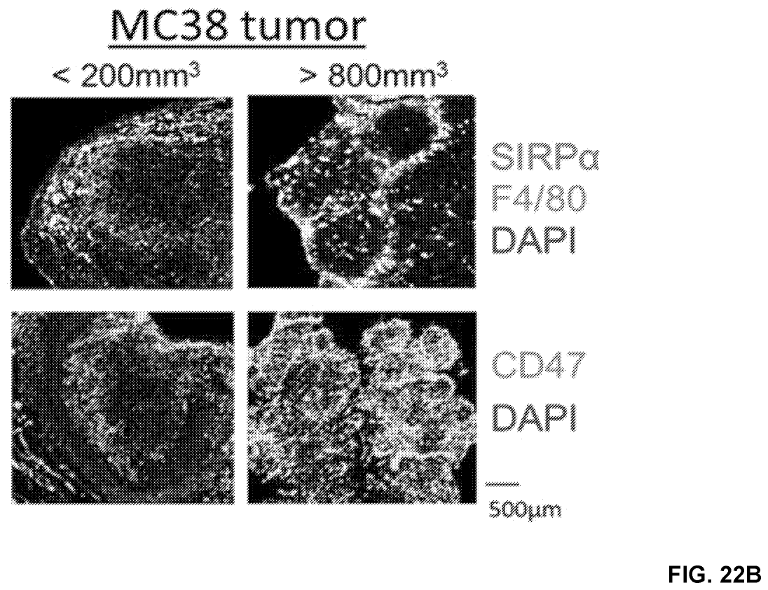

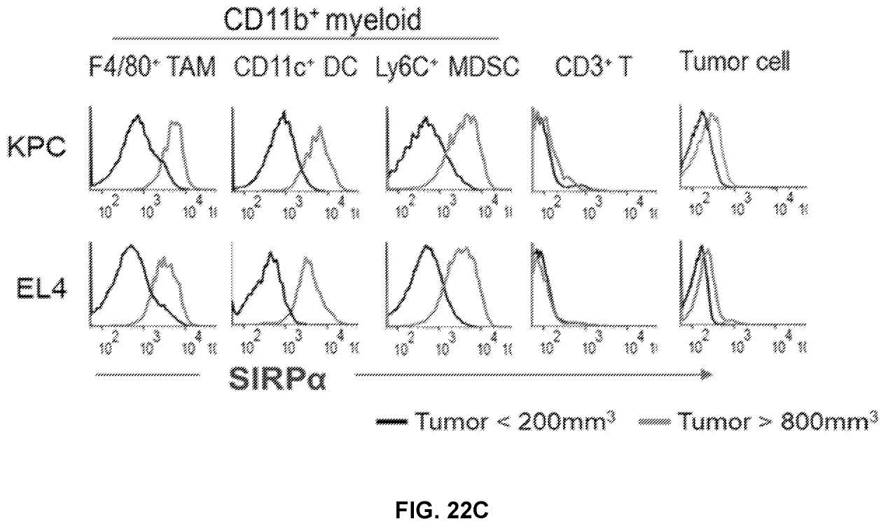

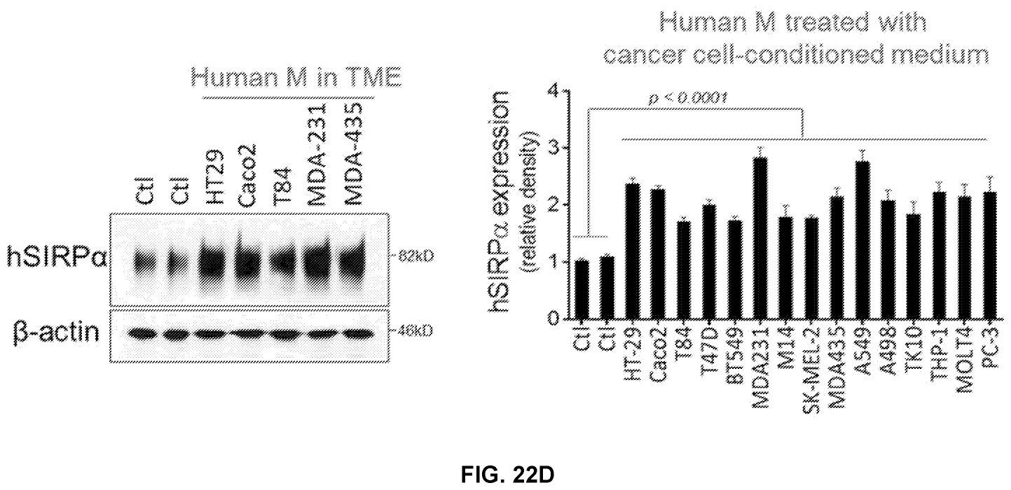

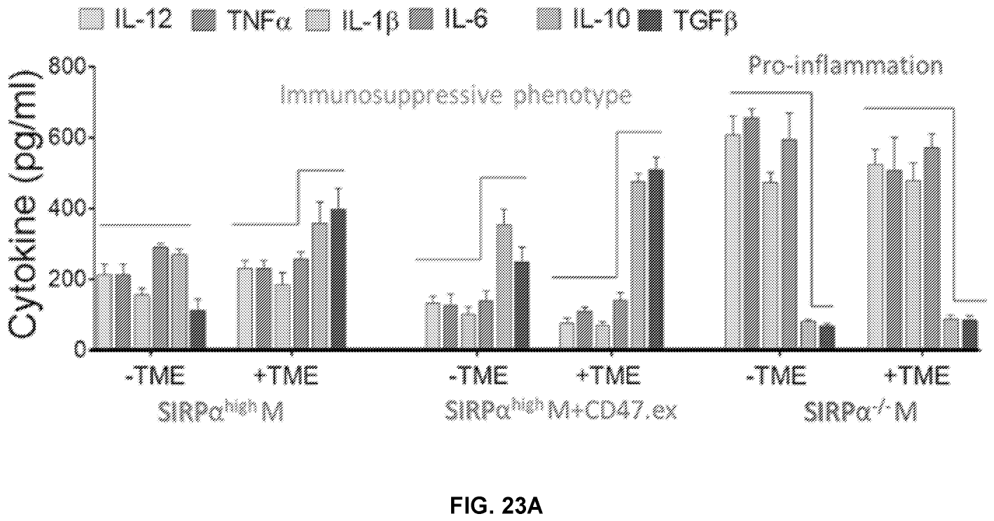

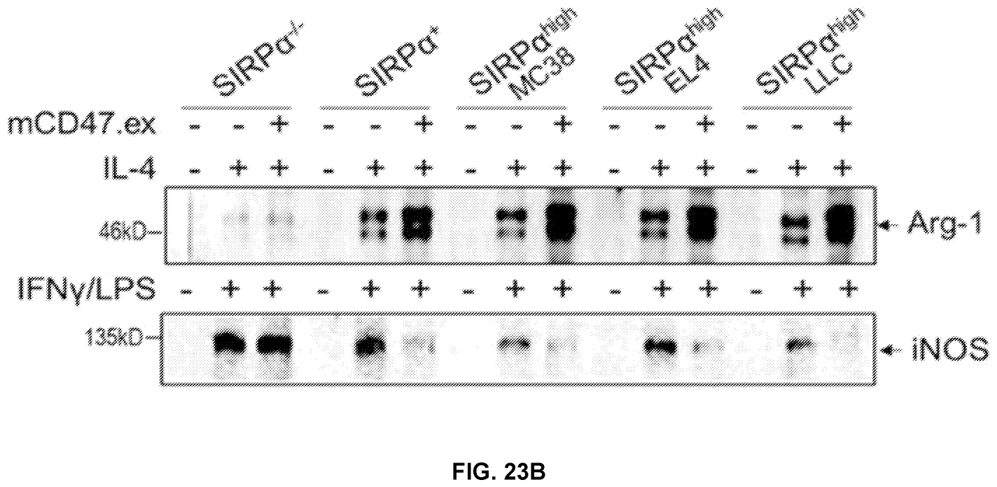

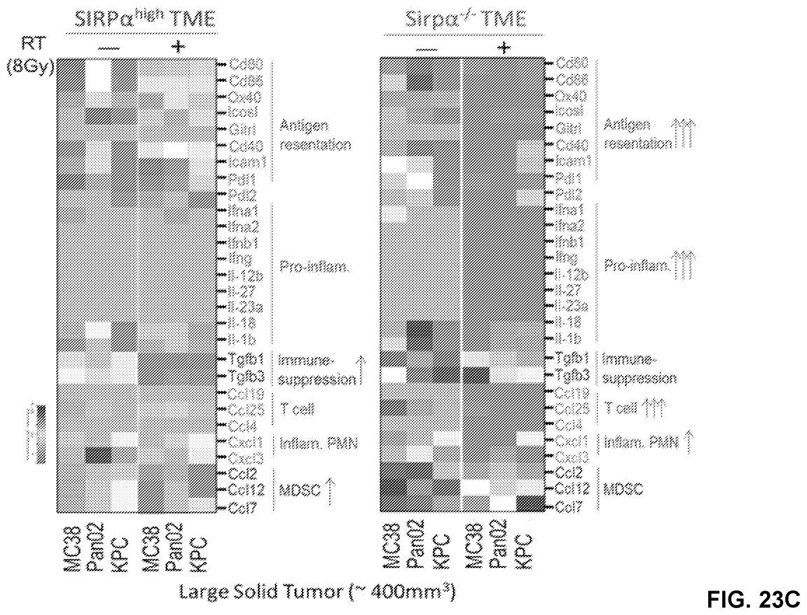

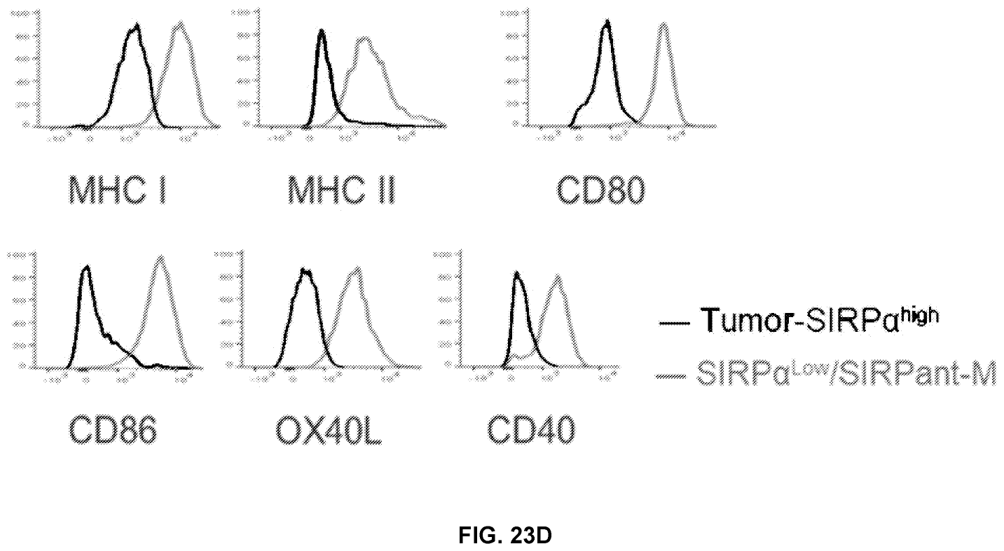

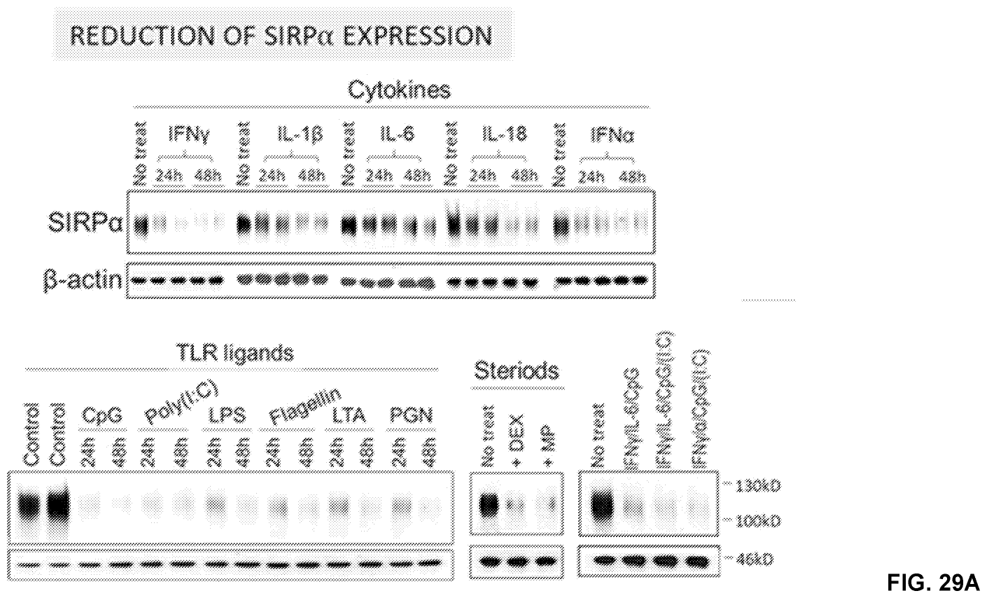

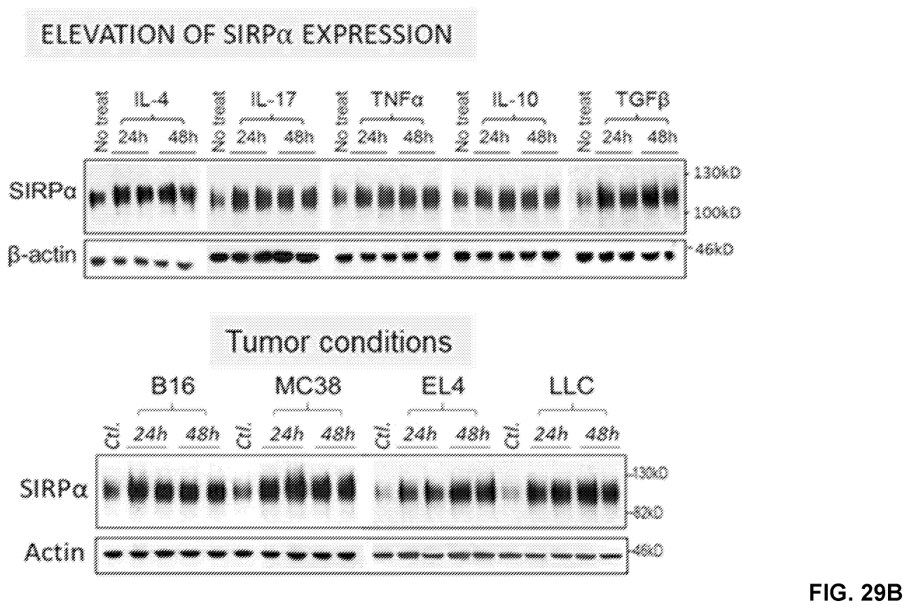

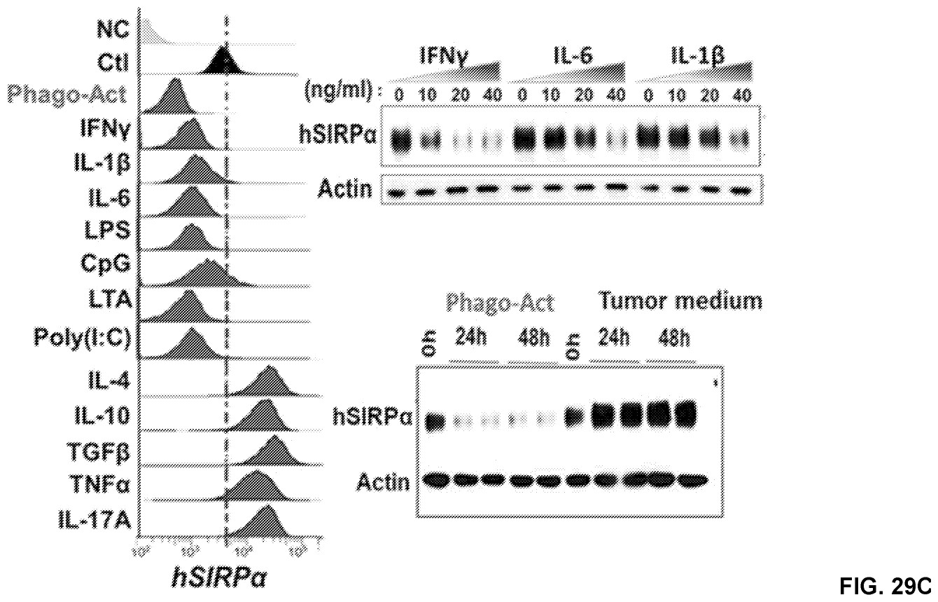

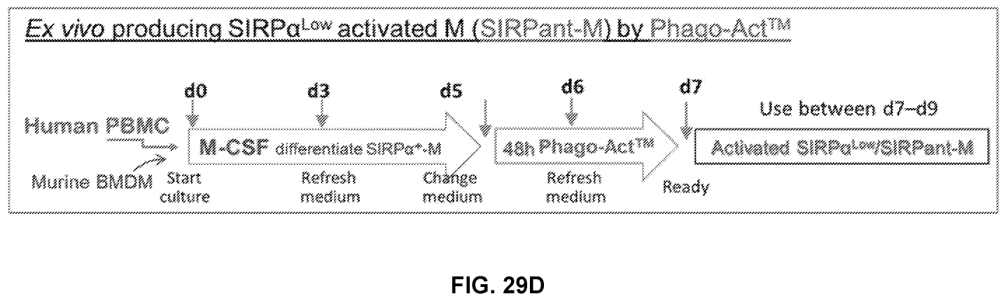

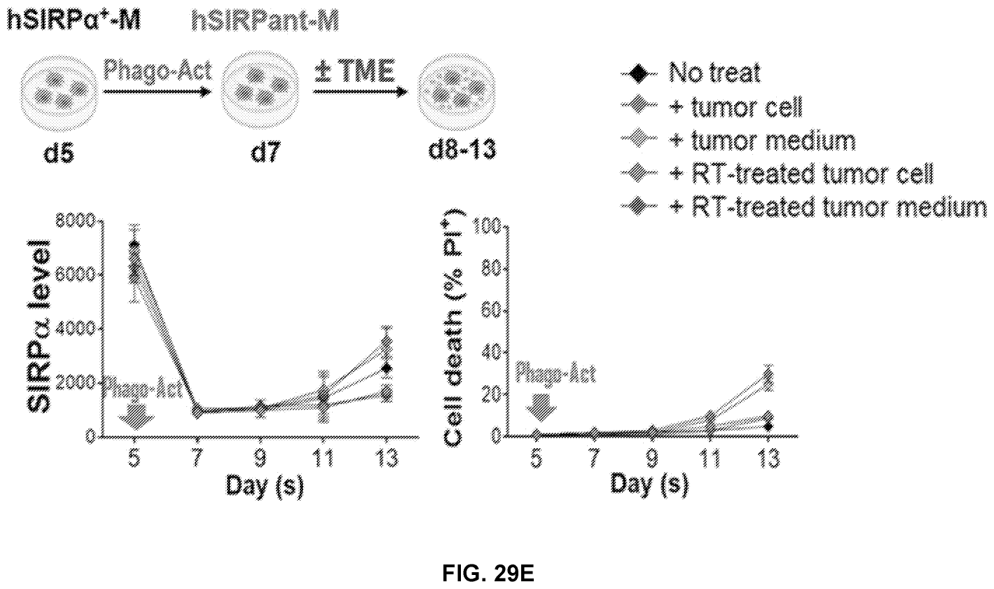

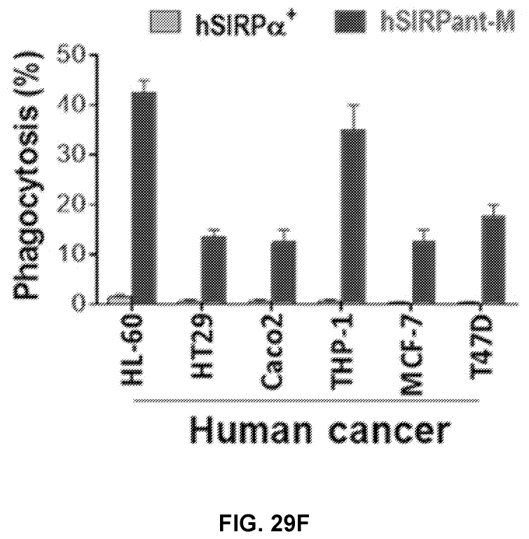

Studying different solid tumors in mice, it was found that SIRPα controls TME immunogenicity by bolstering the immunosuppressive phenotype of TAMs. The expression of SIRPα on TAMs, dendritic cells (DCs) and myeloid-derived suppressor cells (MDSCs) progressively increases as tumors grow (FIG. 22), an effect attributed to both the dynamic nature of SIRPα and that cancer cells and the TME produce factors, e.g., IL-10, IL-4, TGFβ, IL-17, etc., that upregulate SIRPα expression (see FIG. 29). SIRPα expression on macrophages profoundly affects their responses to pro- and anti-inflammatory stimuli and thus determines their subsequent effector functions. Comparing macrophages with a high level of SIRPα (SIRPα^{high}-M) to those without (i.t. Sirpα^{-/-}-M and SIRPα^{low}-M, the latter also termed SIRPant-M) showed that SIRPα^{high}-M preferentially adopt a hyper-immunosuppressive phenotype characterized by elevated expression of IL-10, TGFβ and arginase-1, general resistance to proinflammatory activation and diminished expression of antigen presentation machinery (FIG. 23). Even when exposed to strong proinflammatory stimulation such as the characteristic M1-phenotype treatment LPS plus IFNγ, SIRPα^{high}-M induced only weak expression of proinflammatory molecules but highly expressed IL-10, the amount of which equaled or exceeded the sum of their proinflammatory cytokine production. These dramatic results suggest that high SIRPα expression in the TME is inherently self-reinforced whereby the immunosuppressive TME upregulates SIRPα, which then further drives TAMs to strengthen tumor immunosuppression. Moreover, SIRPα's capacity to strongly inhibit proinflammatory signals implicates its role in TAM resistance to undergo a proinflammatory phenotypic switch in response to therapeutic treatments. Indeed, high SIRPα expression promotes immunosuppression (IL-10) and drives TAM activation towards a wound-healing response under cancer therapies, facilitating tumor recovery and progression. Supporting this notion, LPS/IFNγ-treated SIRPα^{high}-M were found to have increased production of the chemoattractant CCL2, which recruits monocytes/MDSCs to drive wound healing, but minimal secretion of CXCL1/2, which attracts proinflammatory neutrophils that promote tumor tissue damage.

In contrast to SIRPα^{high}-M, Phago-Act™-treated SIRPα^{low} macrophages (also termed SIRPANT-M) exhibited an opposing, predominantly proinflammatory polarization and a poorly immunosuppressive phenotype in response to the same stimuli. Similar to LPS/IFNγ-treated Sirpα^{-/-}-M, SIRPANT-M produced elevated levels of IL-12, IL-1β, IL-6, TNFα, and CXCL1/2, but not CCL2, and exhibited higher expression of antigen presentation machinery including MHC-I, MHC-II and co-stimulatory molecules CD80, CD86, OX40L, CD40, etc. (FIG. 23).

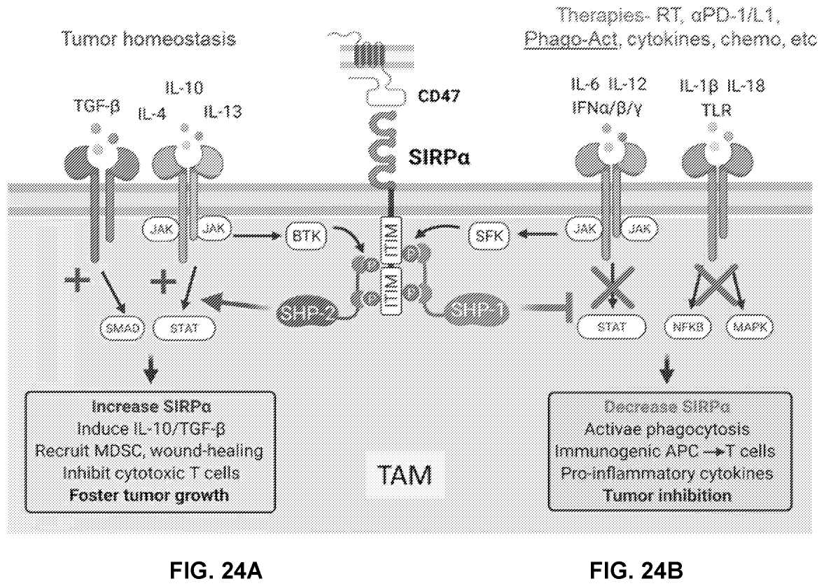

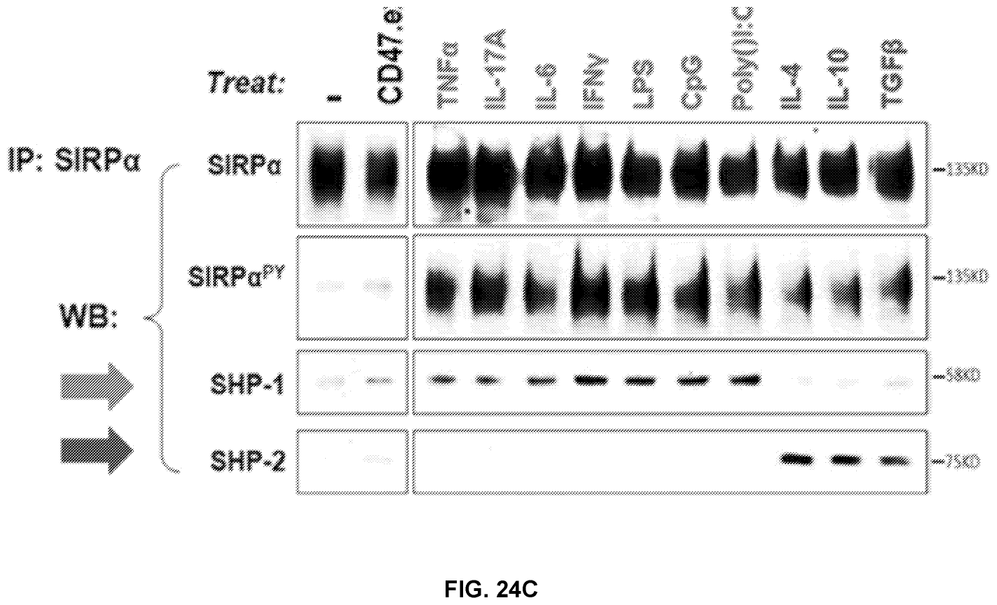

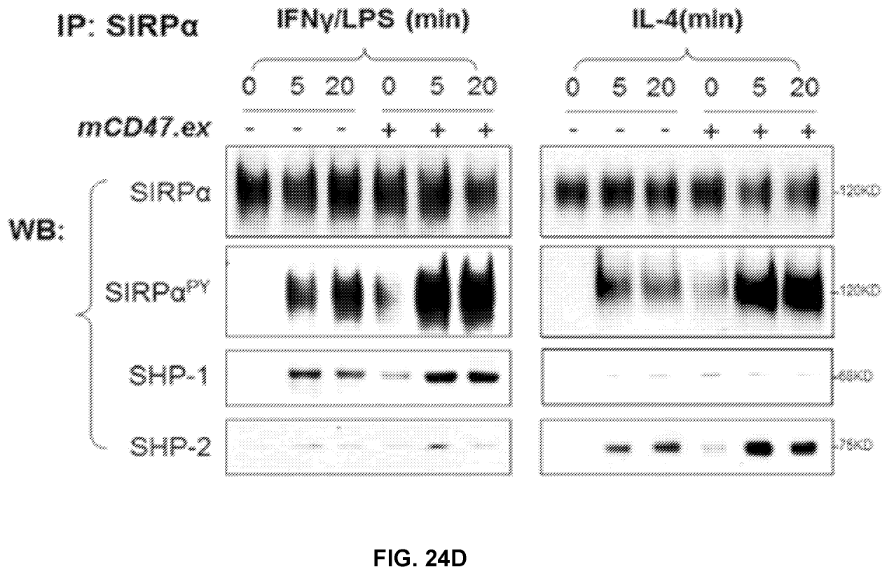

SIRPα Controls Macrophage-Polarizing Signal Transduction Mechanistic studies (FIG. 24) revealed that macrophage immunophenotype and function are regulated by SIRPα via its cytoplasmic ITIMs, which undergo tyrosine phosphorylation upon macrophage stimulation and provide distinct docking sites for SHP-1 or SHP-2, the major cellular tyrosine phosphatases that regulate downstream signaling events. Cytokine-, TLR agonist- or other stimuli-induced tyrosine kinase activities are required for SIRPα ITIMs phosphorylation. Under tumor homeostasis, TAMs are constantly exposed to immunosuppressive cytokines (e.g., IL-4/13, IL-10) that activate Bruton's tyrosine kinase (Btk), which phosphorylates SIRPα ITIMs in a manner that causes exclusive docking of SHP-2, but not SHP-1. This series of events, where immunosuppressive cytokines activate Btk to drive SIRPα-SHP-2 binding, prohibits SHP-2 from inhibiting IL-4/13R or IL-10R, thereby enhancing immunosuppressive signal transduction in macrophages (FIG. 24A). As an additional consequence of this pathway, SIRPα expression further increases in TAMs and thus dominantly controls their phenotype.

Under proinflammatory conditions elicited by cancer therapies, immunomodulatory treatments, cytokines, TLR agonists or other stimuli, Src family tyrosine kinases (SFK) are induced and phosphorylate SIRPα ITIMs (FIG. 24B). Unlike Btk, SFK phosphorylates ITIMs in a pattern that leads to docking and activation of SHP-1. By dephosphorylating multiple proteins, SHP-1 diminishes IFNα/β/γ-mediated JAK-STAT and PI3k-Akt pathways that induce expression of antigen presentation machinery and co-stimulatory molecules (Kalbasi 2020). Likewise, SHP-1 inhibits proinflammatory cytokines/TLR-mediated MAPK and NFκB pathways that activate phagocytosis, drive inflammation and/or exaggerate other proinflammatory signals, including those that downregulate SIRPα expression (see FIG. 29). In established tumors, SIRPα-SHP-1 mediated inhibition of inflammation, along with SIRPα-SHP-2 mediated enhancement of immunosuppression, largely remain intact during cancer therapy and therefore greatly dampen or nearly completely abrogate the efficacy of most treatment modalities. For example, treatment-induced cell damage signals (DAMPs) through TLRs in TAMs whose high SIRPα expression (SIRPα^{high}) skew the response toward strong wound-healing that amplifies production of IL-10, TGFβ and CCL2, the latter attracting MDSC to inhibit T cell-mediated anticancer immunity. FIG. 24 depicts the dichotomous SIRPα regulation mediated by SHP-2 or SHP-1, which either promotes an immunosuppressive macrophage phenotype (via SHP-2) or inhibits proinflammatory macrophage activation and antigen presentation (via SHP-1). CD47 ligation is not required for SIRPα regulation (FIG. 24D); however, CD47 ligation does induce a structural change(s) in SIRPα's cytoplasmic domain that facilitates SIRPα ITIMs phosphorylation by kinases, thereby enhancing SHP-1/2 docking and the strength of subsequent downstream regulation.

Predicated upon the understanding of these mechanisms, SIRPANT's strategy is to manufacture therapeutic SIRPαlow macrophages, SIRPANT-M, via an ex vivo process, thereby avoiding the immunosuppressive TME and strong SIRPα-mediated regulation therein that quench the effect of Phago-Act™ (see FIG. 29). Our previous studies showed that, while Phago-Act™ has the capacity to downregulate SIRPα and activate phagocytosis, injecting Phago-Act™ or other proinflammatory reagents into established tumors achieves a muted response and minimally reduces SIRPα expression on TAMs or controls the tumor. Even multiple injections of Phago-Act™ combined with tumor-directed radiation failed to reduce tumor burden, only moderately curbing tumor growth. These results are typical especially when challenging hard-to-treat cancers such as MC38 colorectal carcinoma and pancreatic ductal adenocarcinoma KPC and PanO2, all of which comprise a highly immunosuppressive TME and thus resist therapies such as tumor radiation (RT), anti-PD-1/L1 checkpoint blockade, and their combination.

### Depleting SIRPα Reprograms the TME and Enables Elimination of Cancer Cells

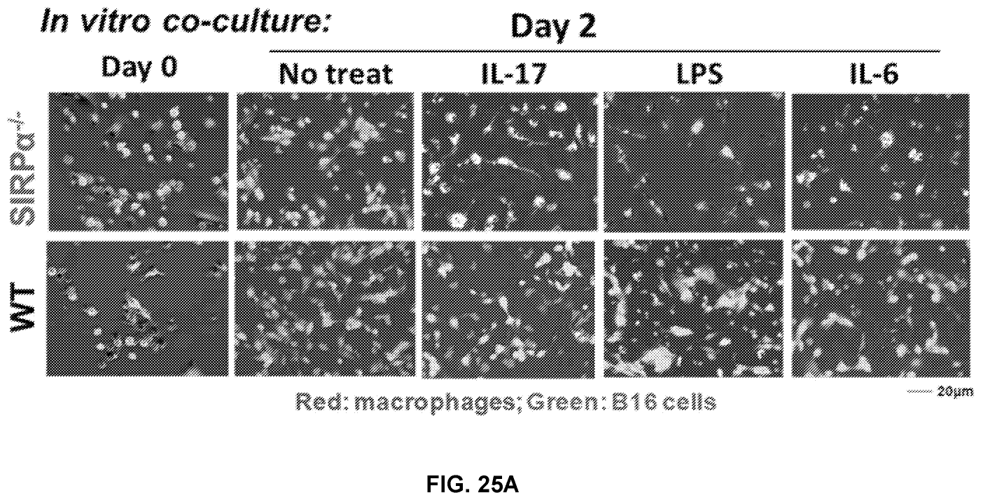

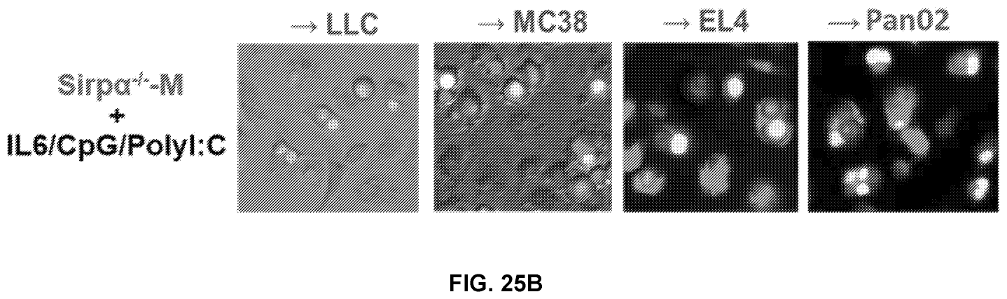

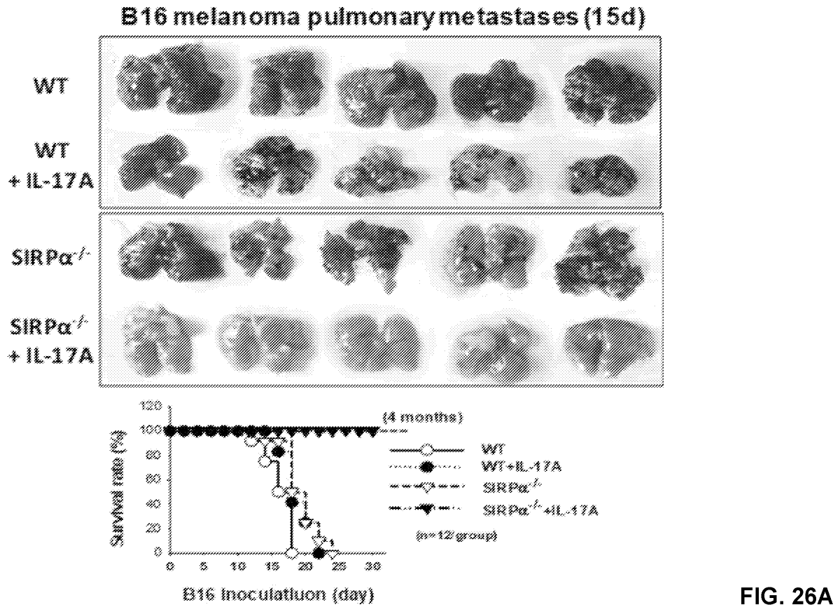

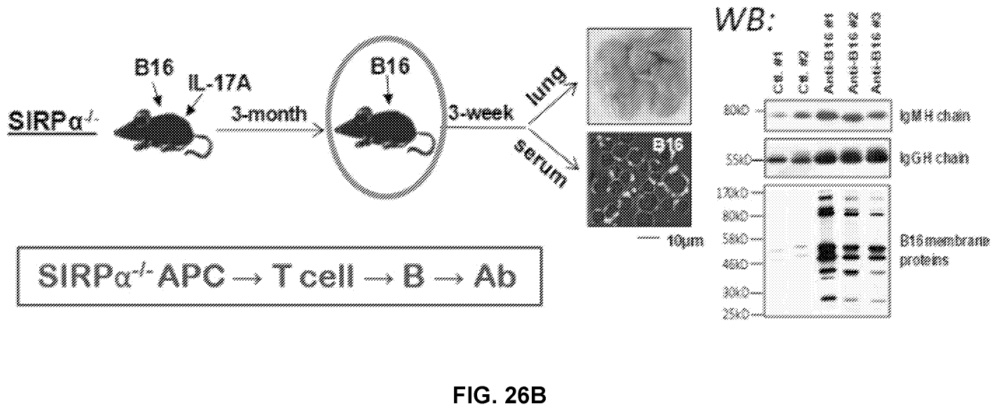

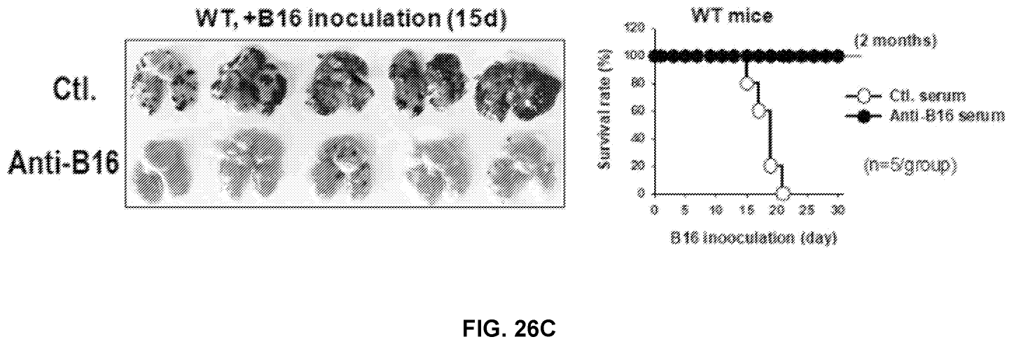

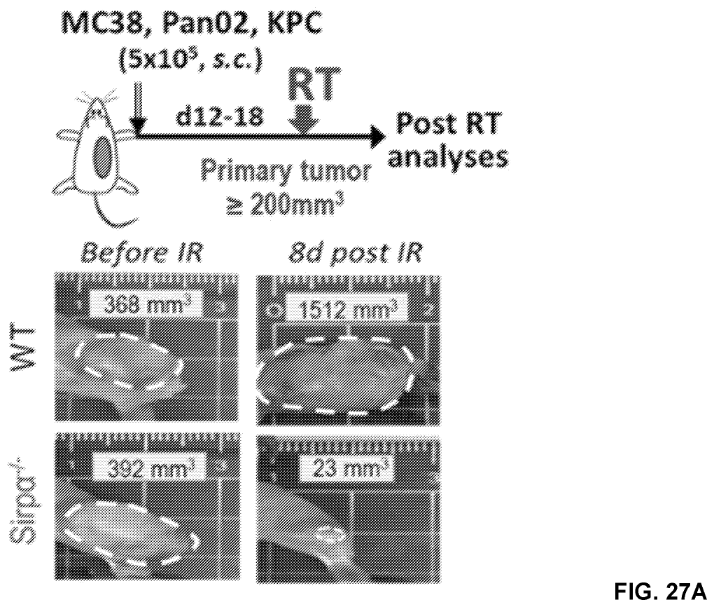

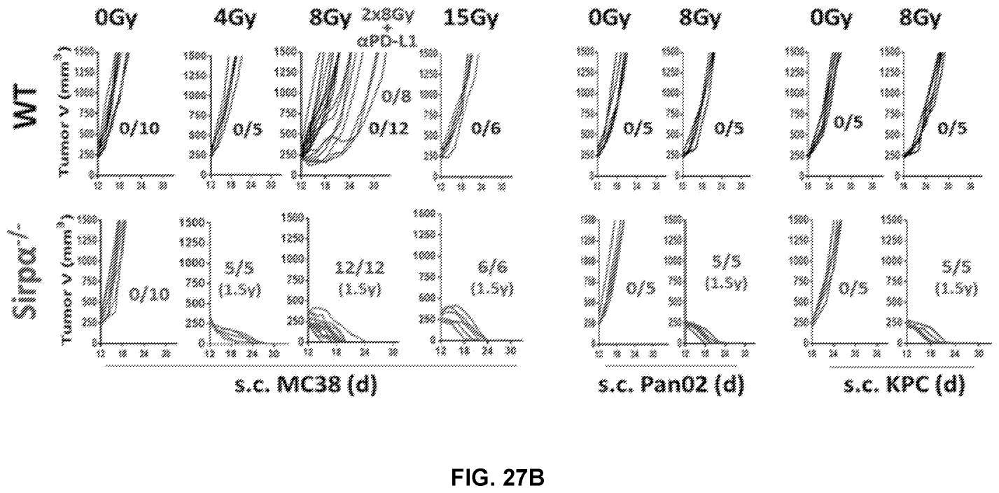

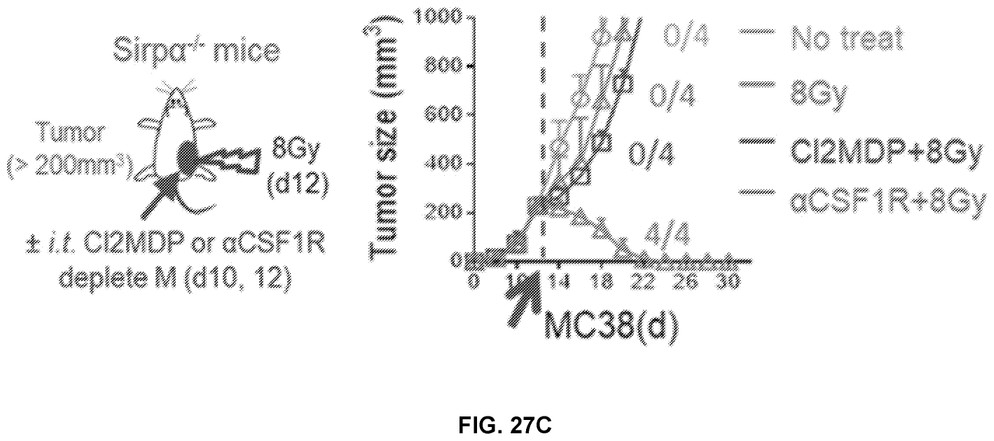

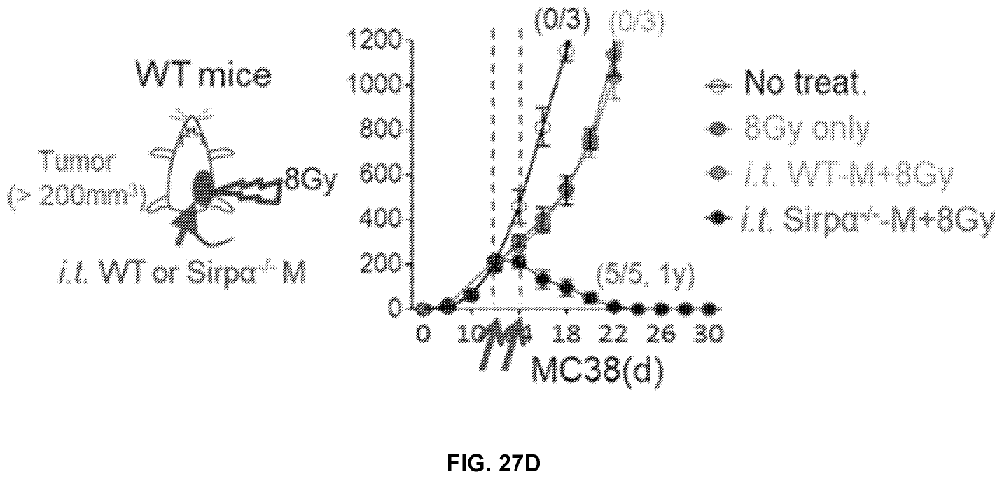

Despite that SIRPα depletion alone does not lead macrophages to phagocytose cancer cells, combining SIRPα depletion with cytokine/TLR agonist-mediated activation turn macrophages into potent cancer-eliminating phagocytes (FIG. 25). Studies using Sirpα^{-/-} mice found that these mice, though manifesting no natural immunity prohibiting tumor formation, frequently exhibit a complete response (CR) to immunomodulatory therapeutic treatments and systemically clear even late-stage cancer with distal lesions (metastases). Multiple syngeneic cancers have been tested in Sirpα^{-/-} mice, and these include melanoma (B16), lymphoma (EL4), Lewis Lung Carcinoma (LLC), colorectal carcinoma (MC38), pancreatic ductal adenocarcinoma (PanO2, KPC), and DSS-AOM-induced spontaneous colorectal cancer. In all cases, treating established tumors in Sirpα^{-/-} mice with simple cytokine plus TLR agonist schemes (FIG. 26), or a fraction of non-ablative X-ray RT (4-15Gy), induced dramatic anticancer responses that led to rapid regression and eventual elimination of large tumors along with non-treated distal lesions (abscopal effect) (FIGS. 27 & 28). Moreover, this strong anticancer response conferred long-lasting anticancer immunity that prevented recurrence. It is worth noting that these solid tumors, especially MC38, KPC, PanO2 and LLC, are notoriously hard-to-treat cancers in preclinical studies, with established tumors>200 mm³ having been shown to resist otherwise effective therapies including immune checkpoint inhibitors, RT and other combinations. In our experiments, administering high doses of RT combined with anti-PD-1 failed to control these tumors in WT mice. Indeed, the degree to which immunomodulatory treatments were efficacious against these tumors in Sirpα^{-/-} mice has not been seen in the literature or found elsewhere.

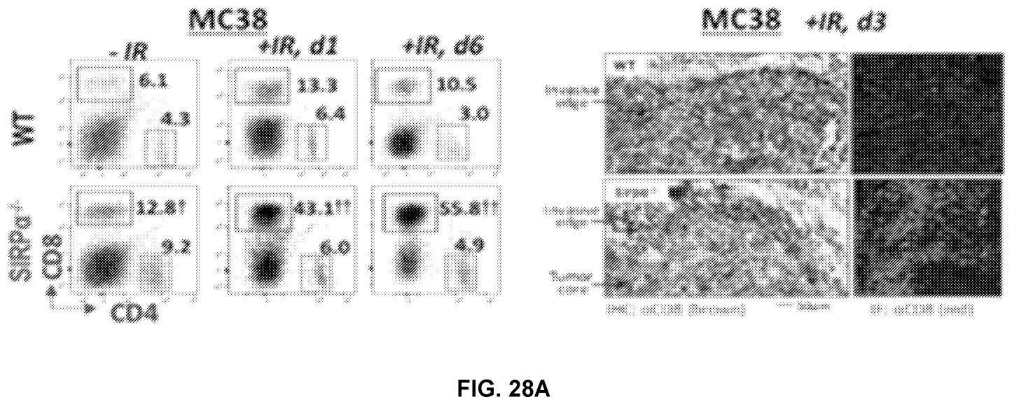

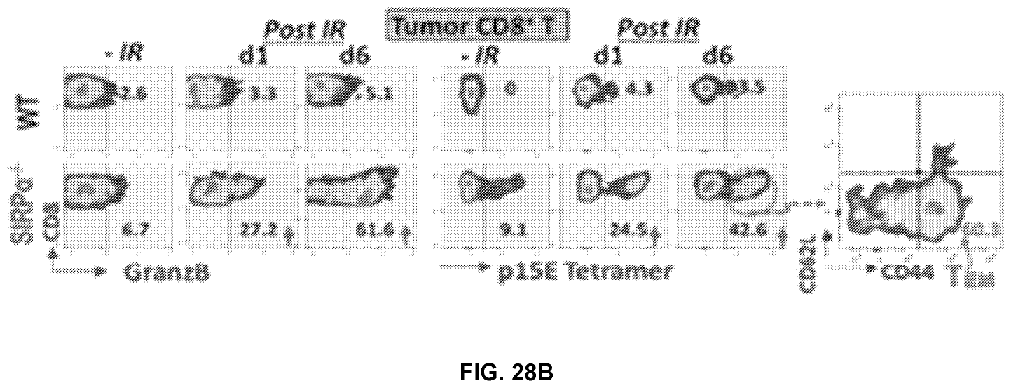

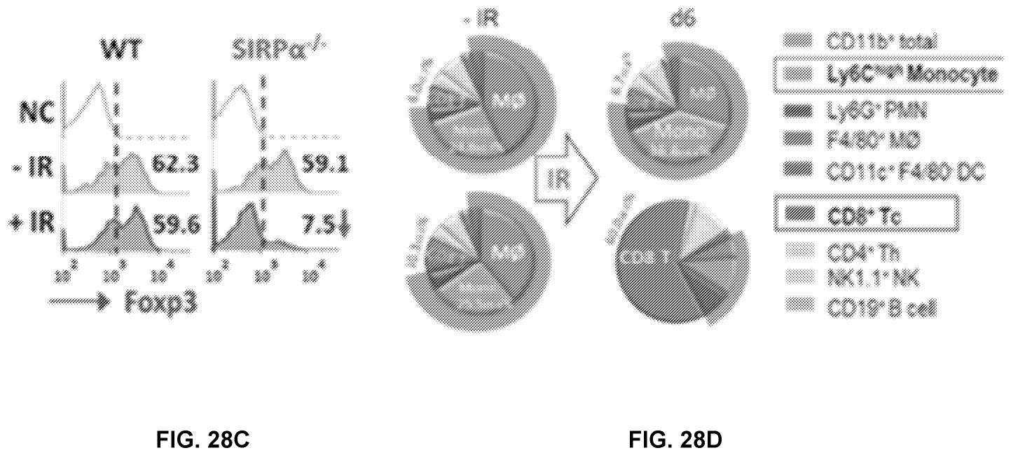

During these studies, it was found that intratumoral Sirpα^{-/-}-M played a critical role in tumor elimination, as depletion of this population abrogated the curative response in treated Sirpα^{-/-} mice (FIG. 27C). Meanwhile, adoptive transfer of Sirpα^{-/-}-M into tumors in WT mice dramatically reversed their resistance to therapies and conferred tumor regression (FIG. 27D). Though intratumoral Sirpα^{-/-}-M predicated the anticancer response, it was found that the therapeutic efficacy leading to tumor elimination was not solely due to enhanced Sirpα^{-/-}-M phagocytosis of cancer cells, but rather was the result of activated Sirpα^{-/-}-M that acquired cancer antigens following phagocytosis and then conducted immunogenic antigen presentation that activated a large number of tumoricidal T cells. The expansion of cytotoxic T cells (CD8+) in the TME rapidly occurred following tumor treatment (24 h-post) and coincided with the TME turning from “Very Cold” to “Very HOT” in terms of CD8 T cell infiltration (FIG. 28A). These CD8 T cells highly expressed molecules indicative of cancer-specificity (p15E), potent tumoricidal capacity (granzyme B), and hallmarks of immune memory (CD44⁺CD62L⁻, T_{EM}), attributes that contributed to T cell-mediated abscopal inhibition and clearance of cancerous lesions (FIG. 28B). Mechanistic studies confirmed that Sirpα^{-/-}-M induce activation of T cells through in situ calling tumor-specific memory T cells (i.e. T_{EM}/T_{RM}), a response that is faster and much more robust than DC-mediated activation of naïve T cells in lymphoid organs. Additionally, the robust proinflammatory features of activated Sirpα^{-/-}-M drove an anticancer response that attracted antitumor neutrophil and cytotoxic NK cells while reducing immunosuppressive Tregs and MDSC, together forming a tumoricidal tissue niche that fostered cancer elimination (FIGS. 28C-28D). In contrast, tumors without Sirpα^{-/-}-M responded to RT by steering the TME toward wound-healing and strengthened immunosuppression by increasing TGFβ and MDSC infiltration. Profiling the transcriptome of various tumors (e.g., MC38, KPC and PanO2) that did or did not comprise Sirpα^{-/-}-M prior to or after RT confirmed that Sirpα^{-/-}-M initiate striking anti-tumor responses and reshape the immune landscape to promote tumor elimination.

### Discovery of a Non-Genetic Approach to Downregulate SIRPα in Macrophages

SIRPANT's strategy employs phagocytosis-activated SIRPα^{low} macrophages, SIRPANT-M, which display characteristics similar to activated Sirpα^{-/-}-M, as the central therapeutic weapon against cancer. The development of SIRPANT-M is based on the finding that IFNγ, although having no ability to activate phagocytosis, drastically reduces SIRPα protein expression in macrophages from mice and humans (FIGS. 29A-29C). Screening other factors further found that cytokines IL-1β, IL-18, IL-6, IFNα and IFNβ, and all TLR agonists tested thus far (LPS, CpG, LTA, flagellin, Poly I:C, PGN, etc.) downregulate SIRPα, while simultaneously activating phagocytosis. Unlike their capacity to rapidly activate phagocytosis (1-6 h), these factors require approximately 2 days to downregulate SIRPα (>90%), the mechanism of which involves cytokines- and TLR-mediated signal transduction leading to induction of three micro RNAs (mir-17/20a/106a) that in turn inhibit SIRPα mRNA translation. Conversely, we found immunosuppressive cytokines IL-10, TGFβ, IL-4 and IL-13, and proinflammatory cytokines IL-17 and TNFα upregulate SIRPα expression, albeit the latter two also activate phagocytosis. Additionally, we also found that dexamethasone (DEX) and methylprednisolone (MP) downregulate SIRPα expression; however, these glucocorticoids potently inhibit macrophage phagocytosis. These comprehensive studies investigating the dynamics of SIRPα expression and macrophage phagocytic activation informed the selection of reagents to generate therapeutically applicable SIRPANT-M. Innumerable iterations have been exhaustively explored, re-tested and optimized under various experimental conditions, from which a cytokine and TLR agonist cocktail became the core of the proprietary reagent, Phago-Act™. In a single step of treatment, Phago-Act™ potently downregulates SIRPα (SIRPα^{low}), activates macrophage phagocytosis towards cancer cells and endows macrophages with an augmented proinflammatory phenotype and the immunogenic antigen presentation capacity.

### Phago-Act™

The proprietary reagent Phago-Act™ contains four components, recombinant human interferon-gamma (IFNγ), recombinant human interferon-alpha A2 (IFNα), CpG oligodeoxynucleotide, and polyinosinic:polycytidylic acid (Poly I:C), used for ex vivo treatment of macrophages of both human and mouse origins. In Phago-Act™, IFNγ can be present in a range of from 40 ng/ml to 200 ng/ml, IFNα can be present in a range of from 40 ng/ml to 200 ng/ml, CpG oligodeoxynucleotide can be present in a range of from 1 μg/ml and 5 μg/ml, and Poly I:C can be present in a range of from 1 μg/ml and 5 μg/ml. In a specific embodiment of Phago-Act™ (IFNγ, is present at a concentration of 100 ng/ml, IFNα is present at a concentration of 100 ng/ml, CpG oligodeoxynucleotide is present at a concentration of 2 μg/ml, and Poly I:C is present at a concentration of 2 μg/ml.

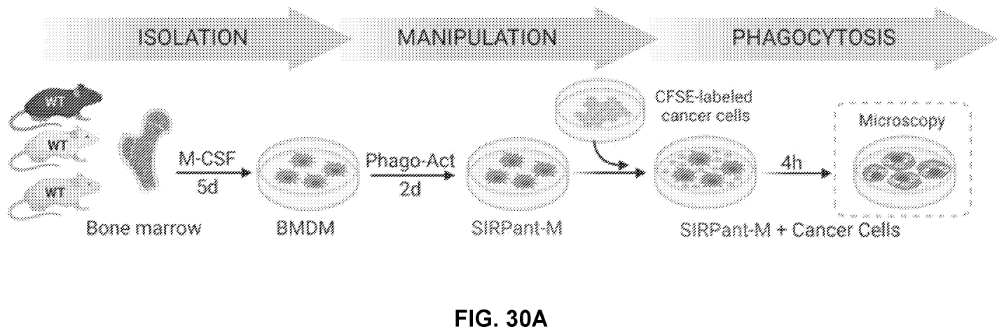

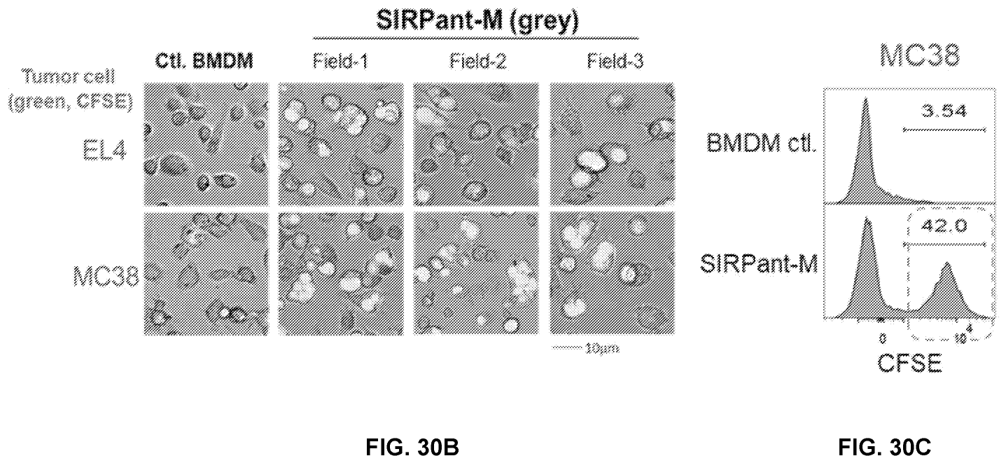

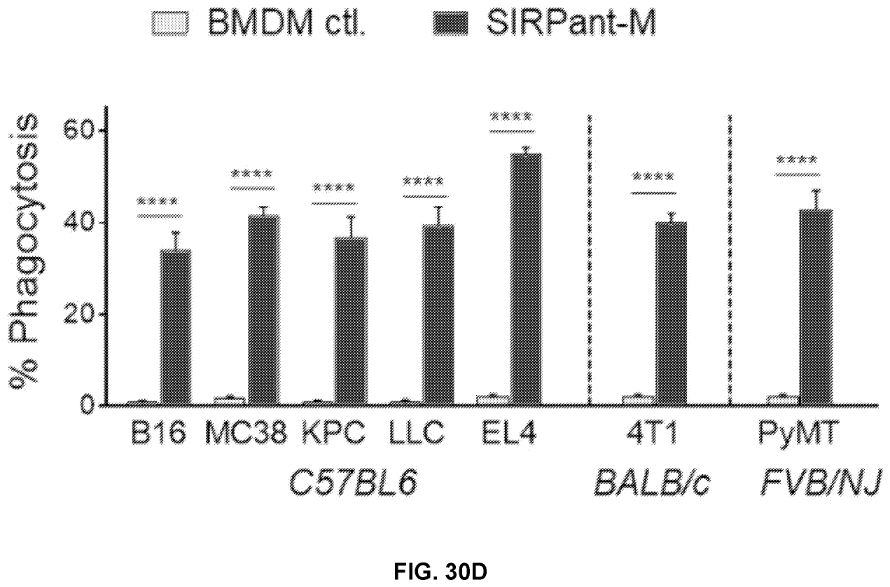

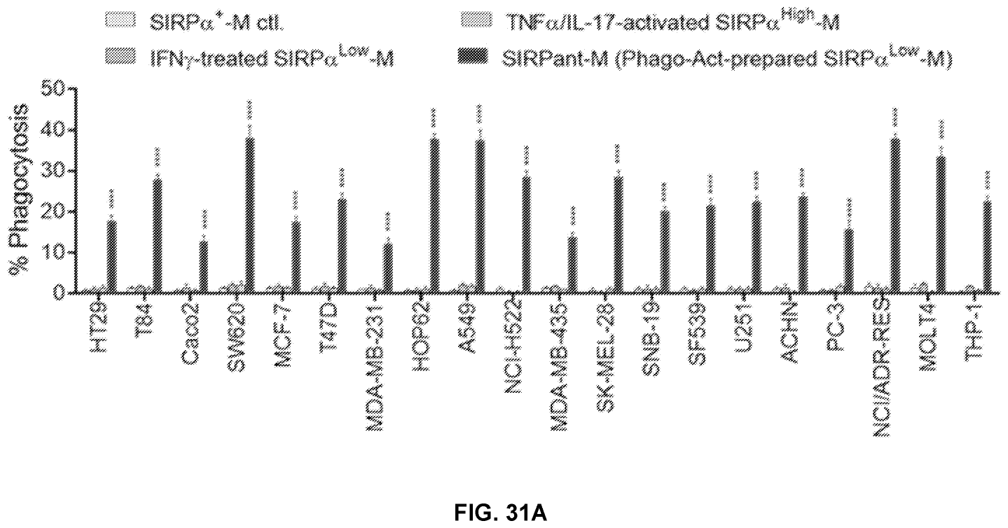

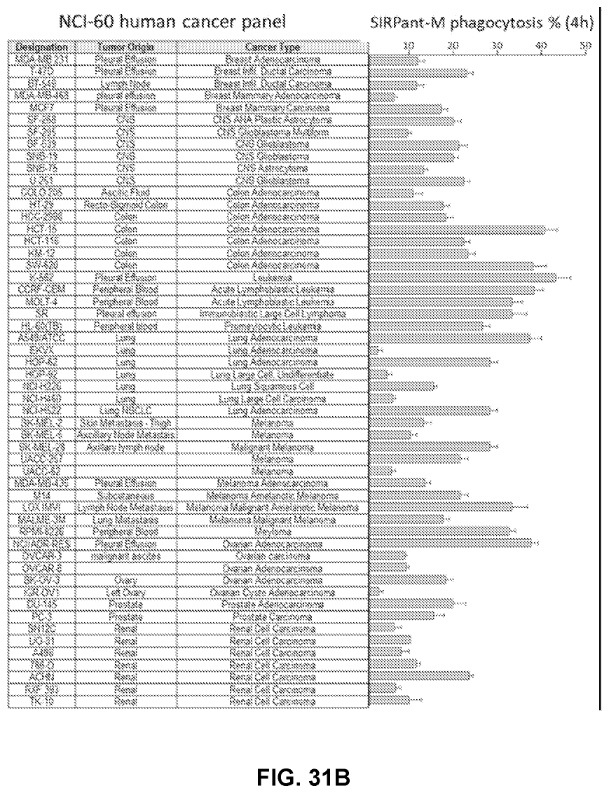

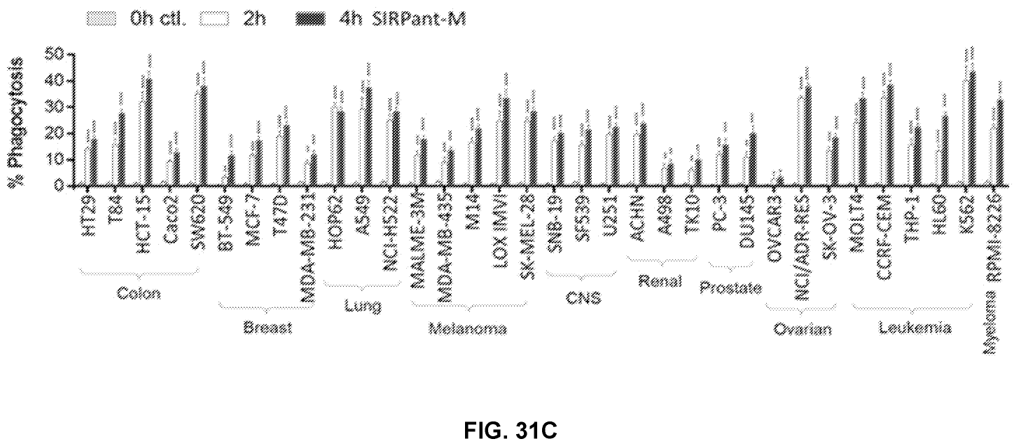

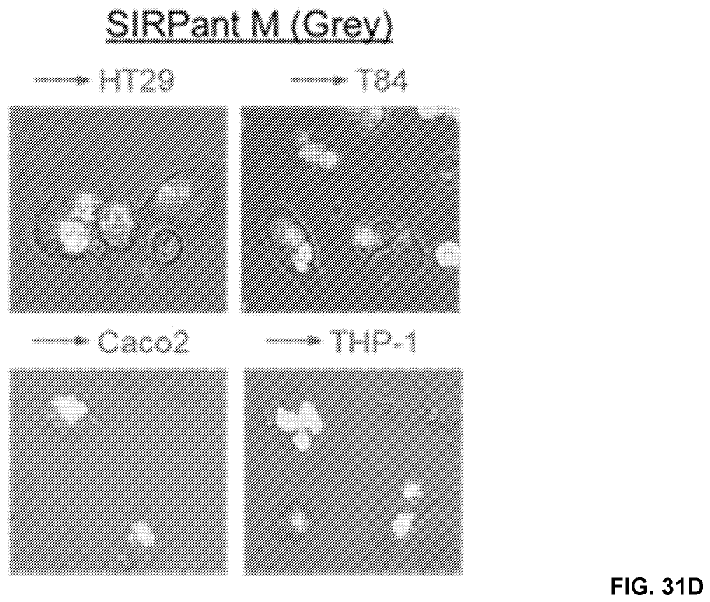

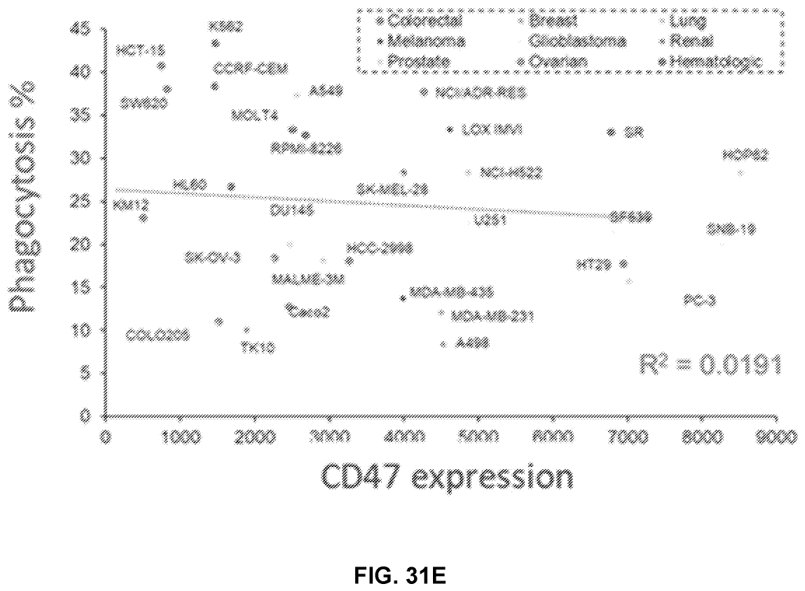

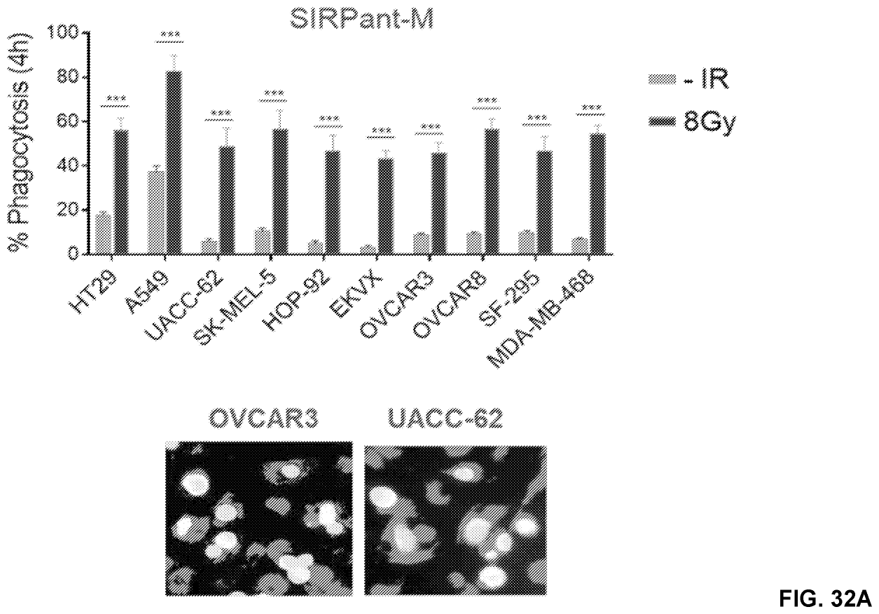

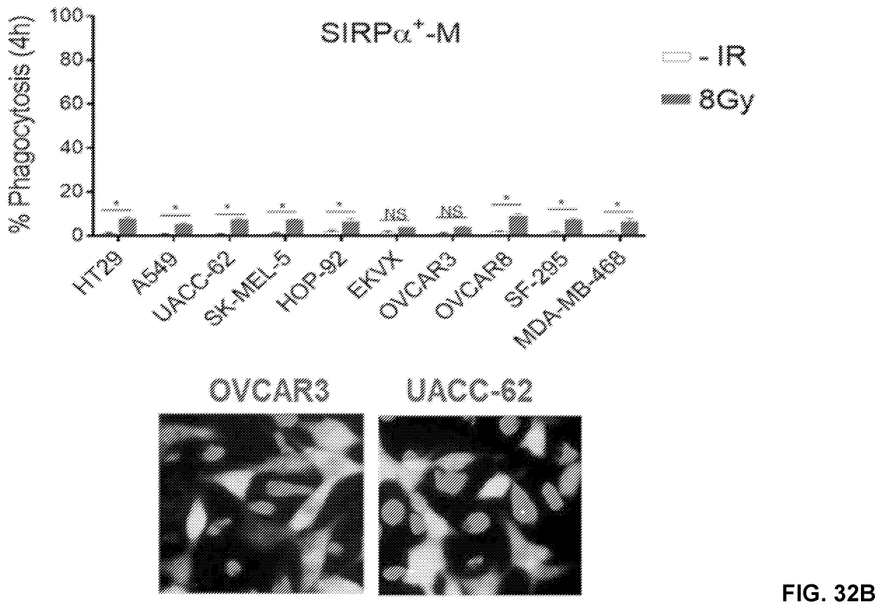

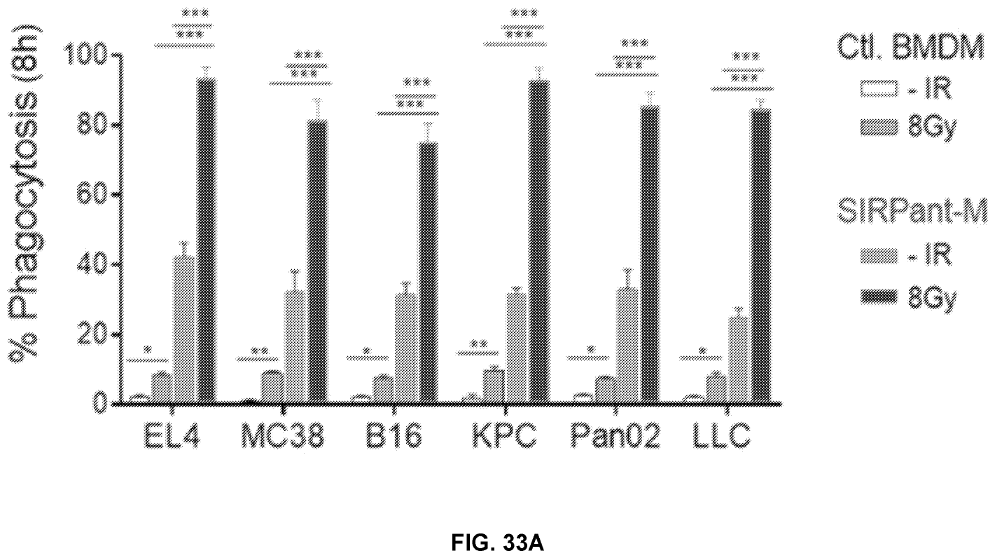

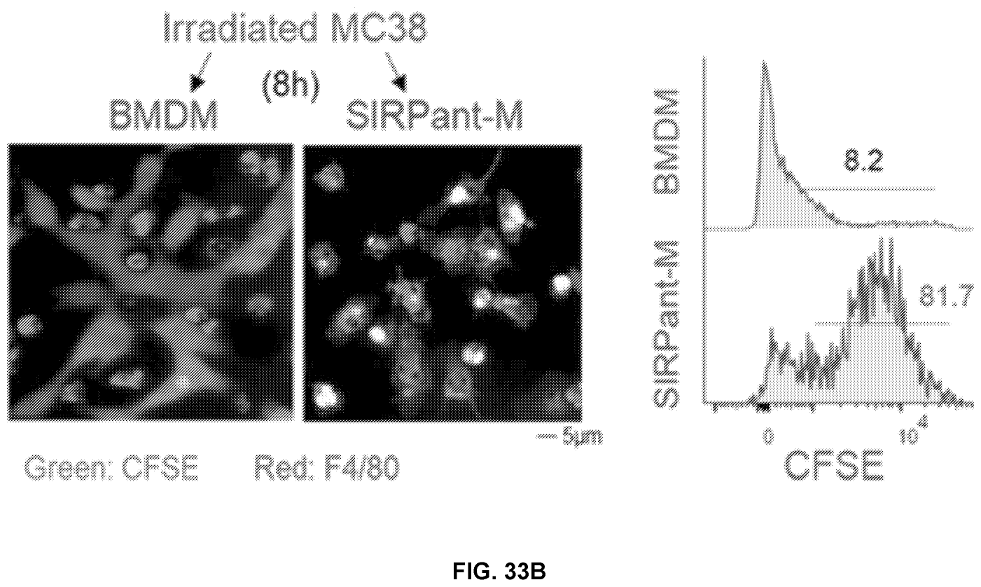

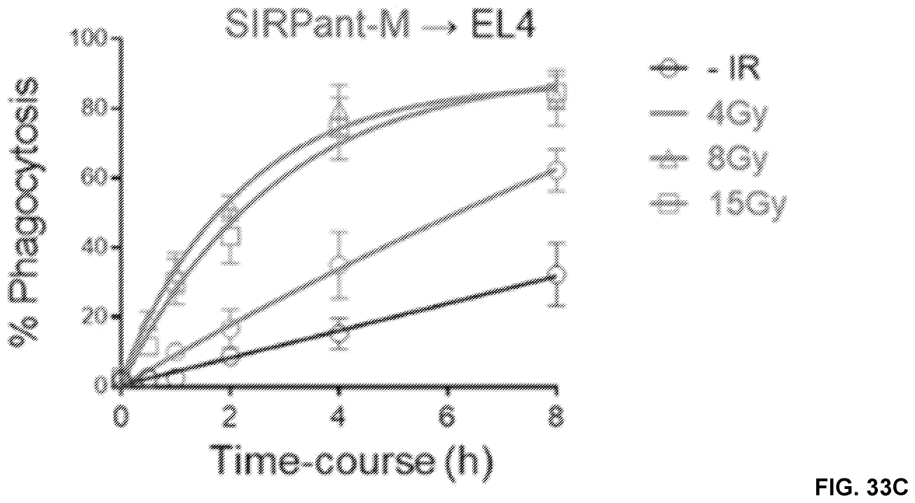

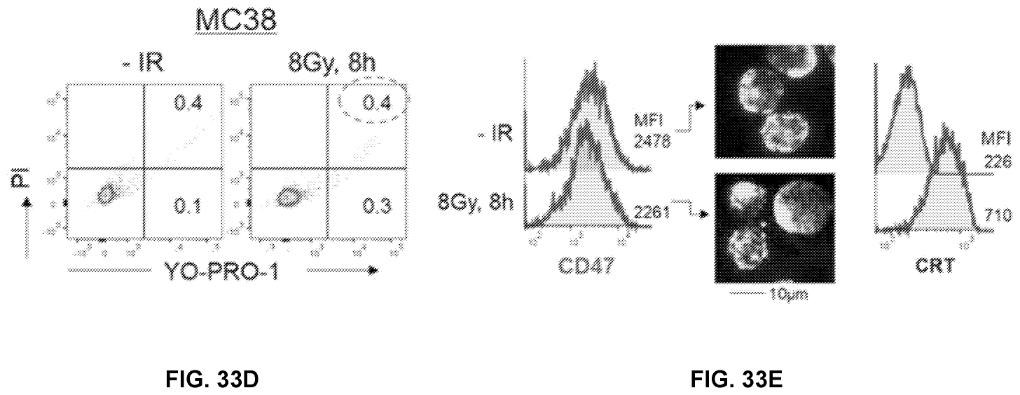

The combination of these reagents is the key intellectual property of SIRPANT technology and are specially prepared under quality control to ensure effectiveness and consistency. To prepare therapeutic-effective autologous SIRPANT-M (SIRPα^{low} activated macrophages), PBMC-derived SIRPα⁺-M prepared from cancer patients with M-CSF are treated with Phago-Act™ for 48 hours (2 days) (FIG. 29D depicts the workflow) to markedly reduce SIRPα expression, producing a population of SIRPα^{low} macrophages phenotypically and functionally similar to that seen when SIRPα is genetically knock out. Not only downregulating SIRPα, the formula of Phago-Act™ also at once bestows macrophages with potent phagocytosis capacity, a hyper-proinflammatory phenotype and increased expression of immunogenic antigen presentation machinery. Ex vivo phenotypic analyses show that SIRPANT-M maintain phenotypic stability and viability for at least three days following completion of Phago-Act™ treatment (FIG. 29E), a period allowing clinical practices to treat patients. Assaying SIRPANT-M phagocytosis confirmed their capacity to engulf a range of cancer cells (FIG. 29F; additional data in the next section Pharmacology FIGS. 30-31). Similar experimental settings were employed to expand the phagocytic assessment of human SIRPANT-M to the entire NCI-60 panel of human cancer cells (FIG. 31). As anticipated, SIRPANT-M demonstrated phagocytosis towards all tested healthy cancer cells, and this phagocytic capacity was not contingent upon cancer cell expression of CD47 (R²=0.0191). Additional testing SIRPANT-M phagocytosis toward X-ray radiation-treated, non-apoptotic cancer cells showed further enhanced phagocytosis, as irradiated cancer cells express DAMPs that promote phagocytosis. In contrast, macrophages without Phago-Act™ treatment (SIRPα⁺-M) prepared from the same donors failed to phagocytose either healthy or irradiated cancer cells. The same method can also be used to produce SIRPANT-M from mice, and murine bone marrow-derived SIRPANT-M of different genetic backgrounds exhibited phagocytosis towards their syngeneic cancer cells, such as C57BL6/J SIRPANT-M→B16, MC38, KPC, etc., BALB/c SIRPANT-M→4T1, and FVB/NJ SIRPANT-M→breast cancer cells isolated from palpable tumors of MMTV-PyMT mice (see FIGS. 30-31).

### SIRPANT-M as an Effective Immunotherapy Against Cancer

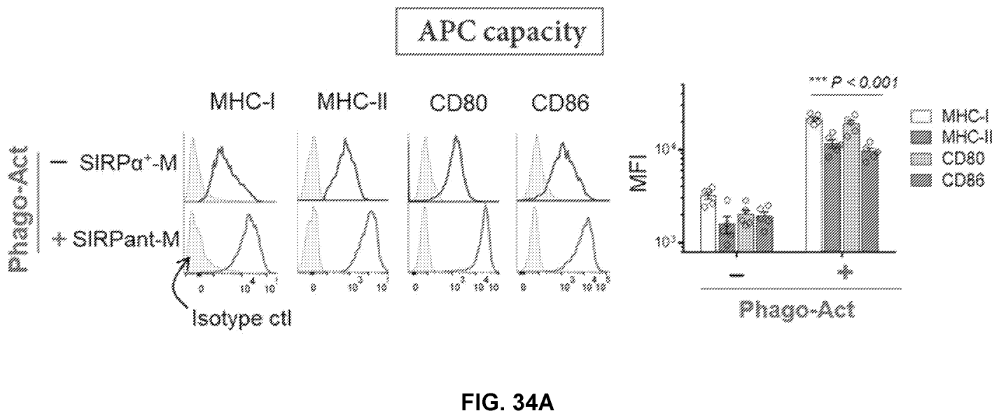

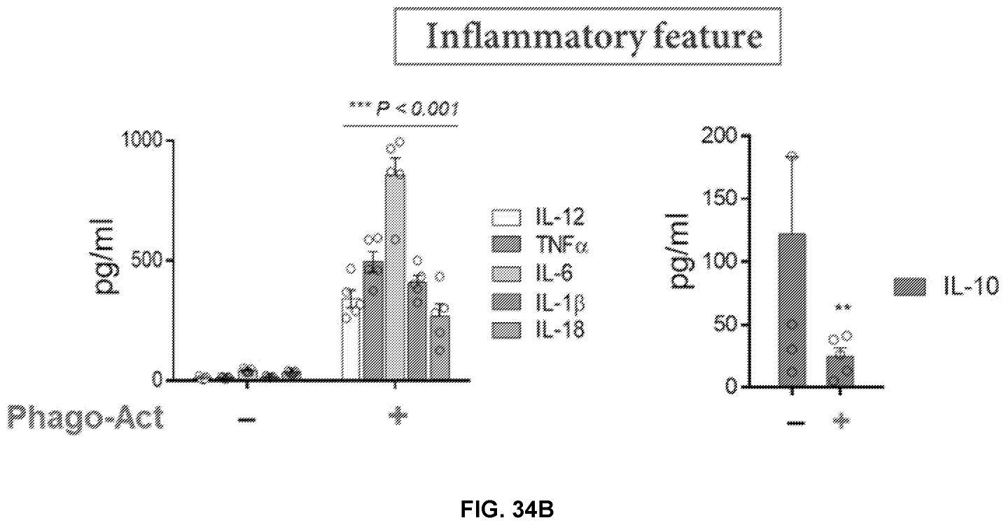

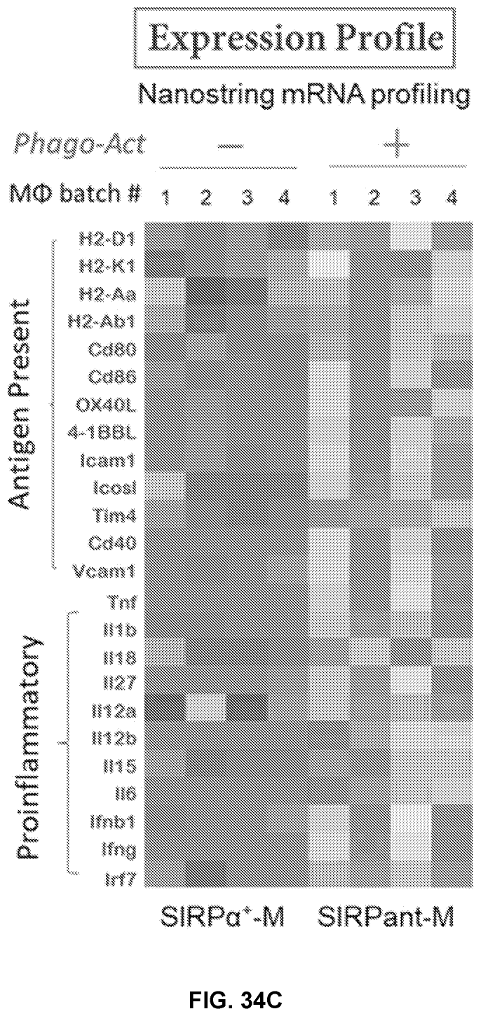

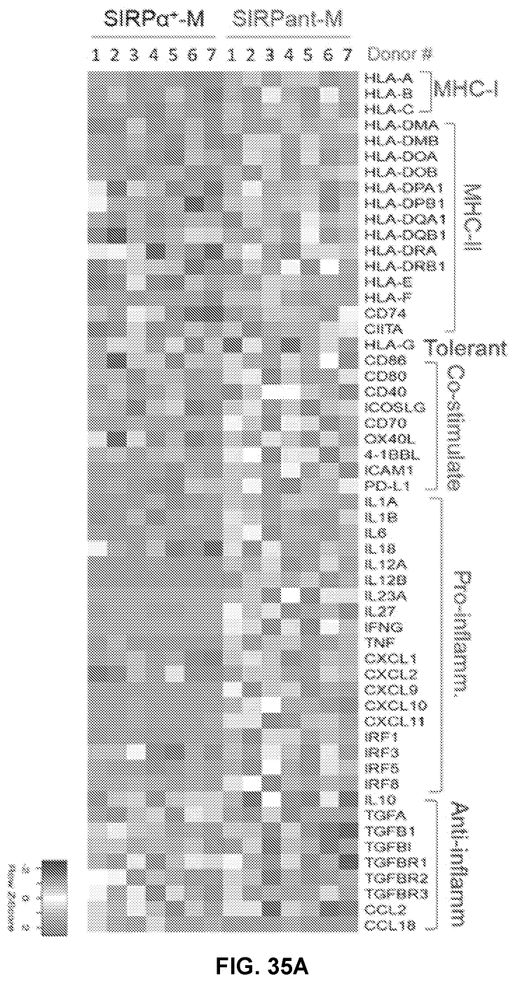

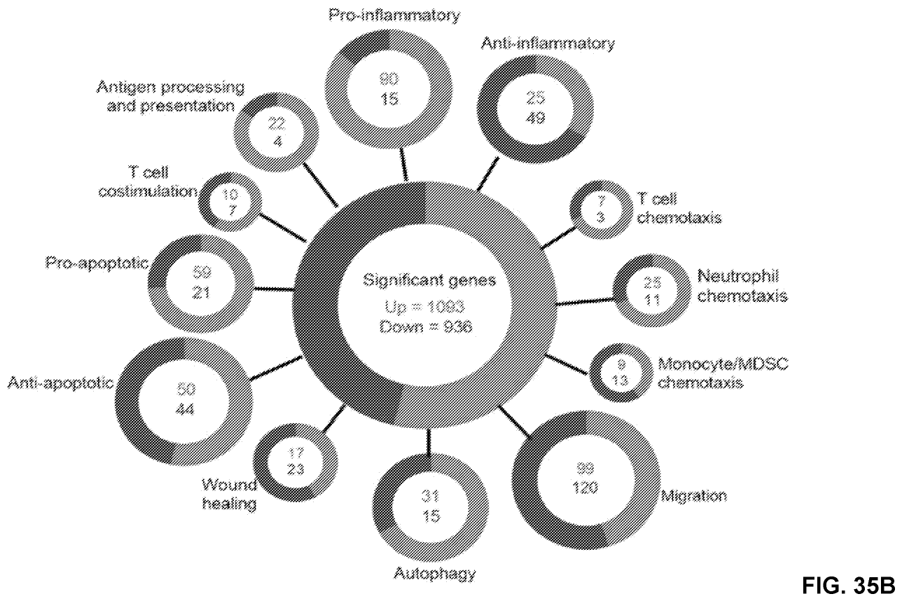

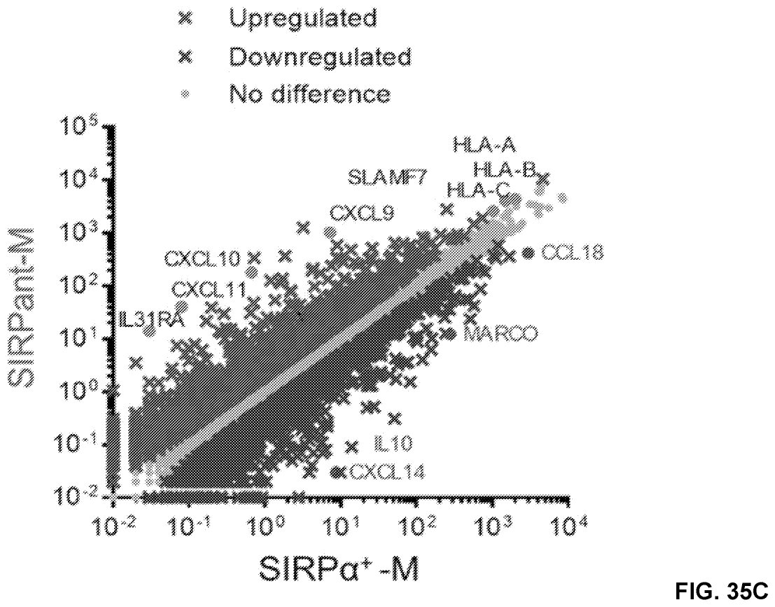

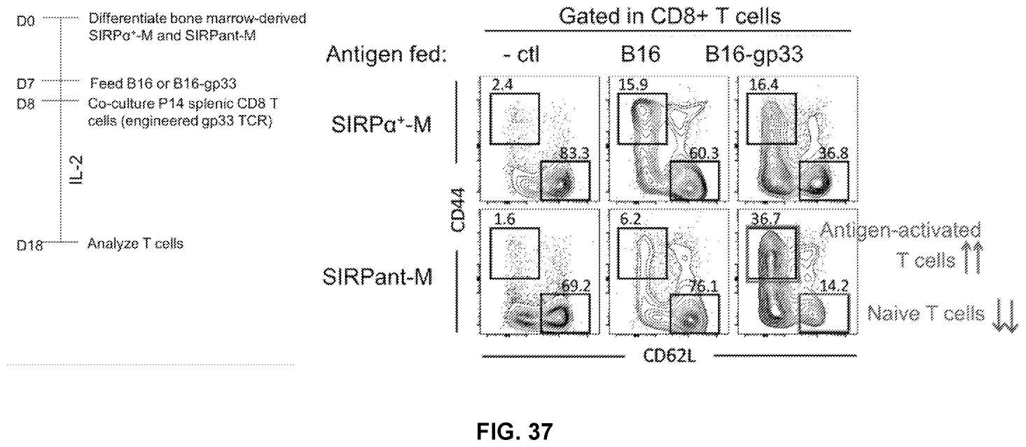

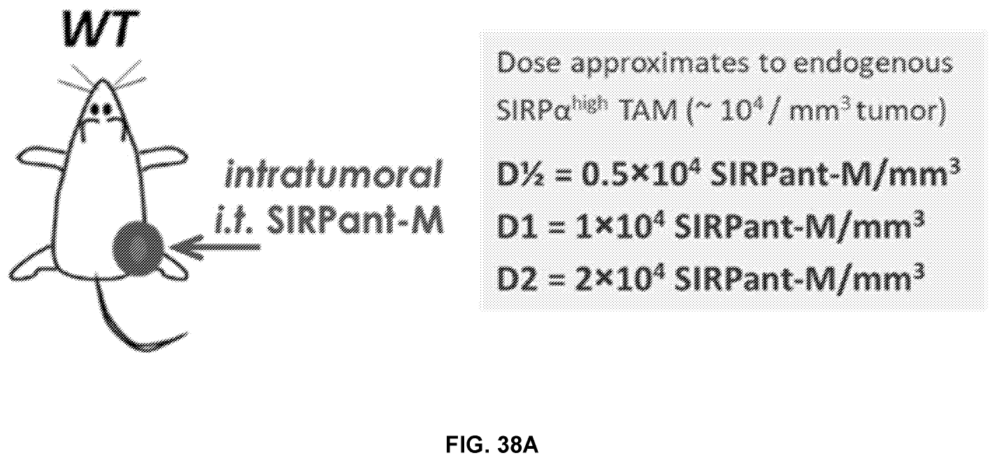

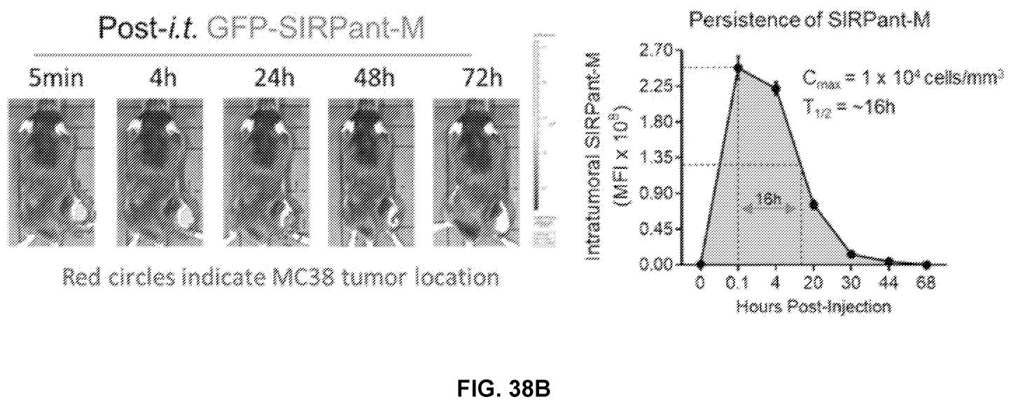

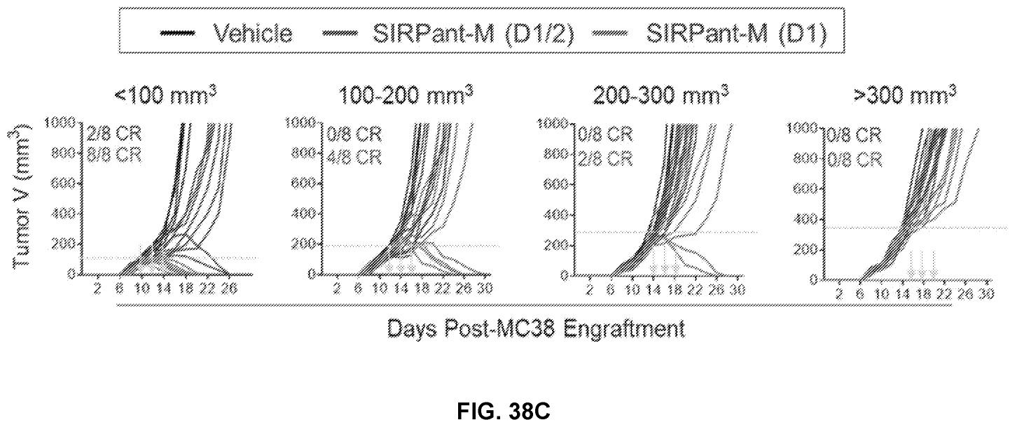

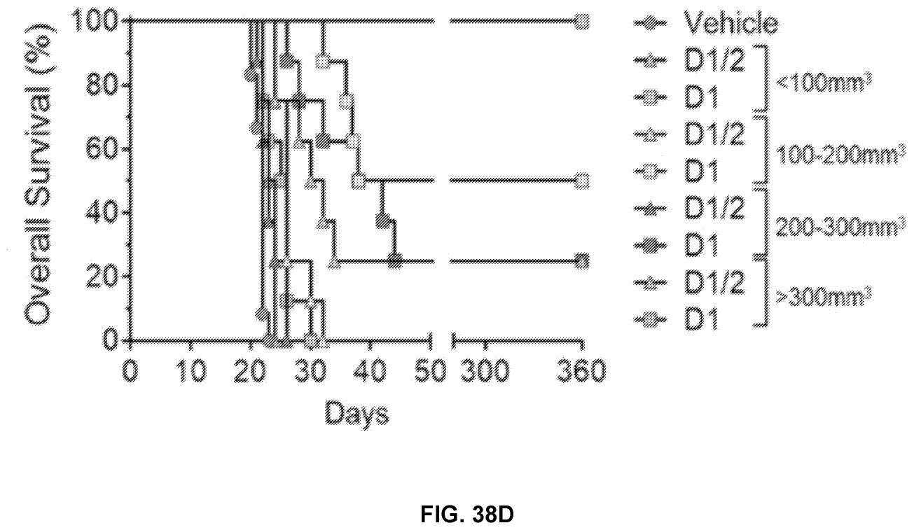

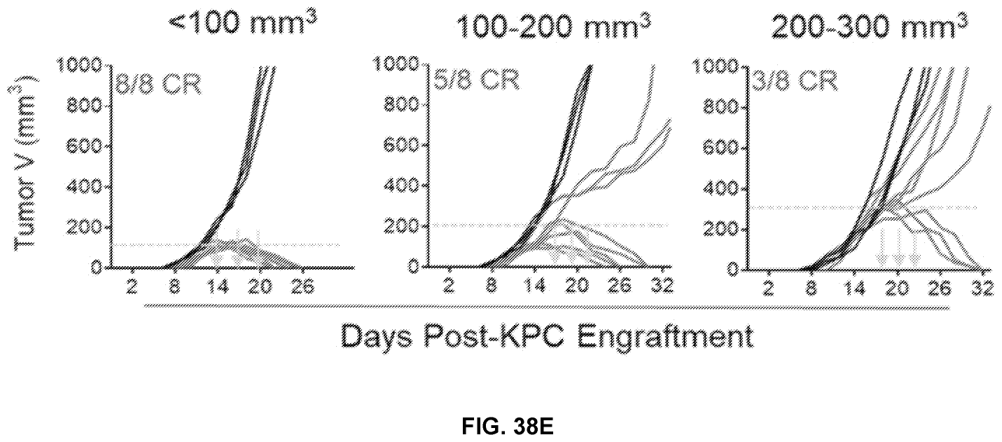

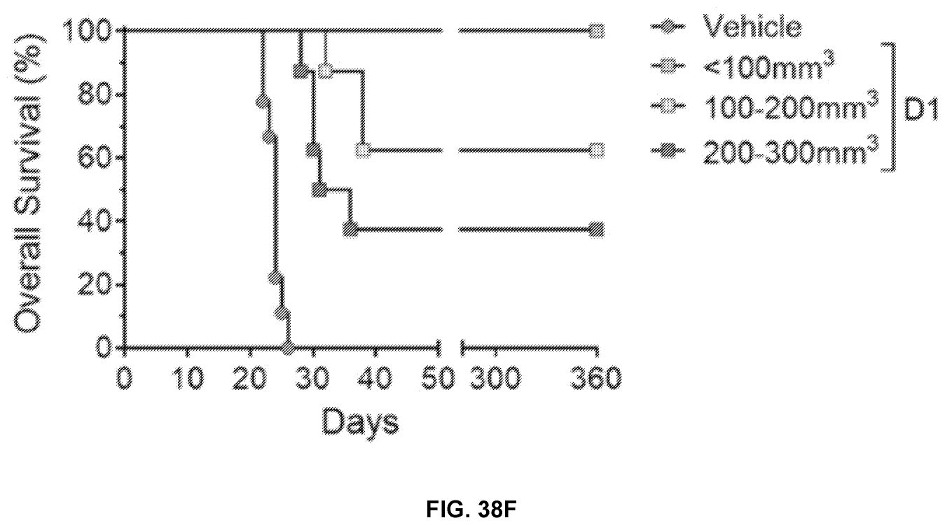

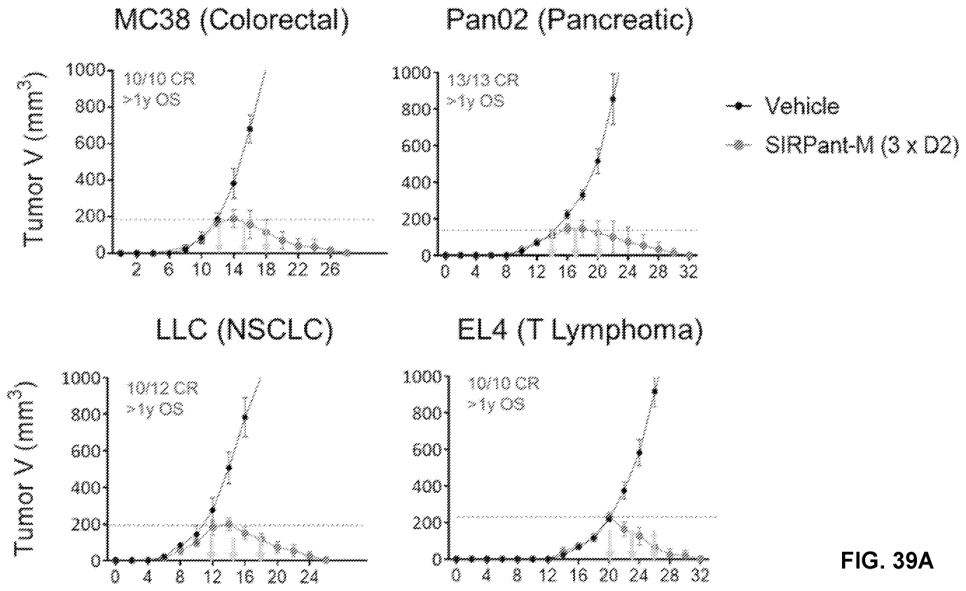

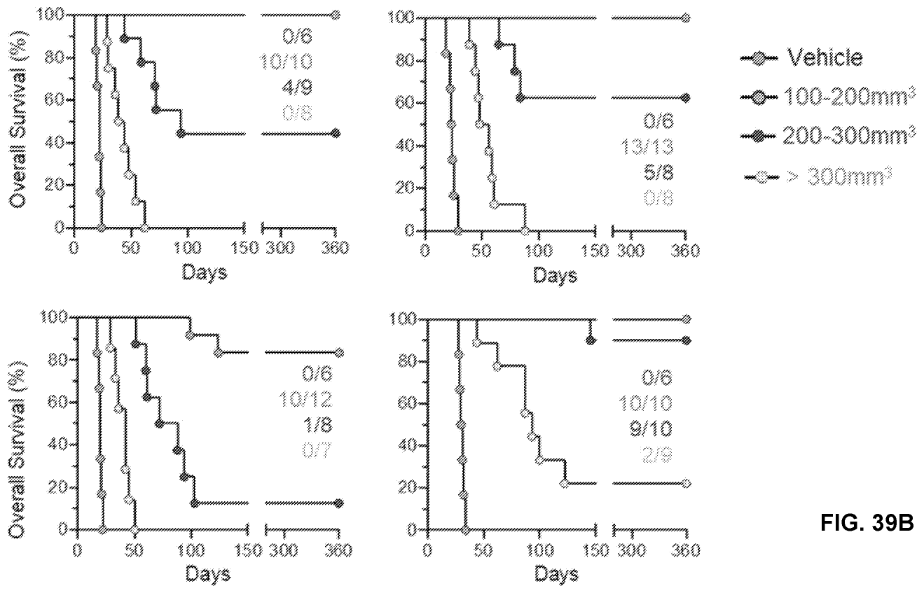

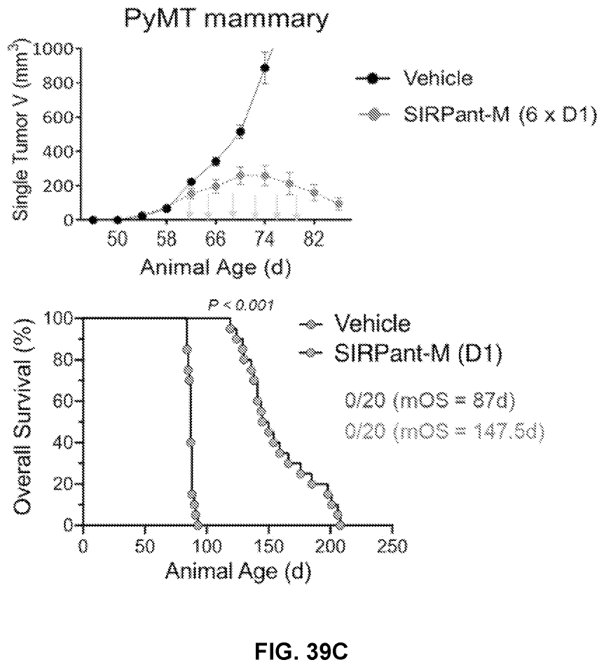

Phago-Act™-produced SIRPANT-M functionally resemble activated Sirpα^{-/-}-M and harbor empowered capabilities that activate both innate and adaptive immunity against cancer. SIRPANT-M has been extensively vetted in vitro in numerous macrophage phenotypic and functional assays that assessed phagocytosis, pro- and anti-inflammatory responses and antigen presentation to activate antigen-specific T cells (FIGS. 34-37). These in vitro studies are complemented by comprehensive in vivo assessment of SIRPANT-M in various murine cancer models across different genetic backgrounds (C57BL6/J, BALB/C, FVB/NJ), with and without adaptive immunity (WT, Rag-1^{-/-}, Nude), and cancers of murine and human origins (FIGS. 38-45). All these studies demonstrate that SIRPANT-M has promise as a highly effective immunotherapy for cancer patients by driving tumor neoantigen-specific, polyclonal and long-lasting T cells and humoral immunity. This therapy does not recapitulate, nor is redundant to, any other therapies in practice or development, but is well-positioned to synergize with immune checkpoint blockade, RT, tumor vaccine and other immunomodulatory regimens. SIRPANT-M differs from CD47 blockade and does not require cancer-specific antibody or other methods for elicit phagocytosis, thereby broadly suitable for many cancers. Indeed, preclinical studies support that SIRPANT-M is a unique, tumor-agnostic therapy applicable to most if not all types of cancer without pre-identification of cancer-specific markers. Additionally, except for a transiently heightened inflammatory response associated with tumor elimination, no or only minimal adverse effects have been found in SIRPANT-M-treated mice, with tumor-eliminated animals generally achieving long-term survival (>1 y post treatment) without recurrence.

### Example 4: SIRPANT-M Pharmacology

SIRPANT-M are autologous SIRPα^{Low} activated macrophages that were generated with Phago-Act™ treatment. The therapeutic efficacy of SIRPANT-M relies on three factors: i) SIRPANT-M's capacity to phagocytose cancer cells, ii) SIRPANT-M's capacity to drive a robust proinflammatory response in the tumor microenvironment, and iii) SIRPANT-M's capacity to present tumor antigens and activate tumor-specific T cells that exert tumoricidal activity. The in vitro studies presented below focused on assessing these SIRPANT-M characteristics.

### Phagocytosis of Cancer Cells

Both murine and human SIRPANT-M were produced following the standard operating procedure outlined in FIG. 29D and then were tested for phagocytosis towards cancer cells of mouse or human origin, respectively.

Method: Total bone marrow cells from mice of different genetic backgrounds (C57BL6/J, BALB/C or FVB/NJ) were differentiated into macrophages (BMDM) in vitro by culturing (RPMI 1640, 10% fetal bovine serum [FBS], 37° C., 5% CO₂) these bone marrow cells in the presence of macrophage colony stimulating factor (M-CSF; 10 ng/ml) for 5 consecutive days. Thereafter, the differentiated macrophages were treated with Phago-Act™ (murine version) for two days to produce SIRPANT-M (FIG. 31A). Phagocytosis assays were conducted by co-culturing SIRPANT-M, or control BMDM, with healthy syngeneic cancer cells (CFSE-labeled) at a 1:2 (BMDM cancer cells) ratio for 4 h (37° C.), followed by assessment and quantification of phagocytosis by fluorescence microscopy and/or flow cytometry (FIGS. 31B & 31C). The genetic background of the cancer cells are as follows: C57BL6/J—B16F10, MC38, KPC, PanO2, LLC and EL4; BALB/C—4T1; FVB/NJ-PyMT breast cancer cells isolated from tumor-bearing MMTV-PyMT mice. SIRPANT-M and control BMDM were tested against genetically matched syngeneic cancer cells. For fluorescent microscopy, phagocytosis was calculated by: (# of BMDM that engulfed at least one cancer cell/100 BMDM in the field)×100. For flow cytometry, phagocytosis was quantified by the frequency of CFSE⁺ BMDM. Statistical significance was determined by Student's t test.

Method: Human PBMC-derived macrophages (SIRPα⁺-M) were treated with Phago-Act™ for two days to produce SIRPANT-M. Additional controls were generated by treating SIRPα⁺-M with other factors (e.g., TNFα/IL-17, or IFNγ). Phagocytosis assays were conducted by co-incubating adherent SIRPANT-M, control SIRPα⁺-M, or other-treated SIRPα⁺-M with healthy human cancer cells (obtained from NCI-60 cell line repository) for varied periods of time (37° C.), followed by assessment and quantification of phagocytosis by fluorescence microscopy and/or flow cytometry. Human cancer cells were labeled with CFSE and were examined for their CD47 expression by flow cytometry to determine whether their CD47 expression impacted the magnitude of phagocytosis. Statistical significance was determined by one-way ANOVA and Dunn's test post-hoc. Correlation assessment between CD47 expression and phagocytosis was determined by linear regression analysis and the Pearson coefficient is shown.

Conclusion: Both murine bone-marrow derived SIRPANT-M and human PBMC-derived SIRPANT-M exhibit proficiency to directly phagocytose cancer cells in vitro. Moreover, the capacity of SIRPANT-M to phagocytose cancer cells occurs irrespective of CD47 expression on cancer cells. These studies confirmed that Phago-Act™ treatment removes CD47-SIRPα-mediated inhibition and provides activation that enables SIRPANT-M to robustly phagocytose cancer cells.

Method: Healthy murine or human cancer cells were treated with non-ablative X-ray radiation (4Gy, 8Gy, or 15Gy), followed by co-incubation with murine or human SIRPANT-M, or control SIRPα+-M/BMDM for various periods of time at 37° C. Thereafter, phagocytosis was quantified by fluorescence microscopy and/or flow cytometry. Statistical significance was determined by either Student t test or one-way ANOVA and Tukey's post-hoc.

Conclusion: Irradiation of cancer cells markedly enhanced their susceptibility to phagocytosis by SIRPANT-M. The data indicate that non-ablative radiation, though maintaining cancer cell viability and CD47 expression, induces damage-associated molecules (such as calreticulin) on cancer cells that augment SIRPANT-M phagocytosis. In contrast, SIRPα⁺-M do not exhibit pronounced improvement of phagocytosis toward irradiated cancer cells, in part due to the presence of CD47-SIRPa inhibition. However, blockade of CD47 by anti-CD47 Ab or CD47 deficiency on cancer cells only partially improves SIRPα⁺-M phagocytosis of irradiated cancer cells, albeit the extent to which irradiated cancer cells are phagocytosed by SIRPANT-M is unmatched.

### Inflammatory Phenotype and Antigen Presentation Machinery

Method: Freshly prepared murine bone marrow-derived macrophages (BMDM, SIRPα⁺-M) were further treated with Phago-Act™ for 48 h to induce SIRPANT-M. Cell culture medium of human PBMC-derived SIRPANT-M (+Phago-Act™) and control SIRPα⁺-M (−Phago-Act™) were collected and assayed for pro- and anti-inflammatory cytokines by ELISA. Flow cytometry was performed to analyze cells surface expression of antigen presentation machinery including MHC-I and -II, and co-stimulatory molecules CD80 and CD86. Total RNAs were prepared for mRNA transcription analyses by Nanostring.

Conclusion: Compared to SIRPα⁺-M, SIRPANT-M exhibit an augmented proinflammatory phenotype characterized by increased expression of proinflammatory cytokines, reduced production of immunosuppressive IL-10, and increased expression of immunogenic antigen presentation machinery including MHC-1/II and co-stimulatory molecules.

Method: Total RNAs were isolated from seven samples (#1-7) of human PBMC-derived SIRPANT-M and donor-matched SIRPα⁺-M. The donors were healthy volunteers and included 4 males and 3 females, among which there were 2 White, 2 Black, 2 Asian and 1 Mixed. These RNA samples were subjected to comprehensive sequencing that analyzed the expression of over 10,000 genes.

Conclusion: Compared to donor-matched SIRPα⁺-M, SIRPANT-M exhibit elevated expression of genetic associated with immunogenic antigen presentation machinery including MHC-1, MHC-II, CIITA, and co-stimulatory molecules (CD80/86/40/70, OX40L, 4-1 BBL, ICAM-1, etc.), but have reduced expression of non-classical, immunotolerance-related HLA-G. SIRPANT-M also increase expression of proinflammatory cytokines and chemokines (IL-1/6/12/18/23/27, IFNα/β/γ, TNFα, CXCL1/2/9/10/11, etc.), while reducing anti-inflammatory IL-10, TGFα/β, TGFβRs and CCL2/18 expression.

### SIRPANT-M Mediate Antigen Presentation and Activate Tumor Antigen-Specific T Cells

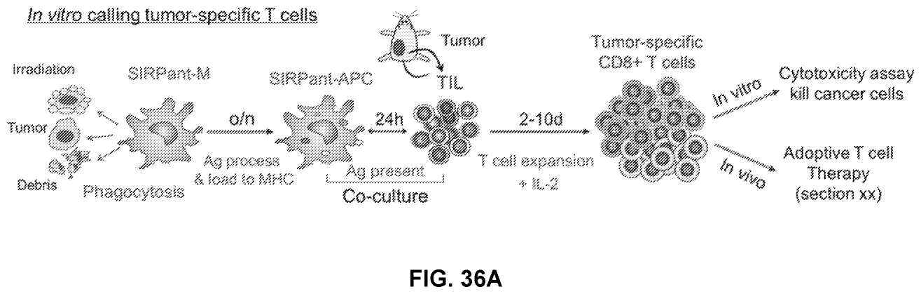

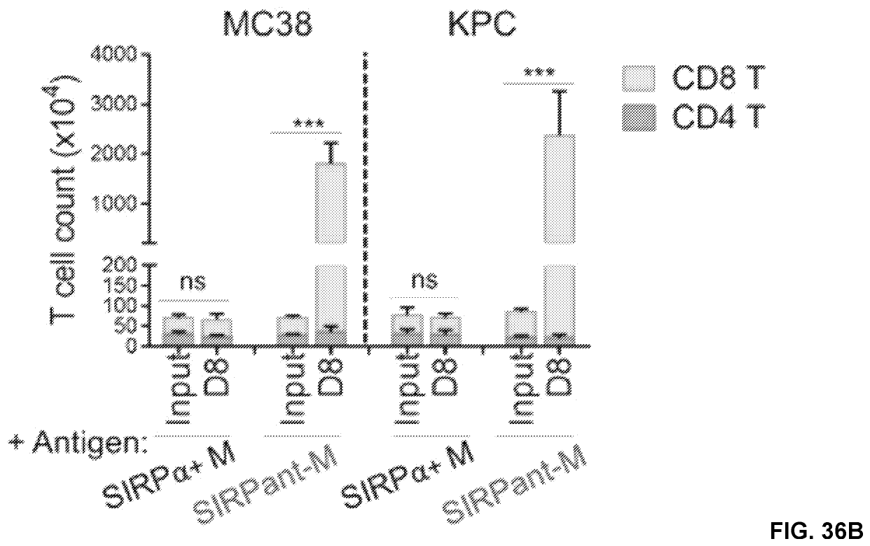

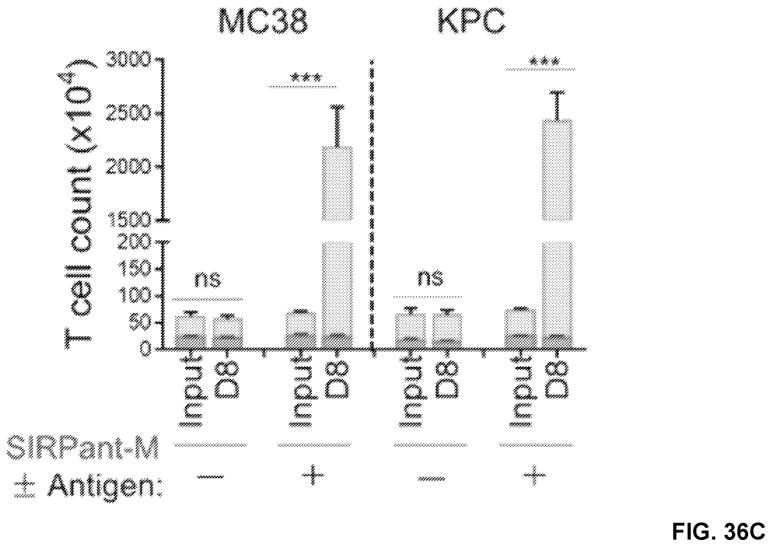

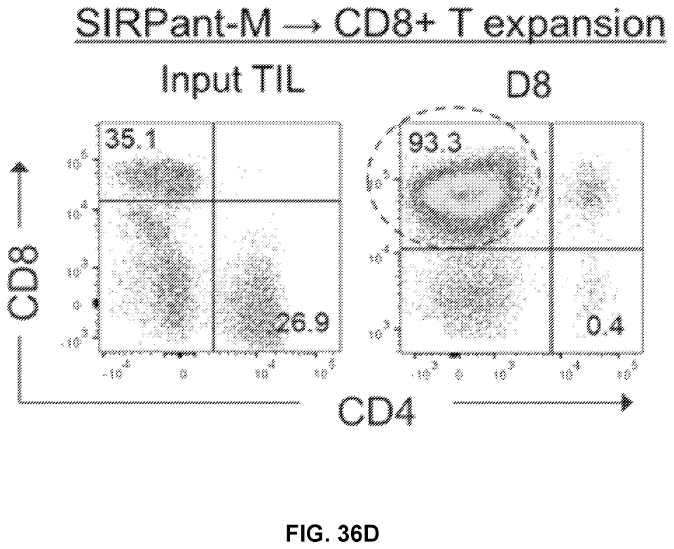

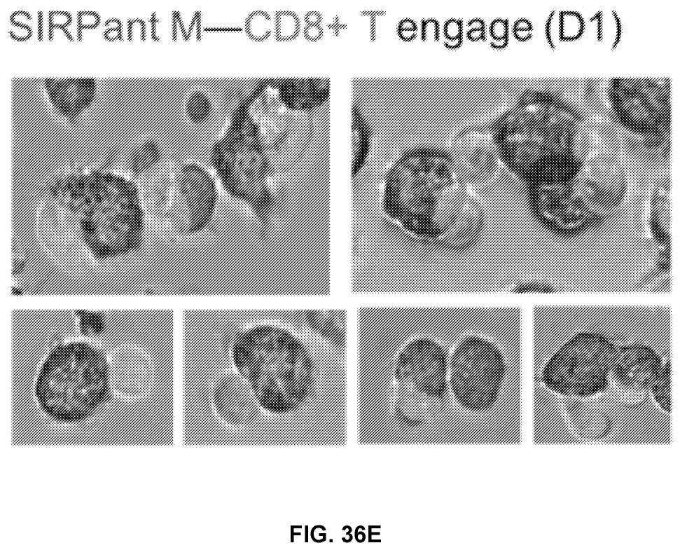

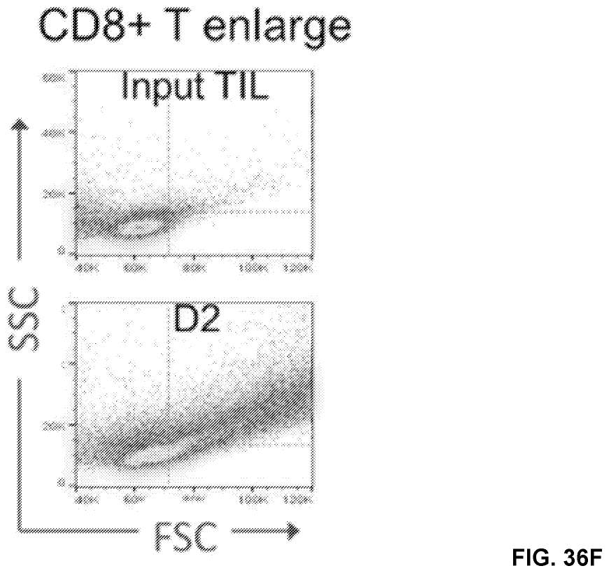

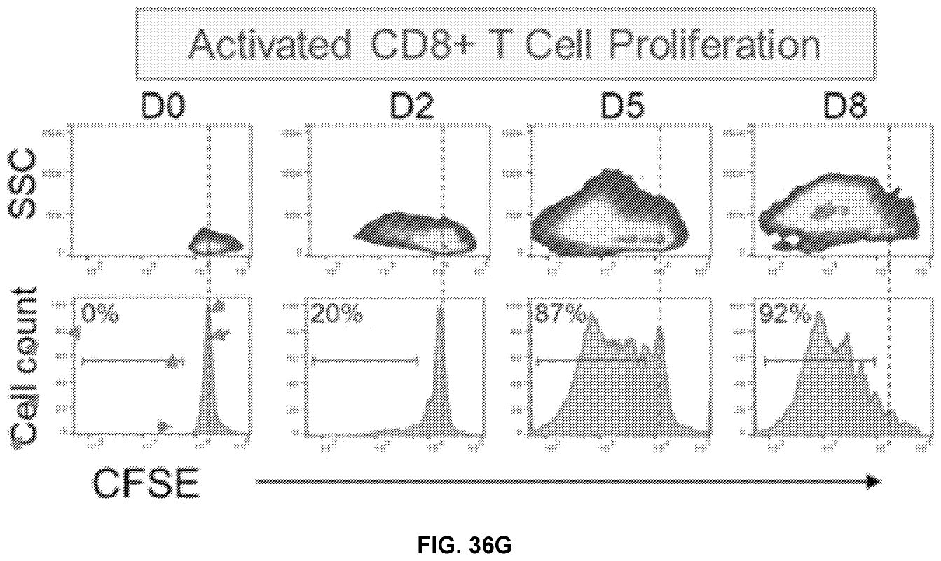

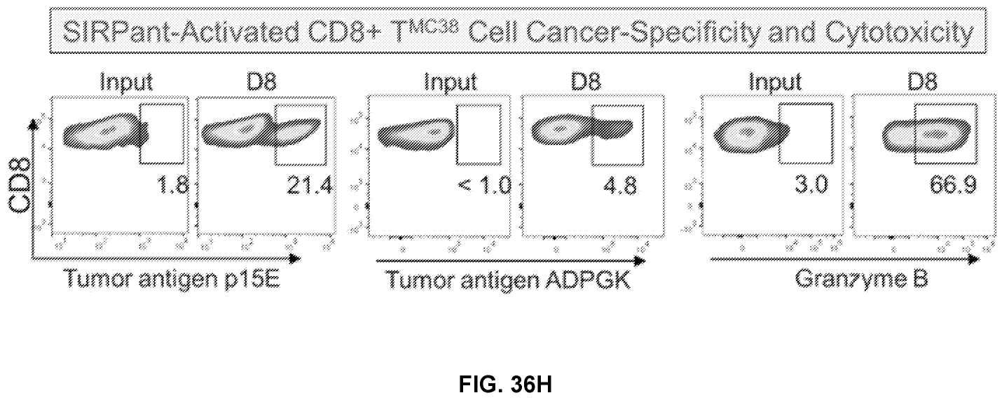

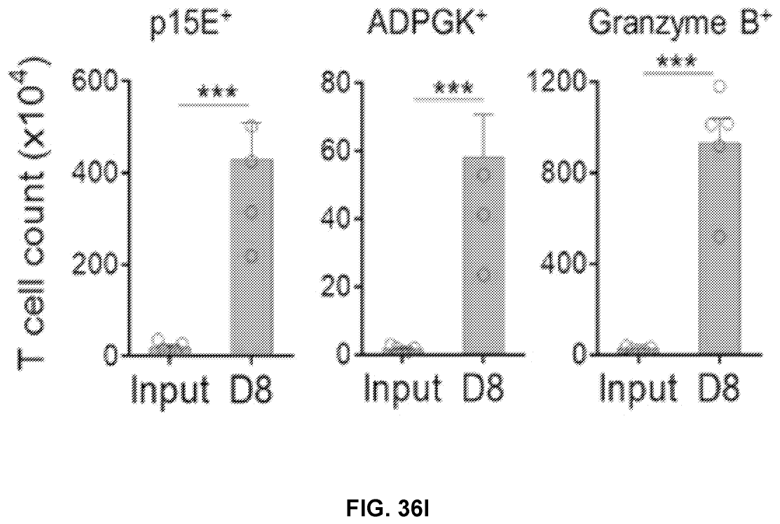

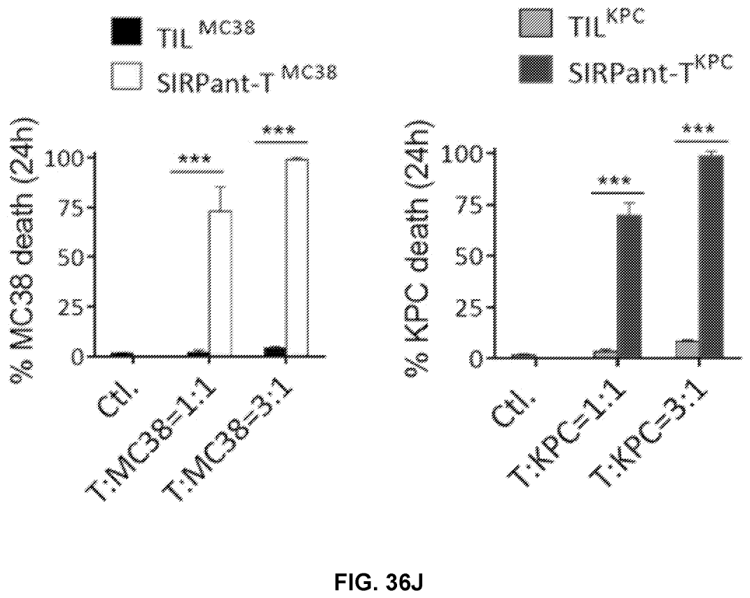

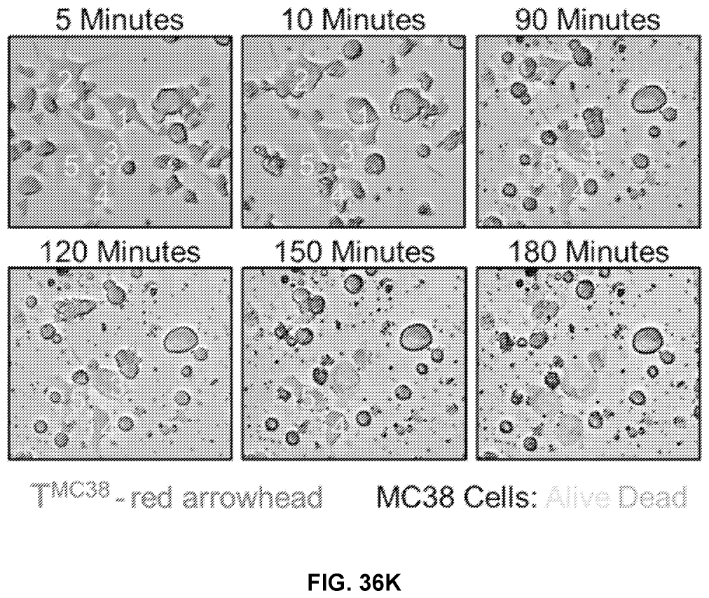

Method: The experimental scheme is shown in FIG. 36A. Murine bone marrow-derived SIRPANT-M, or control BMDM/SIRPα⁺-M, were incubated overnight (˜18 h, 37° C.) with radiation-treated MC38 or KPC tumor cells for macrophages to phagocytose cancer cells and process tumor antigens. Tumor-infiltrating lymphocytes (TIL) were obtained from resected MC38 or KPC tumors following collagenase digestion of tumor tissues, culturing of dissociated cells and collection of the non-adherent cell population, of which the majority were T lymphocytes. Enriched TIL were then added into wells containing tumor antigen-loaded macrophages at a TIL: macrophage ratio of 5:1 (1×10⁶ TIL and 2×10⁵ SIRPANT-M or SIRPα⁺-M per well in a 24-well plate). The SIRPANT-M-TIL co-culture was then maintained (37° C., 5% CO₂) for 8-10 days in RPMI-1640 medium containing 10% FBS, 2 mM L-glutamine and 50 μM β-mercaptoethanol, with 50 IU/ml recombinant IL-2 added on day 2. IL-2-containing medium was replenished every three days and the cell density was maintained below 1×10⁶ cells/ml. To examine T cell activation and expansion, fluorescence microscopy and flow cytometry were performed 24 h after the co-culture (d2) to assess TIL-macrophage engagement and T cell enlargement (FIG. 36E-36F). T cell proliferation was assessed by CFSE dilution at various time points using flow cytometry (FIG. 36G). (TIL were pre-labeled with CFSE prior to co-culture for FIG. 36E-36G). The quantity of CD8 T cells and CD4 T cells were also determined after co-incubation with SIRPα⁺-M/BMDM (FIG. 36B) and SIRPANT-M that had phagocytosed and processed antigen (+Antigen) or when cancer cells were withheld (−Antigen) (FIG. 36C). Additional flow cytometry analyses were performed to determine the frequency and quantity of CD8 T cells expressing granzyme B and reactive to tumor-specific MHC tetramers p15E and ADPGK (FIG. 36H-36I), both of which are indicative of T cell specificity to cancer. In the co-culture containing tumor antigen-loaded SIRPANT-M and TIL, the total T cell number generally increased 20-fold, from the initial 1×10⁶ cells to approximately 2×10⁷ T cells of which >95% were CD8 T cells after 8-10 days of co-culture. Dependent upon the tumor type, the expanded T cells were termed T^{MC38} or T^{KPC}, and were tested for tumoricidal toxicity against respective MC38 or KPC cancer cells in vitro (FIG. 36J-36K), and in vivo by adoptive T cell therapy.

Conclusion: These studies demonstrated: i) tumor-phagocytosed SIRPANT-M are excellent antigen presenting cells (APC), which mediate immunogenic antigen presentation and robustly activate tumor-specific CD8+ cytotoxic T cells (CTL) from TIL; ii) SIRPANT-M activate CD8 T cells through in situ calling of memory tumor-specific T cells (i.e. T_{EM}/T_{RM}) within TIL; iii) SIRPANT-M-mediated antigen presentation preferentially activates tumor-specific CD8+ cytotoxic T cells, but not CD4+T helper cells (Th); iv) SIRPANT-M-activated CD8 T cells highly express granzyme B and exhibit polyclonal cancer-specificity; v) SIRPANT-M-activated CD8 T cells are highly cytotoxic against cancer and rapidly eliminate cancer cells at relatively low effector:target ratios. These conclusions are consistent with in vivo experiments in mouse tumor models.

Method: A similar experimental scheme was followed as detailed in FIG. 36A except that B16 melanoma-specific naïve CD8 T cells were induced. SIRPANT-M or control BMDM/SIRPα⁺-M were co-incubated with parental B16F10 melanoma cells or gp33-expressing B16F10 melanoma cells that were subjected to multiple freeze-thaw cycles to induce immunogenic cell death and provide B16 antigen. B16 antigen-loaded SIRPANT-M or control BMDM/SIRPα⁺-M were then co-incubated with naïve splenic CD8⁺ T cells from P14 transgenic mice that express a TCR specific for the H-2D^{b}-restricted gp33 epitope.

Conclusion: These experiments confirmed that SIRPANT-M, following phagocytosis of tumor antigens, become excellent APCs that conduct antigen presentation to activate antigen-specific naïve CD8+ T cells.

### Example 5: In Vivo Pharmacology Studies

SIRPANT-M's capability to drive anti-cancer response in vivo has been extensively tested in various preclinical cancer models in mice across different genetic backgrounds (C57BL6, BalbC, FVB/NJ). These cancers include lymphoma, colorectal adenocarcinoma, melanoma, lung cancer, pancreatic ductal adenocarcinoma, metastatic breast cancer, carcinogen and inflammation-induced colon cancer, etc. Among these tested cancers, some were late stage, having large tumors with distal lesions (metastases). In all cases, SIRPANT-M upon administration into tumor mass exert potent anti-cancer activity, demonstrating direct phagocytosis of cancer cells and driving proinflammatory response and downstream presentation of tumor-associated neoantigens to activate tumoricidal T cells in an immunogenic manner. Consequently, large numbers of tumor-specific polyclonal cytotoxic T cells are expanded to combat the tumor and distal lesions (metastases), achieving (i) rapid and systemic elimination of solid tumors, and (ii) induction of long-lasting anti-cancer immunity T cell and antibody that prevents cancer recurrence.

The below section demonstrates preclinical cancer treatment studies conducted in mice.

### SIRPANT-M Monotherapy

Treatment: SIRPANT-M intratumoral injection (i.t.)

| | |
|---|---|
| Dosage: | D½ = 0.5 × 10⁴/mm³ tumor mass |
| | D1 = 1 × 10⁴/mm³ tumor mass |
| | D2 = 2 × 10⁴/mm³ tumor mass |

Cancer type: i. Colorectal adenocarcinoma MC38-C57BL6 syngeneic engraft, ii. Pancreatic ductal adenocarcinoma (PDA) KPC-C57BL6 syngeneic engraft, iii. Pancreatic ductal adenocarcinoma (PDA) PanO2-C57BL6 syngeneic engraft, iv. Lung cancer LLC-C57BL6 syngeneic engraft, v. Lymphoma EL4-C57BL6 syngeneic engraft, and vi. MMTV-PyMT triple negative metastatic breast cancer-FVB/NJ spontaneous.

### Experimental Procedure:

Tumor models: For syngeneic engraft models, healthy cultured EL4, MC38, LLC, KPC, PanO2 cancer cells (5×10⁵) suspended in 50 μl PBS were subcutaneously engrafted into WT C57BL6 mice (6-8 w, male or female). Palpable tumors generally formed after 10-18 days with growth rates dependent on cancer types. Measurements were taken using calipers for the tumor length and width, followed by calculation of the tumor volume (V) with formula: volume=(length×width²)/2. MMTV-PyMT mice were obtained from The Jackson Laboratory (002374 FVB/N-Tg(MMTV-PyVT) 634Mul/J). Female PyVT transgene carriers spontaneously develop palpable mammary tumors at about 2-month of the age (mean latency of 53 d).

SIRPANT-M preparation: Femur bones were obtained from WT C57BL6 mice or male MMTV-PyVT mice. Bone marrow-derived macrophages (BMDM) were produced by M-CSF, followed by treating BMDM with Phago-Act™ (37° C., 48 h) to produce SIRPANT-M. Prior to use, SIRPANT-M were trypsinized from culture dishes, and after wash, these cells were resuspended in PBS at 1×10⁸/ml and used in 0.5-3 h (keep on ice prior to use). Flow cytometry analyses confirmed SIRPANT-M to be SIRPa^{Low} and with increased expression of MHC-1, MHC-II, CD80, and CD86. Only genetically matched SIRPANT-M were used to treat tumors in mice of different background, such that SIRPANT-M prepared from C57BL6 mice were used to treat EL4, MC38, LLC, KPC and PanO2 tumors in C57BL6 mice, SIRPANT-M prepared from FVB/NJ mice were used to treat PyMT breast cancer in mice of the same background.

Tumor treatment: Doses of SIRPANT-M were calculated according to tumors sizes. SIRPANT-M in PBS were i.t. injected into tumors following a multipoint injection manner, e.g. 2-4 injections from different directions or angles of the tumor, with an Exel-Comfort Point insulin syringe needle (29G1/2), a procedure to improve SIRPANT-M diffusion in tumor tissues. The treatment was repeated every three days and a total of 2-3 treatments were given.

### Conclusion of Studies of SIRPANT-M Monotherapy:

SIRPANT-M by i.t. dose-dependently, strongly inhibit tumor growth or induce tumor regression.

SIRPANT-M monotherapy substantially increased animal survival and, for small tumors, conferred complete response with long-term survival.

SIRPANT-M's anti-tumor effect is agnostic to tumor types, demonstrating strong inhibition to all tested tumors.

### SIRPANT-M and Radiotherapy (RT) Combination

| | |
|---|---|
| Treatment Modality: | 1- SIRPANT-M intratumoral injection (i.t.) |
| | 2- Tumor-focused non-ablative X-ray radiation (RT) |
| SIRPANT-M Dose: | D½ = 0.5 × 10⁴/mm³ tumor mass |
| | D1 = 1 × 10⁴/mm³ tumor mass |
| | D2 = 2 × 10⁴/mm³ tumor mass |
| RT Dose: | X-ray 4Gy |
| | X-ray 8Gy |
| | X-ray 15Gy |

Cancer type: i. Colorectal adenocarcinoma MC38-C57BL6 syngeneic engraft; ii. Pancreatic ductal adenocarcinoma (PDA) KPC-C57BL6 syngeneic engraft; iii. Pancreatic ductal adenocarcinoma (PDA) PanO2-C57BL6 syngeneic engraft; iv. Lung cancer LLC-C57BL6 syngeneic engraft; v. Lymphoma EL4-C57BL6 syngeneic engraft; vi. Triple negative breast cancer (TNBC) 4T1-Balb C orthotopic transplant; and vii. MMTV-PyMT triple negative breast cancer (TNBC)-FVB/NJ spontaneous.

### Experimental Procedure:

Tumor models: Same procedures were used to establish syngeneic engraft models of EL4, MC38, LLC, KPC and PanO2 tumors in WT C57BL6 mice as in the last section (monotherapy). To establish distal lesions, engraftments were proceeded with one location (e.g. the right flank) implanted with 5×10⁵ tumor cells for the formation of a primary tumor and with other locations, such as the left flank, the right and/or left armpits and the peritoneal cavity, implanted with 0.5-2×10⁵ tumor cells to form smaller, “distal” lesions. In some experiments, two primary tumors were engrafted along with multiple distal lesions. 4T1 orthotopic breast cancer was established in Balb C mice. For this model, 3×10⁴ 4T1 cells suspended in 50-μl PBS were injected into the mammary fat pad of 6-8 w old female Balb C mice, and palpable tumors generally formed in two weeks following the engraftment. The establishment of MMTV-PyMT triple negative metastatic breast cancer was described in the last section.

SIRPANT-M preparation: The same procedure (FIG. 29D) was taken to prepare bone marrow-derived SIRPANT-M from C57BL6, MMTV-PyVT, or Balb C mice. Only genetically matched SIRPANT-M were used to treat tumors in mice with the same background to ensure syngenecity, such that SIRPANT-M prepared from C57BL6 mice were used to treat EL4, MC38, LLC, KPC and PanO2 tumors in C57BL6 mice, SIRPANT-M prepared from Balb C mice were used to treat 4T1 breast cancer engrafted in Balb C mice, etc.

### Tumor Treatment:

i) SIRPANT-M i.t.—Freshly prepared SIRPANT-M calculated according to the tumors size suspended in PBS were injected into the tumor mass following a multipoint injection manner, e.g. 2-4 injections from different directions or angles of the tumor, with an Exel-Comfort Point insulin syringe needle (29G1/2).
ii) Tumor RT: Tumor-bearing mice under anesthesia with ketamine (17.5 mg/ml, Henry Schein) and xylazine (2.5 mg/ml, Henry Schein) were placed in a customized jig with a lead holder such that only the primary tumor was exposed, followed by irradiation in a RS-2000 biological X-ray irradiator (Rad Source Technology) with a dose rate of 1.2Gy/min (160 kV, 25 mA) to reach 4Gy, 8Gy, 10Gy, or 15Gy.
iii) Combination: SIRPANT-M i.t. was administrated either before or after a fraction of radiation given to the same tumor. We have tested SIRPANT-M i.t. given 0.5 h-48 h prior to, or the same time-period after, the tumor focal RT.

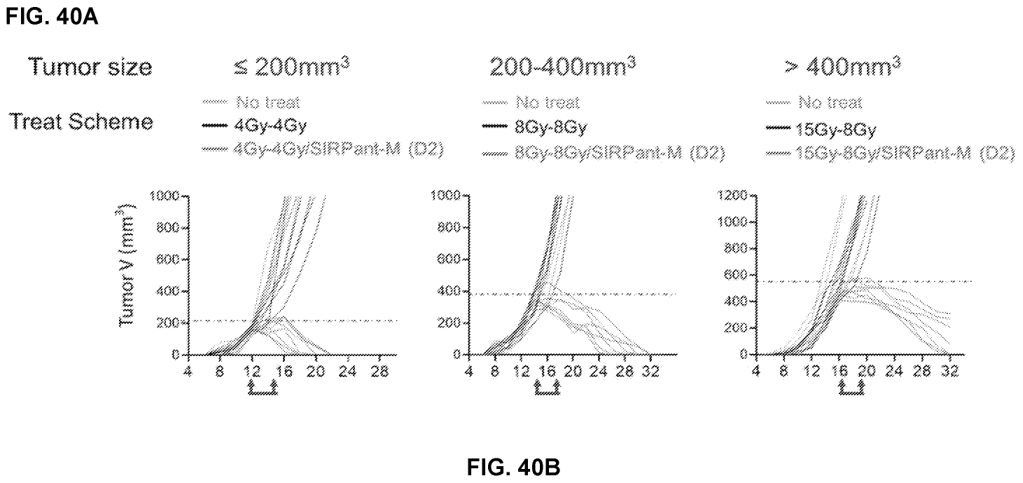

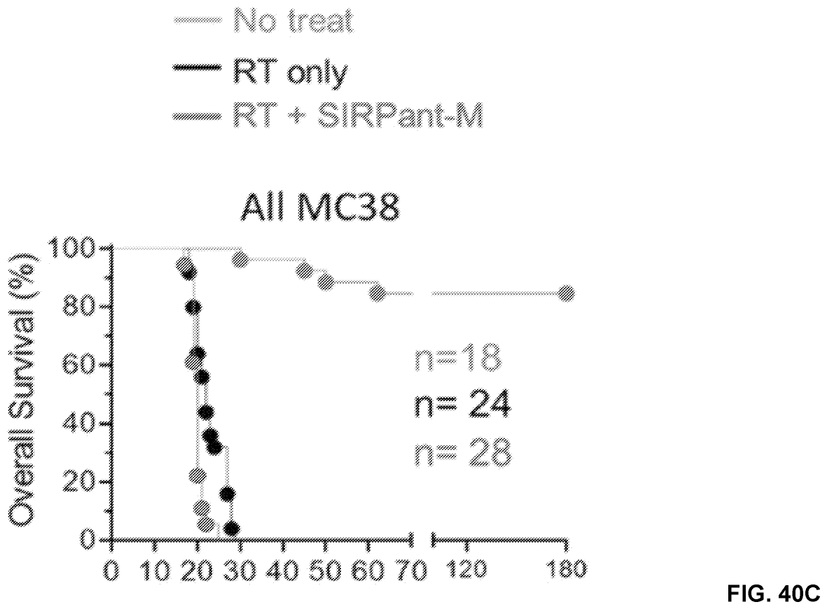

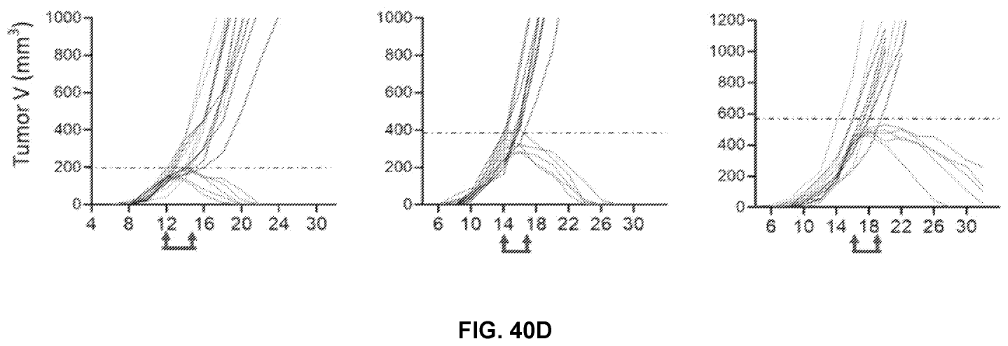

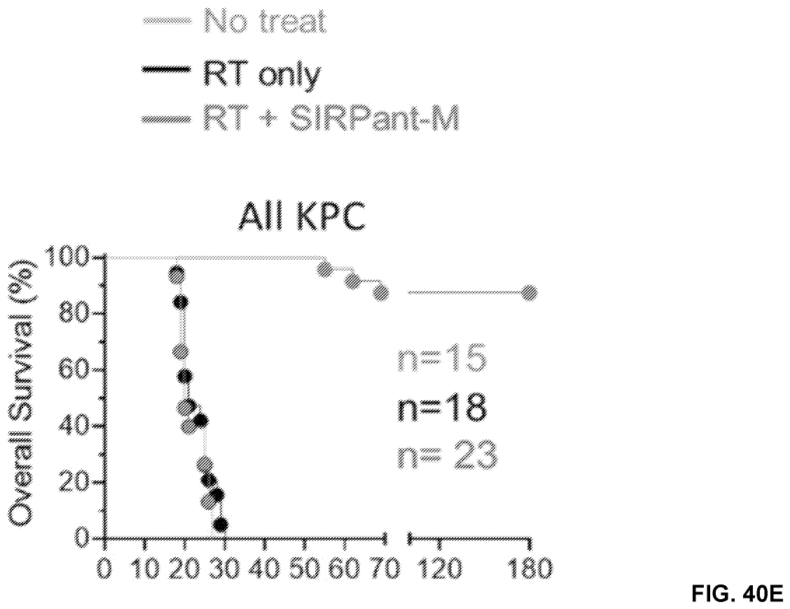

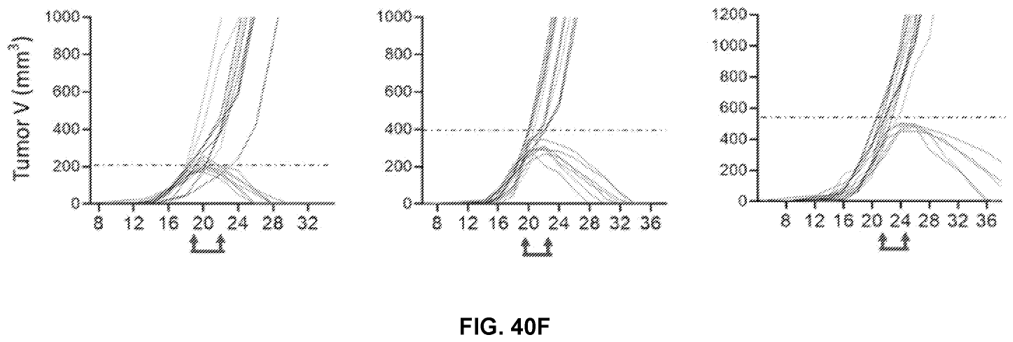

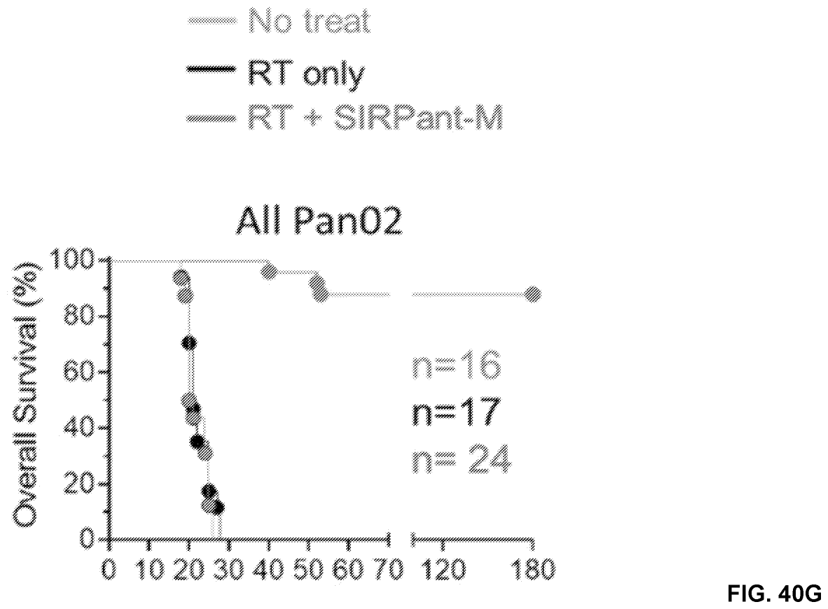

Study-1: Testing SIRPANT-M i.t. combined with RT of varied doses (4Gy, 8Gy or 15Gy) to treat RT-refractory colorectal adenocarcinoma MC38 and pancreatic ductal adenocarcinoma KPC and PanO2 of different stages (varied tumor sizes). Partial data are shown in FIG. 40.

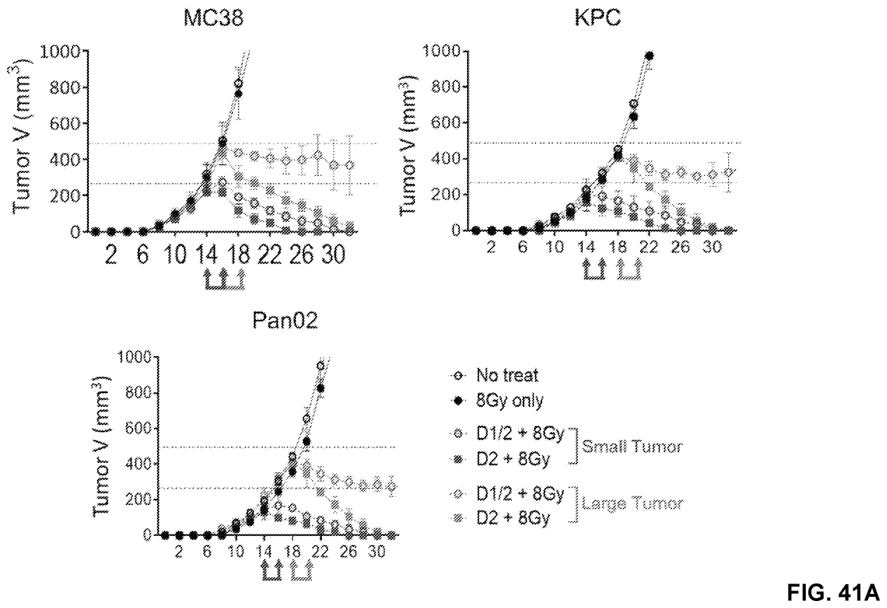

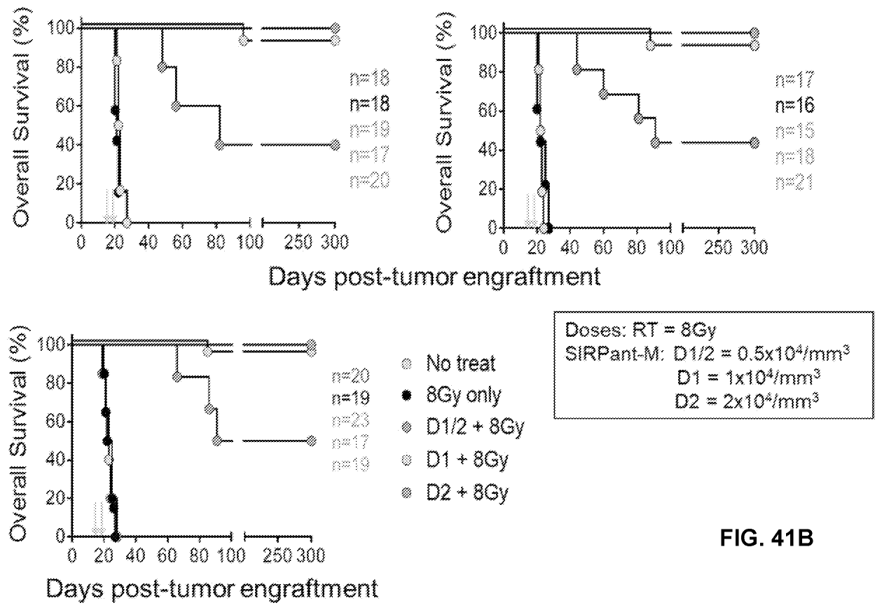

Study-2: Testing 8Gy RT combined with SIRPANT-M at varied doses to treat RT-refractory colorectal adenocarcinoma MC38 and pancreatic cancer KPC and PanO2. FIG. 41 shows partial data of the study.

Study-3: Testing abscopal effects. Given that SIRPANT-M mediate anti-cancer efficacy largely through their immunogenic antigen presentation and activation of tumor-specific T cells, strong abscopal tumoricidal activities are thus anticipated. This study tested SIRPANT-M for the capacity of inducing abscopal effects, leading to suppression and/or clearance of distal cancer lesions (mimic metastases).

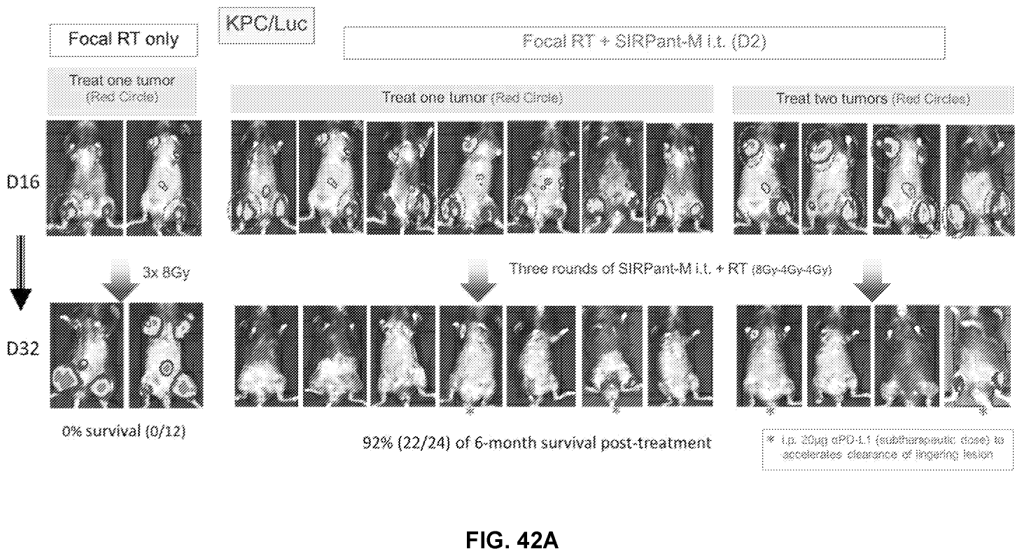

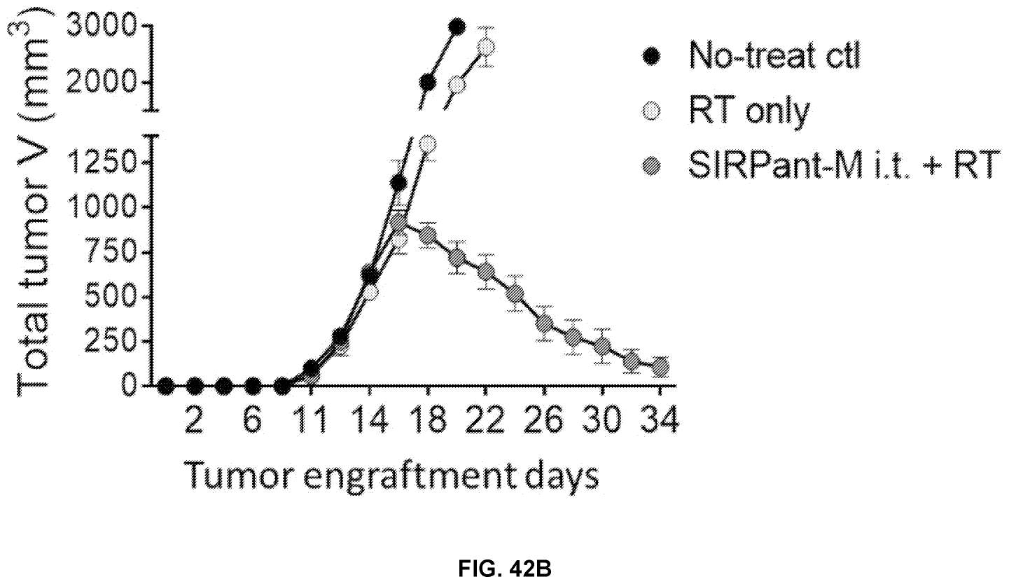

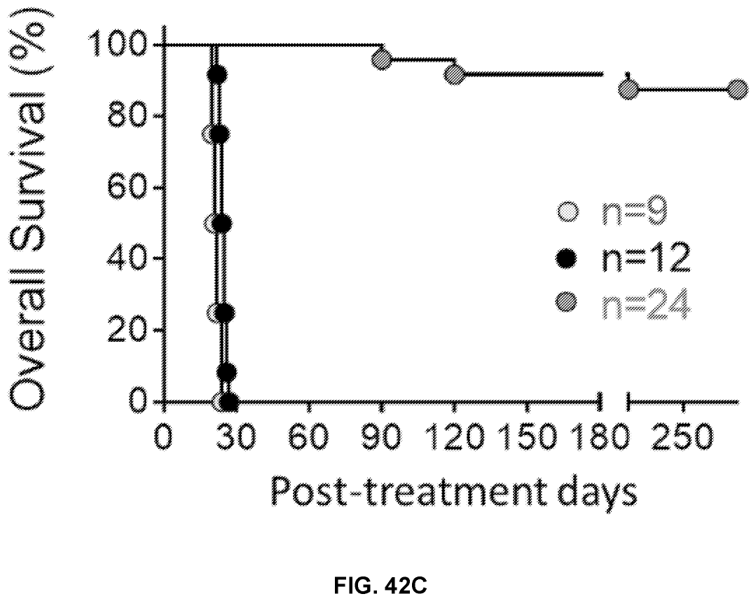

Study-3-1: Testing SIRPANT-M and RT combination for abscopal effects that systemically eliminate KPC pancreatic cancer with distal lesions. KPC/Luc pancreatic adenocarcinoma tumors were simultaneously engrafted in multiple locations with one or two engraftment(s) forming the primary tumor(s). After tumors formation, the primary tumor(s) were treated with SIRPANT-M i.t. plus RT for two or three cycles (3 d apart), following the 8Gy (1^{st})-4Gy-4Gy RT scheme, each with immediate SIRPANT-M i.t. at the D2 dose. other cancer lesions were untreated. Whole body images were taken to monitor primary and systemic KPC tumors for progression, regression, or clearance. Partial data are shown in FIG. 42.

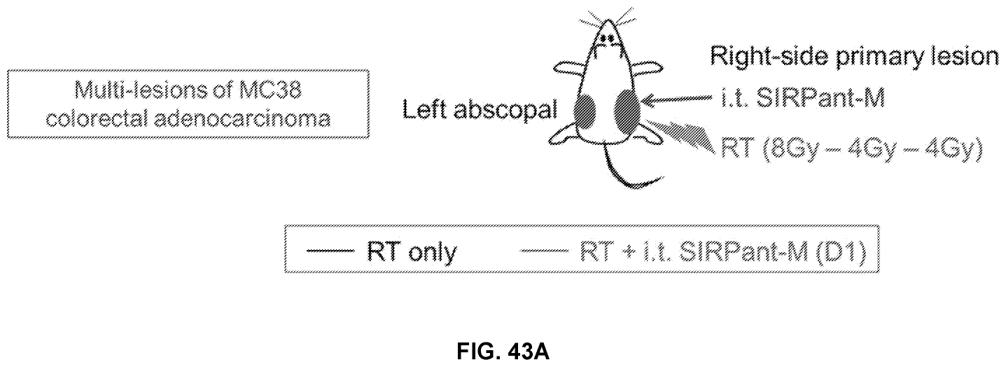

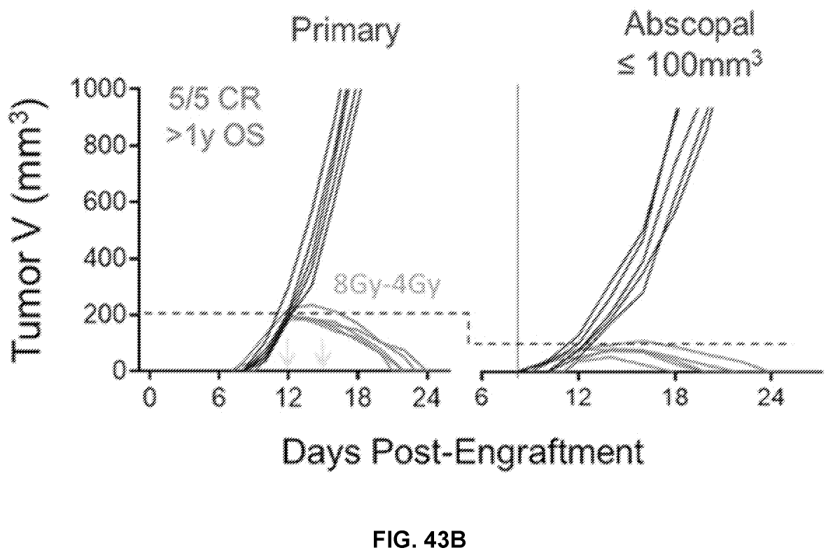

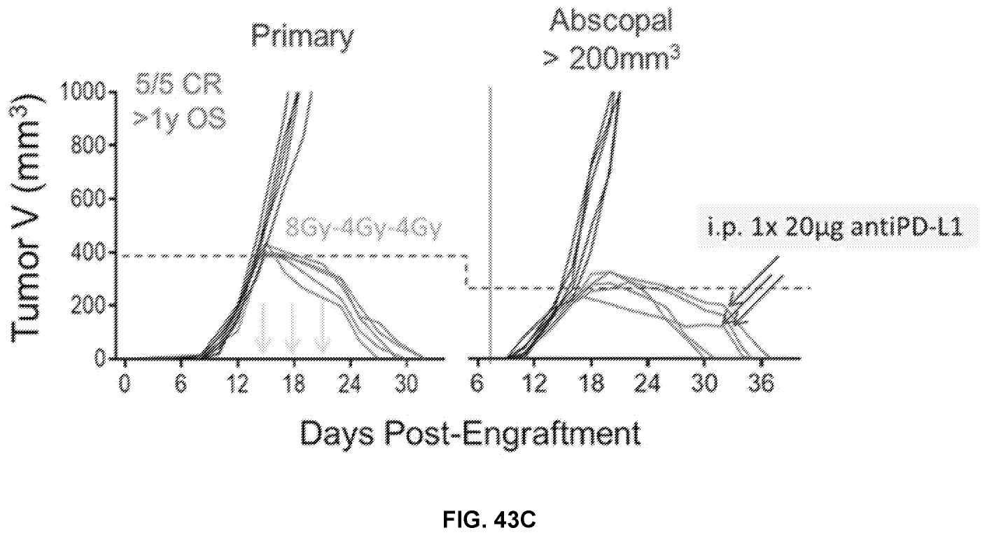

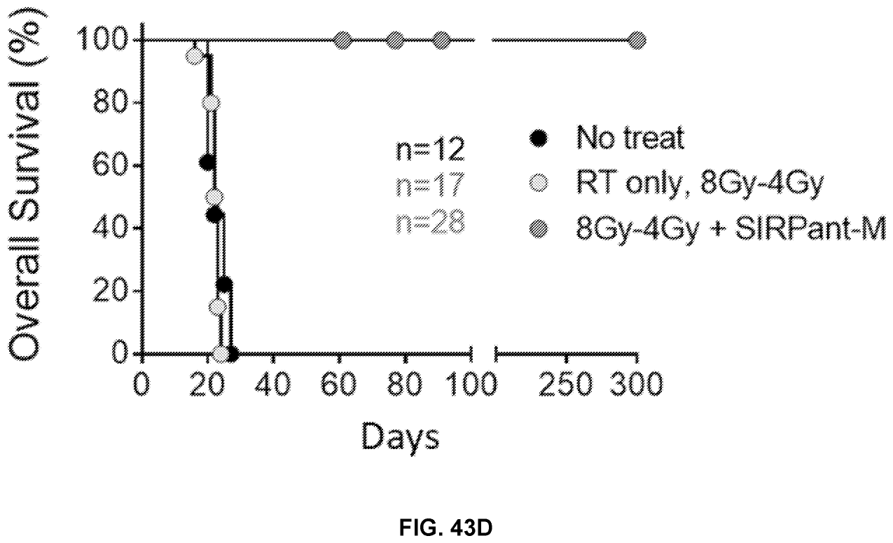

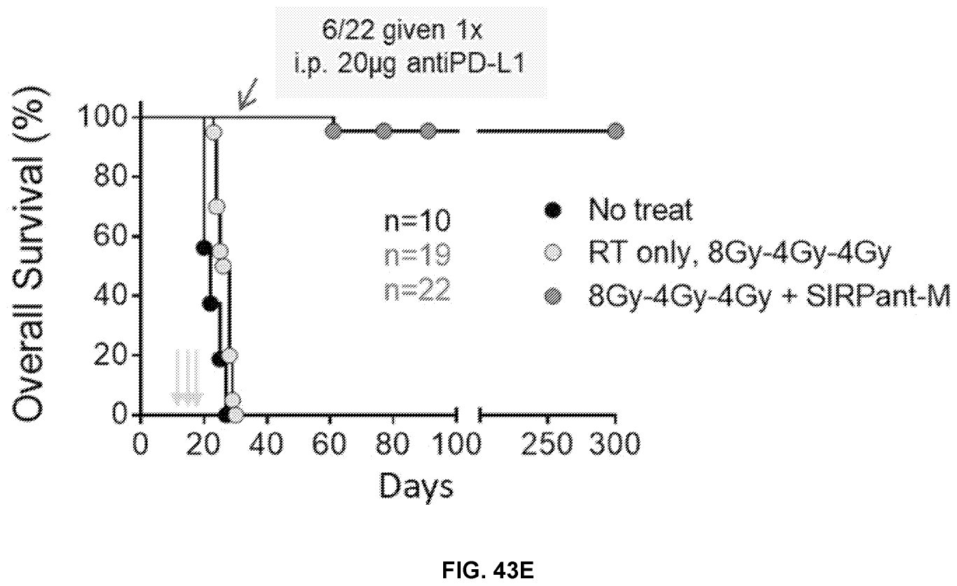

Study-3-2: Testing SIRPANT-M and RT combination for abscopal effects that eliminate MC38 colorectal cancer with distal lesions. In this study, MC38 adenocarcinoma were engrafted in both sides of flanks. After tumors formation, the right-side tumor (primary) was treated with SIRPANT-M i.t. plus RT for two cycles (8Gy for the 1^{st} and 4Gy for the 2^{nd} cycle, 3 d apart), while leaving the left-side tumor untreated. One additional SIRPANT-M and 4Gy RT treatment (3^{rd} cycle) was given to the primary tumor if it remained a volume≥100 mm³ after two cycles of treatment. Tumor volumes were measured for both flanks throughout the treatment to monitor abscopal effects and systemic MC38 tumor elimination. Partial data shown in FIG. 43.

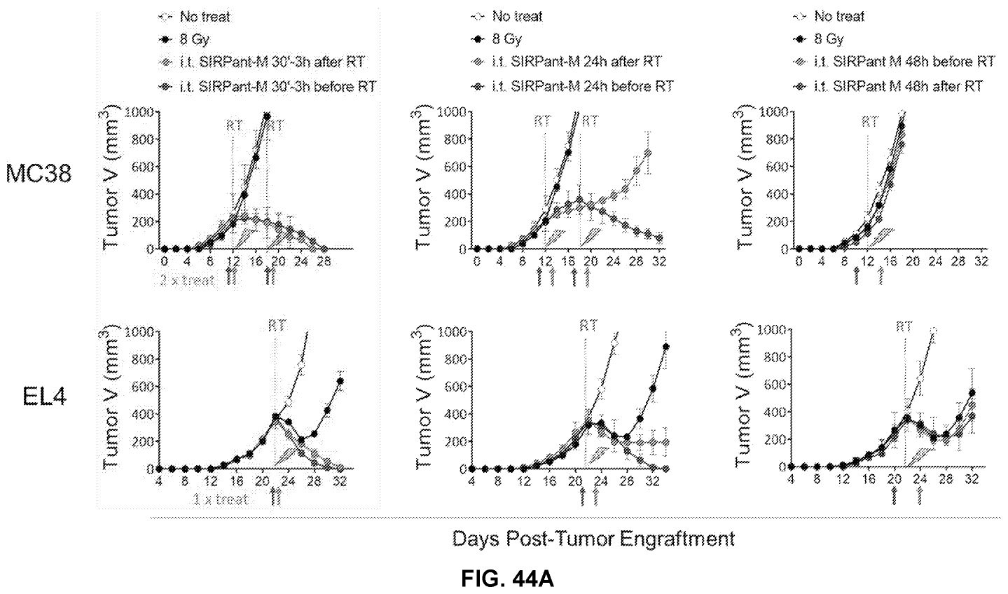

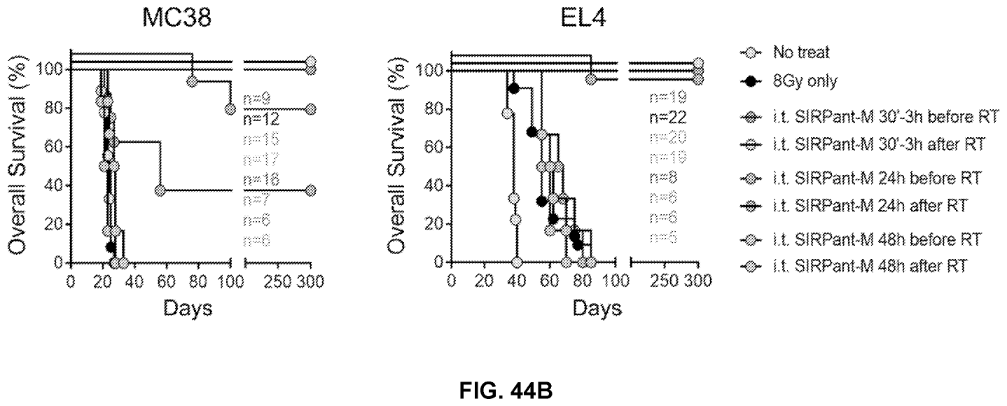

Study-4: Testing timing and sequence of administrating two modalities, SIRPANT-M i.t. and RT. Studies were carried out to compare efficacies of SIRPANT-M i.t. given before and after tumor RT. These studies conclude that the two treatment modalities should be administrated within a short time interval (3 h), and that SIRPANT-M i.t. given before or after tumor RT achieve similar efficacies. Longer time intervals between the two modalities result in reduced treatment effectiveness. FIG. 44 shows data of treating MC38 colorectal cancer and EL4 lymphoma with different orders of the two modalities.

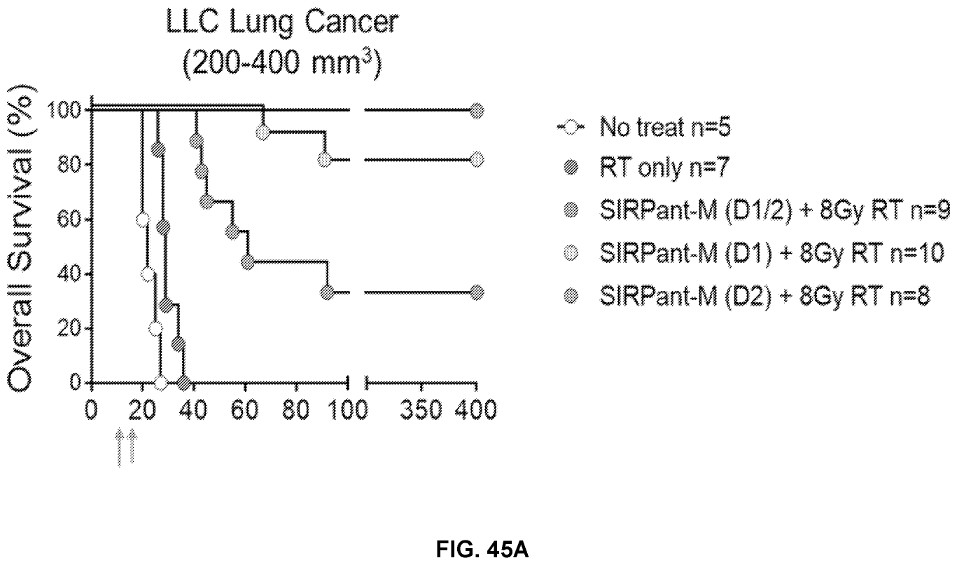

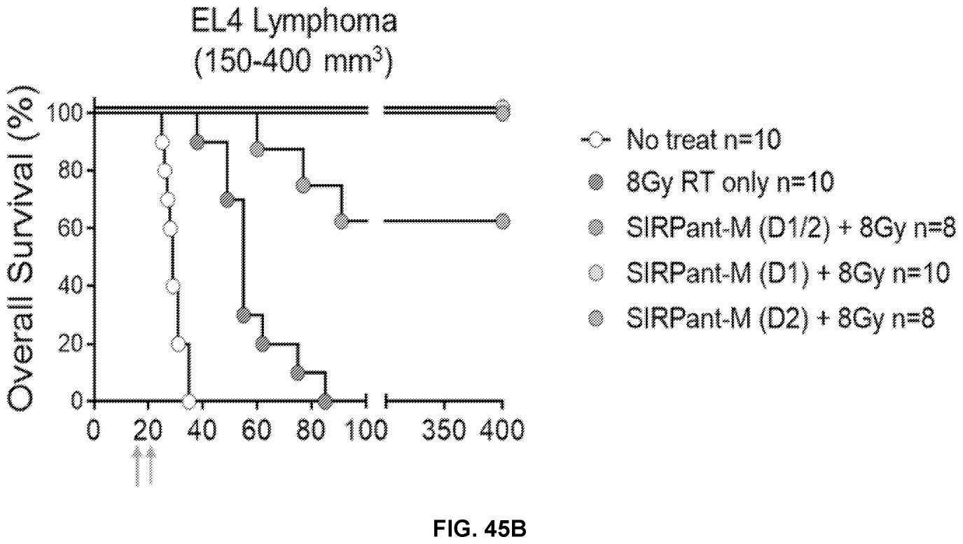

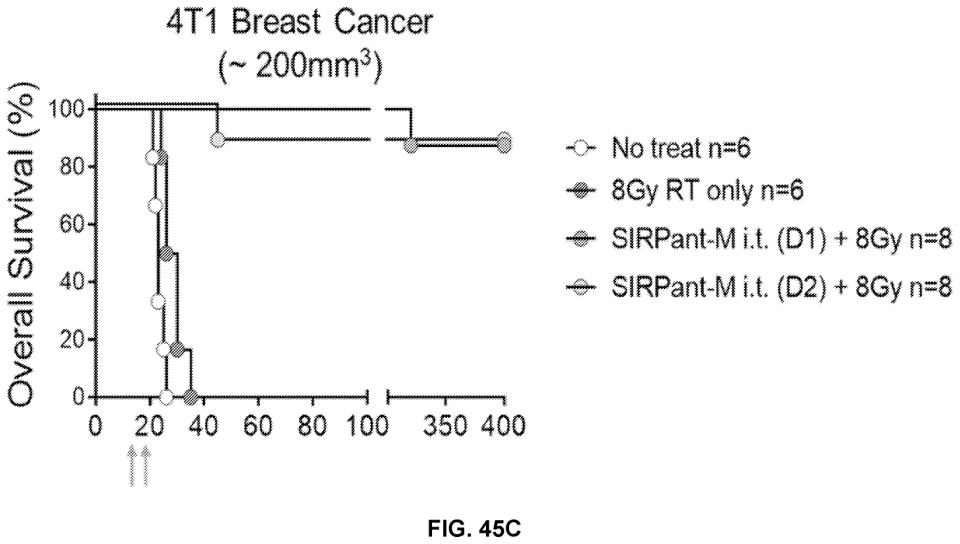

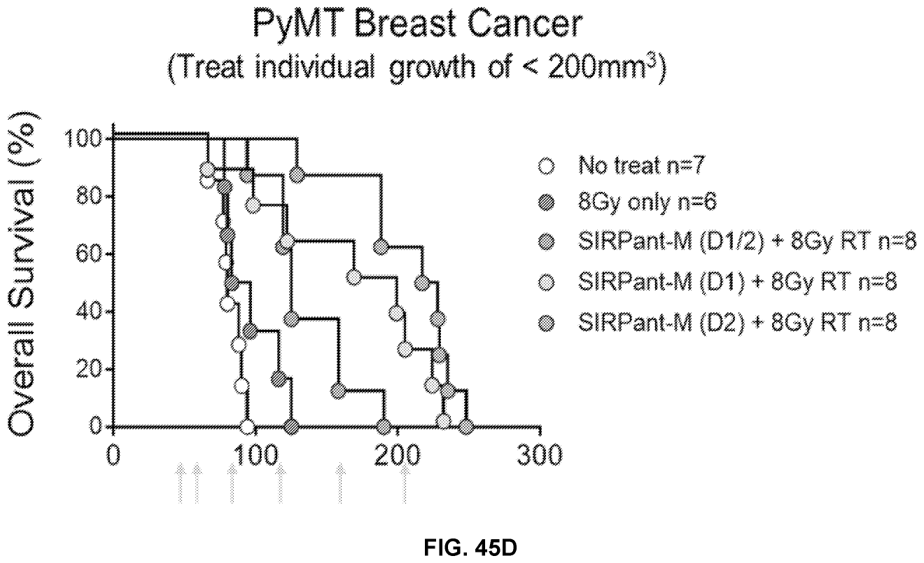

Study-5: Testing SIRPANT-M and RT combination treating other RT-refractory cancers. These studies tested SIRPANT-M i.t. combined with 8Gy RT to treat additional cancers including LLC lung cancer (s. c.), EL4 lymphoma (s. c.), 4T1 orthotopic-engrafted triple negative breast cancer, and PyMT spontaneously occurred triple negative breast cancer in MMTV-PyMT mice. Efficacies of SIRPANT and RT combination were compared to treatments with the same dose of RT only. Partial data are shown in FIG. 45.

### Summary

Both in vitro and in vivo studies confirm that Phago-Act™-produced SIRPANT-M are powerful anti-cancer immune initiators and that the strategy of using SIRPANT-M (SIRPa^{low} activated macrophages) is effective for elimination cancer and metastases. The below table summarizes our in vivo tests using SIRPANT-M at D2 dose administrated by intratumoral injection (i.t.).

TABLE 1
SIRPANT-M Preclinical Therapy to Cancer

| | | | | SIRPANT-M (D2 dose by i.t.) | | |
|---|---|---|---|---|---|---|
| | | | | | | Combine |
| | | RT | Checkpoint | Monotherapy | | 4-15Gy RT |
| Cancer Type | | alone | blockade | (2-3×, 3 d apart) | | (2-3×, 3 d apart) |
| Colon | MC38 | Resist | Resist | <100 mm³ | 95% CR | 100% CR |
| | | | | >200 mm³ | PR | 100% survival |
| | | | | | | (24/24) |
| Pancreatic | KPC s.c. | Resist | Resist | <100 mm³ | 100% CR | 100% CR |
| | | | | >200 mm³ | 10% CR | 92% survival |
| | | | | | | (22/24) |
| | Pan02 s.c. | Resist | Resist | <100 mm³ | 90% CR | 100% CR |
| | | | | >200 mm³ | PR | 100% survival |
| | | | | | | (20/20) |
| Lung | LLC s.c. | Resist | Resist | — | | 100% CR |
| | | | | | | 86% survival |
| | | | | | | (12/14) |
| Lymphoma | EL4 s.c. | PR | PR | 100-400 mm3, 60% | | 100% CR |
| | | | | CR & survival | | 100% survival |
| | | | | | | (12/12) |
| Breast | 4T1 | Resist | Resist | <100 mm³ | 100% CR | 100% survival |
| | orthotopic | | | >200 mm³ | PR | (5/5) |

| | | | | | |
|---|---|---|---|---|---|
| Met Breast | MMTV-PyM | Resist | Resist | In test | Single |
| | spontaneous | | | | lesion CR |
| | | | | | (5/5) |

| | | |
|---|---|---|
| Multi-colon | DSS-AOM | In test |
| | spontaneous | |

NR: no response;
PR: partial response - detectable growth inhibition or partial regression, 0% 6-month survival;
CR: complete response - complete durable regression to clearance;
Survival - post treatment 6-month continuous survival

### Example 6

Given that the mechanism by which SIRPANT-M achieves cancer elimination depends on the tumoricidal activity of activated tumor-specific T cells, combining SIRPANT-M+RT with checkpoint inhibitors that enhance T cell activity would therefore augment the capacity to eliminate tumors and clear distal lesions (metastases). In this Example, these possibilities are tested and the data produced are used to determine the clinical treatment scheme and modalities within the IND protocol. Two lines of experiments test SIRPANT-M+RT±either anti-PD1/L1 or anti-CTLA4 to treat pancreatic adenocarcinoma KPC or colorectal carcinoma MC38 in subcutaneous tumor models (IIB-1 and IIB-2). To closely mimic treating cancer formed in humans, two additional lines of experiments test SIRPANT-M+RT±anti-PD1/L1 or anti-CTLA4 against inflammation (DSS-colitis)- or carcinogen (AOM)-induced colorectal neoplasia/cancer (IIB-3 and IIB-4). In contrast to syngeneic engraftment such as subcutaneous models that pre-dispose an immune response and do not form tumors in their natural location, DSS-AOM-induced colorectal cancer arises at the location of inflammation, is associated with intensified colitis and is induced by the presence of a carcinogen that causes mutations in oncogenes and tumor-suppressor genes. Therefore, this cancer model closely resembles how cancers ‘spontaneously’ form in humans. Examples of such cancers include those formed in the lung, colon, ovarian, breasts, prostate, etc. Testing SIRPANT-M treatment against this spontaneous cancer support its application in a wider variety of cancer patients.

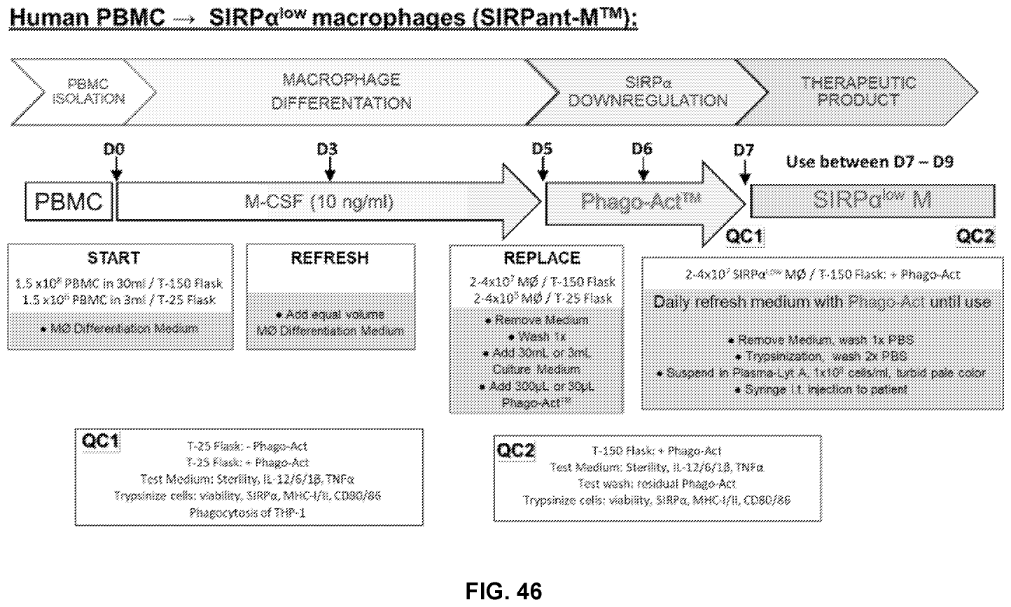

In addition to optimizing cancer treatment strategies, quality control (QC) assays necessary for CMC production of human SIRPα^{low} macrophages are design and tested. The current manufacture of human SIRPα^{low} macrophages from peripheral blood monocytes (PBMC) follows the diagram in FIG. 46, including a 5 d treatment with M-CSF to differentiate macrophages and a 48 h treatment with the proprietary agent “Phago-Act™” to downregulate SIRPα to produce SIRPα^{low} macrophages. Two QC assays, QC1 and QC2, are designed. QC1 is done after 48 h Phago-Act™ treatment to confirm macrophages having achieved the desired phenotype and functionality. QC2 is to be done prior to SIRPα^{low} macrophage administration to the patient, ensuring sterility, cell survival and other clinical therapy-related parameters. The designs of QC1/2 are shown in Table 2 and Table 3 and these assays are tested.

TABLE 2
QC1 quality test for human PBMC-derived SIRPα^{low} MΦ (T25 flasks)

| | | Specification (for a |
|---|---|---|
| Test | Method | typical product) |
| SIRPα^{low} | Flow cytometry with APC-conjugated | >90% cell population with |
| confirmation | anti-SIRPα.ex (e.g. clone 15-414 from BioLegend) | MFI < 4000 ^{a} |
| | Western blot for SIRPα and SIRPβ total expression: | >70% reduction in relative |
| | anti-SIRPα.ex (e.g. clone SE5A5 from BioLegend) | density of SIRPα.ex/ct |
| | anti-SIRPα.ct (in-house manufactured reagent) | Reference to ctl labeling of |
| | anti-SIRPβ (e.g. clone B4B6 from BioLegend) | beta-actin |
| | Beta-actin (e.g. clone Poly6221 from BioLegend) | |
| SIRPα^{low} M | Flow cytometry detect cell surface expression: | MHC-I: SIRPα⁺ M ≤ 3000; |
| phenotype | MHC-I (Pacific Blue-conjugated anti-HLA, B, C; | SIRPα^{low} M ≥ 6000 ^{b} |
| i) APC feature | clone W6/32) | MHC-II: SIRPα⁺ M ≤ 3000; |
| ii) Proinflammatory | MHC-II (PerCP-conjugated anti-HLA-DR; | SIRPα^{low} M ≥ 8000 |
| iii) Phagocytosis | clone L243) | CD80: SIRPα⁺ M ≤ 2000; |
| | CD80 (Brilliant Violet 650-conjugated anti-CD80; | SIRPα^{low} M ≥ 4000 |
| | clone 2D10) | CD86: SIRPα⁺ M ≤ 2000; |
| | CD86 (Brilliant Violet 605-conjugated anti-CD86; | SIRPα^{low} M ≥ 5000 |
| | clone BU63) | ** All neg. labeling of |
| | Flow cytometry detect proinflammatory cytokine | MFI ≤ 100 by isotype ctl |
| | released in culture medium: IL-12, TNFα, IL-1β, | IL-12 ≥ 200 pg/ml ^{c} |
| | and IL-6 (LEGENDplex ™ kits, BioLegend) | TNFα ≥ 200 pg/ml |
| | | IL-1β ≥ 200 pg/ml |
| | | IL-6 ≥ 500 pg/ml |
| | THP-1 cells adding to adherent M culture for | >70% phagocytosis |
| | 1 h phagocytosis assay | Reference to <5% of |
| | | SIRPα+ M phagocytosis |

^{a, b, c} sample figures associated with SOP

TABLE 3
QC2 release testing for human PBMC-derived SIRPα^{low} MΦ

| | | Specification (for a |
|---|---|---|
| Test | Method | typical product) |
| Viability | ¹PI staning; ²Trypan blue exclusion | ≥95% viable |
| Sterility | ¹21 CFR610.12 (14 day test) | Negative at day 7 post-release |
| | ²Rapid test method (e.g. Bactec/BacTAlert) | |
| Endotoxin | Limulus amebocyte lysate; Endosafe | <5 EU Kg⁻¹ h⁻¹ |
| (optional) | | |
| Mycoplasma | PCR/MycoAlert | Negative |
| (optional) | | |
| Residual | ELISA detect cytokines in final | Undetectable (<1 pg/ml) |
| Phago-Act ™ | product supernatant | |
| SIRPα^{low} | Flow cytometry with | >90% cell population with |
| confirmation | APC-conjugated anti- SIRPα.ex | MFI < 4000 ^{a} |
| (same as QC1) | (e.g. clone 15-414 from BioLegend) | Reference to neg. labeling |
| | | of MFI 100 labeled by |
| | | isotype ctl; pos. ctl |
| | | labeling of SIRPα⁺ M with |
| | | MFI > 14,000 |
| SIRPα^{low} M | Flow cytometry detect cell surface expression: | MHC-I: SIRPα⁺ M ≤ 3000; |
| phenotype: (same | MHC-I (Pacific Blue-conjugated anti-HLA, | SIRPα^{low} M ≥ 6000 ^{b} |
| as QC2) | B, C; clone W6/32) | MHC-II: SIRPα⁺ M ≤ 3000; |
| i) APC feature | MHC-II (PerCP-conjugated anti-HLA-DR; | SIRPα^{low} M ≥ 8000 |
| ii) Proinflammatory | clone L243) | CD80: SIRPα⁺ M ≤ 2000; |
| iii) Optional: | CD80 (Brilliant Violet 650-conjugated | SIRPα^{low} M ≥ 4000 |
| Phagocytosis | anti-CD80; clone 2D10) | CD86: SIRPα⁺ M ≤ 2000; |
| | CD86 (Brilliant Violet 605-conjugated | SIRPα^{low} M ≥ 5000 |
| | anti-CD86; clone BU63) | ** All neg. labeling of |
| | Flow cytometry detect proinflammatory | MFI ≤ 100 by isotype ctl |
| | cytokine released in culture medium: | IL-12 ≥ 200 pg/ml ^{c} |
| | IL-12, TNFα, IL-1β, and IL-6 | TNFα ≥ 200 pg/ml |
| | (LEGENDplex ™ kits, BioLegend) | IL-1β ≥ 200 pg/ml |
| | | IL-6 ≥ 500 pg/ml |
| | THP-1 cells adding to adherent M culture | >70% phagocytosis |
| | for 1 h phagocytosis assay | Reference to <5% of |
| | | SIRPα⁺ M phagocytosis |

^{a, b, c} sample figures associated with SOP

### Example 7: Inhibiting SHP-1 Downstream of SIRPα as a Potential Therapy Against Cancer

SIRPα mediates inhibitory regulation in macrophages through activation of the SH-domain containing tyrosine phosphatase SHP-1, which then mediates broad protein dephosphorylation and terminates multiple cytokine- and TLR-mediated activation pathways. In addition to downregulating SIRPα SHP-1 inhibition was also tested as an alternative approach to deplete the SIRPα-SHP-1 mediated inhibition.

The SHP-1 inhibitor TPI-1 (Kundu et al., J Immunol 2010 184:6529-6536) was purchased from Cayman Chemical (also available from Selleck Chemicals). TPI-1 was used as a single agent, or in combination with RT to treat subcutaneously established colorectal cancer (CRC) MC38 and pancreatic ductal adenocarcinoma (PDA) KPC.

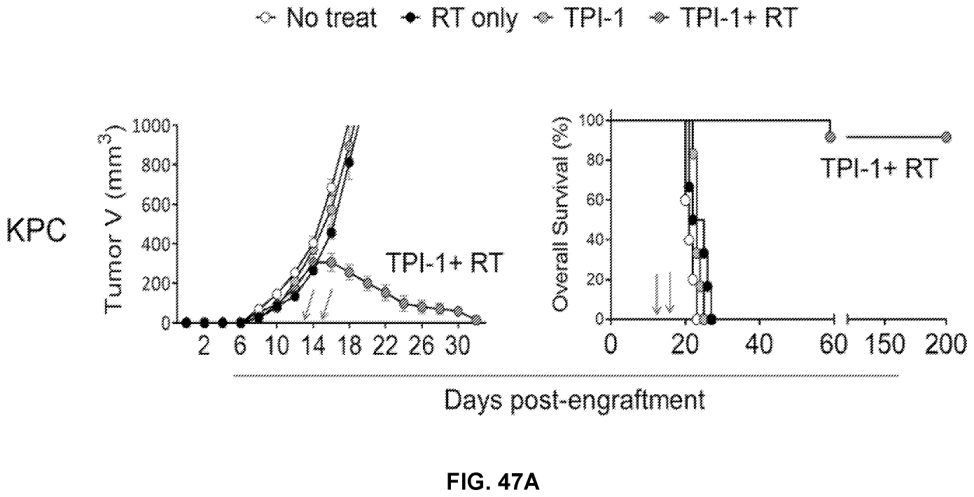

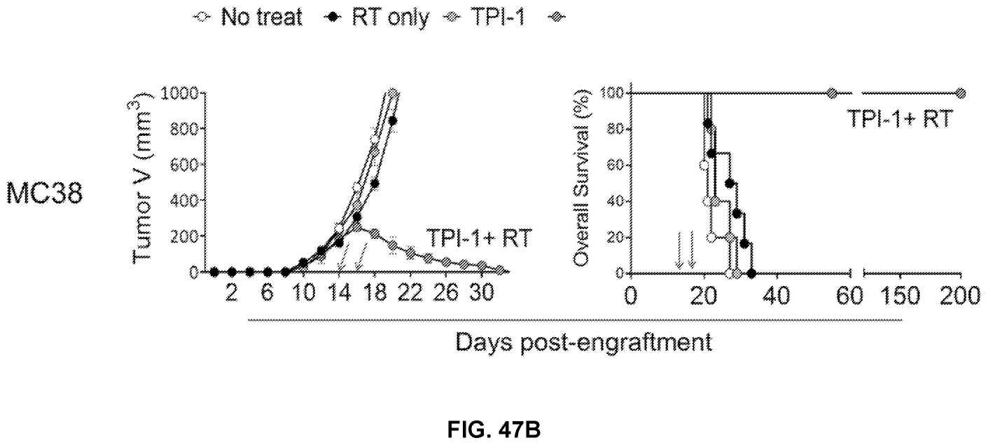

Test SHP-1 inhibitor TPI-1 to treat CRC and PDA tumors in vivo Once MC38 or KPC tumors reached approximately 200 mm³, 20 μg TPI-1 in 50 μl PBS was intratumorally injected into tumors (the dosage was calculated according to 1 mg/kg body weight). The treatment was repeated 2 days later. For combination treatment, mice intratumorally injected with TPI were given 30 min to allow TPI to diffuse within tumor tissues, followed by a fraction of local 8Gy X-ray radiation. This TPI+8Gy RT treatment was repeated after 2 days. Controls were tumors without treatment (No treat) or treated with 8Gy RT (RT only). Tumor volumes were measured every other day and calculated using the formula for a prolate spheroid (V=a2b/2), where a and b are tumor width and length (mm), respectively. Tumor treatment-induced in immune landscape changes in the TME was examined 48 h after the treatment. KPC tumor was also imaged by bioluminescence imager. FIG. 47A shows the treatment results of KPC, and FIG. 47B shows results of MC38.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### NUMBERED EMBODIMENTS

The following list of embodiments is included herein for illustration purposes only and is not intended to be comprehensive or limiting. The subject matter to be claimed is expressly not limited to the following embodiments.

Embodiment 1. A method for producing activated SIRPα^{low} macrophages, comprising
(a) isolating monocytes from peripheral blood mononuclear cells (PBMC) in a biological sample;
(b) differentiate the monocytes in vitro to produce macrophages; and
(c) contacting the macrophages with an SIRPα inhibitor; and
(d) contacting the macrophages with macrophage activating agent,
thereby generating a population of macrophages with marked reduction of SIRPα cell-surface expression (SIRPα^{low}), relative to untreated macrophages,
wherein the SIRPα^{low} macrophages have activated phagocytosis towards cancer cells, increased proinflammatory response, and increased immunogenic antigen presentation.

Embodiment 2. The method of claim 1, wherein the SIRPα inhibitor suppresses the expression of SIRPα, diminishes the abundance of SIRPα on the surface of a cell, inhibits the activity of SIRPα, disrupts the interaction between SIRPα and CD47, or a combination thereof.

Embodiment 3. The method of claim 1 or claim 2, wherein the SIRPα inhibitor comprises a cytokine, a TLR ligand, a glucocorticoid, or a combination thereof.

Embodiment 4. The method of any one of claims 1-3, wherein the SIRPα inhibitor is selected from the group consisting of IFNα, IFNβ, IFNγ, IL-1, IL-6, IL-12, IL-18, LPS, CpG, Poly I:C, LTA, PGN, flagellin, Pam3CSK4, zymosan, and HMGB1.

Embodiment 5. The method of any one of claims 1-4, wherein the macrophage activating agent comprises a cytokine, a phorbol ester, a TLR ligand, or a combination thereof.

Embodiment 6. The method of claim 5, wherein the cytokine is selected from the group consisting of IFNα, IFNβ, IL-6, IL-1, IL-17, IL-18, TNFα, and IL-12.

Embodiment 7. The method of claim 5, wherein the phorbol ester comprises phorbol 12-myristate 13-acetate (PMA).

Embodiment 8. The method of claim 5, wherein the TLR ligand is selected from the group consisting of LPS, CpG, Poly I:C, LTA, PGN, flagellin, Pam3CSK4, zymosan, and HMGB1.

Embodiment 9. The method of claim 3, wherein the glucocorticoid comprises methylprednisolone or dexamethasone.

Embodiment 10. The method of any one of claims 1-9, wherein the SIRPα inhibitor and macrophage activating agent are contacted with the macrophages sequentially.

Embodiment 11. The method of any one of claims 1-9, wherein the SIRPα inhibitor and macrophage activating agent are contacted with the macrophages simultaneously or concurrently.

Embodiment 12. The method of any one of claims 1-9 and 11, wherein the SIRPα inhibitor and macrophage activating agent are present in the same composition.

Embodiment 13. The method of any one of claims 1-12, wherein the composition comprises recombinant human interferon-gamma (IFNγ), recombinant human interferon-alphaA2 (IFNα), CpG oligodeoxynucleotide, and polyinosinic:polycytidylic acid (Poly I:C).

Embodiment 14. The method of any one of claims 1-13, wherein the SIRPα inhibitor comprises a SHP-1 inhibitor.

Embodiment 15. The method of claim 14, wherein the SHP-1 inhibitor is selected from the group consisting of TPI-1 (2-(2,5-Dichlorophenyl)-1,4-benzoquinone), TPI-1a1 (2-(2,5-Dichlorophenyl)-2,4-benzoquinone), TPI-1a2 (2-(3-chlorophenyl)-1,4-benzoquinone), TPI-1a3 (2-phenylnaphthoquinone), TPI-1a4 (2-(4-ethoxyphenyl)-1,4-benzoquinone), TPI-1a5 (2-(4-methoxyphenyl)-1,4-benzoquinone), SSG (Sodium Stibogluconate), PTP Inhibitor I (2-bromo-1-(4-hydroxyphenyl)-ethanone), PTP Inhibitor II (2-bromo-1-(4-methoxyphenyl)-ethanone), PTP Inhibitor III (2-[4-(2-bromoacetyl)phenoxy]-acetic acid), PTP Inhibitor IV (N,N′-[1,4-phenylenebis[(1-methylethylidene)-4,1-phenylene]]bis[1,1,1-trifluoro-methanesulfonamide), NSC 23922 (3-Aminocholestane), and NSC 87877 (8-hydroxy-7-[2-(6-sulfo-2-naphthalenyl)diazenyl]-5-quinolinesulfonic acid).

Embodiment 16. The method of any one of claims 1-15, further comprising contacting the macrophages with a SHP-1 inhibitor.

Embodiment 17. The method of claim 16, wherein the SHP-1 inhibitor is an irreversible SHP-1 inhibitor.

Embodiment 18. A composition comprising activated SIRPα^{low} macrophages produced by the method of any one of claims 1-17.

Embodiment 19. A method for producing in vitro expanded tumor-specific peripheral blood T (PBT) cells, comprising:
(a) isolating peripheral blood T (PBT) cells from a biological sample;
(b) in vitro co-culturing activated SIRPα^{low} macrophages produced by the method of claim 1 with cells from the tumor biopsy to produce tumor-fed SIRPα^{low} macrophages;
(c) in vitro co-culturing the tumor-fed SIRPα^{low} macrophages with isolated PBT cells to expand the number of tumor-specific T cells, thereby producing in vitro expanded tumor-specific PBT cells.

Embodiment 20. A composition comprising in vitro expanded tumor-specific PBT cells produced by the method of claim 19.

Embodiment 21. A method for producing in vitro expanded tumor-specific T cells from tumor infiltrating T lymphocyte (TIL), comprising:
(a) isolating tumor infiltrating T lymphocyte (TIL) cells from a tumor biopsy;
(b) in vitro co-culturing activated SIRPα^{low} macrophages produced by the method of claim 1 with tumor cells from the tumor biopsy to produce tumor-fed SIRPα^{low} macrophages;
(c) in vitro co-culturing the tumor-fed SIRPα^{low} macrophages with isolated TIL cells to expand the number of tumor-specific T cells, thereby producing in vitro expanded tumor-specific T cells from TIL.

Embodiment 22. A composition comprising in vitro expanded tumor-specific T cells from TIL produced by the method of claim 21.

Embodiment 23. A method for treating a tumor in a subject, comprising administering to the subject to a therapeutically effective amount of the composition of claim 18, the in vitro expanded tumor-specific PBT cells of claim 20, the in vitro expanded tumor-specific T cells from TIL of claim 22, or any combination thereof.

Embodiment 24. The method of claim 23, further comprising treating the subject with tumor-directed irradiation.

Embodiment 25. The method of claim 23 or claim 24, further comprising administering to the subject to a therapeutically effective amount of an immune checkpoint inhibitor.

Embodiment 26. The method of claim 25, wherein the immune checkpoint inhibitor comprises anti-PD1, anti-PD-L1, anti-CTLA4 antibodies, or a combination thereof.

Embodiment 27. The method of any one of claims 23-26, wherein the subject is refractory to PD-1 blockade.

Embodiment 28. The method of any one of claims 23-27, further comprising treating the subject with an oncolytic virus.

Embodiment 29. The method of claim 28, wherein the oncolytic virus is a vesicular stomatitis virus.

Embodiment 30. A composition comprising recombinant human interferon-gamma (IFNγ), recombinant human interferon-alphaA2 (IFNα), a CpG oligodeoxynucleotide, and polyinosinic:polycytidylic acid (Poly I:C).

Embodiment 31. The composition of claim 30, wherein the IFNγ is present at a concentration in the range of about 40 ng/ml to about 200 ng/ml.

Embodiment 32. The composition of claim 30 or claim 31, wherein the IFNγ is present at a concentration of about 100 ng/mL.

Embodiment 33. The composition of any one of claims 30-32, wherein the IFNα is present at a concentration in the range of about 40 ng/ml to about 200 ng/ml.

Embodiment 34. The composition of any one of claims 30-33, wherein the IFNα is present at a concentration of about 100 ng/mL.

Embodiment 35. The composition of any one of claims 30-34, wherein the CpG oligodeoxynucleotide is present at a concentration in the range of about 1 μg/ml to about 5 μg/ml.

Embodiment 36. The composition of any one of claims 30-35, wherein the CpG oligodeoxynucleotide is present at a concentration of 2 μg/ml.

Embodiment 37. The composition of any one of claims 30-36, wherein the Poly I:C is present at a concentration in the range of about 1 μg/ml to about 5 μg/ml. 38. The composition of any one of claims 30-37, wherein the Poly I:C is present at a concentration of about 2 μg/ml.

Embodiment 39. The composition of any one of claims 30-38, wherein the composition comprises about 100 ng/ml IFNγ, about 100 ng/ml IFNα, about 2 μg/ml CpG oligodeoxynucleotide, and about 2 μg/ml Poly I:C.

Embodiment 40. A composition comprising activated SIRPα^{low} macrophages produced by a method comprising contacting macrophages with an effective amount of the composition of any one of claims 30-39.

Embodiment 41. A method of producing one or more activated SIRPα^{low} macrophages, comprising:
(a) providing one or more macrophages;
(b) bringing the one or more macrophages in contact with the composition of any one of claims 30-40, thereby producing one or more activated SIRPα^{low} macrophages.

Embodiment 42. The method of claim 41, wherein step (a) comprises: (i) collecting a biological sample, comprising one or more peripheral blood mononuclear cells (PBMC), from the subject; (ii) isolating one or more monocytes from the PBMC; and (iii) culturing the one or more monocytes in vitro to produce one or more macrophages.

Embodiment 43. The method of claim 42, wherein step (iii) comprises culturing the one or more monocytes in the presence of a macrophage differentiation-promoting factor.

Embodiment 44. The method of claim 43, wherein the macrophage differentiation-promoting factor comprises macrophage colony stimulating factor (M-CSF), GM-CSF, IL-6, human serum, IL-4, IL-10, IFN-α, IL-1, TGF-β, or any combination thereof.

Embodiment 45. The method of any one of claims 42-44, wherein the biological sample is blood or serum.

Embodiment 46. The method of any one of claims 41-45, wherein the macrophages are bone marrow-derived macrophages or monocyte-derived macrophages.

Embodiment 47. A composition, comprising: the activated SIRPα^{low} macrophages produced by the method of any one of claims 41-46.

Embodiment 48. A method for treating a cancer in a subject, comprising administering to the subject to a therapeutically effective amount of the composition of claim 40 or claim 47.

Embodiment 49. The method of any one of claims 23-29 and claim 48, further comprising administering to the subject one or more damage-associated molecular patterns (DAMPs).

Embodiment 50. The method of claim 49, wherein the one or more DAMPs comprises high-mobility group box 1 protein (HMGB1), heat shock protein (HSP), SNAP-associated protein (SNAPIN), versican, biglycan, decorin, eosinophil-derived neurotoxin, surfactant protein A/D, β-defensin 3, histone, serum amyloid A (SAA), β amyloid (Aβ), β2-glycoprotein I, mRNA, tenascin-C, S100 proteins, high-mobility group box 1 protein (HMGN1), biglycan, decorin, heparin sulfate, hyaluronic acid, fibrinogen, fibronectin, β-defensin 2, surfactant protein A/D, lactoferrin, neutrophil elastase, peroxiredoxin, histone, serum amyloid A (SAA), ox-LDL, IgG-ribonucleoprotein complex, microRNAs, mtDNA, F-actin, Sin3A-associated protein 130, β-glucosylceramide, N-glycans, monosodium urate (MSU), glucose, cholesterol crystals, ATP, oxidized 1-palmitoyl-2-arachidonylsn-glycero-3-phosphocholine (ox-PAPC), RNA transcribed from Alu elements (Alu-RNA), endogenous 5′ppp RNA, unedited long self-dsRNA, endogenous retroviral RNA, cytoplasmic DNA, damaged DNA in the nucleus, advanced glycation end products (AGEs), DNA, HSP70, peptidoglycan recognition protein 1 (PGLYRP1), actin, phosphatidic acid (PA), phosphatidylcholine (PC), phosphatidylethanolamine (PE), phosphatidylglycerol (PG), phosphatidylinositol (PI), phosphatidylserine (PS), cardiolipin, sulfatide, sphingomyelin, apolipoprotein A1 (APOA1), apolipoprotein A2 (APOA2), apolipoprotein B (APOB), apolipoprotein E (APOE), apolipoprotein J (APOJ), low-density lipoprotein (LDL), high-density lipoprotein (HDL), very-low-density lipoprotein (VLDL), Lp(a), HSP60, N-formylated peptides, cathepsin G, FAM19A4, annexin 1, Aβ42, serum amyloid A (SAA), low-density lipoprotein (LL-37) and other peptides, ATP, UTP, UDP, ADP, cyclic-GMP-AMP (cGAMP), Calcium ion, ROS, or any combination thereof.

Embodiment 51. The method of any one of claims 23-29 and claims 48-50, further comprising administering to the subject, an anti-CD155 antibody, an anti-CD112 antibody, an anti-Fap2 antibody, an anti-TGIT antibody, an anti-CD96 antibody, an anti-CD112R antibody, an anti-DNAM-1 antibody, an anti-TIM-3 antibody, an anti-LAG3 antibody, or any combination thereof.

Embodiment 52. The method of claim 51, wherein the anti-TIGIT antibody is tiragolumab, BMS-986207, BGB-A1217, OP-313M32, AB154, ASP8374, MK-7684, or any combination thereof.

1. A method for producing activated SIRPα^{low} macrophages, comprising
(a) isolating monocytes from peripheral blood mononuclear cells (PBMC) in a biological sample;
(b) differentiate the monocytes in vitro to produce macrophages; and
(c) contacting the macrophages with an SIRPα inhibitor; and
(d) contacting the macrophages with macrophage activating agent, thereby generating a population of macrophages with marked reduction of SIRPα cell-surface expression (SIRPα^{low}), relative to untreated macrophages,
wherein the SIRPα^{low} macrophages have activated phagocytosis towards cancer cells, increased proinflammatory response, and increased immunogenic antigen presentation,
wherein the SIRPα inhibitor comprises a cytokine, a TLR ligand, a glucocorticoid, or a combination thereof.

2. (canceled)

3. (canceled)

4. The method of claim 1, wherein the SIRPα inhibitor is selected from the group consisting of IFNα, IFNβ, IFNγ, IL-1, IL-6, IL-12, IL-18, LPS, CpG, Poly I:C, LTA, PGN, flagellin, Pam3CSK4, zymosan, and HMGB1.

5. The method of claim 1, wherein the macrophage activating agent comprises a cytokine, a phorbol ester, a TLR ligand, or a combination thereof.

6. The method of claim 5, wherein the cytokine is selected from the group consisting of IFNα, IFNβ, IL-6, IL-1, IL-17, IL-18, TNFα, and IL-12.

7. The method of claim 5, wherein the phorbol ester comprises phorbol 12-myristate 13-acetate (PMA).

8. The method of claim 5, wherein the TLR ligand is selected from the group consisting of LPS, CpG, Poly I:C, LTA, PGN, flagellin, Pam3CSK4, zymosan, and HMGB1.

9. The method of claim 3, wherein the glucocorticoid comprises methylprednisolone or dexamethasone.

10. The method of claim 1, wherein the SIRPα inhibitor and macrophage activating agent are contacted with the macrophages sequentially.

11. The method of claim 1, wherein the SIRPα inhibitor and macrophage activating agent are contacted with the macrophages simultaneously or concurrently.

12. The method of claim 11, wherein the SIRPα inhibitor and macrophage activating agent are present in the same composition.

13. The method of claim 1, wherein the composition comprises recombinant human interferon-gamma (IFNγ), recombinant human interferon-alpha A2 (IFNα), CpG oligodeoxynucleotide, and polyinosinic:polycytidylic acid (Poly I:C).

14. The method of claim 1, wherein the SIRPα inhibitor comprises a SHP-1 inhibitor.

15. The method of claim 14, wherein the SHP-1 inhibitor is selected from the group consisting of TPI-1 (2-(2,5-Dichlorophenyl)-1,4-benzoquinone), TPI-1a1 (2-(2,5-Dichlorophenyl)-2,4-benzoquinone), TPI-1a2 (2-(3-chlorophenyl)-1,4-benzoquinone), TPI-1a3 (2-phenylnaphthoquinone), TPI-1a4 (2-(4-ethoxyphenyl)-1,4-benzoquinone), TPI-1a5 (2-(4-methoxyphenyl)-1,4-benzoquinone), SSG (Sodium Stibogluconate), PTP Inhibitor I (2-bromo-1-(4-hydroxyphenyl)-ethanone), PTP Inhibitor II (2-bromo-1-(4-methoxyphenyl)-ethanone), PTP Inhibitor III (2-[4-(2-bromoacetyl)phenoxy]-acetic acid), PTP Inhibitor IV (N,N′-[1,4-phenylenebis[(1-methylethylidene)-4,1-phenylene]]bis[1,1,1-trifluoro-methanesulfonamide), NSC 23922 (3-Aminocholestane), and NSC 87877 (8-hydroxy-7-[2-(6-sulfo-2-naphthalenyl)diazenyl]-5-quinolinesulfonic acid).

16. The method of claim 1, further comprising contacting the macrophages with a SHP-1 inhibitor.

17. The method of claim 16, wherein the SHP-1 inhibitor is an irreversible SHP-1 inhibitor.

18. A composition comprising activated SIRPα^{low} macrophages produced by the method of claim 1.

19. A method for producing in vitro expanded tumor-specific T cells, comprising:
(a) isolating peripheral blood T (PBT) cells or tumor infiltrating T lymphocyte (TIL) cells from a biological sample;
(b) in vitro co-culturing activated SIRPα^{low} macrophages produced by the method of claim 1 with cells from the tumor biopsy to produce tumor-fed SIRPα^{low} macrophages;
(c) in vitro co-culturing the tumor-fed SIRPα^{low} macrophages with isolated PBT cells to expand the number of tumor-specific T cells, thereby producing in vitro expanded tumor-specific T cells.

20-22. (canceled)

23. A method for treating a tumor in a subject, comprising administering to the subject to a therapeutically effective amount of the composition of claim 18.

24-29. (canceled)

30. A composition comprising recombinant human interferon-gamma (IFNγ), recombinant human interferon-alpha A2 (IFNα), a CpG oligodeoxynucleotide, and polyinosinic:polycytidylic acid (Poly I:C).

31-52. (canceled)
